(19) United States
(12) Patent Application Publication
(10) Pub. No.: US 2025/0205367 A1
(43) Pub. Date: Jun. 26, 2025

(54) COMPOSITIONS COMPRISING A MUSCLE ACTIVE PROMOTER AND METHODS OF USING THE SAME

(71) Applicant: Duke University, Durham, NC (US)

(72) Inventors: Dwight Koeberl, Durham, NC (US); Baodong Sun, Durham, NC (US); Benjamin Arnson, Durham, NC (US)

(21) Appl. No.: 18/988,652

(22) Filed: Dec. 19, 2024

Related U.S. Application Data
(60) Provisional application No. 63/612,421, filed on Dec. 20, 2023.

(51) Int. Cl. A61K 48/00; A61K 31/195; A61K 38/46; A61K 38/47; A61P 3/00; C12N 9/22; C12N 15/11; C12N 15/86; C12N 15/90

(52) U.S. Cl. CPC A61K 48/0058; A61K 31/195; A61K 38/465; A61K 38/47; A61P 3/00; C12N 9/22; C12N 15/111; C12N 15/86; C12N 15/907; C12Y 301/03009; C12Y 302/0102; C12N 2310/20; C12N 2750/14143; C12N 2830/50

(57) ABSTRACT

Disclosed herein are novel promoters that drive expression of a gene product preferentially in the heart and in skeletal muscle. Disclosed herein are vectors comprising these novel promoters and vectors comprising these novel promoters. Disclosed herein are methods of gene editing and gene therapy that employ these novel promoters and novel vectors.

### CROSS-REFERENCE TO RELATED APPLICATIONS

This application claims priority to U.S. Provisional Application No. 63/612,421 filed 20 Dec. 2024, which is incorporated herein in its entirety.

### STATEMENT REGARDING FEDERAL FUNDING

This invention was made with Government support under Federal Grant No. DK105434 awarded by the National Institute of Diabetes and Digestive and Kidney Diseases (NIH/NIDDK). The Federal Government has certain rights to this invention.

### REFERENCE TO THE SEQUENCE LISTING

The Sequence Listing submitted 19 Dec. 2024 as an XML file named “24-3025-US_Sequence Listing”, created on 19 Dec. 2024 and having a size of 319,488 bytes is hereby incorporated by reference pursuant to 37 C.F.R. § 1.52(e)(5).

### BACKGROUND

There is a critical unmet need for the development of improved therapies to stably treat patients having a pathogenic disease or disorder, especially those that disrupt multiple metabolic systems including the heart, the liver, the kidneys, and the skeletal muscles.

Consequently, the present disclosure provides compositions comprising novel promoters and methods of gene editing and gene therapy, which can be used alone or in combination with other treatments.

### BRIEF DESCRIPTION OF THE FIGURES

FIG. 1A-FIG. 1I show genome editing in infant G6pc −/− mice. FIG. 1A shows groups of mice treated with or without bezafibrate (Beza). Two rAAV9 vectors were administered at 12 days of age (AAV-G6PC-SpCRISPR (CRISPR) and AAV-Donor (Donor)) and the latter contains a human G6PC transgene flanked by mouse DNA flanked by Exon 1 and Intron 1 of the mouse G6pc gene, which permitted integration of the transgene as shown in FIG. 1B. Vector doses were as follows: “Low Donor+CRISPR”=Donor (2E+12 vg/kg) and CRISPR (4E+11 vg/kg); “Low Donor”=Donor (2E+12 vg/kg). “Medium Donor+CRISPR”=Donor (8E+12 vg/kg) and CRISPR (1.6E+12 vg/kg). “Medium Donor”=Donor (8E+12 vg/kg). Endpoints included: blood glucose after 8 hours of fasting (FIG. 1C); blood glucose at baseline for GTT (FIG. 1D), and at 120 minutes for GTT (FIG. 1E); liver G6Pase activity (FIG. 1F) and glycogen content (FIG. 1G); and vector DNA (FIG. 1H) and vector RNA quantification (FIG. 1I). Individual values and mean shown, * means p<0.05, ** means p<0.01, **** means p<0.0001 for multiple t tests.

FIG. 2A-FIG. 2B shows the twelve-week genome editing study. FIG. 2A shows the treatment design. The drug, bezafibrate, or vehicle was administered from day 5 to day 26 of life, and vector was administered on day 12+/−2 of life. Tissue collection was performed at 12 weeks of age. FIG. 2B shows Kaplan-Meier survival analysis. Vector doses were as follows: “Low D+C”=Donor (2E+12 vg/kg) and CRISPR (4E+11 vg/kg); “High D+C”=Donor (1.6E+13 vg/kg) and CRISPR (3.2E+12 vg/kg). “Low D”=Donor (2E+12 vg/kg). “High D”=Donor (1.6E+13 vg/kg). “High C”=CRISPR (3.2E+12 vg/kg).

FIG. 3A-FIG. 3D show correction of hypoglycemia. Blood glucose after 8 hours fasting performed two weeks following vector administration (FIG. 3A) and 11 weeks following vector administration (FIG. 3B). Blood glucose determined at baseline after a 4 hour fast (FIG. 3C), and at 120 minutes after dextrose administration (FIG. 3D). Individual values and mean are shown, * means p<0.05, ** means p<0.01, *** means p<0.001, **** means p<0.0001, compared with High D+C+Beza group as determined by one-way ANOVA with Dunnett's multiple comparisons test.

FIG. 4A-FIG. 4F shows the endpoints for genome editing in the murine GSD Ia liver. Donor-treated groups collected at Week 12, CRISPR-only at Day 3. The results are Liver G6Pase(FIG. 4A); liver glycogen content (FIG. 4B); G6PC transgene DNA (FIG. 4C); G6PC transgene RNA (FIG. 4D); Cas9 DNA (FIG. 4E); Cas9 RNA (FIG. 4F). Individual values and mean are shown. * means p<0.05, ** means p<0.01, *** means p<0.001, ****=p<0.0001, compared with High D+C+Beza group as determined by one-way ANOVA with Dunnett's multiple comparisons test.

FIG. 5A-FIG. 5C shows the quantification of indels, HDR, and off-target effects. PCR-detection of integrated G6PC transgene in comparison with a standard curve from purified integration PCR fragment in mouse genomic DNA (FIG. 5A). Surveyor assay for CRISPR vector treated mice (FIG. 5B). Arrows indicate bands for indels. WT is negative control. CG control for the Surveyor shows ˜50% cleavage. Histograms show mean+/−SD. ** means p<0.01 as determined by a t test. Off-target effects determined by NGS of predicted sites with two and 3 differences from the guide sequence from 5 treated mice in the High D+C+Beza group and from an untreated control mouse (FIG. 5C).

FIG. 6A-FIG. 6B show weight following treatment. Individual values and mean are shown at 4 weeks (FIG. 6A) following treatment and 12 weeks (FIG. 6B) following treatment. P values indicated as follows: **=p<0.01; ****=p<0.0001, compared with High D+C+Beza group as determined by one-way ANOVA with Dunnett's multiple comparisons test.

FIG. 7A-FIG. 7D show biomarker testing 12 weeks following treatment. Individual values and mean are shown for ALT (FIG. 7A), cholesterol (FIG. 7B), creatinine (FIG. 7C), and triglycerides (FIG. 7D). P values indicated as follows: **=p<0.01; ****=p<0.0001, compared with High D+C+Beza group as determined by one-way ANOVA with Dunnett's multiple comparisons test.

FIG. 8A-FIG. 8H show liver vacuolation following genome editing. Photomicrographs of mouse liver 200×, Masson's Trichrome Stain. FIG. 8A shows untreated GSD Ia. FIG. 8B shows high D+C+Beza. FIG. 8C shows High D+Beza. FIG. 8D shows WT. FIG. 8E shows Low D+C+Beza. FIG. 8F shows Low D+Beza. Vacuolation was quantified in liver sections. The number of vacuoles (FIG. 8G) and vacuole size (in pixels) (FIG. 8H) of liver histology samples from wild type mice (n=3) and GSD Ia treatment groups: GSD Ia Untreated (UT; n=3), High D+C+Beza (n=3), High D+Beza (n=4), Low D+C+Beza (n=4) and Low D+Beza (n=4). All images were all taken at equal resolution and magnification. Three representative regions of interest from H&E samples were quantified per mouse. Data is represented as Mean+−SD. Significance was validated with one-way ANOVA test.

FIG. 9A-FIG. 9B shows ELISA for anti-SpCas9 IgG in GSD Ia mice after vector administration. FIG. 9A shows serum from mice either treated with High dose Donor and CRISPR vectors and bezafibrate (High D+C+Beza; n=6) or Donor vector with bezafibrate (H D+Beza; n=6) was collected 12 weeks post-treatment and analyzed by indirect ELISA for SpCas9 reactive antibodies. 450 nm absorbance values are plotted for serum tested at a 1:100 dilution. Dots represent values from individual mice. As assay controls, serum from a GSD Iacarrier mouse pre- and post-vaccination with SpCas9 was included (n=1). Statistical analysis was performed using Welch's t-test to compare only the absorbance of Donor+CRISPR and Donor only groups. P value ns=not significant. FIG. 9B shows CD3 staining of liver sections was performed 4 weeks post-treatment from mice treated with High dose Donor and CRISPR vectors (High D+C; n=10) or High dose Donor vector (High D; n=4). Wildtype age-matched and 12 day old, untreated GSD Ia liver samples were included as controls. Statistical analysis with One-way ANOVA. P value ns=not significant.

FIG. 10A-FIG. 10C shows three-month-old GAA-KO mice that were injected intravenously (1E13 vector genomes (vg)/kg body weight) with the AAV9-G6PC-hGAA or AAV9-hSk-CRM4-G6PC-hGAA vector and analyzed after 3 weeks. Control group was untreated (UT) GAA-KO mice. FIG. 10A shows GAA activity in tissues. FIG. 10B shows glycogen content in tissues. FIG. 10C shows anti-hGAA antibody detection by ELISA. Mice treated with enzyme replacement therapy (ERT) were used as positive controls.

FIG. 11A-FIG. 11D show GAA activities in 4 different tissues 3 weeks after I.V. AAV injection (1×10¹³ vg/kg). Mice were 3 months old at injection. N=4 for untreated controls, n=5 for AAV-G6Pc(356)-GAA treated and AAV-CRM4-G6Pc(356)-GAA treated mice; data for heart GAA (FIG. 11A), diaphragm GAA (FIG. 11B), quadriceps GAA (FIG. 11C), and liver GAA (FIG. 11D) are shown as mean±SD; one-way ANOVA was performed for each tissue. *p<0.05, **p<0.01, ***p<0.001, ****p<0.0001. Wild-type (WT, n=4) values are for reference and are not included in statistic analysis. Addition of CRM4 enhancer elevated GAA activity in heart but not skeletal muscles.

FIG. 12 shows the effect of AAV treatment in glycogen reduction in tissues (diaphragm (left), heart (middle), and quadriceps (right) examined 3 weeks after I.V. AAV injection (1×10¹³ vg/kg). Mice were 3 months old at injection. n=4 for untreated controls and wild-type controls, n=5 for AAV-G6Pc(356)-GAA treated and AAV-CRM4-G6Pc(356)-GAA treated mice; data are shown as mean±SD; one-way ANOVA was performed for each tissue. *p<0.05, **p<0.01, ***p<0.001, ****p<0.0001. Addition of CRM4 enhancer enhanced glycogen reduction in heart but not in skeletal muscles.

FIG. 13 shows that AAV-CRM4-G6Pc(356)-GAA treatment improved muscle strength as indicated by grip strength test in a 12-week study.

FIG. 14A-FIG. 14D show GAA activity in tissues following AAV9-G6PC(303)-GAA vector administration injection (1×10¹³ vg/kg). Mice were 3 months old at injection. Heart (FIG. 14A), diaphragm (FIG. 14B), quadriceps (FIG. 14C), and brain (FIG. 14D) are shown. N=4 for untreated controls and wild-type controls, n=12 for AAV-G6Pc(303)-GAA treated GAA-KO mice. FIG. 14E-FIG. 14H show the glycogen content in tissues following AAV9-G6PC(303)-GAA vector administration injection (1×10¹³ vg/kg). Mice were 3 months old at injection. Heart (FIG. 14E), diaphragm (FIG. 14F), quadriceps (FIG. 14G), and brain (FIG. 14H) are shown. N=4 for untreated controls and wild-type controls, n=12 for AAV-G6Pc(303)-GAA treated GAA-KO mice. FIG. 14I-FIG. 14K show liver analysis and muscle function testing following AAV9-G6PC(303)-GAA vector administration injection (1×10¹³ vg/kg). Mice were 3 months old at injection. Liver GAA activity (FIG. 14I), and glycogen content (FIG. 14J) are shown. Grip strength (FIG. 14K) was performed at 12 weeks. N=4 for untreated controls and wild-type controls, n=12 for AAV-G6Pc(303)-GAA treated GAA-KO mice. Data are shown as mean±SD; one-way ANOVA was performed for each tissue. *p<0.05, **p<0.01, ***p<0.001, ****p<0.0001. Wild-type (WT and n=4) values are for reference and are not included in statistical analysis.

FIG. 15A-FIG. 15E show GAA activity in various tissues (heart (FIG. 15A), diaphragm (FIG. 15B), quadriceps (FIG. 15C), gastrocnemius (FIG. 15D), and liver (FIG. 15E)) following AAV9-G6PC(356)CRM4-GAA vector administration injection (1×10¹³ vg/kg) and/or AAV-G6PC(356)-GAA vector administration injection (1×10¹³ vg/kg).

FIG. 16A shows GAA activities in tissues 12 weeks after I.V. AAV injection (1×10¹³ vg/kg). Mice were 3 months old at injection. N=4 for untreated controls, n=5 for AAV-G6Pc(303)-GAA treated mice and n=6 for AAV-MyoG6Pc-GAA (AAV-CRM4-G6Pc(356)-GAA) treated mice; data are shown as mean±SD; one-way ANOVA was performed for each tissue. *p<0.05, **p<0.01, ***p<0.001, ****p<0.0001. AAV-MyoG6Pc-GAA did not enhance GAA expression in heart or skeletal muscles. There was no effect in the brain. FIG. 16B shows AAV treatment reduce glycogen in muscles to wild-type level 12 weeks after I.V. AAV injection (1×10¹³ vg/kg). Mice were 3 months old at injection. N=4 for untreated controls, n=6 for AAV-MyoG6Pc-GAA (AAV-CRM4-G6Pc(356)-GAA) treated mice; data are shown as mean±SD; one-way ANOVA was performed for each tissue. *p<0.05, **p<0.01, ***p<0.001, ****p<0.0001. There was no detectable effect in the brain.

FIG. 17A-FIG. 17C shows GAA mRNA in tissues of (male) mice treated with AAV. FIG. 17A shows GAA mRNA copies in quadriceps 3 weeks after AAV injection (n=4 each group). FIG. 17B shows GAA mRNA copies in heart 3 weeks after AAV injection (n=4 each group). FIG. 17C shows GAA mRNA copies in tissues 12 weeks after injection of AAV-G6Pc303-GAA. N=3 each. RNA was extracted using Qiagen RNeasy Mini Kit; first strand cDNA was synthesized using Invitrogen M-MLY reverse transcriptase and random primer; real-time PCR was performed with iTaq Universal SRBR Green Supermix (BioRad) and primers to GAA (forward: cagagcagtgcccacacag (SEQ ID NO: 140), reverse: gtagcccatttcagaggagctc (SEQ ID NO:141) and primers to β-actin (AGAGGGAAATCGTGCGTGAC (SEQ ID NO:129); CAATAGTGATGACCTGGCCGT (SEQ ID NO:130). A linearized plasmid was used for generating a standard curve for calculating GAA mRNA copy number.

FIG. 18 shows a Cas9 vector (7465 bp) comprising a minG6PC promoter driving SpCas9.

FIG. 19 shows a G6PC vector (6505 bp) comprising a 5′ homology arm to mG6PC, a 3′ homology arm to mG6PC, and a human G6PC donor template.

FIG. 20 shows a AAV vector (6233 bp) comprising CRM4G6PC promoter with a human GAA template.

FIG. 21 shows a AAV vector (5740 bp) comprising G6PC303 promoter with a human GAA template.

FIG. 22 shows a AAV vector (5793 bp) comprising G6PC356 promoter with a human GAA template.

FIG. 23 shows a AAV vector (7285 bp) comprising G6PC303 promoter with SaCas9.

### BRIEF SUMMARY

Disclosed herein is a nucleic acid sequence for a G6PC minimal promoter. Disclosed herein is a nucleic acid sequence for a human G6PC minimal promoter. Disclosed herein is a nucleic acid sequence for a non-human G6PC minimal promoter.

Disclosed herein is a nucleic acid sequence for a promoter comprising the sequence set forth in SEQ ID NO:02 or SEQ ID NO:03.

Disclosed herein is a nucleic acid sequence for a promoter that can be incorporated into an AAV vector comprising a nucleic acid sequence encoding a Cas9 endonuclease.

Disclosed herein is a nucleic acid sequence for a promoter comprising the sequence set forth in SEQ ID NO:02 or SEQ ID NO:03 and that can be incorporated into an AAV vector comprising a nucleic acid sequence encoding a Cas9 endonuclease.

Disclosed herein is a nucleic acid sequence for a promoter that can be incorporated into an AAV vector comprising a nucleic acid sequence for a transgene or cDNA.

Disclosed herein is a nucleic acid sequence for a promoter comprising the sequence set forth in SEQ ID NO:02 or SEQ ID NO:03 and that can be incorporated into an AAV vector comprising a nucleic acid sequence for a transgene or cDNA.

Disclosed herein is a hybrid promoter. Disclosed herein is a hybrid promoter comprising a G6PC min promoter and a human skeletal muscle-specific transcriptional cis-regulatory module. Disclosed herein is a hybrid promoter comprising a G6PC min promoter and a human skeletal muscle-specific enhancer.

Disclosed herein is a hybrid promoter comprising a G6PC min promoter and a hSk-CRM4 regulatory module. Disclosed herein is a hybrid promoter comprising a hSk-CRM4 regulatory module and a G6PC min promoter. Disclosed herein is a hybrid promoter comprising a hSk-CRM4 regulatory module and a G6PC min promoter. Disclosed herein is a hybrid promoter comprising a G6PC min promoter and a hSk-CRM4 regulatory module.

Disclosed herein is a hybrid promoter comprising a G6PC min promoter and a hSk-CRM4 regulatory module separated by a spacer or a linker. Disclosed herein is a nucleic acid sequence for a hybrid promoter comprising the sequence set forth in any one of SEQ ID NO:06-SEQ ID NO:09. Disclosed herein is a nucleic acid sequence for a hybrid promoter comprising the sequence set forth in any one of SEQ ID NO:10-SEQ ID NO:13.

Disclosed herein is a nucleic acid sequence for a hybrid promoter that can be incorporated into an AAV vector comprising a nucleic acid sequence encoding a Cas9 endonuclease.

Disclosed herein is a nucleic acid sequence for a hybrid promoter comprising the sequence set forth in any one of SEQ ID NO:06-SEQ ID NO:13 and that can be incorporated into an AAV vector comprising a nucleic acid sequence encoding a Cas9 endonuclease.

Disclosed herein is a nucleic acid sequence for a hybrid promoter that can be incorporated into an AAV vector comprising a nucleic acid sequence for a transgene or cDNA.

Disclosed herein is a nucleic acid sequence for a hybrid promoter comprising the sequence set forth in any one of SEQ ID NO:06-SEQ ID NO:13 and that can be incorporated into an AAV vector comprising a nucleic acid sequence for a transgene or cDNA.

Disclosed herein is a nucleic acid sequence for a transgene for correcting GSDIa or a fragment thereof. Disclosed herein is a nucleic acid sequence for a cDNA for correcting GSDIa or a fragment thereof. Disclosed herein is a nucleic acid sequence for a transgene for correcting GSDII or a fragment thereof. Disclosed herein is a nucleic acid sequence for a cDNA for correcting GSDII or a fragment thereof.

Disclosed herein is a donor vector comprising from 5′ to 3′ the following components: a first disclosed ITR, a disclosed 5′ homology arm, a disclosed promoter or disclosed hybrid promoter that is operably linked to a disclosed transgene or a disclosed cDNA, a polyA sequence, a disclosed 3′ homology arm, a promoter that is operably linked to a gRNA, and a second disclosed ITR. Disclosed herein is a donor vector comprising from 5′ to 3′ the following components: a first disclosed ITR, a disclosed 5′ homology arm, a disclosed G6PC min promoter or disclosed hybrid promoter comprising a G6PC min promoter and a hSk-CRM4 regulatory module that is operably linked to a disclosed transgene or a disclosed cDNA, a polyA sequence, a disclosed 3′ homology arm, a U6 promoter that is operably linked to a gRNA, and a second disclosed ITR.

Disclosed herein is a CRISPR vector method of gene editing. In an aspect, a CRISPR vector can comprise a nucleic acid sequence for a Cas9 nuclease. In an aspect, a CRISPR vector can comprise a disclosed G6PC promoter or a disclosed hybrid promoter.

Disclosed herein is a CRISPR vector comprising from 5′ to 3′ the following components: a first disclosed ITR, a disclosed promoter or disclosed hybrid promoter that is operably linked to a disclosed Cas9 endonuclease, a disclosed polyA sequence, and a second disclosed ITR. Disclosed herein is a CRISPR vector comprising from 5′ to 3′ the following components: a first disclosed ITR, a disclosed G6PC min promoter or disclosed hybrid promoter comprising a G6PC min promoter and a hSk-CRM4 regulatory module that is operably linked to a disclosed Cas9 endonuclease, a polyA sequence, and a second disclosed ITR.

Disclosed herein is a gene editing system for stably integrating a transgene into one or more cells, comprising (i) a disclosed donor nucleic acid molecule comprising from 5′ to 3′ a first disclosed ITR, a disclosed 5′ homology arm, a disclosed promoter or disclosed hybrid promoter that is operably linked to a disclosed transgene or a disclosed cDNA, a polyA sequence, a disclosed 3′ homology arm, a promoter that is operably linked to a gRNA, and a second disclosed ITR, and (ii) a disclosed CRISPR nucleic acid molecule comprising from 5′ to 3′ a first disclosed ITR, a disclosed promoter or disclosed hybrid promoter that is operably linked to a disclosed Cas9 endonuclease, a disclosed polyA sequence, and a second disclosed ITR.

Disclosed herein is a gene editing system for stably integrating a transgene into one or more cells, comprising (i) a disclosed donor nucleic acid molecule comprising from 5′ to 3′ a first disclosed ITR, a disclosed 5′ homology arm, a disclosed G6PC min promoter or disclosed hybrid promoter comprising a G6PC min promoter and a hSk-CRM4 regulatory module that is operably linked to a disclosed transgene or a disclosed cDNA, a polyA sequence, a disclosed 3′ homology arm, a U6 promoter that is operably linked to a gRNA, and a second disclosed ITR, and (ii) a disclosed CRISPR nucleic acid molecule comprising from 5′ to 3′ a first disclosed ITR, a disclosed G6PC min promoter or disclosed hybrid promoter comprising a G6PC min promoter and a hSk-CRM4 regulatory module that is operably linked to a disclosed Cas9 endonuclease, a polyA sequence, and a second disclosed ITR.

Disclosed herein is a pharmaceutical formulation comprising a disclosed donor vector and a pharmaceutically acceptable carrier. Disclosed herein is a pharmaceutical formulation comprising a disclosed CRISPR vector and a pharmaceutically acceptable carrier.

Disclosed herein is a method of repairing a defective gene, the method comprising contacting cells with a disclosed donor nucleic acid molecule and a disclosed CRISPR nucleic acid molecule, wherein, following expression of the nucleic acid molecules, the defective gene is repaired in the cells.

Disclosed herein is a method of repairing a defective gene, the method comprising contacting cells with a disclosed donor vector and a disclosed CRISPR vector, wherein, following expression of the nucleic acid sequences, the defective gene is repaired in the cells.

Disclosed herein is a method of repairing a defective gene, the method comprising contacting cells with a disclosed gene editing system, wherein, following expression of the nucleic acid sequences, the defective gene is repaired in the cells.

Disclosed herein is an in vivo method for treating a subject having a genetic disease or genetic disorder, the method comprising administering to the subject a therapeutically effective amount of a disclosed donor vector; and administering to the subject therapeutically effective amount of a disclosed vector; wherein the Cas9 creates a double-strand break (DSB) on both sides of a mutation or defect in the gene locus that results in a permanent integration of a disclosed transgene, thereby repairing the defect underlying the genetic disease or disorder.

Disclosed herein is an in vivo method for treating a subject having GSDIa, the method comprising administering to the subject a therapeutically effective amount of a disclosed donor vector; and administering to the subject therapeutically effective amount of a disclosed vector; wherein the Cas9 creates a double-strand break (DSB) on both sides of a mutation or defect in the G6PC locus that results in a permanent integration of a disclosed G6PC transgene, thereby repairing the defect underlying GSDIa.

Disclosed herein is an in vivo method for treating a subject having GSDII, the method comprising administering to the subject a therapeutically effective amount of a disclosed donor vector; and administering to the subject therapeutically effective amount of a disclosed vector; wherein the Cas9 creates a double-strand break (DSB) on both sides of a mutation or defect in the GAA gene locus that results in a permanent integration of a disclosed GAA transgene, thereby repairing the defect underlying GSDII.

### DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

In an aspect, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

In an aspect, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

In an aspect, “isolated” refers to a nucleic acid molecule or a nucleic acid sequence that has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. Isolated proteins or nucleic acids, or cells containing such, in some examples are at least 50% pure, such as at least 75%, at least 80%, at least 90%, at least 95%, at least 98%, or at least 100% pure.

In an aspect, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a disease or disorder, be suspected of having a disease or disorder, or be at risk of developing a disease or disorder (e.g., a genetic disease or a genetic disorder such as GSDIa or GSDII).

In an aspect, a “regulatory element” can refer to promoters, enhancers, internal ribosomal entry sites (IRES), and other expression control elements (e.g., transcription termination signals, such as polyadenylation signals and poly-U sequences). Regulatory elements can include those that direct constitutive expression of a nucleotide sequence in many types of host cells and those that direct expression of the nucleotide sequence only in certain host cells (e.g., tissue-specific regulatory sequences).

In an aspect, the term “diagnosed” means having been subjected to an examination by a person of skill, for example, a physician, and found to have a condition that can be diagnosed or treated by one or more of the disclosed isolated nucleic acid molecules, disclosed vectors, disclosed pharmaceutical formulations, or a combination thereof, or by one or more of the disclosed methods. For example, “diagnosed with a disease or disorder” means having been subjected to an examination by a person of skill, for example, a physician, and found to have a condition (e.g., a GSD or a genetic disease or a genetic disorder) that can be treated by one or more of the disclosed isolated nucleic acid molecules, disclosed vectors, disclosed pharmaceutical formulations, or a combination thereof, or by one or more of the disclosed methods. For example, “suspected of having a disease or disorder” can mean having been subjected to an examination by a person of skill, for example, a physician, and found to have a condition (e.g., Pompe disease or a genetic disease or disorder) that can likely be treated by one or more of by one or more of the disclosed isolated nucleic acid molecules, disclosed vectors, disclosed pharmaceutical formulations, or a combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” refers to a subject afflicted with a disease or disorder (e.g., a genetic disease or a genetic disorder). In an aspect, a patient can refer to a subject that has been diagnosed with or is suspected of having a disease or disorder. In an aspect, a patient can refer to a subject that has been diagnosed with or is suspected of having a disease or disorder and is seeking treatment or receiving treatment for a genetic disease or genetic disorder.

In an aspect, the phrase “identified to be in need of treatment for a disease or disorder,” or the like, refers to selection of a subject based upon need for treatment of the disease or disorder. For example, a subject can be identified as having a need for treatment of a disease or disorder (e.g., such as a GSD like GSDIa or GSDII) based upon an earlier diagnosis by a person of skill and thereafter subjected to treatment for the genetic disease or disorder. In an aspect, the identification can be performed by a person different from the person making the diagnosis. In an aspect, the administration can be performed by one who performed the diagnosis.

In an aspect, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having a disease or disorder like Pompe disease or a genetic disease or disorder). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels. In an aspect, a native or control level can be a pre-disease or pre-disorder level.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a disease or disorder can reduce the severity of an established a disease or disorder in a subject by 1%-100% as compared to a control (such as, for example, an individual not having Pompe disease or a genetic disease or disorder). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or disorder (such as a genetic disease or disorder). For example, treating a disease or disorder can reduce one or more symptoms of a disease or disorder in a subject by 1%-100% as compared to a control (such as, for example, an individual not having a genetic disease or disorder). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or disorder (such as Pompe disease). It is understood that treatment does not necessarily refer to a cure or complete ablation or eradication of a disease or disorder. However, in an aspect, treatment can refer to a cure or complete ablation or eradication of a disease or disorder.

In an aspect, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease or disorder having chromatin deregulation and/or chromatin dysregulation is intended. The words “prevent”, “preventing”, and “prevention” also refer to prophylactic or preventative measures for protecting or precluding a subject (e.g., an individual) not having a given a disease or disorder (such as Pompe disease or a genetic disease or disorder) or a related complication from progressing to that complication.

In an aspect, the terms “administering” and “administration” refer to any method of providing one or more of the disclosed isolated nucleic acid molecules, disclosed vectors, disclosed pharmaceutical formulations, or a combination thereof to a subject. Such methods are well known to those skilled in the art and include, but are not limited to, the following: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed isolated nucleic acid molecule, a disclosed vector, a disclosed pharmaceutical composition, a disclosed therapeutic agent, a disclosed immune modulator, a disclosed proteasome inhibitor, a disclosed small molecule, and/or a disclosed endonuclease can comprise administration directly into the CNS or the PNS. Administration can be continuous or intermittent. Administration can comprise a combination of one or more route.

In an aspect, the skilled person can determine an efficacious dose, an efficacious schedule, and an efficacious route of administration for one or more of the disclosed isolated nucleic acid molecules, disclosed vectors, disclosed pharmaceutical formulations, or a combination thereof to treat or prevent a disease or disorder (such as genetic disease or disorder). In an aspect, the skilled person can also alter, change, or modify an aspect of an administering step to improve efficacy of one or more of the disclosed isolated nucleic acid molecules, disclosed vectors, disclosed pharmaceutical formulations, or a combination thereof.

By “determining the amount” is meant both an absolute quantification of a particular analyte (e.g., an mRNA sequence containing a particular tag) or a determination of the relative abundance of a particular analyte (e.g., an amount as compared to a mRNA sequence including a different tag). The phrase includes both direct or indirect measurements of abundance (e.g., individual mRNA transcripts may be quantified or the amount of amplification of an mRNA sequence under certain conditions for a certain period may be used a surrogate for individual transcript quantification) or both.

In an aspect, “modifying the method” can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, by changing the duration of time one or more of the disclosed isolated nucleic acid molecules, disclosed vectors, disclosed pharmaceutical formulations, or a combination are administered to a subject, or by substituting for one or more of the disclosed components and/or reagents with a similar or equivalent component and/or reagent. The same applies to all disclosed therapeutic agents, immune modulators, immunosuppressive agents, proteosome inhibitors, etc.

In an aspect, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

In an aspect, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

In an aspect, “concurrently” means (1) simultaneously in time, or (2) at different times during the course of a common treatment schedule.

“Efficiency” when used in describing viral production, replication or packaging refers to useful properties of the method: in particular, the growth rate and the number of virus particles produced per cell. “High efficiency” production indicates production of at least 100 viral particles per cell; e.g., at least about 10,000 or at least about 100,000 particles per cell, over the course of the culture period specified.

In an aspect, the term “contacting” refers to bringing one or more of the disclosed isolated nucleic acid molecules, disclosed vectors, disclosed pharmaceutical formulations, or a combination thereof together with a target area or intended target area in such a manner that the one or more of the disclosed isolated nucleic acid molecules, disclosed vectors, disclosed pharmaceutical formulations, or a combination thereof exert an effect on the intended target or targeted area either directly or indirectly. A target area can comprise one or more cells, and in an aspect, one or more cells can be in a subject. A target area or intended target area can be one or more of a subject's organs (e.g., skeletal muscles, lungs, heart, liver, kidney, brain, etc.). In an aspect, a target area or intended target area can be any cell or any organ infected by a disease or disorder (such as a genetic disease or disorder). In an aspect, a target area or intended target area can be any organ, tissue, or cells that are affected by a disease or disorder (such as Pompe disease or a genetic disease or disorder).

In an aspect, “determining” can refer to measuring or ascertaining the presence and severity of a disease or disorder, such as, for example, a genetic disease or disorder. Methods and techniques used to determine the presence and/or severity of a disease or disorder are typically known to the medical arts. For example, the art is familiar with the ways to identify and/or diagnose the presence, severity, or both of a disease or disorder (such as, for example, a genetic disease or disorder).

In an aspect, “effective amount” and “amount effective” can refer to an amount that is sufficient to achieve the desired result such as, for example, the treatment and/or prevention of a disease or disorder (e.g., a genetic disease or disorder such as Pompe disease) or a suspected disease or disorder. In an aspect, the terms “effective amount” and “amount effective” can refer to an amount that is sufficient to achieve the desired an effect on an undesired condition (e.g., a disease or disorder such as Pompe disease). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed isolated nucleic acid molecule, a disclosed vector, or a disclosed pharmaceutical formulation; that (i) treats the particular disease, condition, or disorder (e.g., a genetic disease or disorder such as Pompe disease), (ii) attenuates, ameliorates, or eliminates one or more symptoms of the particular disease, condition, or disorder e.g., a genetic disease or disorder such as Pompe disease), or (iii) delays the onset of one or more symptoms of the particular disease, condition, or disorder described herein (e.g., a genetic disease or disorder such as Pompe disease). The specific therapeutically effective dose level for any particular patient will depend upon a variety of factors including the disorder being treated and the severity of the disorder; the disclosed isolated nucleic acid molecules, disclosed vectors, disclosed pharmaceutical formulations employed; the disclosed methods employed; the age, body weight, general health, sex and diet of the patient; the time of administration; the route of administration; the rate of excretion of the disclosed isolated nucleic acid molecules, disclosed vectors, or disclosed pharmaceutical formulations employed; the duration of the treatment; drugs used in combination or coincidental with the disclosed isolated nucleic acid molecules, disclosed vectors, or disclosed pharmaceutical formulations employed, and other like factors well known in the medical arts. For example, it is well within the skill of the art to start doses of the disclosed isolated nucleic acid molecules, disclosed vectors, or disclosed pharmaceutical formulations at levels lower than those required to achieve the desired therapeutic effect and to gradually increase the dosage until the desired effect is achieved. If desired, then the effective daily dose can be divided into multiple doses for purposes of administration. Consequently, a single dose of the disclosed isolated nucleic acid molecules, disclosed vectors, or disclosed pharmaceutical formulations can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a disease or disorder due to a missing, deficient, and/or mutant protein or enzyme.

In an aspect, “nucleic acid expression cassette” can refer to an isolated nucleic acid molecule that includes one or more transcriptional control elements (e.g., promoters, enhancers, and/or regulatory elements, polyadenylation sequences, and introns) that are operably linked to and direct gene expression in one or more desired cell types, tissues, or organs (e.g., liver, brain, kidney, skeletal muscle, etc.). A nucleic acid expression cassette can contain a transgene (e.g., G6PC or GAA or a portion thereof), although it is also envisaged that a nucleic acid expression cassette directs expression of an endogenous gene in a cell into which the nucleic acid sequence is inserted thereof), and a 3′ untranslated region (e.g., a polyadenylation site).

In an aspect, “small molecule” can refer to any organic or inorganic material that is not a polymer. Small molecules exclude large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weight of over 2,000, 3,000, 4,000, 5,000, 6,000, 7,000, 8,000, 9,000, or 10,000). In an aspect, a “small molecule”, for example, can be a drug that can enter cells easily because it has a low molecular weight. In an aspect, a small molecule can be used in conjunction with a disclosed composition in a disclosed method.

In an aspect, “homology directed repair” or “HDR” can occur either non-conservatively or conservatively. The non-conservative method is composed of the single-strand annealing (SSA) pathway and is more error prone. The conservative methods, characterized by the accurate repair of the DSB by means of a homologous donor (e.g., sister chromatid, plasmid, etc.), are composed of three pathways: the classical double-strand break repair (DSBR), synthesis-dependent strand-annealing (SDSA), and break-induced repair (BIR). For example, in the classical DSBR pathway, the 3′ ends invade an intact homologous template to serve as a primer for DNA repair synthesis, ultimately leading to the formation of double Holliday junctions (dHJs). dHJs are four-stranded branched structures that form when elongation of the invasive strand “captures” and synthesizes DNA from the second DSB end. The individual HJs are resolved via cleavage in one of two ways. Each junction resolution could happen on the crossing strand (horizontally at the purple arrows) or on the non-crossing strand (vertically at the orange arrows). If resolved dissimilarly (e.g., one junction is resolved on the crossing strand and the other on the non-crossing strand), then a crossover event will occur; however, if both HJs are resolved in the same manner, this results in a non-crossover event. DSBR is semi-conservative, as crossover events are most common.

In an aspect, “transduction” or “transducing” are terms referring to a process for the introduction of an exogenous polynucleotide, e.g., a transgene in rAAV vector, into a host cell leading to expression of the polynucleotide, e.g., the transgene in the cell. The process can include one or more of (i) endocytosis of the chimeric virus, (ii) escape from endosomes or other intracellular compartments in the cytosol of a cell, (iii) trafficking of the viral particle or viral genome to the nucleus, (iv) uncoating of the virus particles, and (v) generation of expressible double stranded AAV genome forms, including circular intermediates. The rAAV expressible double stranded form may persist as a nuclear episome or optionally may integrate into the host genome. Methods used for the introduction of the exogenous polynucleotide include well-known techniques such as transfection, lipofection, viral infection, transformation, and electroporation, as well as non-viral gene delivery techniques. The introduced polynucleotide may be stably or transiently maintained in the host cell.

In an aspect, “operably linked” means that expression of a gene or a transgene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

In an aspect, “peptide,” “polypeptide,” and “protein” are used interchangeably, and refer to a compound comprised of amino acid residues covalently linked by peptide bonds. A protein must contain at least two amino acids and there is no limitation on the maximum number of amino acids that can comprise a protein's sequence. The term “peptide” can refer to a short chain of amino acids including, for example, natural peptides, recombinant peptides, synthetic peptides, or any combination thereof. Proteins and peptides can include, for example, biologically active fragments, substantially homologous polypeptides, oligopeptides, homodimers, heterodimers, variants of polypeptides, modified polypeptides, derivatives, analogs, and fusion proteins, among others.

A cell is said to be “stably” altered, transduced or transformed with a genetic sequence if the sequence is available to perform its function during extended culture of the cell in vitro. In some examples, such a cell is “inheritably” altered in that a genetic alteration is introduced which is also inheritable by progeny of the altered cell.

In an aspect, “nucleic acid” or “oligonucleotide” or “polynucleotide” means at least two nucleotides covalently linked together. The depiction of a single strand can also define the sequence of the complementary strand. Thus, a nucleic acid can encompass the complementary strand of a depicted single strand. Many variants of a nucleic acid can be used for the same purpose as a given nucleic acid. Thus, a nucleic acid can encompass substantially identical nucleic acids and complements thereof. A single strand can provide a probe that can hybridize to a target sequence under stringent hybridization conditions. Thus, a nucleic acid can encompass a probe that hybridizes under stringent hybridization conditions. A nucleic acid can be single-stranded, or double-stranded, or can contain portions of both double-stranded and single-stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods. In an aspect, the terms “nucleic acid,” “nucleic acid molecule,” “nucleic acid construct,” “nucleotide sequence”, and “polynucleotide” can refer to RNA or DNA that is linear or branched, single or double stranded, or a hybrid thereof. The term can encompass RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

In an aspect, “synthetic” nucleic acid or polynucleotide, refers to a nucleic acid or polynucleotide that is not found in nature but is constructed by the hand of man and therefore is not a product of nature.

In an aspect, a “polynucleotide” is a sequence of nucleotide bases, and may be RNA, DNA, or DNA-RNA hybrid sequences (including both naturally occurring and non-naturally occurring nucleotides).

In an aspect, a “fragment” or “portion” of a nucleotide sequence can be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising, consisting essentially of, or consisting of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the disclosure can be, where appropriate, included in a larger polynucleotide of which it is a constituent. In an aspect, a fragment or portion of a nucleotide sequence or nucleic acid sequence can comprise the sequence encoding an exon having one or more mutations or defects.

A “fragment” or “portion” of an amino acid sequence can be understood to mean an amino acid sequence of reduced length relative (e.g., reduced by 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, or more amino acids) to a reference amino acid sequence and comprising, consisting essentially of, or consisting of an amino acid sequence of contiguous amino acids identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference amino acid sequence. Such an amino acid fragment or portion according to the disclosure can be, where appropriate, included in a larger amino acid sequence of which it is a constituent.

In an aspect, a “heterologous” or a “recombinant” nucleotide or amino acid sequence as used interchangeably herein can refer to a nucleotide or an amino acid sequence not naturally associated with a host cell into which it is introduced, including non-naturally occurring multiple copies of a naturally occurring nucleotide or amino acid sequence.

In an aspect, “complement” or “complementary” means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

In an aspect, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native (endogenous) or foreign (exogenous) and can be a natural or a synthetic sequence. By foreign or exogenous, it is intended that the transcriptional initiation region is not found in the wild-type host into which the transcriptional initiation region is introduced.

In an aspect, genetic diseases and disorders include, but are not limited to, diseases and disorders due to a defect in the following genes: ABCA1, ABCA12, ABCA13, ABCA2, ABCA3, ABCA4, ABCA5, ABCC1, ABCC2, ABCC6, ABCC8, ABCC9, ACAN, ADAMTS13, ADCY10, ADGRV1, AGL, AGRN, AHDC1, ALK, ALMS1, ALPK3, ALS2, ANAPC1, ANK1, ANK2, ANK3, ANKRD11, ANKRD26, APC, APC2, APOB, ARFGEF2, ARHGAP31, ARHGEF10, ARHGEF18, ARID1A, ARID1B, ARID2, ASH1L, ASPM, ASXL1, ASXL2, ASXL3, ATM, ATP7A, ATP7B, ATR, ATRX, BAZ1A, BAZ2B, BCOR, BCORL1, BDP1, BLM, BPTF, BRCA1, BRCA2, BRD4, BRWD3, C2CD3, C3, C5, CACNA1A, CACNA1B, CACNA1C, CACNA1D, CACNA1E, CACNA1F, CACNA1G, CACNA1H, CACNA1S, CAD, CAMTA1, CARMIL2, CC2D2A, CCDC88A, CCDC88C, CCNB3, CDH23, CDK13, CDK5RAP2, CELSR1, CEMIP2, CENPE, CENPF, CENPJ, CEP152, CEP164, CEP250, CEP290, CFAP43, CFAP44, CFAP65, CFTR/ABCC7, CHD1, CHD2, CHD3, CHD4, CHD7, CHD8, CIC, CIT, CLIP1, CLTC, CNOT1, CNTNAP1, COL11A1, COL11A2, COL12A1, COL17A1, COL18A1, COL1A1, COL1A2, COL27A1, COL2A1, COL3A1, COL4A1, COL4A2, COL4A3, COL4A4, COL4A5, COL4A6, COL5A1, COL5A2, COL6A3, COL7A1, CPAMD8, CPLANE1, CPS1, CPSF1, CRB1, CREBBP, CUBN, CUL7, CUX1, DCC, DCHS1, DEPDC5, DICER1, DIP2B, DLC1, DMD, DMXL2, DNAH1, DNAH11, DNAH17, DNAH2, DNAH5, DNAH7, DNAH8, DNAH9, DNMBP, DNMT1, DOCK2, DOCK3, DOCK6, DOCK7, DOCK8, DSCAM, DSP, DST, DUOX2, DYNC1H1, DYNC2H1, DYSF, EIF2AK4, EP300, EPG5, ERCC6, ERCC6L2, EXPH5, EYS, F5, F8, FANCA, FANCD2, FANCM, FAT1, FAT4, FBN1, FBN2, FLG, FLG2, FLNA, FLNB, FLNC, FLT4, FMN2, FN1, FRAS1, FREM1, FREM2, FSIP2, FYCO1, GLI2, GLI3, GPR179, GREB1L, GRIN2A, GRIN2B, GRIN2D, HCFC1, HECW2, HERC1, HERC2, HFM1, HIVEP1, HIVEP2, HMCN1, HSPG2, HTT, HUWE1, HYDIN, IFT140, IFT172, IGF1R, IGF2R, IGSF1, INSR, INTS1, IQSEC2, ITGB4, ITPR1, ITPR2, JMJD1C, KALRN, KANK1, KAT6A, KAT6B, KDM3B, KDM5B, KDM5C, KDM6A, KDM6B, KDR, KIAA0586, KIAA1109, KIAA1549, KIDINS220, KIF14, KIF1A, KIF1B, KIF21A, KIF26B, KIF7, KMT2A, KMT2B, KMT2C, KMT2D, KMT2E, KNL1, LAMA1, LAMA2, LAMA3, LAMA4, LAMA5, LAMB1, LAMB2, LAMC3, LCT, LOXHD1, LPA, LRBA, LRP1, LRP2, LRP4, LRP5, LRP6, LRPPRC, LRRK1, LRRK2, LTBP2, LTBP4, LYST, MACF1, MADD, MAGI2, MAP1B, MAP3K1, MAPK8IP3, MAPKBP1, MAST1, MBD5, MCM3AP, MED12, MED12L, MED13, MED13L, MED23, MEGF8, MET, MLH3, MPDZ, MSH6, MTOR, MYH10, MYH11, MYH14, MYH2, MYH3, MYH6, MYH7, MYH7B, MYH8, MYH9, MYLK, MYO15A, MYO18B, MYO3A, MYO5A, MYO5B, MYO7A, MYO9A, NALCN, NBAS, NBEA, NBEAL2, NCAPD2, NCAPD3, NEB, NEXMIF, NEXMIF, NF1, NFASC, NHS, NIN, NIPBL, NLRP1, NOTCH1, NOTCH2, NOTCH3, NPHP4, NRXN1, NRXN3, NSD1, NSD2, NUP155, NUP188, NUP205, OBSCN, OBSL1, OTOF, OTOG, OTOGL, PARD3, PBRM1, PCDH15, PCLO, PCNT, PHIP, PI4KA, PIEZO1, PIEZO2, PIK3C2A, PIKFYVE, PKD1, PKD1L1, PKHD1, PLCE1, PLEC, PLEKHG2, PNPLA6, POGZ, POLA1, POLE, POLR1A, POLR2A, POLR3A, PRG4, PRKDC, PRPF8, PRR12, PRX, PTCH1, PTPN23, PTPRF, PTPRJ, PTPRQ, PXDN, QRICH2, RAB3GAP2, RAI1, RALGAPA1, RANBP2, RB1CC1, RELN, RERE, REV3L, RIC1, RIMS1, RIMS2, RNF213, ROBO1, ROBO2, ROBO3, ROS1, RP1, RP1L1, RTTN, RUSC2, RYR1, RYR2, SACS, SAMD9, SAMD9L, SBF2, SCAPER, SCN10A, SCN11A, SCN1A, SCN2A, SCN3A, SCN4A, SCN5A, SCN8A, SCN9A, SETBP1, SETD1A, SETD1B, SETD2, SETD5, SETX, SHANK2, SHANK3, SHROOM4, SI, SIPA1L3, SLIT2, SLX4, SMARCA2, SMARCA4, SMCHD1, SNRNP200, SON, SPEF2, SPEG, SPG11, SPTA1, SPTAN1, SPTB, SPTBN2, SPTBN4, SRCAP, STRC, SVIL, SYNE1, SYNGAP1, SYNJ1, SZT2, TAF1, TANC2, TCF20, TCOF1, TDRD9, TECPR2, TECTA, TENM3, TENM4, TET3, TEX14, TEX15, TG, THOC2, TMEM94, TNC, TNIK, TNR, TNRC6B, TNXB, TOGARAM1, TONSL, TRIO, TRIOBP, TRIP11, TRIP12, TRPM1, TRPM6, TRPM7, TRRAP, TSC2, TTC37, TTN, TUBGCP6, UBR1, UNC80, USH2A, USP9X, VCAN, VPS13A, VPS13B, VPS13C, VPS13D, VWF, WDFY3, WDR19, WDR62, WDR81, WNK1, WRN, ZFHX2, ZFYVE26, ZNF142, ZNF292, ZNF335, ZNF407, ZNF462, ZNF469, or a portion thereof.

In an aspect, genetic diseases and disorders can also include, but are not limited to, diseases and disorders due to a defect in the following gene: dystrophin including mini- and micro-dystrophins (DMD); titin (TTN); titin cap (TCAP) α-sarcoglycan (SGCA), β-sarcoglycan (SGCB), γ-sarcoglycan (SGCG) or 6-sarcoglycan (SGCD); alpha-1-antitrypsin (A1-AT); myosin heavy chain 6 (MYH6); myosin heavy chain 7 (MYH7); myosin heavy chain 11 (MYH11); myosin light chain 2 (ML2); myosin light chain 3 (ML3); myosin light chain kinase 2 (MYLK2); myosin binding protein C (MYBPC3); desmin (DES); dynamin 2 (DNM2); laminin α2 (LAMA2); lamin A/C (LMNA); lamin B (LMNB); lamin B receptor (LBR); dysferlin (DYSF); emerin (EMD); insulin; blood clotting factors, including but not limited to, factor VIII and factor IX; erythropoietin (EPO); lipoprotein lipase (LPL); sarcoplasmic reticulum Ca2++-ATPase (SERCA2A), S100 calcium binding protein A1 (S100A1); myotubularin (MTM); DM1 protein kinase (DMPK); glycogen phosphorylase L (PYGL); glycogen phosphorylase, muscle associated (PYGM); glycogen synthase 1 (GYS1); glycogen synthase 2 (GYS2); α-galactosidase A (GLA); α-N-acetylgalactosaminidase (NAGA); acid α-glucosidase (GAA), sphingomyelinase phosphodiesterase 1 (SMPD1); lysosomal acidlipase (LIPA); collagen type I α1 chain (COL1A1); collagen type I α2 chain (COL1A2); collagen type III α1 chain (COL3A1); collagen type V α1 chain (COL5A1); collagen type V α2 chain (COL5A2); collagen type VI α1 chain (COL6A1); collagen type VI α2 chain (COL6A2); collagen type VI α3 chain (COL6A3); procollagen-lysine 2-oxoglutarate 5-dioxygenase (PLOD1); lysosomal acid lipase (LIPA); frataxin (FXN); myostatin (MSTN); β-N-acetyl hexosaminidase A (HEXA); β-N-acetylhexosaminidase B (HEXB); β-glucocerebrosidase (GBA); adenosine monophosphate deaminase 1 (AMPD1); β-globin (HBB); iduronidase (IDUA); iduronate 2-sulfate (IDS); troponin 1 (TNNI3); troponin T2 (TNNT2); troponin C (TNNC1); tropomyosin 1 (TPM1); tropomyosin 3 (TPM3); N-acetyl-α-glucosaminidase (NAGLU); N-sulfoglucosamine sulfohydrolase (SGSH); heparan-α-glucosaminide N-acetyltransferase (HGSNAT); integrin a 7 (IGTA7); integrin a 9 (IGTA9); glucosamine(N-acetyl)-6-sulfatase (GNS); galactosamine(N-acetyl)-6-sulfatase (GALNS); β-galactosidase (GLB1); β-glucuronidase (GUSB); hyaluronoglucosaminidase 1 (HYAL1); acid ceramidase (ASAHI); galactosylcermidase (GALC); cathepsin A (CTSA); cathepsin D (CTSA); cathepsin K (CTSK); GM2 ganglioside activator (GM2A); arylsulfatase A (ARSA); arylsulfatase B (ARSB); formylglycine-generating enzyme (SUMFI); neuraminidase 1 (NEU1); N-acetylglucosamine-1-phosphate transferase a (GNPTA); N-acetylglucosamine-1-phosphate transferase R (GNPTB); N-acetylglucosamine-1-phosphate transferase γ (GNPTG); mucolipin-1 (MCOLN1); NPC intracellular transporter 1 (NPC1); NPC intracellular transporter 2 (NPC2); ceroid lipofuscinosis 5 (CLN5); ceroid lipofuscinosis 6 (CLN6); ceroid lipofuscinosis 8 (CLN8); palmitoyl protein thioesterase 1 (PPT1); tripeptidyl peptidase 1 (TPP1); battenin (CLN3); DNAJ heat shock protein family 40 member C5 (DNAJC5); major facilitator superfamily domain containing 8 (MFSD8); mannosidase a class 2B member 1 (MAN2B1); mannosidase R (MANBA); aspartylglucosaminidase (AGA); α-L-fucosidase (FUCA1); cystinosin, lysosomal cysteine transporter (CTNS); sialin; solute carrier family 2 member 10 (SLC2A10); solute carrier family 17 member 5 (SLC17A5); solute carrier family 6 member 19 (SLC6A19); solute carrier family 22 member 5 (SLC22A5); solute carrier family 37 member 4 (SLC37A4); lysosomal associated membrane protein 2 (LAMP2); sodium voltage-gated channel α subunit 4 (SCN4A); sodium voltage-gated channel R subunit 4 (SCN4B); sodium voltage-gated channel α subunit 5 (SCN5A); sodium voltage-gated channel α subunit 4 (SCN4A); calcium voltage-gated channel subunit α1c (CACNAlC); calcium voltage-gated channel subunit α1s (CACNAlS); phosphoglycerate kinase 1 (PGK1); phosphoglycerate mutase 2 (PGAM2); amylo-α-1,6-glucosidase,4-α-glucanotransferase (AGL); potassium voltage-gated channel ISK-related subfamily member 1 (KCNE1); potassium voltage-gated channel ISK-related subfamily member 2 (KCNE2); potassium voltage-gated channel subfamily J member 2 (KCNJ2); potassium voltage-gated channel subfamily J member 5 (KCNJ5); potassium voltage-gated channel subfamily H member 2 (KCNH2); potassium voltage-gated channel KQT-like subfamily member 1 (KCNQ1); hyperpolarization-activated cyclic nucleotide-gated potassium channel 4 (HCN4); chloride voltage-gated channel 1 (CLCN1); carnitine palmitoyltransferase 1A (CPT1A); ryanodine receptor 1 (RYR1); ryanodine receptor 2 (RYR2); bridging integrator 1 (BIN1); LARGE xylosyl- and glucuronyltransferase 1 (LARGE1); docking protein 7 (DOK7); fukutin (FKTN); fukutin related protein (FKRP); selenoprotein N (SELENON); protein O-mannosyltransferase 1 (POMT1); protein O-mannosyltransferase 2 (POMT2); protein O-linked mannose N-acetylglucosaminyltransferase 1 (POMGNT1); protein O-linked mannose N-acetylglucosaminyltransferase 2 (POMGNT2); protein-O-mannose kinase (POMK); isoprenoid synthase domain containing (ISPD); plectin (PLEC); cholinergic receptor nicotinic epsilon subunit (CHRNE); choline O-acetyltransferase (CHAT); choline kinase R (CHKB); collagen like tail subunit of asymmetric acetylcholinesterase (COLQ); receptor associated protein of the synapse (RAPSN); four and a half LIM domains 1 (FHL1); β-1,4-glucuronyltransferase 1 (B4GAT1); β-1,3-N-acetylgalactosaminyltransferase 2 (B3GALNT2); dystroglycan 1 (DAGI); transmembrane protein 5 (TMEM5); transmembrane protein 43 (TMEM43); SECIS binding protein 2 (SECISBP2); glucosamine (UDP-N-acetyl)-2-epimerase/N-acetylmannosamine kinase (GNE); anoctamin 5 (ANO5); structural maintenance of chromosomes flexible hinge domain containing 1 (SMCHD1); lactate dehydrogenase A (LDHA); lactate dehydrogenase B (LHDB); calpain 3 (CAPN3); caveolin 3 (CAV3); tripartite motif containing 32 (TRIM32); CCHC-type zinc finger nucleic acid binding protein (CNBP); nebulin (NEB); actin, α1, skeletal muscle (ACTA1); actin, α1, cardiac muscle (ACTC1); actinin α2 (ACTN2); poly(A)-binding protein nuclear 1 (PABPN1); LEM domain-containing protein 3 (LEMD3); zinc metalloproteinase STE24 (ZMPSTE24); microsomal triglyceride transfer protein (MTTP); cholinergic receptor nicotinic α1 subunit (CHRNA1); cholinergic receptor nicotinic α2 subunit (CHRNA2); cholinergic receptor nicotinic α3 subunit (CHRNA3); cholinergic receptor nicotinic α4 subunit (CHRNA4); cholinergic receptor nicotinic α5 subunit (CHRNA5); cholinergic receptor nicotinic α6 subunit (CHRNA6); cholinergic receptor nicotinic α7 subunit (CHRNA7); cholinergic receptor nicotinic α8 subunit (CHRNA8); cholinergic receptor nicotinic α9 subunit (CHRNA9); cholinergic receptor nicotinic α10 subunit (CHRNA10); cholinergic receptor nicotinic 31 subunit (CHRNB1); cholinergic receptor nicotinic β2 subunit (CHRNB2); cholinergic receptor nicotinic β3 subunit (CHRNB3); cholinergic receptor nicotinic 04 subunit (CFHRNB4); cholinergic receptor nicotinic γ subunit (CHRNG1); cholinergic receptor nicotinic α subunit (CHRND); cholinergic receptor nicotinic E subunit (CHRNE1); ATP binding cassette subfamily A member 1 (ABCA1); ATP binding cassette subfamily C member 6 (ABCC6); ATP binding cassette subfamily C member 9 (ABCC9); ATP binding cassette subfamily D member 1 (ABCD1); ATPase sarcoplasmic/endoplasmic reticulum Ca2+ transporting 1 (ATP2A1); ATM serine/threonine kinase (ATM); a tocopherol transferase protein (TTPA); kinesin family member 21A (KIF21A); paired-like homeobox 2a (PHOX2A); heparan sulfate proteoglycan 2 (HSPG2); stromal interaction molecule 1 (STIM1); notch 1 (NOTCH1); notch 3 (NOTCH3); dystrobrevin a (DTNA); protein kinase AMP-activated, noncatalytic γ2 (PRKAG2); cysteine- and glycine-rich protein 3 (CSRP3); viniculin (VCL); myozenin 2 (MyoZ2); myopalladin (MYPN); junctophilin 2 (JPH2); phospholamban (PLN); calreticulin 3 (CALR3); nexilin F-actin-binding protein (NEXN); LIM domain binding 3 (LDB3); eyes absent 4 (EYA4); huntingtin (HTT); androgen receptor (AR); protein tyrosine phosphate non-receptor type 11 (PTPN11); junction plakoglobin (JUP); desmoplakin (DSP); plakophilin 2 (PKP2); desmoglein 2 (DSG2); desmocollin 2 (DSC2); catenin α3 (CTNNA3); NK2 homeobox 5 (NKX2-5); A-kinase anchor protein 9 (AKAP9); A-kinase anchor protein 10 (AKAP10); guanine nucleotide-binding protein α-inhibiting activity polypeptide 2 (GNAI2); ankyrin 2 (ANK2); syntrophin α-1 (SNTAT); calmodulin 1 (CALM1); calmodulin 2 (CALM2); HTRA serine peptidase 1 (HTRA1); fibrillin 1 (FBN1); fibrillin 2 (FBN2); xylosyltransferase 1 (XYLT1); xylosyltransferase 2 (XYLT2); tafazzin (TAZ); homogentisate 1,2-dioxygenase (HGD); glucose-6-phosphatase catalytic subunit (G6PC); 1,4-alpha-glucan enzyme 1 (GBE1); phosphofructokinase, muscle (PFKM); phosphorylase kinase regulatory subunit alpha 1 (PHKA1); phosphorylase kinase regulatory subunit alpha 2 (PHKA2); phosphorylase kinase regulatory subunit beta (PHKB); phosphorylase kinase catalytic subunit gamma 2 (PHKG2); phosphoglycerate mutase 2 (PGAM2); cystathionine-beta-synthase (CBS); methylenetetrahydrofolate reductase (MTHFR); 5-methyltetrahydrofolate-homocysteine methyltransferase (MTR); 5-methyltetrahydrofolate-homocysteine methyltransferase reductase (MTRR); methylmalonic aciduria and homocystinuria, cblD type (MMADHC); mitochondrial DNA, including, but not limited to mitochondrially encoded NADH:ubiquinone oxidoreductase core subunit 1 (MT-ND1); mitochondrially encoded NADH:ubiquinone oxidoreductase core subunit 5 (MT-ND5); mitochondrially encoded tRNA glutamic acid (MT-TE); mitochondrially encoded tRNA histadine (MT-TH); mitochondrially encoded tRNA leucine 1 (MT-TL1); mitochondrially encoded tRNA lysine (MT-TK); mitochondrially encoded tRNA serine 1 (MT-TS1); mitochondrially encoded tRNA valine (MT-TV); mitogen-activated protein kinase 1 (MAP2K1); B-Raf proto-oncogene, serine/threonine kinase (BRAF); raf-1 proto-oncogene, serine/threonine kinase (RAF1); growth factors, including, but not limited to insulin growth factor 1 (IGF-1); transforming growth factor β (TGFβ3); transforming growth factor β receptor, type I (TGFβR1); transforming growth factor 1 receptor, type II (TGFβR2), fibroblast growth factor 2 (FGF2), fibroblast growth factor 4 (FGF4), vascular endothelial growth factor A (VEGF-A), vascular endothelial growth factor B (VEGF-B); vascular endothelial growth factor C (VEGF-C), vascular endothelial growth factor D (VEGF-D), vascular endothelial growth factor receptor 1 (VEGFR1), and vascular endothelial growth factor receptor 2 (VEGFR2); interleukins; immunoadhesins; cytokines; and antibodies.

In an aspect, the term “serotype” is a distinction used to refer to an AAV having a capsid that is serologically distinct from other AAV serotypes. Serologic distinctiveness can be determined by the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes).

In an aspect, “tropism” refers to the specificity of an AAV capsid protein present in an AAV viral particle, for infecting a particular type of cell or tissue. The tropism of an AAV capsid for a particular type of cell or tissue may be determined by measuring the ability of AAV vector particles comprising the hybrid AAV capsid protein to infect or to transduce a particular type of cell or tissue, using standard assays that are well-known in the art such as those disclosed in the examples of the present application. In an aspect, the term “liver tropism” or “hepatic tropism” refers to the tropism for liver or hepatic tissue and cells, including hepatocytes.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of a missing, deficient, and/or mutant protein or enzyme. It should be understood that sequence with substantial sequence identity do not necessarily have the same length and may differ in length. For example, sequences that have the same nucleotide sequence but of which one has additional nucleotides on the 3′- and/or 5′-side are 100% identical.

In an aspect, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

In an aspect, “RNA Editing” can refer to a type of genetic engineering in which an RNA molecule (or ribonucleotides of the RNA) is inserted, deleted, or replaced in the genome of an organism using engineered nucleases, which create site-specific strand breaks at desired locations in the RNA. The induced breaks are repaired resulting in targeted mutations or repairs.

In an aspect, “CRISPR or clustered regularly interspaced short palindromic repeat” is an ideal tool for correction of genetic abnormalities as the system can be designed to target genomic DNA directly. Cas9 is well-known to the art. SpCas9 and SaCas9 are well-known to the art. The CRISPR/Cas methods disclosed herein, such as those that use a Cas9 or SaCas9. can be used to edit the sequence of one or more target RNAs, such as one associated with a disease or disorder disclosed herein (e.g., a genetic disease or disorder such as Pompe disease). For example, in an aspect, a nuclease mediated break in the stem cell DNA can allow for the insertion of one or multiple genes via homology directed repair.

In an aspect, “immune tolerance,” “immunological tolerance,” and “immunotolerance” refers to a state of unresponsiveness or blunted response of the immune system to substances (e.g., a disclosed isolated nucleic acid molecule, a disclosed vector, a disclosed transgene product, a disclosed pharmaceutical formulation, a disclosed therapeutic agent, etc.) that have the capacity to elicit an immune response in a subject. Immune tolerance is induced by prior exposure to a specific antigen. Immune tolerance can be determined in a subject by measuring antibodies against a particular antigen or by liver-restricted transgene expression with a viral vector (such as, for example, AAV). Low or absent antibody titers over time is an indicator of immune tolerance.

For example, in some embodiments, immune tolerance can be established by having IgG antibody titers of less than or equal to about 12,000, 11,500, 11,000, 10,500, 10,000, 9,500, 9,000, 8,500, 8,000, 7,500, 7,000, 6,500, or 6,000 within following gene therapy (such as the administration of the transgene encoding, for example, a missing, deficient, and/or mutant protein or enzyme).

As known to the art, antibodies (Abs) can mitigate AAV infection through multiple mechanisms by binding to AAV capsids and blocking critical steps in transduction such as cell surface attachment and uptake, endosomal escape, productive trafficking to the nucleus, or uncoating as well as promoting AAV opsonization by phagocytic cells, thereby mediating their rapid clearance from the circulation. For example, in humans, serological studies reveal a high prevalence of NAbs in the worldwide population, with about 67% of people having antibodies against AAV1, 72% against AAV2, and approximately 40% against AAV serotypes 5 through 9. Vector immunogenicity represents a major challenge in re-administration of AAV vectors.

In an aspect, also disclosed herein are partial self-complementary parvovirus (e.g., a disclosed AAV) genomes, plasmid vectors encoding the parvovirus genomes, and parvovirus (e.g., a disclosed AAV) particles including such genomes. In an aspect, provided herein is a plasmid vector comprising a nucleotide sequence encoding a disclosed parvovirus genome such as for example, a disclosed AAV. In an aspect, provided herein is a partial self-complementary parvovirus genome including a payload construct, parvovirus ITRs flanking the payload construct, and a self-complementary region flanking one of the ITRs. A self-complementary region can comprise a nucleotide sequence that is complementary to the payload construct. A disclosed self-complementary region can have a length that is less the entire length of the payload construct.

In an aspect, a disclosed self-complementary region of a disclosed parvovirus genome can comprise a minimum length, while still having a length that is less the entire length of the payload construct. In an aspect, a disclosed self-complementary region can comprise at least 50 bases in length, at least 100 bases in length, at least 200 in length, at least 300 bases in length, at least 400 bases in length, at least 500 bases in length, at least 600 bases in length, at least 700 bases in length, at least 800 bases in length, at least 900 bases in length, or at least 1,000 bases in length.

In an aspect, a “self-complementary parvovirus genome” can be a single stranded polynucleotide having, in the 5′ to 3′ direction, a first parvovirus ITR sequence, a heterologous sequence (e.g., payload construct comprising, for example, a desired gene), a second parvovirus ITR sequence, a second heterologous sequence, wherein the second heterologous sequence is complementary to the first heterologous sequence, and a third parvovirus ITR sequence. In contrast to a self-complementary genome, a “partial self-complementary genome” does not include three parvovirus ITRs and the second heterologous sequence that is complementary to the first heterologous sequence has a length that is less than the entire length of the first heterologous sequence (e.g., payload construct). Accordingly, a partial self-complementary genome is a single stranded polynucleotide having, in the 5′ to 3′ direction or the 3′ to 5′ direction, a first parvovirus ITR sequence, a heterologous sequence (e.g., payload construct), a second parvovirus ITR sequence, and a self-complementary region that is complementary to a portion of the heterologous sequence and has a length that is less than the entire length the heterologous sequence.

In an aspect, “immune-modulating” refers to the ability of a disclosed isolated nucleic acid molecules, a disclosed vector, a disclosed pharmaceutical formulation, or a disclosed agent to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

In an aspect, “immune modulator” refers to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions (e.g., antigen specific Treg cells to AAV). In an aspect, a disclosed immune modulator can be bortezomib or SVP-Rapamycin. In an aspect, an immune modulator can be administered by any suitable route of administration including, but not limited to, in utero, intra-CSF, intrathecally, intravenously, subcutaneously, transdermally, intradermally, intramuscularly, orally, transcutaneously, intraperitoneally (IP), or intravaginally. In an aspect, a disclosed immune modulator can be administered using a combination of routes. Administration can also include hepatic intra-arterial administration or administration through the hepatic portal vein (HPV). Administration of an immune modulator can be continuous or intermittent, and administration can comprise a combination of one or more routes.

In an aspect, the term “immunotolerant” refers to unresponsiveness to an antigen (e.g., a vector, a therapeutic protein, a transgene product, etc.). An immunotolerant promoter can reduce, ameliorate, or prevent transgene-induced immune responses that can be associated with gene therapy. Assays known in the art to measure immune responses, such as immunohistochemical detection of cytotoxic T cell responses, can be used to determine whether one or more promoters can confer immunotolerant properties.

In an aspect, the term “package insert” is used to refer to instructions customarily included in commercial packages of therapeutic products, that contain information about the indications, usage, dosage, administration, contraindications and/or warnings concerning the use of such therapeutic products.

In an aspect, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed isolated nucleic acid molecule, a disclosed vector, a disclosed pharmaceutical formulation, or a combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., agent) to a subject having or diagnosed with a disease or disorder (such as a genetic disease or disorder).

Disclosed are the components to be used to prepare the disclosed isolated nucleic acid molecules, disclosed vectors, or disclosed pharmaceutical formulations as well as the disclosed isolated nucleic acid molecules, disclosed vectors, or disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Glycogen Storage Diseases (GSDs)

Glycogen storage disease type Ia (GSD Ia) is the inherited deficiency of glucose-6-phosphatase (G6Pase) caused by pathogenic mutations in the G6PC gene. GSD Ia is associated with life-threatening hypoglycemia, as well as longer term adverse effects including hepatocellularadenoma (HCA) and hepatocellular carcinoma (HCC). While dietary therapy with uncooked cornstarch has succeeded in prolonging the lifespan of people with this condition, it fails to reliably prevent long-term complications of GSD Ia, including short stature, progressive renal failure, HCA, and, less frequently, HCC. The liver and kidney involvement has been attributed to accumulations of glycogen and lipids in these tissues that persists despite dietary therapy. Treatments for HCA and HCC have included resection or live transplantation, and transplantation corrected the biochemical abnormalities associated with GSD Ia, including hypoglycemia and hyperlipidemia.

GSD II, also known as Pompe disease, is caused by impairment in the lysosomal enzyme acid α-glucosidase (GAA). It is classified as two general subtypes, infantile and late onset, with a spectrum of disease involvement in between. Infantile Pompe disease (IPD) is rapidly progressive; patients with IPD typically present in the first few months of life with severe hypotonia, respiratory distress and hypertrophic cardiomyopathy, among other complications. Late-onset Pompe disease (LOPD) is typically slowly progressive primarily involving skeletal muscle without severe cardiomyopathy and encompasses those presenting later than infancy as well as the adult-onset form of the disease.

Enzyme replacement therapy (ERT) with recombinant human GAA (rhGAA, Myozyme and Lumizyme) is the only currently approved treatment for Pompe disease. The advent of ERT has significantly prolonged survival and improved clinical outcomes in patients with Pompe disease, especially in the infantile form, which was previously known to be fatal by the second year of life when untreated. Despite treatment with ERT, however, here remains a multitude of persistent complications that significantly limit clinical outcomes.

### C. Compositions for Gene Editing

### 1. Promoters

a. G6PC Promoters

Disclosed herein is a nucleic acid sequence for a G6PC minimal promoter. Disclosed herein is a nucleic acid sequence for a human G6PC minimal promoter. Disclosed herein is a nucleic acid sequence for a non-human G6PC minimal promoter. In an aspect, a non-human G6PC minimal promoter can comprise a mouse promoter.

Disclosed herein is a nucleic acid sequence for a promoter comprising the sequence set forth in SEQ ID NO:02 or SEQ ID NO:03. Disclosed herein is a nucleic acid sequence for a promoter comprising a fragment of the sequence set forth in SEQ ID NO:02 or SEQ ID NO:03. Disclosed herein is a nucleic acid sequence for a promoter having at least about 75%, at least about 80%, at least about 85%, at least about 90%, at least about 95%, or more than at least 95% identity to the sequence set forth in SEQ ID NO:02 or SEQ ID NO:03.

Disclosed herein is a nucleic acid sequence for a promoter that can be incorporated into an AAV vector comprising a nucleic acid sequence encoding a Cas9 endonuclease. Disclosed herein is a nucleic acid sequence for a promoter comprising the sequence set forth in SEQ ID NO:02 or SEQ ID NO:03 and that can be incorporated into an AAV vector comprising a nucleic acid sequence encoding a Cas9 endonuclease. Disclosed herein is a nucleic acid sequence for a promoter comprising a fragment of the sequence set forth in SEQ ID NO:02 or SEQ ID NO:03 and that can be incorporated into an AAV vector comprising a nucleic acid sequence encoding a Cas9 endonuclease. Disclosed herein is a nucleic acid sequence for a promoter having at least about 75%, at least about 80%, at least about 85%, at least about 90%, at least about 95%, or more than at least 95% identity to the sequence set forth in SEQ ID NO:02 or SEQ ID NO:03 and that can be incorporated into an AAV vector comprising a nucleic acid sequence encoding a Cas9 endonuclease.

Disclosed herein is a nucleic acid sequence for a promoter that can be incorporated into an AAV vector comprising a nucleic acid sequence for a transgene or cDNA. Disclosed herein is a nucleic acid sequence for a promoter comprising the sequence set forth in SEQ ID NO:02 or SEQ ID NO:03 and that can be incorporated into an AAV vector comprising a nucleic acid sequence for a transgene or cDNA. Disclosed herein is a nucleic acid sequence for a promoter comprising a fragment of the sequence set forth in SEQ ID NO:02 or SEQ ID NO:03 and that can be incorporated into an AAV vector comprising a nucleic acid sequence for a transgene or cDNA. Disclosed herein is a nucleic acid sequence for a promoter having at least about 75%, at least about 80%, at least about 85%, at least about 90%, at least about 95%, or more than at least 95% identity to the sequence set forth in SEQ ID NO:02 or SEQ ID NO:03 and that can be incorporated into an AAV vector comprising a nucleic acid sequence for a transgene or cDNA.

In an aspect, a disclosed transgene can comprise GAA. GAA is known to the art as evidenced by the genomic sequence set forth in SEQ ID NO:29, the mRNA sequence set forth in SEQ ID NO:30, and the protein sequence set forth in SEQ ID NO:31. In an aspect, a disclosed GAA transgene can comprise the sequence set forth in any one of SEQ ID NO:32-SEQ ID NO:36. In an aspect, a disclosed GAA transgene can be a repair template or a fragment of the full coding sequence and can comprise the sequence set forth in any one of SEQ ID NO:37-SEQ ID NO:40. The art is also familiar with homologs including murine homologs as evidenced by the sequences set forth in SEQ ID NO:26-SEQ ID NO:28.

In an aspect, a disclosed transgene can comprise 6GPC. 6GPC is known to the art as evidenced by the genomic sequence set forth in SEQ ID NO:17, the mRNA sequence set forth in SEQ ID NO:18, and the protein sequence set forth in SEQ ID NO:19. In an aspect, a disclosed G6PC transgene can comprise the sequence set forth in any one of SEQ ID NO:20, SEQ ID NO:22, SEQ ID NO:23, SEQ ID NO:25, or a fragment thereof. The art is also familiar with homologs including murine homologs. In an aspect, a disclosed encoded G6PC transgene can comprise the sequence of SEQ ID NO:21 or SEQ ID NO:24 or a fragment thereof.

Disclosed herein is a nucleic acid sequence for a promoter that can preferentially drive expression of one or more gene products in kidney tissue, heart tissue, skeletal muscle tissue, liver tissue, or any combination thereof.

Disclosed herein is a nucleic acid sequence for a promoter comprising the sequence set forth in SEQ ID NO:02 or SEQ ID NO:03 and that can preferentially drive expression of one or more gene products in kidney tissue, heart tissue, skeletal muscle tissue, liver tissue, or any combination thereof. Disclosed herein is a nucleic acid sequence for a promoter comprising a fragment of the sequence set forth in SEQ ID NO:02 or SEQ ID NO:03 and that can preferentially drive expression of one or more gene products in kidney tissue, heart tissue, skeletal muscle tissue, liver tissue, or any combination thereof. Disclosed herein is a nucleic acid sequence for a promoter having at least about 75%, at least about 80%, at least about 85%, at least about 90%, at least about 95%, or more than at least 95% identity to the sequence set forth in SEQ ID NO:02 or SEQ ID NO:03 and that can preferentially drive expression of one or more gene products in kidney tissue, heart tissue, skeletal muscle tissue, liver tissue, or any combination thereof.

Disclosed herein is a nucleic acid sequence for a promoter that can preferentially drive expression of one or more gene products in kidney tissue, heart tissue, skeletal muscle tissue, liver tissue, or any combination thereof, wherein the expression of one or more gene products can be used in a gene editing application. Disclosed herein is a nucleic acid sequence for a promoter comprising the sequence set forth in SEQ ID NO:02 or SEQ ID NO:03 and that can preferentially drive expression of one or more gene products in kidney tissue, heart tissue, skeletal muscle tissue, liver tissue, or any combination thereof, wherein the expression of one or more gene products can be used in a gene editing application. Disclosed herein is a nucleic acid sequence for a promoter comprising a fragment of the sequence set forth in SEQ ID NO:02 or SEQ ID NO:03 and that can preferentially drive expression of one or more gene products in kidney tissue, heart tissue, skeletal muscle tissue, liver tissue, or any combination thereof, wherein the expression of one or more gene products can be used in a gene editing application. Disclosed herein is a nucleic acid sequence for a promoter having at least about 75%, at least about 80%, at least about 85%, at least about 90%, at least about 95%, or more than at least 95% identity to the sequence set forth in SEQ ID NO:02 or SEQ ID NO:03 and that can preferentially drive expression of one or more gene products in kidney tissue, heart tissue, skeletal muscle tissue, liver tissue, or any combination thereof, wherein the expression of one or more gene products can be used in a gene editing application.

Disclosed herein is a nucleic acid sequence for a promoter that can preferentially drive expression of one or more gene products in kidney tissue, heart tissue, skeletal muscle tissue, liver tissue, or any combination thereof, wherein the expression of one or more gene products can be used in a gene therapy application. Disclosed herein is a nucleic acid sequence for a promoter comprising the sequence set forth in SEQ ID NO:02 or SEQ ID NO:03 and that can preferentially drive expression of one or more gene products in kidney tissue, heart tissue, skeletal muscle tissue, liver tissue, or any combination thereof, wherein the expression of one or more gene products can be used in a gene therapy application. Disclosed herein is a nucleic acid sequence for a promoter comprising a fragment of the sequence set forth in SEQ ID NO:02 or SEQ ID NO:03 and that can preferentially drive expression of one or more gene products in kidney tissue, heart tissue, skeletal muscle tissue, liver tissue, or any combination thereof, wherein the expression of one or more gene products can be used in a gene therapy application. Disclosed herein is a nucleic acid sequence for a promoter having at least about 75%, at least about 80%, at least about 85%, at least about 90%, at least about 95%, or more than at least 95% identity to the sequence set forth in SEQ ID NO:02 or SEQ ID NO:03 and that can preferentially drive expression of one or more gene products in kidney tissue, heart tissue, skeletal muscle tissue, liver tissue, or any combination thereof, wherein the expression of one or more gene products can be used in a gene therapy application.

Disclosed herein is a nucleic acid sequence for a promoter that can preferentially drive expression of one or more gene products in kidney tissue, heart tissue, skeletal muscle tissue, liver tissue, or any combination thereof in a patient having a glycogen storage disease. Disclosed herein is a nucleic acid sequence for a promoter comprising the sequence set forth in SEQ ID NO:02 or SEQ ID NO:03 and that can preferentially drive expression of one or more gene products in kidney tissue, heart tissue, skeletal muscle tissue, liver tissue, or any combination thereof in a patient having a glycogen storage disease. Disclosed herein is a nucleic acid sequence for a promoter comprising a fragment of the sequence set forth in SEQ ID NO:02 or SEQ ID NO:03 and that can preferentially drive expression of one or more gene products in kidney tissue, heart tissue, skeletal muscle tissue, liver tissue, or any combination thereof in a patient having a glycogen storage disease. Disclosed herein is a nucleic acid sequence for a promoter having at least about 75%, at least about 80%, at least about 85%, at least about 90%, at least about 95%, or more than at least 95% identity to the sequence set forth in SEQ ID NO:02 or SEQ ID NO:03 and that can preferentially drive expression of one or more gene products in kidney tissue, heart tissue, skeletal muscle tissue, liver tissue, or any combination thereof in a patient having a glycogen storage disease.

Disclosed herein is a nucleic acid sequence for a promoter that can preferentially drive expression of one or more gene products in kidney tissue, heart tissue, skeletal muscle tissue, liver tissue, or any combination thereof in a patient having GSDIa or GSDII. Disclosed herein is a nucleic acid sequence for a promoter comprising the sequence set forth in SEQ ID NO:02 or SEQ ID NO:03 and that can preferentially drive expression of one or more gene products in kidney tissue, heart tissue, skeletal muscle tissue, liver tissue, or any combination thereof in a patient having GSDIa or GSDII. Disclosed herein is a nucleic acid sequence for a promoter comprising a fragment of the sequence set forth in SEQ ID NO:02 or SEQ ID NO:03 and that can preferentially drive expression of one or more gene products in kidney tissue, heart tissue, skeletal muscle tissue, liver tissue, or any combination thereof in a patient having GSDIa or GSDII. Disclosed herein is a nucleic acid sequence for a promoter having at least about 75%, at least about 80%, at least about 85%, at least about 90%, at least about 95%, or more than at least 95% identity to the sequence set forth in SEQ ID NO:02 or SEQ ID NO:03 and that can preferentially drive expression of one or more gene products in kidney tissue, heart tissue, skeletal muscle tissue, liver tissue, or any combination thereof in a patient having GSDIa or GSDII.

Disclosed herein is a nucleic acid sequence for a promoter that can preferentially drive expression of one or more gene products in kidney tissue, heart tissue, skeletal muscle tissue, liver tissue, or any combination thereof in a patient having genetic deficiency or genetic disorder. Disclosed herein is a nucleic acid sequence for a promoter comprising a fragment of the sequence set forth in SEQ ID NO:02 or SEQ ID NO:03 and that can preferentially drive expression of one or more gene products in kidney tissue, heart tissue, skeletal muscle tissue, liver tissue, or any combination thereof in a patient having genetic deficiency or genetic disorder. Disclosed herein is a nucleic acid sequence for a promoter comprising the sequence set forth in SEQ ID NO:02 or SEQ ID NO:03 and that can preferentially drive expression of one or more gene products in kidney tissue, heart tissue, skeletal muscle tissue, liver tissue, or any combination thereof in a patient having genetic deficiency or genetic disorder. Disclosed herein is a nucleic acid sequence for a promoter having at least about 75%, at least about 80%, at least about 85%, at least about 90%, at least about 95%, or more than at least 95% identity to the sequence set forth in SEQ ID NO:02 or SEQ ID NO:03 and that can preferentially drive expression of one or more gene products in kidney tissue, heart tissue, skeletal muscle tissue, liver tissue, or any combination thereof in a patient having genetic deficiency or genetic disorder.

Disclosed herein is a nucleic acid sequence for a promoter that can preferentially drive expression of one or more gene products in kidney tissue, heart tissue, skeletal muscle tissue, liver tissue, or any combination thereof in a patient having inherited protein deficiency or inherited protein disorder. Disclosed herein is a nucleic acid sequence for a promoter comprising the sequence set forth in SEQ ID NO:02 or SEQ ID NO:03 and that can preferentially drive expression of one or more gene products in kidney tissue, heart tissue, skeletal muscle tissue, liver tissue, or any combination thereof in a patient having inherited protein deficiency or inherited protein disorder. Disclosed herein is a nucleic acid sequence for a promoter comprising a fragment of the sequence set forth in SEQ ID NO:02 or SEQ ID NO:03 and that can preferentially drive expression of one or more gene products in kidney tissue, heart tissue, skeletal muscle tissue, liver tissue, or any combination thereof in a patient having inherited protein deficiency or inherited protein disorder. Disclosed herein is a nucleic acid sequence for a promoter having at least about 75%, at least about 80%, at least about 85%, at least about 90%, at least about 95%, or more than at least 95% identity to the sequence set forth in SEQ ID NO:02 or SEQ ID NO:03 and that can preferentially drive expression of one or more gene products in kidney tissue, heart tissue, skeletal muscle tissue, liver tissue, or any combination thereof in a patient having inherited protein deficiency or inherited protein disorder.

Disclosed herein is a nucleic acid sequence for a promoter that can preferentially drive expression of one or more gene products in kidney tissue, heart tissue, skeletal muscle tissue, liver tissue, or any combination thereof in a patient having alpha-1-antitrypsin deficiency, phenylketonuria, mitochondrial trifunctional protein deficiency, hemophilia, or muscle dystrophy. Disclosed herein is a nucleic acid sequence for a promoter comprising the sequence set forth in SEQ ID NO:02 or SEQ ID NO:03 and that can preferentially drive expression of one or more gene products in kidney tissue, heart tissue, skeletal muscle tissue, liver tissue, or any combination thereof in a patient having alpha-1-antitrypsin deficiency, phenylketonuria, mitochondrial trifunctional protein deficiency, hemophilia, or muscle dystrophy. Disclosed herein is a nucleic acid sequence for a promoter comprising a fragment of the sequence set forth in SEQ ID NO:02 or SEQ ID NO:03 and that can preferentially drive expression of one or more gene products in kidney tissue, heart tissue, skeletal muscle tissue, liver tissue, or any combination thereof in a patient having alpha-1-antitrypsin deficiency, phenylketonuria, mitochondrial trifunctional protein deficiency, hemophilia, or muscle dystrophy. Disclosed herein is a nucleic acid sequence for a promoter having at least about 75%, at least about 80%, at least about 85%, at least about 90%, at least about 95%, or more than at least 95% identity to the sequence set forth in SEQ ID NO:02 or SEQ ID NO:03 and that can preferentially drive expression of one or more gene products in kidney tissue, heart tissue, skeletal muscle tissue, liver tissue, or any combination thereof in a patient having alpha-1-antitrypsin deficiency, phenylketonuria, mitochondrial trifunctional protein deficiency, hemophilia, or muscle dystrophy.

In an aspect, a disclosed promoter can comprise a G6PC min promoter. In an aspect, a disclosed G6PC min promoter can comprise the sequence set forth in SEQ ID NO:02 or SEQ ID NO:03. In an aspect, a disclosed G6PC min promoter can comprise a fragment of sequence set forth in SEQ ID NO:02 or SEQ ID NO:03. In an aspect, a disclosed G6PC min promoter can have at least about 75%, at least about 80%, at least about 85%, at least about 90%, at least about 95%, or more than at least 95% identity to the sequence set forth in SEQ ID NO:02 or SEQ ID NO:03.

In an aspect, a disclosed promoter can be part of a hybrid promoter. In an aspect, a disclosed hybrid promoter can comprise a human skeletal muscle-specific transcriptional cis-regulatory module. In an aspect, a disclosed hybrid promoter can comprise a human skeletal muscle-specific enhancer. In an aspect, a disclosed human skeletal muscle-specific transcriptional cis-regulatory module or enhancer can comprise Sk-CRM4. In an aspect, a disclosed Sk-CRM4 can comprise the sequence set forth in SEQ ID NO:04 or SEQ ID NO:05. In an aspect, a disclosed Sk-CRM4 can comprise a fragment of the sequence set forth in SEQ ID NO:04 or SEQ ID NO:05. In an aspect, a disclosed Sk-CRM4 can comprise a sequence having at least about 75%, at least about 80%, at least about 85%, at least about 90%, at least about 95%, or more than at least 95% identity to the sequence set forth in SEQ ID NO:04 or SEQ ID NO:05.

In an aspect, a disclosed promoter comprising (i) the sequence set forth in SEQ ID NO:02 or SEQ ID NO:03, (ii) a fragment of the sequence set forth in SEQ ID NO:02 or SEQ ID NO:03, or (iii) at least about 75%, at least about 80%, at least about 85%, at least about 90%, at least about 95%, or more than at least 95% identity to the sequence set forth in SEQ ID NO:02 or SEQ ID NO:03 can be used in a disclosed method. In an aspect, a disclosed promoter comprising (i) the sequence set forth in SEQ ID NO:02 or SEQ ID NO:03, (ii) a fragment of the sequence set forth in SEQ ID NO:02 or SEQ ID NO:03, or (iii) at least about 75%, at least about 80%, at least about 85%, at least about 90%, at least about 95%, or more than at least 95% identity to the sequence set forth in SEQ ID NO:02 or SEQ ID NO:03 can be used to drive the expression of a disclosed transgene or a disclosed cDNA in a disclosed method.

In an aspect, a disclosed promoter comprising (i) the sequence set forth in SEQ ID NO:02 or SEQ ID NO:03, (ii) a fragment of the sequence set forth in SEQ ID NO:02 or SEQ ID NO:03, or (iii) at least about 75%, at least about 80%, at least about 85%, at least about 90%, at least about 95%, or more than at least 95% identity to the sequence set forth in SEQ ID NO:02 or SEQ ID NO:03 can be used to generate a depot of the therapeutic expression of a transgene.

In an aspect, a disclosed promoter comprising (i) the sequence set forth in SEQ ID NO:02 or SEQ ID NO:03, (ii) a fragment of the sequence set forth in SEQ ID NO:02 or SEQ ID NO:03, or (iii) at least about 75%, at least about 80%, at least about 85%, at least about 90%, at least about 95%, or more than at least 95% identity to the sequence set forth in SEQ ID NO:02 or SEQ ID NO:03 can be used to generate a depot of the therapeutic expression of a transgene in a subject's liver and/or in a subject's skeletal muscle.

In an aspect, a disclosed promoter comprising (i) the sequence set forth in SEQ ID NO:02 or SEQ ID NO:03, (ii) a fragment of the sequence set forth in SEQ ID NO:02 or SEQ ID NO:03, or (iii) at least about 75%, at least about 80%, at least about 85%, at least about 90%, at least about 95%, or more than at least 95% identity to the sequence set forth in SEQ ID NO:02 or SEQ ID NO:03 can be used in a disclosed method of repairing a defective gene (e.g., GAA or G6PC) can restore the functionality and/or structural integrity of a missing, deficient, and/or mutant protein or enzyme (e.g., GAA or G6PC).

In an aspect, a disclosed method can comprise restoring one or more aspects of cellular homeostasis and/or cellular functionality and/or metabolic dysregulation (such as, for example, glycogen accumulation). In an aspect, restoring one or more aspect of cellular homeostasis and/or cellular functionality and/or metabolic dysregulation comprises restoring the functionality and/or structural integrity of a missing, deficient, and/or mutant protein or enzyme (such as, for example, G6PC or GAA).

In an aspect, restoring one or more aspects of cellular homeostasis and/or cellular functionality can comprise one or more of the following: (i) correcting cell starvation in one or more cell types; (ii) improving, enhancing, restoring, and/or preserving mitochondrial functionality and/or structural integrity; (iii) improving, enhancing, restoring, and/or preserving organelle functionality and/or structural integrity; (iv) correcting enzyme dysregulation; (v) reversing, inhibiting, preventing, stabilizing, and/or slowing the rate of progression of the multi-systemic manifestations of a genetic disease or disorder; (vi) reversing, inhibiting, preventing, stabilizing, and/or slowing the rate of progression of a genetic disease or disorder, (vii) normalizing aspects of the autophagy pathway (such as, for example, correcting, preventing, reducing, and/or ameliorating autophagy), or (viii) any combination thereof. In an aspect, restoring one or more aspects of cellular homeostasis can comprise improving, enhancing, restoring, and/or preserving one or more aspects of cellular structural and/or functional integrity.

In an aspect, a disclosed promoter comprising (i) the sequence set forth in SEQ ID NO:02 or SEQ ID NO:03, (ii) a fragment of the sequence set forth in SEQ ID NO:02 or SEQ ID NO:03, or (iii) at least about 75%, at least about 80%, at least about 85%, at least about 90%, at least about 95%, or more than at least 95% identity to the sequence set forth in SEQ ID NO:02 or SEQ ID NO:03 can be used to preferentially drive expression of one or more gene products in kidney tissue, heart tissue, skeletal muscle tissue, liver tissue, or any combination thereof.

In an aspect, a disclosed promoter comprising (i) the sequence set forth in SEQ ID NO:02 or SEQ ID NO:03, (ii) a fragment of the sequence set forth in SEQ ID NO:02 or SEQ ID NO:03, or (iii) at least about 75%, at least about 80%, at least about 85%, at least about 90%, at least about 95%, or more than at least 95% identity to the sequence set forth in SEQ ID NO:02 or SEQ ID NO:03 can be used increase (protein or gene) expression of GAA or G6PC in kidney tissue, heart tissue, skeletal muscle tissue, liver tissue, or any combination thereof.

In an aspect, a disclosed promoter comprising (i) the sequence set forth in SEQ ID NO:02 or SEQ ID NO:03, (ii) a fragment of the sequence set forth in SEQ ID NO:02 or SEQ ID NO:03, or (iii) at least about 75%, at least about 80%, at least about 85%, at least about 90%, at least about 95%, or more than at least 95% identity to the sequence set forth in SEQ ID NO:02 or SEQ ID NO:03 can be used to decrease glycogen content in kidney tissue, heart tissue, skeletal muscle tissue, liver tissue, or any combination thereof.

In an aspect, a disclosed promoter comprising (i) the sequence set forth in SEQ ID NO:02 or SEQ ID NO:03, (ii) a fragment of the sequence set forth in SEQ ID NO:02 or SEQ ID NO:03, or (iii) at least about 75%, at least about 80%, at least about 85%, at least about 90%, at least about 95%, or more than at least 95% identity to the sequence set forth in SEQ ID NO:02 or SEQ ID NO:03 can be used to decrease the level of triglycerides in kidney tissue, heart tissue, skeletal muscle tissue, liver tissue, or any combination thereof.

In an aspect, a disclosed promoter comprising (i) the sequence set forth in SEQ ID NO:02 or SEQ ID NO:03, (ii) a fragment of the sequence set forth in SEQ ID NO:02 or SEQ ID NO:03, or (iii) at least about 75%, at least about 80%, at least about 85%, at least about 90%, at least about 95%, or more than at least 95% identity to the sequence set forth in SEQ ID NO:02 or SEQ ID NO:03 can be used to improve hepatocyte morphology and/or to decrease hepatocyte vacuolation.

In an aspect, a disclosed promoter comprising (i) the sequence set forth in SEQ ID NO:02 or SEQ ID NO:03, (ii) a fragment of the sequence set forth in SEQ ID NO:02 or SEQ ID NO:03, or (iii) at least about 75%, at least about 80%, at least about 85%, at least about 90%, at least about 95%, or more than at least 95% identity to the sequence set forth in SEQ ID NO:02 or SEQ ID NO:03 can be used to restore normal blood glucose levels in a patient or a subject in need thereof.

In an aspect, a disclosed promoter comprising (i) the sequence set forth in SEQ ID NO:02 or SEQ ID NO:03, (ii) a fragment of the sequence set forth in SEQ ID NO:02 or SEQ ID NO:03, or (iii) at least about 75%, at least about 80%, at least about 85%, at least about 90%, at least about 95%, or more than at least 95% identity to the sequence set forth in SEQ ID NO:02 or SEQ ID NO:03 can be used to reduce the subject's or patient's risk of developing long-term adverse effects.

In an aspect, a disclosed promoter comprising (i) the sequence set forth in SEQ ID NO:02 or SEQ ID NO:03, (ii) a fragment of the sequence set forth in SEQ ID NO:02 or SEQ ID NO:03, or (iii) at least about 75%, at least about 80%, at least about 85%, at least about 90%, at least about 95%, or more than at least 95% identity to the sequence set forth in SEQ ID NO:02 or SEQ ID NO:03 can be used to increase survival of a patient or a subject in need thereof.

In an aspect, a disclosed promoter comprising (i) the sequence set forth in SEQ ID NO:02 or SEQ ID NO:03, (ii) a fragment of the sequence set forth in SEQ ID NO:02 or SEQ ID NO:03, or (iii) at least about 75%, at least about 80%, at least about 85%, at least about 90%, at least about 95%, or more than at least 95% identity to the sequence set forth in SEQ ID NO:02 or SEQ ID NO:03 can be used to restore the functionality and/or structural integrity of a missing, deficient, and/or mutant protein or enzyme (such as, for example, G6PC or GAA).

In an aspect, a disclosed promoter comprising (i) the sequence set forth in SEQ ID NO:02 or SEQ ID NO:03, (ii) a fragment of the sequence set forth in SEQ ID NO:02 or SEQ ID NO:03, or (iii) at least about 75%, at least about 80%, at least about 85%, at least about 90%, at least about 95%, or more than at least 95% identity to the sequence set forth in SEQ ID NO:02 or SEQ ID NO:03 can be used to restore one or more aspects of cellular homeostasis and/or cellular functionality and/or metabolic dysregulation (such as, for example, glycogen accumulation). In an aspect, restoring one or more aspect of cellular homeostasis and/or cellular functionality and/or metabolic dysregulation comprises restoring the functionality and/or structural integrity of a missing, deficient, and/or mutant protein or enzyme (such as, for example, GAA or G6PC).

In an aspect, restoring one or more aspects of cellular homeostasis and/or cellular functionality can comprise one or more of the following: (i) correcting cell starvation in one or more cell types; (ii) improving, enhancing, restoring, and/or preserving mitochondrial functionality and/or structural integrity; (iii) improving, enhancing, restoring, and/or preserving organelle functionality and/or structural integrity; (iv) correcting enzyme dysregulation; (v) reversing, inhibiting, preventing, stabilizing, and/or slowing the rate of progression of the multi-systemic manifestations of a genetic disease or disorder; (vi) reversing, inhibiting, preventing, stabilizing, and/or slowing the rate of progression of a genetic disease or disorder, (vii) normalizing aspects of the autophagy pathway (such as, for example, correcting, preventing, reducing, and/or ameliorating autophagy), or (viii) any combination thereof. In an aspect, restoring one or more aspects of cellular homeostasis can comprise improving, enhancing, restoring, and/or preserving one or more aspects of cellular structural and/or functional integrity.

In an aspect, a disclosed G6PC promoter can drive supraphysiologic GAA expression in a subject's liver and/or a subject's skeletal muscle. In an aspect, a disclosed G6PC promoter can drive supraphysiologic G6PC expression in a subject's liver and/or a subject's skeletal muscle. In an aspect, a disclosed G6PC promoter can restore normal glycogen content in a subject's heart, diaphragm, quadriceps, or any combination thereof. In an aspect, a disclosed G6PC promoter can improve a subject's muscle strength.

In an aspect, a disclosed G6PC promoter can drive supraphysiologic expression of a gene having one or more pathogenetic defects in a subject's liver and/or a subject's skeletal muscle. In an aspect, a disclosed G6PC promoter can restore normal gene expression in a subject's heart, diaphragm, quadriceps, or any combination thereof. In an aspect, a disclosed G6PC promoter can improve a subject's muscle strength.

In an aspect, a disclosed promoter can ensure persistent transgene expression.

b. Hybrid Promoters

Disclosed herein is a hybrid promoter. Disclosed herein is a hybrid promoter comprising a G6PC min promoter and a human skeletal muscle-specific transcriptional cis-regulatory module. Disclosed herein is a hybrid promoter comprising a G6PC min promoter and a human skeletal muscle-specific enhancer. Disclosed herein is a hybrid promoter comprising a G6PC min promoter and a hSk-CRM4 regulatory module. In an aspect of a disclosed hybrid promoter, a disclosed G6PC min promoter can comprise the sequence set forth in SEQ ID NO:02 or SEQ ID NO:03. In an aspect of a disclosed hybrid promoter, a disclosed G6PC min promoter can comprise a fragment of the sequence set forth in SEQ ID NO:02 or SEQ ID NO:03. In an aspect of a disclosed hybrid promoter, a disclosed G6PC min promoter can comprise a sequence having at least about 75%, at least about 80%, at least about 85%, at least about 90%, at least about 95%, or more than at least 95% identity to the sequence set forth in SEQ ID NO:02 or SEQ ID NO:03.

In an aspect of a disclosed hybrid promoter, a disclosed human skeletal muscle-specific transcriptional cis-regulatory module or enhancer can comprise Sk-CRM4. In an aspect of a disclosed hybrid promoter, a disclosed Sk-CRM4 regulatory module can comprise the sequence set forth in SEQ ID NO:04 or SEQ ID NO:05. In an aspect of a disclosed hybrid promoter, a disclosed Sk-CRM4 regulatory module can comprise a fragment of the sequence set forth in SEQ ID NO:04 or SEQ ID NO:05. In an aspect of a disclosed hybrid promoter, a disclosed Sk-CRM4 regulatory module can comprise a sequence having at least about 75%, at least about 80%, at least about 85%, at least about 90%, at least about 95%, or more than at least 95% identity to the sequence set forth in SEQ ID NO:04 or SEQ ID NO:05,

Disclosed herein is a hybrid promoter comprising a hSk-CRM4 regulatory module and a G6PC min promoter. Disclosed herein is a hybrid promoter comprising a hSk-CRM4 regulatory module and a G6PC min promoter. Disclosed herein is a hybrid promoter comprising a G6PC min promoter and a hSk-CRM4 regulatory module. Disclosed herein is a hybrid promoter comprising a G6PC min promoter and a hSk-CRM4 regulatory module separated by a spacer or a linker. In an aspect, a disclosed spacer or linker can comprise the sequence set forth in SEQ ID NO:41,

Disclosed herein is a nucleic acid sequence for a hybrid promoter comprising the sequence set forth in any one of SEQ ID NO:06-SEQ ID NO:09. Disclosed herein is a nucleic acid sequence for a hybrid promoter comprising a fragment of the sequence set forth in any one of SEQ ID NO:06-SEQ ID NO:09. Disclosed herein is a nucleic acid sequence for a hybrid promoter having at least about 75%, at least about 80%, at least about 85%, at least about 90%, at least about 95%, or more than at least 95% identity to the sequence set forth in any one of SEQ ID NO:06-SEQ ID NO:09.

Disclosed herein is a nucleic acid sequence for a hybrid promoter comprising the sequence set forth in any one of SEQ ID NO:10-SEQ ID NO:13. Disclosed herein is a nucleic acid sequence for a hybrid promoter comprising a fragment of the sequence set forth in any one of SEQ ID NO:10-SEQ ID NO:13. Disclosed herein is a nucleic acid sequence for a hybrid promoter having at least about 75%, at least about 80%, at least about 85%, at least about 90%, at least about 95%, or more than at least 95% identity to the sequence set forth in any one of SEQ ID NO:10-SEQ ID NO:13.

Disclosed herein is a nucleic acid sequence for a hybrid promoter that can be incorporated into an AAV vector comprising a nucleic acid sequence encoding a Cas9 endonuclease.

Disclosed herein is a nucleic acid sequence for a hybrid promoter comprising the sequence set forth in any one of SEQ ID NO:06-SEQ ID NO:13 and that can be incorporated into an AAV vector comprising a nucleic acid sequence encoding a Cas9 endonuclease. Disclosed herein is a nucleic acid sequence for a hybrid promoter comprising a fragment of the sequence set forth in any one of SEQ ID NO:06-SEQ ID NO:13 and that can be incorporated into an AAV vector comprising a nucleic acid sequence encoding a Cas9 endonuclease. Disclosed herein is a nucleic acid sequence for a hybrid promoter having at least about 75%, at least about 80%, at least about 85%, at least about 90%, at least about 95%, or more than at least 95% identity to the sequence set forth in any one of SEQ ID NO:06-SEQ ID NO:13 and that can be incorporated into an AAV vector comprising a nucleic acid sequence encoding a Cas9 endonuclease.

Disclosed herein is a nucleic acid sequence for a hybrid promoter that can be incorporated into an AAV vector comprising a nucleic acid sequence for a transgene or cDNA.

Disclosed herein is a nucleic acid sequence for a hybrid promoter comprising the sequence set forth in any one of SEQ ID NO:06-SEQ ID NO:13 and that can be incorporated into an AAV vector comprising a nucleic acid sequence for a transgene or cDNA. Disclosed herein is a nucleic acid sequence for a hybrid promoter comprising a fragment of the sequence set forth in any one of SEQ ID NO:06-SEQ ID NO:13 and that can be incorporated into an AAV vector comprising a nucleic acid sequence for a transgene or cDNA. Disclosed herein is a nucleic acid sequence for a hybrid promoter having at least about 75%, at least about 80%, at least about 85%, at least about 90%, at least about 95%, or more than at least 95% identity to the sequence set forth in any one of SEQ ID NO:06-SEQ ID NO:13 and that can be incorporated into an AAV vector comprising a nucleic acid sequence for a transgene or cDNA.

In an aspect, a disclosed transgene can comprise GAA. GAA is known to the art as evidenced by the genomic sequence set forth in SEQ ID NO:29, the mRNA sequence set forth in SEQ ID NO:30, and the protein sequence set forth in SEQ ID NO:31. In an aspect, a disclosed GAA transgene can comprise the sequence set forth in any one of SEQ ID NO:32-SEQ ID NO:36. In an aspect, a disclosed GAA transgene can be a repair template or a fragment of the full coding sequence and can comprise the sequence set forth in any one of SEQ ID NO:37-SEQ ID NO:40. The art is also familiar with homologs including murine homologs as evidenced by the sequences set forth in SEQ ID NO:26-SEQ ID NO:28.

In an aspect, a disclosed transgene can comprise 6GPC. 6GPC is known to the art as evidenced by the genomic sequence set forth in SEQ ID NO:17, the mRNA sequence set forth in SEQ ID NO:18, and the protein sequence set forth in SEQ ID NO:19. In an aspect, a disclosed G6PC transgene can comprise the sequence set forth in any one of SEQ ID NO:20, SEQ ID NO:22, SEQ ID NO:23, SEQ ID NO:25, or a fragment thereof. The art is also familiar with homologs including murine homologs. In an aspect, a disclosed encoded G6PC transgene can comprise the sequence of SEQ ID NO:21 or SEQ ID NO:24 or a fragment thereof.

Disclosed herein is a nucleic acid sequence for a hybrid promoter that can preferentially drive expression of one or more gene products in kidney tissue, heart tissue, skeletal muscle tissue, liver tissue, or any combination thereof.

Disclosed herein is a nucleic acid sequence for a hybrid promoter comprising the sequence set forth in any one of SEQ ID NO:06-SEQ ID NO:13 and that can preferentially drive expression of one or more gene products in kidney tissue, heart tissue, skeletal muscle tissue, liver tissue, or any combination thereof. Disclosed herein is a nucleic acid sequence for a hybrid promoter comprising a fragment of the sequence set forth in any one of SEQ ID NO:06-SEQ ID NO:13 and that can preferentially drive expression of one or more gene products in kidney tissue, heart tissue, skeletal muscle tissue, liver tissue, or any combination thereof. Disclosed herein is a nucleic acid sequence for a hybrid promoter having at least about 75%, at least about 80%, at least about 85%, at least about 90%, at least about 95%, or more than at least 95% identity to the sequence set forth in any one of SEQ ID NO:06-SEQ ID NO:13 and that can preferentially drive expression of one or more gene products in kidney tissue, heart tissue, skeletal muscle tissue, liver tissue, or any combination thereof.

Disclosed herein is a nucleic acid sequence for a hybrid promoter that can preferentially drive expression of one or more gene products in kidney tissue, heart tissue, skeletal muscle tissue, liver tissue, or any combination thereof, wherein the expression of one or more gene products can be used in a gene editing application.

Disclosed herein is a nucleic acid sequence for a hybrid promoter comprising the sequence set forth in any one of SEQ ID NO:06-SEQ ID NO:13 and that can preferentially drive expression of one or more gene products in kidney tissue, heart tissue, skeletal muscle tissue, liver tissue, or any combination thereof, wherein the expression of one or more gene products can be used in a gene editing application. Disclosed herein is a nucleic acid sequence for a hybrid promoter comprising a fragment of the sequence set forth in any one of SEQ ID NO:06-SEQ ID NO:13 and that can preferentially drive expression of one or more gene products in kidney tissue, heart tissue, skeletal muscle tissue, liver tissue, or any combination thereof, wherein the expression of one or more gene products can be used in a gene editing application. Disclosed herein is a nucleic acid sequence for a hybrid promoter having at least about 75%, at least about 80%, at least about 85%, at least about 90%, at least about 95%, or more than at least 95% identity to the sequence set forth in any one of SEQ ID NO:06-SEQ ID NO:13 and that can preferentially drive expression of one or more gene products in kidney tissue, heart tissue, skeletal muscle tissue, liver tissue, or any combination thereof, wherein the expression of one or more gene products can be used in a gene editing application.

Disclosed herein is a nucleic acid sequence for a hybrid promoter that can preferentially drive expression of one or more gene products in kidney tissue, heart tissue, skeletal muscle tissue, liver tissue, or any combination thereof, wherein the expression of one or more gene products can be used in a gene therapy application.

Disclosed herein is a nucleic acid sequence for a hybrid promoter comprising the sequence set forth in any one of SEQ ID NO:06-SEQ ID NO:13 and that can preferentially drive expression of one or more gene products in kidney tissue, heart tissue, skeletal muscle tissue, liver tissue, or any combination thereof, wherein the expression of one or more gene products can be used in a gene therapy application. Disclosed herein is a nucleic acid sequence for a hybrid promoter comprising a fragment of the sequence set forth in any one of SEQ ID NO:06-SEQ ID NO:13 and that can preferentially drive expression of one or more gene products in kidney tissue, heart tissue, skeletal muscle tissue, liver tissue, or any combination thereof, wherein the expression of one or more gene products can be used in a gene therapy application. Disclosed herein is a nucleic acid sequence for a hybrid promoter having at least about 75%, at least about 80%, at least about 85%, at least about 90%, at least about 95%, or more than at least 95% identity to the sequence set forth in any one of SEQ ID NO:06-SEQ ID NO:13 and that can preferentially drive expression of one or more gene products in kidney tissue, heart tissue, skeletal muscle tissue, liver tissue, or any combination thereof, wherein the expression of one or more gene products can be used in a gene therapy application.

Disclosed herein is a nucleic acid sequence for a hybrid promoter that can preferentially drive expression of one or more gene products in kidney tissue, heart tissue, skeletal muscle tissue, liver tissue, or any combination thereof in a patient having a glycogen storage disease.

Disclosed herein is a nucleic acid sequence for a hybrid promoter comprising the sequence set forth in any one of SEQ ID NO:06-SEQ ID NO:13 and that can preferentially drive expression of one or more gene products in kidney tissue, heart tissue, skeletal muscle tissue, liver tissue, or any combination thereof in a patient having a glycogen storage disease. Disclosed herein is a nucleic acid sequence for a hybrid promoter comprising a fragment of the sequence set forth in any one of SEQ ID NO:06-SEQ ID NO:13 and that can preferentially drive expression of one or more gene products in kidney tissue, heart tissue, skeletal muscle tissue, liver tissue, or any combination thereof in a patient having a glycogen storage disease. Disclosed herein is a nucleic acid sequence for a hybrid promoter having at least about 75%, at least about 80%, at least about 85%, at least about 90%, at least about 95%, or more than at least 95% identity to the sequence set forth in any one of SEQ ID NO:06-SEQ ID NO:13 and that can preferentially drive expression of one or more gene products in kidney tissue, heart tissue, skeletal muscle tissue, liver tissue, or any combination thereof in a patient having a glycogen storage disease.

Disclosed herein is a nucleic acid sequence for a hybrid promoter that can preferentially drive expression of one or more gene products in kidney tissue, heart tissue, skeletal muscle tissue, liver tissue, or any combination thereof in a patient having GSDIa or GSDII.

Disclosed herein is a nucleic acid sequence for a hybrid promoter comprising the sequence set forth in any one of SEQ ID NO:06-SEQ ID NO:13 and that can preferentially drive expression of one or more gene products in kidney tissue, heart tissue, skeletal muscle tissue, liver tissue, or any combination thereof in a patient having GSDIa or GSDII. Disclosed herein is a nucleic acid sequence for a hybrid promoter comprising a fragment of the sequence set forth in any one of SEQ ID NO:06-SEQ ID NO:13 and that can preferentially drive expression of one or more gene products in kidney tissue, heart tissue, skeletal muscle tissue, liver tissue, or any combination thereof in a patient having GSDIa or GSDII. Disclosed herein is a nucleic acid sequence for a hybrid promoter having at least about 75%, at least about 80%, at least about 85%, at least about 90%, at least about 95%, or more than at least 95% identity to the sequence set forth in any one of SEQ ID NO:06-SEQ ID NO:13 and that can preferentially drive expression of one or more gene products in kidney tissue, heart tissue, skeletal muscle tissue, liver tissue, or any combination thereof in a patient having GSDIa or GSDII.

Disclosed herein is a nucleic acid sequence for a hybrid promoter that can preferentially drive expression of one or more gene products in kidney tissue, heart tissue, skeletal muscle tissue, liver tissue, or any combination thereof in a patient having genetic deficiency or genetic disorder.

Disclosed herein is a nucleic acid sequence for a hybrid promoter comprising a fragment of the sequence set forth in any one of SEQ ID NO:06-SEQ ID NO:13 and that can preferentially drive expression of one or more gene products in kidney tissue, heart tissue, skeletal muscle tissue, liver tissue, or any combination thereof in a patient having genetic deficiency or genetic disorder. Disclosed herein is a nucleic acid sequence for a hybrid promoter comprising the sequence set forth in any one of SEQ ID NO:06-SEQ ID NO:13 and that can preferentially drive expression of one or more gene products in kidney tissue, heart tissue, skeletal muscle tissue, liver tissue, or any combination thereof in a patient having genetic deficiency or genetic disorder. Disclosed herein is a nucleic acid sequence for a hybrid promoter having at least about 75%, at least about 80%, at least about 85%, at least about 90%, at least about 95%, or more than at least 95% identity to the sequence set forth in any one of SEQ ID NO:06-SEQ ID NO:13 and that can preferentially drive expression of one or more gene products in kidney tissue, heart tissue, skeletal muscle tissue, liver tissue, or any combination thereof in a patient having genetic deficiency or genetic disorder.

Disclosed herein is a nucleic acid sequence for a hybrid promoter that can preferentially drive expression of one or more gene products in kidney tissue, heart tissue, skeletal muscle tissue, liver tissue, or any combination thereof in a patient having inherited protein deficiency or inherited protein disorder.

Disclosed herein is a nucleic acid sequence for a hybrid promoter comprising the sequence set forth in any one of SEQ ID NO:06-SEQ ID NO:13 and that can preferentially drive expression of one or more gene products in kidney tissue, heart tissue, skeletal muscle tissue, liver tissue, or any combination thereof in a patient having inherited protein deficiency or inherited protein disorder. Disclosed herein is a nucleic acid sequence for a hybrid promoter comprising a fragment of the sequence set forth in any one of SEQ ID NO:06-SEQ ID NO:13 and that can preferentially drive expression of one or more gene products in kidney tissue, heart tissue, skeletal muscle tissue, liver tissue, or any combination thereof in a patient having inherited protein deficiency or inherited protein disorder. Disclosed herein is a nucleic acid sequence for a hybrid promoter having at least about 75%, at least about 80%, at least about 85%, at least about 90%, at least about 95%, or more than at least 95% identity to the sequence set forth in any one of SEQ ID NO:06-SEQ ID NO:13 and that can preferentially drive expression of one or more gene products in kidney tissue, heart tissue, skeletal muscle tissue, liver tissue, or any combination thereof in a patient having inherited protein deficiency or inherited protein disorder.

Disclosed herein is a nucleic acid sequence for a hybrid promoter that can preferentially drive expression of one or more gene products in kidney tissue, heart tissue, skeletal muscle tissue, liver tissue, or any combination thereof in a patient having alpha-1-antitrypsin deficiency, phenylketonuria, mitochondrial trifunctional protein deficiency, hemophilia, or muscle dystrophy.

Disclosed herein is a nucleic acid sequence for a hybrid promoter comprising the sequence set forth in any one of SEQ ID NO:06-SEQ ID NO:13 and that can preferentially drive expression of one or more gene products in kidney tissue, heart tissue, skeletal muscle tissue, liver tissue, or any combination thereof in a patient having alpha-1-antitrypsin deficiency, phenylketonuria, mitochondrial trifunctional protein deficiency, hemophilia, or muscle dystrophy. Disclosed herein is a nucleic acid sequence for a hybrid promoter comprising a fragment of the sequence set forth in any one of SEQ ID NO:06-SEQ ID NO:13 and that can preferentially drive expression of one or more gene products in kidney tissue, heart tissue, skeletal muscle tissue, liver tissue, or any combination thereof in a patient having alpha-1-antitrypsin deficiency, phenylketonuria, mitochondrial trifunctional protein deficiency, hemophilia, or muscle dystrophy. Disclosed herein is a nucleic acid sequence for a hybrid promoter having at least about 75%, at least about 80%, at least about 85%, at least about 90%, at least about 95%, or more than at least 95% identity to the sequence set forth in any one of SEQ ID NO:06-SEQ ID NO:13 and that can preferentially drive expression of one or more gene products in kidney tissue, heart tissue, skeletal muscle tissue, liver tissue, or any combination thereof in a patient having alpha-1-antitrypsin deficiency, phenylketonuria, mitochondrial trifunctional protein deficiency, hemophilia, or muscle dystrophy.

In an aspect, a disclosed hybrid promoter can be used in a disclosed method.

In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 can be used to drive the expression of a disclosed transgene or a disclosed cDNA in a disclosed method.

In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 can be used to drive the expression of a disclosed transgene or a disclosed cDNA in a disclosed method.

In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 can be used to generate a depot of the therapeutic expression of a transgene.

In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 can be used to generate a depot of the therapeutic expression of a transgene in a subject's liver and/or in a subject's skeletal muscle.

In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 can be used in a disclosed method of repairing a defective gene (e.g., GAA or G6PC) can restore the functionality and/or structural integrity of a missing, deficient, and/or mutant protein or enzyme (e.g., GAA or G6PC). In an aspect, a disclosed method can comprise restoring one or more aspects of cellular homeostasis and/or cellular functionality and/or metabolic dysregulation (such as, for example, glycogen accumulation). In an aspect, restoring one or more aspect of cellular homeostasis and/or cellular functionality and/or metabolic dysregulation comprises restoring the functionality and/or structural integrity of a missing, deficient, and/or mutant protein or enzyme (such as, for example, G6PC or GAA).

In an aspect, restoring one or more aspects of cellular homeostasis and/or cellular functionality can comprise one or more of the following: (i) correcting cell starvation in one or more cell types; (ii) improving, enhancing, restoring, and/or preserving mitochondrial functionality and/or structural integrity; (iii) improving, enhancing, restoring, and/or preserving organelle functionality and/or structural integrity; (iv) correcting enzyme dysregulation; (v) reversing, inhibiting, preventing, stabilizing, and/or slowing the rate of progression of the multi-systemic manifestations of a genetic disease or disorder; (vi) reversing, inhibiting, preventing, stabilizing, and/or slowing the rate of progression of a genetic disease or disorder, (vii) normalizing aspects of the autophagy pathway (such as, for example, correcting, preventing, reducing, and/or ameliorating autophagy), or (viii) any combination thereof. In an aspect, restoring one or more aspects of cellular homeostasis can comprise improving, enhancing, restoring, and/or preserving one or more aspects of cellular structural and/or functional integrity.

In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 can be used to preferentially drive expression of one or more gene products in kidney tissue, heart tissue, skeletal muscle tissue, liver tissue, or any combination thereof.

In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 can be used increase (protein or gene) expression of GAA or G6PC in kidney tissue, heart tissue, skeletal muscle tissue, liver tissue, or any combination thereof.

In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 can be used to decrease glycogen content in kidney tissue, heart tissue, skeletal muscle tissue, liver tissue, or any combination thereof.

In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 can be used to decrease the level of triglycerides in kidney tissue, heart tissue, skeletal muscle tissue, liver tissue, or any combination thereof.

In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 improve hepatocyte morphology and/or to decrease hepatocyte vacuolation.

In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 can be used to restore normal blood glucose levels in a patient or a subject in need thereof.

In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 can be used to reduce the subject's or patient's risk of developing long-term adverse effects.

In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 can be used to increase survival of a patient or a subject in need thereof.

In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 can be used to restore the functionality and/or structural integrity of a missing, deficient, and/or mutant protein or enzyme (such as, for example, G6PC or GAA).

In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 can be used to restore one or more aspects of cellular homeostasis and/or cellular functionality and/or metabolic dysregulation (such as, for example, glycogen accumulation). In an aspect, restoring one or more aspect of cellular homeostasis and/or cellular functionality and/or metabolic dysregulation comprises restoring the functionality and/or structural integrity of a missing, deficient, and/or mutant protein or enzyme (such as, for example, GAA or G6PC).

In an aspect, a disclosed hybrid promoter can drive supraphysiologic GAA expression in a subject's liver and/or a subject's skeletal muscle. In an aspect, a disclosed hybrid promoter can drive supraphysiologic G6PC expression in a subject's liver and/or a subject's skeletal muscle. In an aspect, a disclosed hybrid promoter can restore normal glycogen content in a subject's heart, diaphragm, quadriceps, or any combination thereof. In an aspect, a disclosed hybrid promoter can improve a subject's muscle strength.

In an aspect, a disclosed hybrid promoter can drive supraphysiologic expression of a gene having one or more pathogenetic defects in a subject's liver and/or a subject's skeletal muscle. In an aspect, a disclosed hybrid promoter can restore normal gene expression in a subject's heart, diaphragm, quadriceps, or any combination thereof. In an aspect, a disclosed hybrid promoter can improve a subject's muscle strength.

In an aspect, a disclosed hybrid promoter can ensure persistent transgene expression.

2. Transgenes and cDNA

Disclosed herein is a nucleic acid sequence for a transgene for correcting GSDIa or a fragment thereof. Disclosed herein is a nucleic acid sequence for a cDNA for correcting GSDIa or a fragment thereof. Disclosed herein is a nucleic acid sequence for a transgene for correcting GSDII or a fragment thereof. Disclosed herein is a nucleic acid sequence for a cDNA for correcting GSDII or a fragment thereof.

In an aspect, a disclosed transgene can be used to repair or replace any mutation or defect in the gene encoding glucose-6-phosphatase catalytic subunit (G6PC) or can be for one or more mutations or defects in the gene encoding glucose-6-phosphatase catalytic subunit (G6PC). In an aspect, a disclosed transgene can be used to repair or replace any mutation or defect in the gene encoding alpha acid glucosidase (GAA) or can be for one or more mutations or defects in the gene encoding alpha acid glucosidase (GAA). In an aspect, a disclosed transgene can encode G6PC or a fragment thereof. In an aspect, a disclosed transgene can encode GAA or a fragment thereof. In an aspect, a disclosed encoded G6PC can be a wild-type or functional G6PC or a fragment thereof. In an aspect, a disclosed encoded GAA can be a wild-type or functional GAA or a fragment thereof.

In an aspect, a disclosed transgene can comprise GAA. GAA is known to the art as evidenced by the genomic sequence set forth in SEQ ID NO:29, the mRNA sequence set forth in SEQ ID NO:30, and the protein sequence set forth in SEQ ID NO:31. In an aspect, a disclosed GAA transgene can comprise the sequence set forth in any one of SEQ ID NO:32-SEQ ID NO:36. In an aspect, a disclosed GAA transgene can be a repair template or a fragment of the full coding sequence and can comprise the sequence set forth in any one of SEQ ID NO:37-SEQ ID NO:40. The art is also familiar with homologs including murine homologs as evidenced by the sequences set forth in SEQ ID NO:26-SEQ ID NO:28.

In an aspect, a disclosed transgene can comprise 6GPC. 6GPC is known to the art as evidenced by the genomic sequence set forth in SEQ ID NO:17, the mRNA sequence set forth in SEQ ID NO:18, and the protein sequence set forth in SEQ ID NO:19. In an aspect, a disclosed G6PC transgene can comprise the sequence set forth in any one of SEQ ID NO:20, SEQ ID NO:22, SEQ ID NO:23, SEQ ID NO:25, or a fragment thereof. The art is also familiar with homologs including murine homologs. In an aspect, a disclosed encoded G6PC transgene can comprise the sequence of SEQ ID NO:21 or SEQ ID NO:24 or a fragment thereof.

In an aspect, a disclosed encoded protein can be wild-type or functional. In an aspect, a nucleic sequence for a disclosed transgene or a disclosed cDNA can comprise at least about 300 bp to at least about 4000 bp. In an aspect, a nucleic sequence for a disclosed transgene or a disclosed cDNA can comprise at least about 300 bp, at least about 400 bp, at least about 500 bp, at least about 600 bp, at least about 700 bp, at least about 800 bp, at least about 900 bp, at least about 1000 bp, at least about 1100 bp, at least about 1200 bp, at least about 1300 bp, at least about 1400 bp, at least about 1500 bp, at least about 1600 bp, at least about 1700 bp, at least about 1800 bp, at least about 1900 bp, at least about 2000 bp, at least about 2100 bp, at least about 2200 bp, at least about 2300 bp, at least about 2400 bp, at least about 2500 bp, at least about 2600 bp, at least about 2700 bp, at least about 2800 bp, at least about 2900 bp, at least about 3000 bp, at least about 3100 bp, at least about 3200 bp, at least about 3300 bp, at least about 3400 bp, at least about 3500 bp, at least about 3600 bp, at least about 3700 bp, at least about 3800 bp, at least about 3900 bp, or at least 4000 bp.

In an aspect, a disclosed transgene can encode the protein set forth in SEQ ID NO:19 or or a fragment thereof. In an aspect, a disclosed transgene can encode the protein set forth in SEQ ID NO:31 or a fragment thereof. In an aspect, a disclosed cDNA can comprise the sequence set forth in SEQ ID NO:20 or a fragment thereof. In an aspect, a disclosed cDNA can comprise the sequence set forth in any one of SEQ ID NO:32-SEQ ID NO:36 or a fragment thereof. In an aspect, a disclosed cDNA can comprise a sequence having at least about 50%, at least about 60%, at least about 70%, at least about 80%, at least about 90%, at least about 95%, or more identity to the sequence set forth in SEQ ID NO:20 or a fragment thereof. In an aspect, a disclosed cDNA can comprise a sequence having at least about 50%, at least about 60%, at least about 70%, at least about 80%, at least about 90%, at least about 95%, or more identity to the sequence set forth in any one of SEQ ID NO:36-SEQ ID NO:36 or a fragment thereof.

In an aspect, a disclosed cDNA can be used to correct and/or redress and/or reverse and/or treat alpha-1-antitrypsin deficiency, phenylketonuria, mitochondrial trifunctional protein deficiency, hemophilia, or muscle dystrophy. In an aspect, a disclosed cDNA can be used to correct and/or redress and/or reverse and/or treat an inherited protein deficiency or an inherited protein disorder. In an aspect, a disclosed cDNA can be used to correct and/or redress and/or reverse and/or treat a genetic deficiency or a genetic disorder.

In an aspect, a disclosed transgene can be used to correct and/or redress and/or reverse and/or treat alpha-1-antitrypsin deficiency, phenylketonuria, mitochondrial trifunctional protein deficiency, hemophilia, or muscle dystrophy. In an aspect, a disclosed transgene can be used to correct and/or redress and/or reverse and/or treat inherited protein deficiency or inherited protein disorder. In an aspect, a disclosed transgene can be used to correct and/or redress and/or reverse and/or treat a genetic deficiency or a genetic disorder.

In an aspect, a disclosed transgene can be used to correct and/or redress and/or reverse and/or treat a movement disorder. In an aspect, movement disorder can include neurological diseases or disorders that involve the motor and movement systems, resulting in a range of abnormalities that affect the speed, quality, and ease of movement. Movement disorders are often caused by or related to abnormalities in brain structure and/or function. Movement disorders include, but are not limited to (i) tremors: including, but not limited to, the tremor associated with Parkinson's Disease, physiologic tremor, benign familial tremor, cerebellar tremor, rubral tremor, toxic tremor, metabolic tremor, and senile tremor; (ii) chorea, including, but not limited to, chorea associated with Huntington's Disease, Wilson's Disease, ataxia telangiectasia, infection, drug ingestion, or metabolic, vascular or endocrine etiology (e.g., chorea gravidarum or thyrotoxicosis); (iii) ballism (defined herein as abruptly beginning, repetitive, wide, flinging movements affecting predominantly the proximal limb and girdle muscles); (iv) athetosis (defined herein as relatively slow, twisting, writhing, snake-like movements and postures involving the trunk, neck, face and extremities); (v) dystonia (defined herein as a movement disorder consisting of twisting, turning tonic skeletal muscle contractions, most, but not all of which are initiated distally); (vi) paroxysmal choreoathetosis and tonic spasm; (vii) tics (defined herein as sudden, behaviorally related, irregular, stereotyped, repetitive movements of variable complexity); (viii) tardive dyskinesia; (ix) akathesia, (x) muscle rigidity, defined herein as resistance of a muscle to stretch; (xi) postural instability; (xii) bradykinesia; (xiii) difficulty in initiating movements; (xiv) muscle cramps; (xv) dyskinesias and (xvi) myoclonus. In an aspect, a movement disorder comprises a dystonia.

In an aspect, a disclosed transgene can be used to correct and/or redress and/or reverse and/or treat a pathogenetic defect in skeletal muscle of a subject. In an aspect, a disclosed transgene can be used to correct and/or redress and/or reverse and/or treat a pathogenetic defect in cardiac tissue of a subject. In an aspect, a disclosed transgene can be used to correct and/or redress and/or reverse and/or treat a pathogenetic defect in kidney tissue and/or liver tissue of a subject. In an aspect, a disclosed transgene can encode a human gene product. In an aspect, a disclosed cDNA can encode a human gene product. In an aspect, a disclosed transgene can encode a non-human gene product. In an aspect, a disclosed cDNA can encode a non-human gene product.

### 3. Vectors

Disclosed herein is a vector comprising a disclosed nucleic acid sequence. Disclosed herein is a vector comprising a nucleic acid sequence for a disclosed promoter. Disclosed herein is a vector comprising a nucleic acid sequence for a disclosed hybrid promoter. In an aspect, a disclosed vector can ensure persistent transgene expression.

a. Donor Vectors

Disclosed herein is a donor vector. In an aspect, a donor vector can comprise a disclosed G6PC promoter or a disclosed hybrid promoter. In an aspect, a donor vector can comprise a nucleic acid sequence for a transgene or a fragment thereof. In an aspect, a donor vector can comprise a nucleic acid sequence for a cDNA or a fragment thereof. In an aspect of a disclosed donor vector, a disclosed G6PC promoter or a disclosed hybrid promoter can be operably linked to a disclosed transgene or a disclosed cDNA. In an aspect of a disclosed donor vector, a disclosed G6PC promoter or a disclosed hybrid promoter can be operably linked to GAA transgene or a fragment thereof. In an aspect of a disclosed donor vector, a disclosed G6PC promoter or a disclosed hybrid promoter can be operably linked to GAA cDNA or a fragment thereof. In an aspect of a disclosed donor vector, a disclosed G6PC promoter or a disclosed hybrid promoter can be operably linked to G6PC transgene or a fragment thereof. In an aspect of a disclosed donor vector, a disclosed G6PC promoter or a disclosed hybrid promoter can be operably linked to G6PC cDNA or a fragment thereof.

In an aspect of a disclosed donor vector, a disclosed G6PC promoter can comprise the sequence set forth in SEQ ID NO:02 or SEQ ID NO:03. In an aspect of a disclosed donor vector, a disclosed G6PC promoter can comprise a fragment of the sequence set forth in SEQ ID NO:02 or SEQ ID NO:03. In an aspect of a disclosed donor vector, a disclosed G6PC promoter can comprise a sequence having at least about 75%, at least about 80%, at least about 85%, at least about 90%, at least about 95%, or more than at least 95% identity to the sequence set forth in SEQ ID NO:02 or SEQ ID NO:03.

In an aspect of a disclosed donor vector, a disclosed hybrid promoter can comprise the sequence set forth in any one of SEQ ID NO:06-SEQ ID NO:09. In an aspect of a disclosed donor vector, a disclosed hybrid promoter can comprise a fragment of the sequence set forth in any one of SEQ ID NO:06-SEQ ID NO:09. In an aspect of a disclosed donor vector, a disclosed hybrid promoter can comprise a sequence having at least about 75%, at least about 80%, at least about 85%, at least about 90%, at least about 95%, or more than at least 95% identity to the sequence set forth in any one of SEQ ID NO:06-SEQ ID NO:09.

In an aspect of a disclosed donor vector, a disclosed hybrid promoter can comprise the sequence set forth in any one of SEQ ID NO:10-SEQ ID NO:13. In an aspect of a disclosed donor vector, a disclosed hybrid promoter can comprise a fragment of the sequence set forth in any one of SEQ ID NO:10-SEQ ID NO:13. In an aspect of a disclosed donor vector, a disclosed hybrid promoter can comprise a sequence having at least about 75%, at least about 80%, at least about 85%, at least about 90%, at least about 95%, or more than at least 95% identity to the sequence set forth in any one of SEQ ID NO:10-SEQ ID NO:13.

In an aspect of a disclosed donor vector, a disclosed hybrid promoter can comprise a human skeletal muscle-specific transcriptional cis-regulatory module. In an aspect of a disclosed donor vector, a disclosed hybrid promoter can comprise a human skeletal muscle-specific enhancer. In an aspect, a disclosed human skeletal muscle-specific transcriptional cis-regulatory module or enhancer can comprise Sk-CRM4. In an aspect, a disclosed Sk-CRM4 can comprise the sequence set forth in SEQ ID NO:04 or SEQ ID NO:05. In an aspect, a disclosed Sk-CRM4 can comprise a fragment of the sequence set forth in SEQ ID NO:04 or SEQ ID NO:05. In an aspect, a disclosed Sk-CRM4 can comprise a sequence having at least about 75%, at least about 80%, at least about 85%, at least about 90%, at least about 95%, or more than at least 95% identity to the sequence set forth in SEQ ID NO:04 or SEQ ID NO:05.

In an aspect, a disclosed donor vector can preferentially drive expression of one or more gene products in kidney tissue, heart tissue, skeletal muscle tissue, liver tissue, or any combination thereof in a patient having genetic deficiency or genetic disorder. In an aspect, a disclosed donor vector can preferentially drive expression of one or more gene products in kidney tissue, heart tissue, skeletal muscle tissue, liver tissue, or any combination thereof in a patient having inherited protein deficiency or inherited protein disorder. In an aspect, a disclosed donor vector can preferentially drive expression of one or more gene products in kidney tissue, heart tissue, skeletal muscle tissue, liver tissue, or any combination thereof in a patient having alpha-1-antitrypsin deficiency, phenylketonuria, mitochondrial trifunctional protein deficiency, hemophilia, or muscle dystrophy.

In an aspect, a disclosed transgene can comprise GAA or fragment thereof or fragment thereof. In an aspect, a disclosed cDNA can comprise GAA or fragment thereof fragment thereof. In an aspect, a disclosed transgene can comprise G6PC or fragment thereof. In an aspect, a disclosed cDNA can comprise G6PC or fragment thereof.

In an aspect, a disclosed transgene can comprise a gene product that can be used in a gene editing application. In an aspect, a disclosed cDNA can comprise a gene product that can be used in a gene editing application. In an aspect, a disclosed cDNA can comprise a gene product that is relevant to alpha-1-antitrypsin deficiency, phenylketonuria, mitochondrial trifunctional protein deficiency, hemophilia, or muscle dystrophy. In an aspect, a disclosed cDNA can comprise a gene product that is relevant to an inherited protein deficiency or inherited protein disorder.

In an aspect, a disclosed donor vector can comprise a nucleic acid sequence for one or more homologous arms.

In an aspect, a disclosed first homologous sequence can comprise at least about 100 bp to at least about 2500 bp. In an aspect, a disclosed second homologous sequence can comprise at least about 100 bp to at least about 2500 bp. In an aspect, a disclosed first homologous sequence can comprise at least about 100 bp, at least about 200 bp, at least about 300 bp, at least about 400 bp, at least about 500 bp, at least about 600 bp, at least about 700 bp, at least about 800 bp, at least about 900 bp, at least about 1000 bp, at least about 1100 bp, at least about 1200 bp, at least about 1300 bp, at least about 1400 bp, at least about 1500 bp, at least about 1600 bp, at least about 1700 bp, at least about 1800 bp, at least about 1900 bp, at least about 2000 bp, at least about 2100 bp, at least about 2200 bp, at least about 2300 bp, at least about 2400 bp, or at least about 2500 bp. In an aspect, a disclosed second homologous sequence can comprise at least about 100 bp, at least about 200 bp, at least about 300 bp, at least about 400 bp, at least about 500 bp, at least about 600 bp, at least about 700 bp, at least about 800 bp, at least about 900 bp, at least about 1000 bp, at least about 1100 bp, at least about 1200 bp, at least about 1300 bp, at least about 1400 bp, at least about 1500 bp, at least about 1600 bp, at least about 1700 bp, at least about 1800 bp, at least about 1900 bp, at least about 2000 bp, at least about 2100 bp, at least about 2200 bp, at least about 2300 bp, at least about 2400 bp, or at least about 2500 bp. In an aspect, a first disclosed homologous sequence can comprise about 175 bp to about 250 bp or about 200 bp to about 225 bp. In an aspect, a disclosed second homologous sequence can comprise about 175 bp to about 250 bp or about 200 bp to about 225 bp.

In an aspect, a disclosed donor vector can comprise a donor sequence. In an aspect, a disclosed donor sequence can comprise a disclosed transgene or a disclosed cDNA. In an aspect, a pair of a disclosed homologous arms can be used to direct to a disclosed transgene or a disclosed cDNA to the Cas induced break in the targeted locus for integration. In an aspect, a pair of a disclosed homologous arms can be used to direct to a disclosed sequence to the Cas induced break in the targeted locus for integration. In an aspect, a pair of a disclosed homologous arms can be used to direct to a disclosed transgene or a disclosed cDNA to the Cas induced break in the targeted locus for homology-directed repair (HDR). In an aspect, a pair of a disclosed homologous arms can be used to direct to a disclosed sequence to the Cas induced break in the targeted locus for homology-directed repair (HDR).

In an aspect of a disclosed donor vector, a disclosed homologous arm can comprise a sequence 5′ to the sequence of a disclosed transgene. In an aspect of a disclosed donor vector, a disclosed homologous arm can comprise a sequence 3′ to the sequence of a disclosed transgene. In an aspect of a disclosed donor vector, a disclosed homologous arm can assist with the integration of a disclosed transgene. In an aspect of a disclosed donor vector, a disclosed pair of homologous arms can assist with the integration of a disclosed transgene. In an aspect of a disclosed donor vector, a disclosed homologous arm can facilitate the integration of a disclosed transgene. In an aspect of a disclosed donor vector, a disclosed pair of homologous arms can facilitate the integration of a disclosed transgene. In an aspect, a disclosed homologous arm can contain a 5′ UTR genomic sequence of a disclosed transgene. In an aspect, a disclosed homologous arm can contain an intron 1 genomic sequence of a disclosed transgene. In an aspect, a disclosed homologous arm can contain a 5′ UTR genomic sequence of a disclosed cDNA. In an aspect, a disclosed homologous arm can contain an intron 1 genomic sequence of a disclosed cDNA. In an aspect, a disclosed homologous arm can comprise a sequence homologous to a sequence 5′ of the targeted locus. In an aspect, a disclosed homologous arm can comprise a sequence homologous to a sequence 3′ of the targeted locus. In an aspect of a disclosed donor vector, a disclosed targeted locus can comprise G6PC or GAA.

In an aspect of a disclosed donor vector, a disclosed targeted locus can comprise a gene product that can be used in a gene editing application. In an aspect of a disclosed donor vector, a disclosed targeted locus can comprise a gene product that is relevant to alpha-1-antitrypsin deficiency, phenylketonuria, mitochondrial trifunctional protein deficiency, hemophilia, or muscle dystrophy. In an aspect of a disclosed donor vector, a disclosed targeted locus can comprise a gene product that is relevant to an inherited protein deficiency or inherited protein disorder. In an aspect of a disclosed donor vector, a disclosed targeted locus can be the focus of a gene editing application or a gene therapy application.

In an aspect, a disclosed first homologous sequence can comprise the sequence of a disclosed wild-type transgene or a portion thereof, and/or a disclosed second homologous sequence can comprise the sequence of the disclosed wild-type transgene or a portion thereof, wherein the absence of, mutation of, defect of, or dysfunction of the transgene creates and/or contributes to a genetic deficiency or genetic disorder.

In an aspect, a disclosed first homologous sequence can comprise the sequence of a wild-type G6PC or a portion thereof, and a disclosed second homologous sequence can comprise the sequence of a wild-type G6PC or a portion thereof, or any combination thereof.

In an aspect, a disclosed first homologous sequence can comprise the sequence of a wild-type GAA or a portion thereof, and a disclosed second homologous sequence can comprise the sequence of a wild-type GAA or a portion thereof, or any combination thereof.

In an aspect, a disclosed first homologous sequence can comprise the sequence of a disclosed wild-type transgene or a portion thereof, and/or a disclosed second homologous sequence can comprise the sequence of the disclosed wild-type transgene or a portion thereof, wherein the absence of, mutation of, defect of, or dysfunction of the transgene creates and/or contributes to an inherited protein deficiency or inherited protein disorder.

In an aspect, a disclosed first homologous sequence can comprise the sequence of a disclosed wild-type transgene or a portion thereof, and/or a disclosed second homologous sequence can comprise the sequence of the disclosed wild-type transgene or a portion thereof, wherein the absence of, mutation of, defect of, or dysfunction of the transgene creates and/or contributes to alpha-1-antitrypsin deficiency, phenylketonuria, mitochondrial trifunctional protein deficiency, hemophilia, or muscle dystrophy

In an aspect of a disclosed donor vector, a disclosed homologous arm can comprise the sequence set forth in SEQ ID NO:91 or SEQ ID NO:92 or a fragment thereof or portion thereof. In an aspect of a disclosed donor vector, a disclosed homologous arm can comprise the sequence set forth in any one of SEQ ID NO:94-SEQ ID NO:96 or a fragment thereof or portion thereof. In an aspect, a disclosed 5′ homologous arm can comprise the sequence set forth in SEQ ID NO:91-SEQ ID NO:92 or a fragment thereof or portion thereof. In an aspect, a disclosed 3′ homologous arm can comprise the sequence set forth in any one of SEQ ID NO:94-SEQ ID NO:96 or a fragment thereof or portion thereof.

In an aspect of a disclosed donor vector, a disclosed homologous arm can comprise the sequence set forth in any one of SEQ ID NO:97-SEQ ID NO:99 or a fragment thereof or portion thereof. In an aspect of a disclosed donor vector, a disclosed homologous arm can comprise the sequence set forth in any one of SEQ ID NO:100-SEQ ID NO:102 or a fragment thereof or portion thereof. In an aspect, a disclosed 5′ homologous arm can comprise the sequence set forth in SEQ ID NO:97-SEQ ID NO:99 or a fragment thereof or portion thereof. In an aspect, a disclosed 3′ homologous arm can comprise the sequence set forth in any one of SEQ ID NO:100-SEQ ID NO:102 or a fragment thereof or portion thereof.

In an aspect, a donor vector can comprise one or more inverted terminal repeats (ITRs). In an aspect, a donor vector can comprise a 5′ ITR and/or a 3′ ITR. ITRs are known to the skilled person in the art. In an aspect, a disclosed ITR can comprise the sequence set forth in any one of SEQ ID NO:113-SEQ ID NO:116 or a fragment thereof or portion thereof. In an aspect, a disclosed ITR can comprise an ITR fragment comprising the sequence set forth in any one of SEQ ID NO:117-SEQ ID NO:119 or a fragment thereof or portion thereof.

In an aspect, a donor vector can comprise one or more polyA sequences. PolyA sequences are known to the skilled person in the art. In an aspect, a disclosed polyA sequence can comprise the sequence set forth in any one of SEQ ID NO:105-SEQ ID NO:110 or a fragment thereof or portion thereof.

In an aspect, a disclosed donor vector can comprise a guide RNA (gRNA). In an aspect, a disclosed gRNA can comprise a specific RNA sequence that recognizes the target DNA region of interest and can direct a disclosed Cas nuclease there for editing. In an aspect, a disclosed gRNA can comprise 2 parts: (i) crispr RNA (crRNA), which is a 17-20 nucleotide sequence complementary to the targeted DNA or targeted locus, and (ii) a tracr RNA, which serves as a binding scaffold for a Cas nuclease. In an aspect, a targeted DNA or targeted locus can be a gene having one or more mutations or defects that contribute to one or more genetic diseases or disorders. In an aspect, a targeted DNA or targeted locus can be subjected to a Cas9 induced double-stranded break. In an aspect, a gRNA scaffold can comprise the sequence set forth in SEQ ID NO:103 or SEQ ID NO:104.

In an aspect, a disclosed tracrRNA sequence can comprise nucleotides that hybridize to a minimum CRISPR repeat sequence in a cell. A minimum tracrRNA sequence and a minimum CRISPR repeat sequence may form a duplex, i.e., a base-paired double-stranded structure. Together, the minimum tracrRNA sequence and the minimum CRISPR repeat can bind to an RNA-guided endonuclease. At least a part of the minimum tracrRNA sequence can hybridize to the minimum CRISPR repeat sequence. In an aspect, a disclosed minimum tracrRNA sequence can be at least about 30%, about 40%, about 50%, about 60%, about 65%, about 70%, about 75%, about 80%, about 85%, about 90%, about 95%, or 100% complementary to the minimum CRISPR repeat sequence. For example, in an aspect, a disclosed minimum tracrRNA sequence can have a length from about 7 nucleotides to about 100 nucleotides. In an aspect, a disclosed minimum tracrRNA sequence can be from about 7 nucleotides (nt) to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt long. In an aspect, a disclosed minimum tracrRNA sequence can be at least about 60% identical to a reference minimum tracrRNA (e.g., wild type, tracrRNA from S. pyogenes) sequence over a stretch of at least 6, 7, or 8 contiguous nucleotides. In an aspect, for example, the minimum tracrRNA sequence can be at least about 65% identical, about 70% identical, about 75% identical, about 80% identical, about 85% identical, about 90% identical, about 95% identical, about 98% identical, about 99% identical or 100% identical to a reference minimum tracrRNA sequence over a stretch of at least 6, 7, or 8 contiguous nucleotides.

In an aspect, a targeted DNA or targeted locus can comprise G6PC or GAA. In an aspect, a targeted DNA or targeted locus can comprise G6PC or GAA. In an aspect, a targeted DNA or targeted locus can be a gene having one or more mutations or defects that contribute to alpha-1-antitrypsin deficiency, phenylketonuria, mitochondrial trifunctional protein deficiency, hemophilia, or muscle dystrophy.

In an aspect, a disclosed gRNA can target a PAM sequence at or near the targeted DNA or targeted locus. In an aspect, a spacer sequence can be designed to hybridize to a target polynucleotide that is located 5′ of a PAM of a disclosed Cas9 endonuclease. In an aspect, a disclosed spacer can match the targeted sequence or can have mismatches. In an aspect, S. pyogenes Cas9 recognizes a PAM that comprises the sequence 5′-NRG-3′, where R comprises either A or G, where N is any nucleotide and N is immediately 3′ of the target nucleic acid sequence targeted by the spacer sequence. In an aspect, S. aureus Cas9 recognizes a PAM that comprises the sequence 5′-NNGRRT-3′ (where R represents A or G) an NN is immediately 3′ of the target nucleic acid sequence targeted by the spacer sequence. PAMs are known to the skilled person in the art. In an aspect, a disclosed gRNA can target a PAM sequence at or near any mutation or defect in the G6PC gene. In an aspect, a disclosed gRNA can target a PAM sequence at or near any mutation or defect in the GAA gene. In an aspect, a disclosed mutation or defect in the GAA gene can comprise the IVS1 variant of Pompe disease, the ΔT525 variant of Pompe disease, or the 1826DupA variant of Pompe disease. In an aspect, a disclosed gRNA can target a PAM sequence at or near any mutation or defect in a gene having one or more mutations or defects that contribute to one or more genetic diseases or disorders. In an aspect, a disclosed gRNA can target a PAM sequence at or near any mutation or defect in a gene having one or more mutations or defects that contribute to alpha-1-antitrypsin deficiency, phenylketonuria, mitochondrial trifunctional protein deficiency, hemophilia, or muscle dystrophy.

In an aspect, a disclosed gRNA targeting G6PC can comprise the sequence set forth in any one of SEQ ID NO:57-SEQ ID NO:66.

Exemplary SaCas9 gRNAs for
Editing Human G6PC

| gRNA | PAM | Mutant PAM |
|---|---|---|
| TGATGGACAGACATTGCGAGA | GCGAAT | GCctAT |
| (SEQ ID NO: 57) | (SEQ ID | (SEQ ID |
| | NO: 79) | NO: 83) |
| GCATGAGTAGCCCGTGGCTTC | CTGAAT | CTctAT |
| (SEQ ID NO: 58) | (SEQ ID | (SEQ ID |
| | NO: 80) | NO: 84) |
| TCAGAGAGAGGGGTTGGAAGC | ATGAGT | ATctGT |
| (SEQ ID NO: 59) | (SEQ ID | (SEQ ID |
| | NO: 81) | NO: 85) |
| AGATGATCCAAAGTCAGAGAG | AGGGGT | AGccGT |
| (SEQ ID NO: 60) | (SEQ ID | (SEQ ID |
| | NO: 82) | NO: 86) |

Exemplary SpCas9 gRNAs for
Editing Human G6PC

| gRNA | PAM |
|---|---|
| TCTACGTCCTCTTCCCCATC (SEQ ID NO:61) | TGG |
| TTGCTGATCTCCTCTCTATA (SEQ ID NO:62) | TGG |
| GTGGTAAGAACCATATAGAG (SEQ ID NO:63) | AGG |
| GAGGAGATCAGCAAGAAAAG (SEQ ID NO:64) | AGG |
| AGATCAGCAAGAAAAGAGGC (SEQ ID NO:65) | TGG |
| CTGGGGAAAGCAACTTCTGA (SEQ ID NO: 66) | TGG |

In an aspect, a disclosed gRNA targeting GAA can comprise the sequence set forth in SEQ ID NO:69 or SEQ ID NO:70. In an aspect, a disclosed gRNA can target a specific defect in GAA and can comprise the sequence set forth in SEQ ID NO:72 or SEQ ID NO:77. In an aspect, a disclosed gRNA can target a specific defect in GAA and can comprise the sequence set forth in SEQ ID NO:71. In an aspect, a disclosed gRNA can target a specific defect in GAA and can comprise the sequence set forth in SEQ ID NO:73 or SEQ ID NO:74.

In an aspect, a disclosed donor vector can comprise a promoter operably linked to a disclosed gRNA. In an aspect, a disclosed promoter operably linked to a disclosed gRNA can comprise a RNA polymerase III (Poly III) promoter. In an aspect, a disclosed Poly III promoter can comprise a U3 promoter, a U6 promoter, or a H1 promoter. In an aspect, a disclosed promoter operably linked to a disclosed gRNA can comprise a glutamine tRNA promoter. In an aspect, a disclosed promoter operably linked to a disclosed gRNA can comprise a U6 promoter. In an aspect, a disclosed U6 promoter can comprise the sequence set forth in any one of SEQ ID NO:120-SEQ ID NO:122, or a sequence having at least about 50%, at least about 60%, at least about 70%, at least about 80%, at least about 90%, at least about 95%, or more identity to the sequence set forth in any one of SEQ ID NO:120-SEQ ID NO:122.

In an aspect, a disclosed hybrid promoter can be used in a disclosed method. In an aspect, a disclosed hybrid promoter comprising (i) the sequence set forth in any one of SEQ ID NO:06-SEQ ID NO:13, (ii) a fragment of the sequence set forth in any one of SEQ ID NO:06-SEQ ID NO:13, or (iii) at least about 75%, at least about 80%, at least about 85%, at least about 90%, at least about 95%, or more than at least 95% identity to the sequence set forth in any one of SEQ ID NO:06-SEQ ID NO:13 can be used to drive the expression of a disclosed transgene or a disclosed cDNA in a disclosed method.

In an aspect, a disclosed donor vector can be used in a gene editing application. In an aspect, a disclosed donor vector can be used with a disclosed CRISPR vector. In an aspect, a disclosed donor vector can be used in a gene therapy application. In an aspect, a disclosed donor vector can be used in the absence of a disclosed CRISPR vector. In an aspect, a disclosed donor vector can comprise one or more disclosed ITRs, one or more disclosed homology arms, one or more disclosed polyA sequences, one or more disclosed promoters, one or more disclosed gRNAs.

Disclosed herein is a donor vector comprising from 5′ to 3′ the following components: a first disclosed ITR, a disclosed 5′ homology arm, a disclosed promoter or disclosed hybrid promoter that is operably linked to a disclosed transgene or a disclosed cDNA, a polyA sequence, a disclosed 3′ homology arm, a promoter that is operably linked to a gRNA, and a second disclosed ITR.

Disclosed herein is a donor vector comprising from 5′ to 3′ the following components: a first disclosed ITR, a disclosed 5′ homology arm, a disclosed G6PC min promoter or disclosed hybrid promoter comprising a G6PC min promoter and a hSk-CRM4 regulatory module that is operably linked to a disclosed transgene or a disclosed cDNA, a polyA sequence, a disclosed 3′ homology arm, a U6 promoter that is operably linked to a gRNA, and a second disclosed ITR.

In an aspect of a disclosed donor vector, a disclosed nucleic acid sequence can be CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

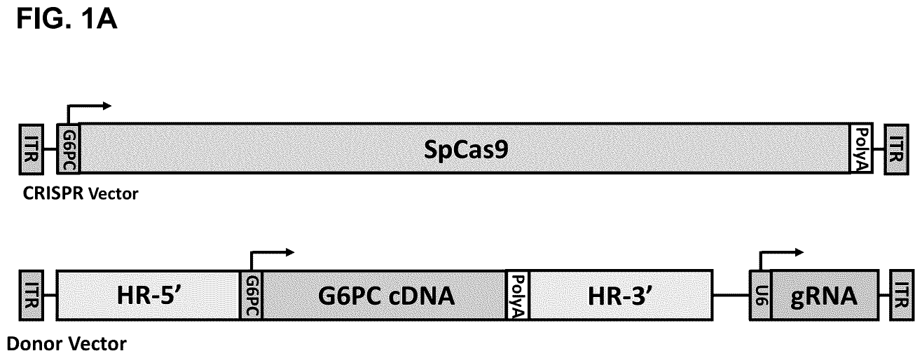

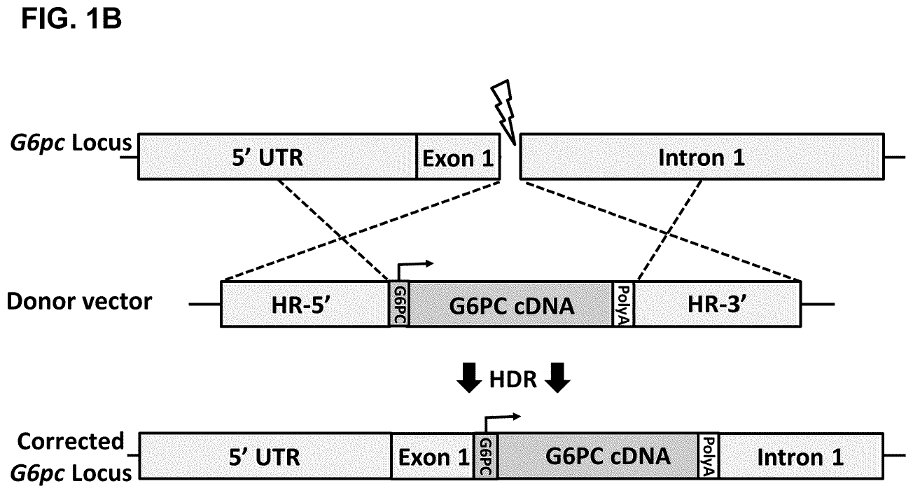

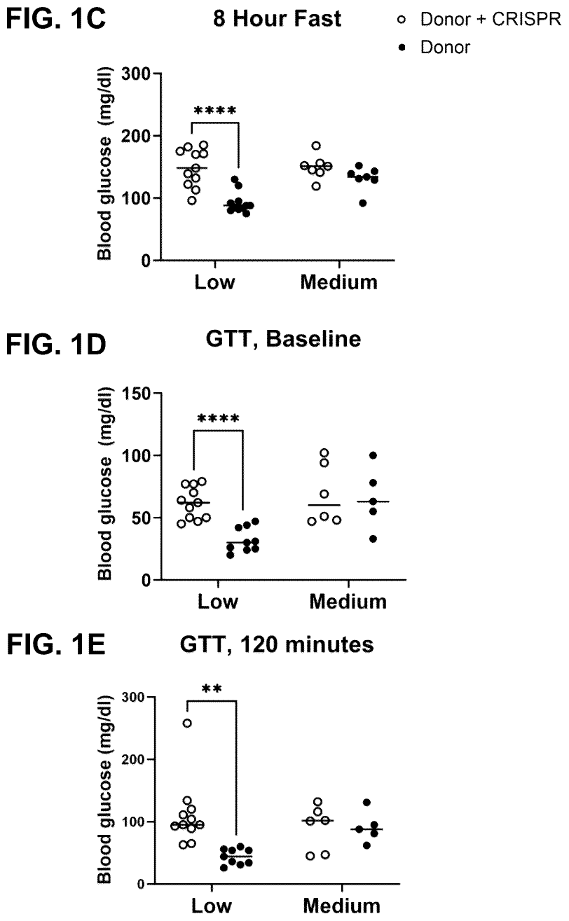

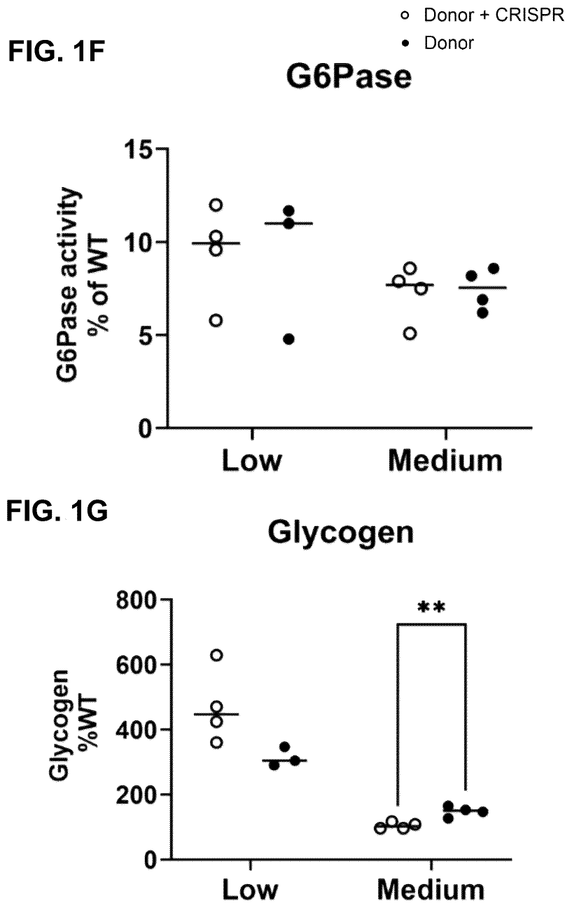

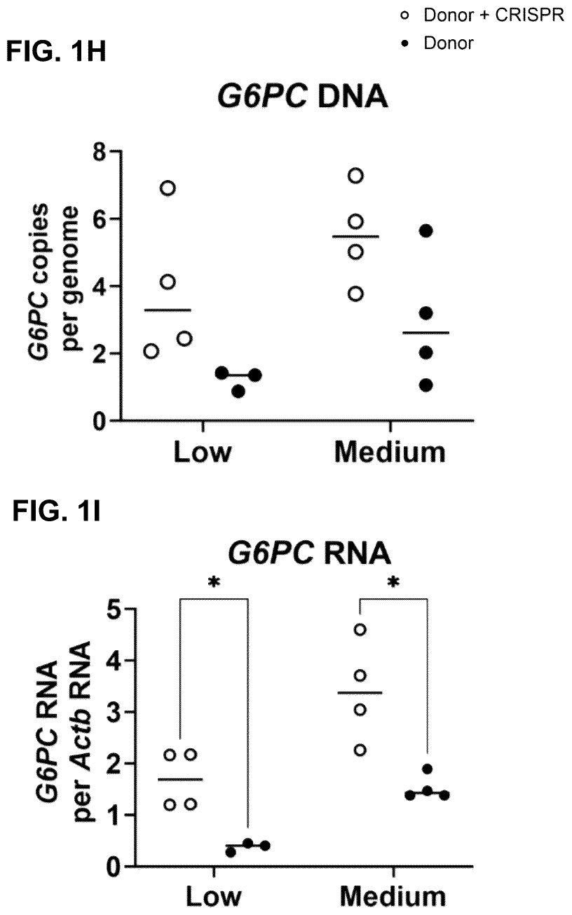

In an aspect, a disclosed donor vector can comprise the construct set forth in FIG. 1A.

In an aspect, a disclosed viral vector can be an adeno-associated virus (AAV) vector In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but not limited to, AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, a disclosed AAV vector can be AAV-Rh74 or a related variant (e.g., capsid variants like RHM4-1). In an aspect, a disclosed AAV vector can be a recombinant AAV vector (rAAV). In an aspect, a disclosed rAAV vector can be rAAV9.

In an aspect of a disclosed donor vector, a disclosed nucleic acid sequence can have a coding sequence that is less than about 4.5 kilobases.

In an aspect, a disclosed donor vector can comprise a nuclear localization signal (NLS). In an aspect, a disclosed NLS can comprise the sequence set forth in SEQ ID NO: 143 or SEQ ID NO:144. In an aspect, a disclosed donor vector can comprise one or more hemagglutinin (HA) tags. In an aspect, a disclosed HA tag can comprise the sequence set forth in SEQ ID NO:111 or SEQ ID NO:112. In an aspect, a disclosed donor vector can comprise a Rosa26 fragment (e.g., SEQ ID NO:145).

In an aspect, a disclosed donor vector can preferentially drive expression of one or more gene products in kidney tissue, heart tissue, skeletal muscle tissue, liver tissue, or any combination thereof. In an aspect, a disclosed donor vector can increase (protein or gene) expression of GAA or G6PC in kidney tissue, heart tissue, skeletal muscle tissue, liver tissue, or any combination thereof. In an aspect, a disclosed donor vector can decrease glycogen content in kidney tissue, heart tissue, skeletal muscle tissue, liver tissue, or any combination thereof.

In an aspect, a disclosed donor vector can decrease the level of triglycerides in kidney tissue, heart tissue, skeletal muscle tissue, liver tissue, or any combination thereof. In an aspect, a disclosed donor vector can improve hepatocyte morphology and/or can decrease hepatocyte vacuolation. In an aspect, a disclosed donor vector can restore normal blood glucose levels in a patient or a subject in need thereof. In an aspect, a disclosed donor vector can reduce the subject's or patient's risk of developing long-term adverse effects. In an aspect, a disclosed donor vector can improve hypoglycemia and/or decrease hyperlipidemia in a subject. In an aspect, a disclosed donor vector can increase survival of a patient or a subject in need thereof. In an aspect, a disclosed donor vector can restore the functionality and/or structural integrity of a missing, deficient, and/or mutant protein or enzyme (such as, for example, G6PC or GAA).

In an aspect, a disclosed donor vector can restore one or more aspects of cellular homeostasis and/or cellular functionality and/or metabolic dysregulation (such as, for example, glycogen accumulation). In an aspect, restoring one or more aspect of cellular homeostasis and/or cellular functionality and/or metabolic dysregulation comprises restoring the functionality and/or structural integrity of a missing, deficient, and/or mutant protein or enzyme (such as, for example, GAA or G6PC).

In an aspect, restoring one or more aspects of cellular homeostasis and/or cellular functionality can comprise one or more of the following: (i) correcting cell starvation in one or more cell types; (ii) improving, enhancing, restoring, and/or preserving mitochondrial functionality and/or structural integrity; (iii) improving, enhancing, restoring, and/or preserving organelle functionality and/or structural integrity; (iv) correcting enzyme dysregulation; (v) reversing, inhibiting, preventing, stabilizing, and/or slowing the rate of progression of the multi-systemic manifestations of a genetic disease or disorder; (vi) reversing, inhibiting, preventing, stabilizing, and/or slowing the rate of progression of a genetic disease or disorder, (vii) normalizing aspects of the autophagy pathway (such as, for example, correcting, preventing, reducing, and/or ameliorating autophagy), or (viii) any combination thereof. In an aspect, restoring one or more aspects of cellular homeostasis can comprise improving, enhancing, restoring, and/or preserving one or more aspects of cellular structural and/or functional integrity.

In an aspect, restoring the activity and/or functionality of a missing, deficient, and/or mutant protein or enzyme (such as, for example, G6PC or GAA) can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme such as, for example, G6PC or GAA). In an aspect, restoration can be a partial or incomplete restoration. In an aspect, restoration can be complete or near complete restoration such that the level of expression, activity, and/or functionality is similar to that of a wild-type or control level.

In an aspect, a disclosed donor vector can be used to correct and/or redress and/or reverse and/or treat a pathogenetic defect in skeletal muscle of a subject. In an aspect, a disclosed donor vector can be used to correct and/or redress and/or reverse and/or treat a pathogenetic defect in cardiac tissue of a subject. In an aspect, a disclosed donor vector can be used to correct and/or redress and/or reverse and/or treat a pathogenetic defect in kidney tissue and/or liver tissue of a subject.

In an aspect, a disclosed donor vector can be used to correct and/or redress and/or reverse and/or treat a movement disorder. In an aspect, movement disorder can include neurological diseases or disorders that involve the motor and movement systems, resulting in a range of abnormalities that affect the speed, quality, and ease of movement. Movement disorders are often caused by or related to abnormalities in brain structure and/or function. Movement disorders include, but are not limited to (i) tremors: including, but not limited to, the tremor associated with Parkinson's Disease, physiologic tremor, benign familial tremor, cerebellar tremor, rubral tremor, toxic tremor, metabolic tremor, and senile tremor; (ii) chorea, including, but not limited to, chorea associated with Huntington's Disease, Wilson's Disease, ataxia telangiectasia, infection, drug ingestion, or metabolic, vascular or endocrine etiology (e.g., chorea gravidarum or thyrotoxicosis); (iii) ballism (defined herein as abruptly beginning, repetitive, wide, flinging movements affecting predominantly the proximal limb and girdle muscles); (iv) athetosis (defined herein as relatively slow, twisting, writhing, snake-like movements and postures involving the trunk, neck, face and extremities); (v) dystonia (defined herein as a movement disorder consisting of twisting, turning tonic skeletal muscle contractions, most, but not all of which are initiated distally); (vi) paroxysmal choreoathetosis and tonic spasm; (vii) tics (defined herein as sudden, behaviorally related, irregular, stereotyped, repetitive movements of variable complexity); (viii) tardive dyskinesia; (ix) akathesia, (x) muscle rigidity, defined herein as resistance of a muscle to stretch; (xi) postural instability; (xii) bradykinesia; (xiii) difficulty in initiating movements; (xiv) muscle cramps; (xv) dyskinesias and (xvi) myoclonus. In an aspect, a movement disorder comprises a dystonia.

In an aspect, a disclosed donor vector can drive supraphysiologic GAA expression in a subject's liver and/or a subject's skeletal muscle. In an aspect, a disclosed donor vector can drive supraphysiologic G6PC expression in a subject's liver and/or a subject's skeletal muscle. In an aspect, a disclosed donor vector can restore normal glycogen content in a subject's heart, diaphragm, quadriceps, or any combination thereof. In an aspect, a disclosed donor vector can improve a subject's muscle strength.

In an aspect, a disclosed donor vector does not elicit an immune response.

In an aspect, a disclosed donor vector can ensure persistent transgene expression.

In an aspect, a disclosed donor vector can achieve episomal expression of a disclosed transgene.

b. CRISPR Vectors

Disclosed herein is a CRISPR vector. Disclosed herein is a CRISPR vector for use in a disclosed method of gene editing. In an aspect, a CRISPR vector can comprise a nucleic acid sequence for a Cas9 nuclease. In an aspect, a CRISPR vector can comprise a disclosed G6PC promoter or a disclosed hybrid promoter.

In an aspect of a disclosed CRISPR vector, a disclosed G6PC promoter can comprise the sequence set forth in SEQ ID NO:02 or SEQ ID NO:03 In an aspect of a disclosed CRISPR vector, a disclosed G6PC promoter can comprise a fragment of the sequence set forth in SEQ ID NO:02 or SEQ ID NO:03 In an aspect of a disclosed CRISPR vector, a disclosed G6PC promoter can comprise a sequence having at least about 75%, at least about 80%, at least about 85%, at least about 90%, at least about 95%, or more than at least 95% identity to the sequence set forth in SEQ ID NO:02 or SEQ ID NO:03.

In an aspect of a disclosed CRISPR vector, a disclosed hybrid promoter can comprise the sequence set forth in any one of SEQ ID NO:06-SEQ ID NO:09. In an aspect of a disclosed CRISPR vector, a disclosed hybrid promoter can comprise a fragment of the sequence set forth in any one of SEQ ID NO:06-SEQ ID NO:09. In an aspect of a disclosed CRISPR vector, a disclosed hybrid promoter can comprise a sequence having at least about 75%, at least about 80%, at least about 85%, at least about 90%, at least about 95%, or more than at least 95% identity to the sequence set forth in any one of SEQ ID NO:06-SEQ ID NO:09.

In an aspect of a disclosed CRISPR vector, a disclosed hybrid promoter can comprise the sequence set forth in any one of SEQ ID NO:10-SEQ ID NO:13. In an aspect of a disclosed CRISPR vector, a disclosed hybrid promoter can comprise a fragment of the sequence set forth in any one of SEQ ID NO:10-SEQ ID NO:13. In an aspect of a disclosed CRISPR vector, a disclosed hybrid promoter can comprise a sequence having at least about 75%, at least about 80%, at least about 85%, at least about 90%, at least about 95%, or more than at least 95% identity to the sequence set forth in any one of SEQ ID NO:10-SEQ ID NO:13.

In an aspect of a disclosed CRISPR vector, a disclosed hybrid promoter can comprise a human skeletal muscle-specific transcriptional cis-regulatory module. In an aspect of a disclosed CRISPR vector, a disclosed hybrid promoter can comprise a human skeletal muscle-specific enhancer. In an aspect, a disclosed human skeletal muscle-specific transcriptional cis-regulatory module or enhancer can comprise Sk-CRM4. In an aspect, a disclosed Sk-CRM4 can comprise the sequence set forth in SEQ ID NO:04 or SEQ ID NO:05. In an aspect, a disclosed Sk-CRM4 can comprise a fragment of the sequence set forth in SEQ ID NO:04 or SEQ ID NO:05.

In an aspect, a disclosed Sk-CRM4 can comprise a sequence having at least about 75%, at least about 80%, at least about 85%, at least about 90%, at least about 95%, or more than at least 95% identity to the sequence set forth in SEQ ID NO:04 or SEQ ID NO:05. In an aspect of a disclosed CRISPR vector, a disclosed G6PC promoter or a disclosed hybrid promoter can be operably linked to a disclosed Cas9 nuclease.

In an aspect, a disclosed Cas9 nuclease can comprise a SpCas9 nuclease (Streptococcus Pyogenes). In an aspect, a disclosed SaCas9 nuclease can comprise the sequence set forth in SEQ ID NO: or a fragment thereof. In an aspect, a disclosed SpCas9 nuclease can comprise a sequence having about 60%, about 65%, about 70%, about 75%, about 80%, about 85%, about 90%, about 95%, or more than 95% identity to the sequence set forth in SEQ ID NO:41 or a fragment thereof. In an aspect, a disclosed encoded SpCas9 nucleus can comprise the sequence of SEQ ID NO:44 or a fragment thereof.

In an aspect, a disclosed Cas9 nuclease can comprise a SaCas9 nuclease (Staphylococcus Aureus). In an aspect, a disclosed SaCas9 nuclease can comprise the sequence set forth in SEQ ID NO:42 or SEQ ID NO:43 or a fragment thereof. In an aspect, a disclosed SaCas9 nuclease can comprise a sequence having about 60%, about 65%, about 70%, about 75%, about 80%, about 85%, about 90%, about 95%, or more than 95% identity to the sequence set forth in SEQ ID NO:42 or SEQ ID NO:43 or a fragment thereof. In an aspect, a disclosed encoded SaCas9 nucleus can comprise the sequence of SEQ ID NO:45 or SEQ ID NO:46 or a fragment thereof.

In an aspect, a CRISPR vector can comprise one or more inverted terminal repeats (ITRs). In an aspect, a CRISPR vector can comprise a 5′ ITR and/or a 3′ ITR. ITRs are known to the skilled person in the art. In an aspect, a disclosed ITR can comprise the sequence set forth in any one of SEQ ID NO:113-SEQ ID NO:116 or a fragment thereof or portion thereof. In an aspect, a disclosed ITR can comprise an ITR fragment comprising the sequence set forth in any one of SEQ ID NO:117-SEQ ID NO:119 or a fragment thereof or portion thereof. In an aspect, a CRISPR vector can comprise one or more polyA sequences. PolyA sequences are known to the skilled person in the art. In an aspect, a disclosed polyA sequence can comprise the sequence set forth in any one of SEQ ID NO:105-SEQ ID NO:110 or a fragment thereof or portion thereof.

In an aspect, a disclosed CRISPR vector comprising a disclosed Cas9 nuclease can create a double-strand break (DSB) at or near a mutation or defect in the targeted locus that results in a permanent integration of a disclosed transgene or cDNA. In an aspect, a disclosed CRISPR vector comprising a disclosed Cas9 nuclease can create a double-strand break (DSB) at or near a mutation or defect in the targeted GAA locus that results in a permanent integration of a disclosed transgene or cDNA. In an aspect, a disclosed mutation or defect in the GAA gene can comprise the IVS1 variant of Pompe disease, the ΔT525 variant of Pompe disease, or the 1826DupA variant of Pompe disease.

In an aspect, a disclosed CRISPR vector comprising a disclosed Cas9 nuclease can create a double-strand break (DSB) at or near a mutation or defect in the targeted G6PC locus that results in a permanent integration of a disclosed transgene or cDNA. In an aspect, a disclosed CRISPR vector comprising a disclosed Cas9 nuclease can create a double-strand break (DSB) at or near a mutation or defect in the targeted locus associated with or related to a genetic deficiency or genetic disorder that results in a permanent integration of a disclosed transgene or cDNA. In an aspect, a disclosed CRISPR vector comprising a disclosed Cas9 nuclease can create a double-strand break (DSB) at or near a mutation or defect in the targeted locus associated with or related to an inherited protein deficiency or inherited protein disorder that results in a permanent integration of a disclosed transgene or cDNA. In an aspect, a disclosed CRISPR vector comprising a disclosed Cas9 nuclease can create a double-strand break (DSB) at or near a mutation or defect in the targeted locus associated with or related to alpha-1-antitrypsin deficiency, phenylketonuria, mitochondrial trifunctional protein deficiency, hemophilia, or muscle dystrophy that results in a permanent integration of a disclosed transgene or cDNA.

In an aspect, a disclosed CRISPR vector can be used in a gene editing application. In an aspect, a disclosed CRISPR vector can be used with a disclosed donor vector.

In an aspect, a disclosed CRISPR vector can comprise one or more disclosed ITRs, one or more disclosed polyA sequences, a disclosed Cas9 endonuclease, one or more disclosed promoters, one or more disclosed gRNAs.

Disclosed herein is a CRISPR vector comprising from 5′ to 3′ the following components: a first disclosed ITR, a disclosed promoter or disclosed hybrid promoter that is operably linked to a disclosed Cas9 endonuclease, a disclosed polyA sequence, and a second disclosed ITR. Disclosed herein is a CRISPR vector comprising from 5′ to 3′ the following components: a first disclosed ITR, a disclosed G6PC min promoter or disclosed hybrid promoter comprising a G6PC min promoter and a hSk-CRM4 regulatory module that is operably linked to a disclosed Cas9 endonuclease, a polyA sequence, and a second disclosed ITR.

In an aspect, a disclosed CRISPR vector can comprise the construct set forth in FIG. 1A.

In an aspect, a disclosed CRISPR vector can be an adeno-associated virus (AAV) vector In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but not limited to, AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, a disclosed AAV vector can be AAV-Rh74 or a related variant (e.g., capsid variants like RHM4-1). In an aspect, a disclosed AAV vector can be a recombinant AAV vector (rAAV). In an aspect, a disclosed rAAV vector can be rAAV9.

In an aspect of a disclosed CRISPR vector, a disclosed nucleic acid sequence can have a coding sequence that is less than about 4.5 kilobases.

In an aspect, a disclosed CRISPR vector can comprise a nuclear localization signal (NLS). In an aspect, a disclosed NLS can comprise the sequence set forth in SEQ ID NO:143 or SEQ ID NO:144. In an aspect, a disclosed CRISPR vector can comprise one or more hemagglutinin (HA) tags. In an aspect, a disclosed HA tag can comprise the sequence set forth in SEQ ID NO:111 or SEQ ID NO:112. In an aspect, a CRISPR donor vector can comprise a Rosa26 fragment (e.g., SEQ ID NO:145).

In combination with a disclosed donor vector, a disclosed CRISPR vector can be used in a method of effecting gene editing.

In combination with a disclosed donor vector, a disclosed CRISPR vector can be used in a method of preferentially driving expression of one or more gene products in kidney tissue, heart tissue, skeletal muscle tissue, liver tissue, or any combination thereof. In combination with a disclosed donor vector, a disclosed CRISPR vector can be used in a method of increasing (protein or gene) expression of GAA or G6PC in kidney tissue, heart tissue, skeletal muscle tissue, liver tissue, or any combination thereof. In combination with a disclosed donor vector, a disclosed CRISPR vector can be used in a method of decreasing glycogen content in kidney tissue, heart tissue, skeletal muscle tissue, liver tissue, or any combination thereof. In combination with a disclosed donor vector, a disclosed CRISPR vector can be used in a method of decreasing the level of triglycerides in kidney tissue, heart tissue, skeletal muscle tissue, liver tissue, or any combination thereof. In combination with a disclosed donor vector, a disclosed CRISPR vector can be used in a method of improving hepatocyte morphology and/or decreasing hepatocyte vacuolation. In combination with a disclosed donor vector, a disclosed CRISPR vector can be used in a method of restoring normal blood glucose levels in a patient or a subject in need thereof. In combination with a disclosed donor vector, a disclosed CRISPR vector can be used in a method of reducing the subject's or patient's risk of developing long-term adverse effects. In combination with a disclosed donor vector, a disclosed CRISPR vector can be used in a method of improving hypoglycemia and/or decreasing hyperlipidemia in a subject. In combination with a disclosed donor vector, a disclosed CRISPR vector can be used in a method of increasing the survival of a patient or a subject in need thereof.

In combination with a disclosed donor vector, a disclosed CRISPR vector can be used in a method of restoring the functionality and/or structural integrity of a missing, deficient, and/or mutant protein or enzyme (such as, for example, G6PC or GAA). In combination with a disclosed donor vector, a disclosed CRISPR vector can be used in a method of restoring one or more aspects of cellular homeostasis and/or cellular functionality and/or metabolic dysregulation (such as, for example, glycogen accumulation). In an aspect, restoring one or more aspect of cellular homeostasis and/or cellular functionality and/or metabolic dysregulation comprises restoring the functionality and/or structural integrity of a missing, deficient, and/or mutant protein or enzyme (such as, for example, GAA or G6PC).

In an aspect, restoring one or more aspects of cellular homeostasis and/or cellular functionality can comprise one or more of the following: (i) correcting cell starvation in one or more cell types; (ii) improving, enhancing, restoring, and/or preserving mitochondrial functionality and/or structural integrity; (iii) improving, enhancing, restoring, and/or preserving organelle functionality and/or structural integrity; (iv) correcting enzyme dysregulation; (v) reversing, inhibiting, preventing, stabilizing, and/or slowing the rate of progression of the multi-systemic manifestations of a genetic disease or disorder; (vi) reversing, inhibiting, preventing, stabilizing, and/or slowing the rate of progression of a genetic disease or disorder, (vii) normalizing aspects of the autophagy pathway (such as, for example, correcting, preventing, reducing, and/or ameliorating autophagy), or (viii) any combination thereof. In an aspect, restoring one or more aspects of cellular homeostasis can comprise improving, enhancing, restoring, and/or preserving one or more aspects of cellular structural and/or functional integrity.

In an aspect, restoring the activity and/or functionality of a missing, deficient, and/or mutant protein or enzyme (such as, for example, G6PC or GAA) can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme such as, for example, G6PC or GAA). In an aspect, restoration can be a partial or incomplete restoration. In an aspect, restoration can be complete or near complete restoration such that the level of expression, activity, and/or functionality is similar to that of a wild-type or control level.

In combination with a disclosed donor vector, a disclosed CRISPR vector can be used in a method of correcting and/or redressing and/or reversing and/or treating a pathogenetic defect in skeletal muscle of a subject. In combination with a disclosed donor vector, a disclosed CRISPR vector can be used in a method of correcting and/or redressing and/or reversing and/or treating a pathogenetic defect in cardiac tissue of a subject. In combination with a disclosed donor vector, a disclosed CRISPR vector can be used in a method of correcting and/or redressing and/or reversing and/or treating a pathogenetic defect in kidney tissue and/or liver tissue of a subject.

In combination with a disclosed donor vector, a disclosed CRISPR vector can be used in a method of correcting and/or redressing and/or reversing and/or treating a movement disorder. In an aspect, movement disorder can include neurological diseases or disorders that involve the motor and movement systems, resulting in a range of abnormalities that affect the speed, quality, and ease of movement. Movement disorders are often caused by or related to abnormalities in brain structure and/or function. Movement disorders include, but are not limited to (i) tremors: including, but not limited to, the tremor associated with Parkinson's Disease, physiologic tremor, benign familial tremor, cerebellar tremor, rubral tremor, toxic tremor, metabolic tremor, and senile tremor; (ii) chorea, including, but not limited to, chorea associated with Huntington's Disease, Wilson's Disease, ataxia telangiectasia, infection, drug ingestion, or metabolic, vascular or endocrine etiology (e.g., chorea gravidarum or thyrotoxicosis); (iii) ballism (defined herein as abruptly beginning, repetitive, wide, flinging movements affecting predominantly the proximal limb and girdle muscles); (iv) athetosis (defined herein as relatively slow, twisting, writhing, snake-like movements and postures involving the trunk, neck, face and extremities); (v) dystonia (defined herein as a movement disorder consisting of twisting, turning tonic skeletal muscle contractions, most, but not all of which are initiated distally); (vi) paroxysmal choreoathetosis and tonic spasm; (vii) tics (defined herein as sudden, behaviorally related, irregular, stereotyped, repetitive movements of variable complexity); (viii) tardive dyskinesia; (ix) akathesia, (x) muscle rigidity, defined herein as resistance of a muscle to stretch; (xi) postural instability; (xii) bradykinesia; (xiii) difficulty in initiating movements; (xiv) muscle cramps; (xv) dyskinesias and (xvi) myoclonus. In an aspect, a movement disorder comprises a dystonia.

In an aspect, a disclosed CRISPR vector can drive supraphysiologic GAA expression in a subject's liver and/or a subject's skeletal muscle. In an aspect, a disclosed CRISPR vector can drive supraphysiologic G6PC expression in a subject's liver and/or a subject's skeletal muscle. In an aspect, a disclosed CRISPR vector can restore normal glycogen content in a subject's heart, diaphragm, quadriceps, or any combination thereof. In an aspect, a disclosed CRISPR vector can improve a subject's muscle strength.

In an aspect, a disclosed CRISPR vector does not elicit an immune response.

In an aspect, a disclosed CRISPR vector can ensure persistent transgene expression.

### 4. Gene Editing Systems

Disclosed herein is a gene editing system for stably integrating a transgene into one or more cells, comprising (i) a disclosed donor nucleic acid molecule comprising from 5′ to 3′ a first disclosed ITR, a disclosed 5′ homology arm, a disclosed promoter or disclosed hybrid promoter that is operably linked to a disclosed transgene or a disclosed cDNA, a polyA sequence, a disclosed 3′ homology arm, a promoter that is operably linked to a gRNA, and a second disclosed ITR, and (ii) a disclosed CRISPR nucleic acid molecule comprising from 5′ to 3′ a first disclosed ITR, a disclosed promoter or disclosed hybrid promoter that is operably linked to a disclosed Cas9 endonuclease, a disclosed polyA sequence, and a second disclosed ITR.

Disclosed herein is a gene editing system for stably integrating a transgene into one or more cells, comprising (i) a disclosed donor nucleic acid molecule comprising from 5′ to 3′ a first disclosed ITR, a disclosed 5′ homology arm, a disclosed G6PC min promoter or disclosed hybrid promoter comprising a G6PC min promoter and a hSk-CRM4 regulatory module that is operably linked to a disclosed transgene or a disclosed cDNA, a polyA sequence, a disclosed 3′ homology arm, a U6 promoter that is operably linked to a gRNA, and a second disclosed ITR, and (ii) a disclosed CRISPR nucleic acid molecule comprising from 5′ to 3′ a first disclosed ITR, a disclosed G6PC min promoter or disclosed hybrid promoter comprising a G6PC min promoter and a hSk-CRM4 regulatory module that is operably linked to a disclosed Cas9 endonuclease, a polyA sequence, and a second disclosed ITR.

Disclosed herein is a gene editing system for stably integrating a transgene into one or more cells, comprising a disclosed donor vector and a disclosed CRISPR vector, wherein the one or more cells are in the kidney, the heart, the liver, or the skeletal muscle.

Disclosed herein is a gene editing system for stably integrating a transgene into one or more cells, comprising a disclosed donor vector and a disclosed CRISPR vector.

Disclosed herein is a gene editing system for stably integrating a transgene into one or more cells, comprising a disclosed donor vector and a disclosed CRISPR vector, wherein the one or more cells are in the kidney, the heart, the liver, or the skeletal muscle. Donor vectors are disclosed supra. CRISPR vectors are disclosed supra.

In an aspect of a disclosed gene editing system, a disclosed donor vector can comprise from 5′ to 3′ the following components: a first disclosed ITR, a disclosed 5′ homology arm, a disclosed promoter or disclosed hybrid promoter that is operably linked to a disclosed transgene or a disclosed cDNA, a polyA sequence, a disclosed 3′ homology arm, a promoter that is operably linked to a gRNA, and a second disclosed ITR. In an aspect of a disclosed gene editing system, a disclosed donor vector can comprise from 5′ to 3′ the following components: a first disclosed ITR, a disclosed 5′ homology arm, a disclosed G6PC min promoter or disclosed hybrid promoter comprising a G6PC min promoter and a hSk-CRM4 regulatory module that is operably linked to a disclosed transgene or a disclosed cDNA, a polyA sequence, a disclosed 3′ homology arm, a U6 promoter that is operably linked to a gRNA, and a second disclosed ITR. In an aspect of a disclosed gene editing system, a disclosed CRISPR vector can comprise from 5′ to 3′ the following components: a first disclosed ITR, a disclosed promoter or disclosed hybrid promoter that is operably linked to a disclosed Cas9 endonuclease, a disclosed polyA sequence, and a second disclosed ITR. In an aspect of a disclosed gene editing system, a disclosed CRISPR vector can comprise from 5′ to 3′ the following components: a first disclosed ITR, a disclosed G6PC min promoter or disclosed hybrid promoter comprising a G6PC min promoter and a hSk-CRM4 regulatory module that is operably linked to a disclosed Cas9 endonuclease, a polyA sequence, and a second disclosed ITR.

In an aspect, a disclosed gene editing system can comprise a donor vector such as the construct set forth in FIG. 1A. In an aspect, a disclosed gene editing system can comprise a CRISPR vector such as the construct set forth in FIG. 1A.

In an aspect, a disclosed gene editing system can create a double-strand break (DSB) at or near a mutation or defect in the targeted locus that results in a permanent integration of a disclosed transgene or cDNA. In an aspect, a disclosed gene editing system can create a double-strand break (DSB) at or near a mutation or defect in the targeted GAA locus that results in a permanent integration of a disclosed transgene or cDNA. In an aspect, a disclosed gene editing system can create a double-strand break (DSB) at or near a mutation or defect in the targeted G6PC locus that results in a permanent integration of a disclosed transgene or cDNA.

In an aspect, a disclosed gene editing system can create a double-strand break (DSB) at or near a mutation or defect in the targeted locus associated with or related to a genetic deficiency or genetic disorder that results in a permanent integration of a disclosed transgene or cDNA.

In an aspect, a disclosed gene editing system can create a double-strand break (DSB) at or near a mutation or defect in the targeted locus associated with or related to an inherited protein deficiency or inherited protein disorder that results in a permanent integration of a disclosed transgene or cDNA. In an aspect, a disclosed gene editing system can create a double-strand break (DSB) at or near a mutation or defect in the targeted locus associated with or related to alpha-1-antitrypsin deficiency, phenylketonuria, mitochondrial trifunctional protein deficiency, hemophilia, or muscle dystrophy that results in a permanent integration of a disclosed transgene or cDNA.

In an aspect, a disclosed CRISPR vector can be used in a gene editing application. In an aspect, a disclosed CRISPR vector can be used with a disclosed donor vector.

In an aspect, a disclosed gene editing system can preferentially drive expression of one or more gene products in kidney tissue, heart tissue, skeletal muscle tissue, liver tissue, or any combination thereof. In an aspect, a disclosed gene editing system can increase (protein or gene) expression of GAA or G6PC in kidney tissue, heart tissue, skeletal muscle tissue, liver tissue, or any combination thereof. In an aspect, a disclosed gene editing system can decrease glycogen content in kidney tissue, heart tissue, skeletal muscle tissue, liver tissue, or any combination thereof. In an aspect, a disclosed gene editing system can decrease the level of triglycerides in kidney tissue, heart tissue, skeletal muscle tissue, liver tissue, or any combination thereof. In an aspect, a disclosed gene editing system can improve hepatocyte morphology and/or can decrease hepatocyte vacuolation. In an aspect, a disclosed gene editing system can restore normal blood glucose levels in a patient or a subject in need thereof. In an aspect, a disclosed gene editing system can reduce the subject's or patient's risk of developing long-term adverse effects. In an aspect, a disclosed gene editing system an improve hypoglycemia and/or decrease hyperlipidemia in a subject. In an aspect, a disclosed gene editing system can increase survival of a patient or a subject in need thereof.

In an aspect, a disclosed gene editing system can be used to correct and/or redress and/or reverse and/or treat a movement disorder. In an aspect, movement disorder can include neurological diseases or disorders that involve the motor and movement systems, resulting in a range of abnormalities that affect the speed, quality, and ease of movement. Movement disorders are often caused by or related to abnormalities in brain structure and/or function. Movement disorders include, but are not limited to (i) tremors: including, but not limited to, the tremor associated with Parkinson's Disease, physiologic tremor, benign familial tremor, cerebellar tremor, rubral tremor, toxic tremor, metabolic tremor, and senile tremor; (ii) chorea, including, but not limited to, chorea associated with Huntington's Disease, Wilson's Disease, ataxia telangiectasia, infection, drug ingestion, or metabolic, vascular or endocrine etiology (e.g., chorea gravidarum or thyrotoxicosis); (iii) ballism (defined herein as abruptly beginning, repetitive, wide, flinging movements affecting predominantly the proximal limb and girdle muscles); (iv) athetosis (defined herein as relatively slow, twisting, writhing, snake-like movements and postures involving the trunk, neck, face and extremities); (v) dystonia (defined herein as a movement disorder consisting of twisting, turning tonic skeletal muscle contractions, most, but not all of which are initiated distally); (vi) paroxysmal choreoathetosis and tonic spasm; (vii) tics (defined herein as sudden, behaviorally related, irregular, stereotyped, repetitive movements of variable complexity); (viii) tardive dyskinesia; (ix) akathesia, (x) muscle rigidity, defined herein as resistance of a muscle to stretch; (xi) postural instability; (xii) bradykinesia; (xiii) difficulty in initiating movements; (xiv) muscle cramps; (xv) dyskinesias and (xvi) myoclonus. In an aspect, a movement disorder comprises a dystonia.

In an aspect, a disclosed gene editing system can restore the functionality and/or structural integrity of a missing, deficient, and/or mutant protein or enzyme (such as, for example, G6PC or GAA).

In an aspect, a disclosed gene editing system can restore one or more aspects of cellular homeostasis and/or cellular functionality and/or metabolic dysregulation (such as, for example, glycogen accumulation). In an aspect, restoring one or more aspect of cellular homeostasis and/or cellular functionality and/or metabolic dysregulation comprises restoring the functionality and/or structural integrity of a missing, deficient, and/or mutant protein or enzyme (such as, for example, GAA or G6PC).

In an aspect, restoring one or more aspects of cellular homeostasis and/or cellular functionality can comprise one or more of the following: (i) correcting cell starvation in one or more cell types; (ii) improving, enhancing, restoring, and/or preserving mitochondrial functionality and/or structural integrity; (iii) improving, enhancing, restoring, and/or preserving organelle functionality and/or structural integrity; (iv) correcting enzyme dysregulation; (v) reversing, inhibiting, preventing, stabilizing, and/or slowing the rate of progression of the multi-systemic manifestations of a genetic disease or disorder; (vi) reversing, inhibiting, preventing, stabilizing, and/or slowing the rate of progression of a genetic disease or disorder, (vii) normalizing aspects of the autophagy pathway (such as, for example, correcting, preventing, reducing, and/or ameliorating autophagy), or (viii) any combination thereof. In an aspect, restoring one or more aspects of cellular homeostasis can comprise improving, enhancing, restoring, and/or preserving one or more aspects of cellular structural and/or functional integrity.

In an aspect, restoring the activity and/or functionality of a missing, deficient, and/or mutant protein or enzyme (such as, for example, G6PC or GAA) can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme such as, for example, G6PC or GAA). In an aspect, restoration can be a partial or incomplete restoration. In an aspect, restoration can be complete or near complete restoration such that the level of expression, activity, and/or functionality is similar to that of a wild-type or control level.

In an aspect, a disclosed gene editing system can be used to correct and/or redress and/or reverse and/or treat a pathogenetic defect in skeletal muscle of a subject. In an aspect, a disclosed gene editing system can be used to correct and/or redress and/or reverse and/or treat a pathogenetic defect in cardiac tissue of a subject. In an aspect, a disclosed gene editing system can be used to correct and/or redress and/or reverse and/or treat a pathogenetic defect in kidney tissue and/or liver tissue of a subject.

In an aspect, a disclosed gene editing system can drive supraphysiologic GAA expression in a subject's liver and/or a subject's skeletal muscle. In an aspect, a disclosed gene editing system can drive supraphysiologic G6PC expression in a subject's liver and/or a subject's skeletal muscle. In an aspect, a disclosed gene editing system can restore normal glycogen content in a subject's heart, diaphragm, quadriceps, or any combination thereof. In an aspect, a disclosed gene editing system can improve a subject's muscle strength.

In an aspect, a disclosed gene editing system does not elicit an immune response.

In an aspect of a disclosed gene editing system, a disclosed Donor vector and a disclosed CRISPR vector can be delivered in a ratio (e.g., “Donor” to “CRISPR”). In an aspect, a ratio of the Donor vector to the CRISPR vector can be from about 10:1 to about 1:1, from about 9:1 to about 1:1, from about 8:1 to about 1:1, from about 7:1 to about 1:1, from about 6:1 to about 1:1, from about 5:1 to about 1:1, from about 4:1 to about 1:1, from about 3:1 to about 1:1, from about 2:1 to about 1:1. In an aspect, a ratio of the Donor vector to the CRISPR vector can be from 10:1 to 1:1, from 9:1 to 1:1, from 8:1 to 1:1, from 7:1 to 1:1, from 6:1 to 1:1, from 5:1 to 1:1, from 4:1 to 1:1, from 3:1 to 1:1, from 2:1 to 1:1. In an aspect, the ratio of the Donor vector to the CRISPR vector can be about 10:1, about 9:1, about 8:1, about 7:1, about 6:1, about 5:1, about 4:1, about 3:1, about 2:1, or about 1:1. In an aspect, the ratio of the Donor vector to the CRISPR vector can be 10:1, 9:1, 8:1, 7:1, 6:1, 5:1, 4:1, 3:1, 2:1, or 1:1. In an aspect, the ratio of the Donor vector to the CRISPR vector can be about 4:1, about 2:1, or about 1:1.

In an aspect of a disclosed gene editing system, a disclosed CRISPR vector and a disclosed Donor vector can be delivered in a ratio (e.g., “CRISPR” to “Donor”). In an aspect, a ratio of the CRISPR vector to the Donor vector can be from about 10:1 to about 1:1, from about 9:1 to about 1:1, from about 8:1 to about 1:1, from about 7:1 to about 1:1, from about 6:1 to about 1:1, from about 5:1 to about 1:1, from about 4:1 to about 1:1, from about 3:1 to about 1:1, from about 2:1 to about 1:1. In an aspect, a ratio of the CRISPR vector to the Donor vector can be from 10:1 to 1:1, from 9:1 to 1:1, from 8:1 to 1:1, from 7:1 to 1:1, from 6:1 to 1:1, from 5:1 to 1:1, from 4:1 to 1:1, from 3:1 to 1:1, from 2:1 to 1:1. In an aspect, the ratio of the CRISPR vector to the Donor vector can be about 10:1, about 9:1, about 8:1, about 7:1, about 6:1, about 5:1, about 4:1, about 3:1, about 2:1, or about 1:1. In an aspect, the ratio of the CRISPR vector to the Donor vector can be 10:1, 9:1, 8:1, 7:1, 6:1, 5:1, 4:1, 3:1, 2:1, or 1:1. In an aspect, the ratio of the CRISPR vector to the Donor vector can be about 4:1, about 2:1, or about 1:1.

In an aspect, a disclosed gene editing system can ensure persistent transgene expression.

### 5. Pharmaceutical Formulations

Disclosed herein is a pharmaceutical formulation comprising a disclosed isolated nucleic acid molecule. Disclosed herein is a pharmaceutical formulation comprising a disclosed isolated nucleic acid molecule and a pharmaceutically acceptable carrier. Disclosed herein is a pharmaceutical formulation comprising a disclosed donor vector. Disclosed herein is a pharmaceutical formulation comprising a disclosed donor vector and a pharmaceutically acceptable carrier. Disclosed herein is a pharmaceutical formulation comprising a disclosed CRISPR vector. Disclosed herein is a pharmaceutical formulation comprising a disclosed CRISPR vector and a pharmaceutically acceptable carrier.

In an aspect, a disclosed pharmaceutical formulation can repair a defective G6PC gene. In an aspect, a disclosed pharmaceutical formulation can repair a defective GAA gene. In an aspect, a disclosed pharmaceutical formulation can be used in a method of treating a GSDIa or GSDII or in a method of validating a gene editing system.

In an aspect, a disclosed pharmaceutical formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof.

In an aspect, a disclosed composition can comprise one or more immune modulators. In an aspect, a disclosed composition can comprise one or more proteasome inhibitors. In an aspect, a disclosed composition can comprise one or more immunosuppressives or immunosuppressive agents. In an aspect, an immunosuppressive agent can be anti-thymocyte globulin (ATG), cyclosporine (CSP), mycophenolate mofetil (MMF), or a combination thereof. In an aspect, a disclosed formulation can comprise an anaplerotic agent (such as, for example, C7 compounds like triheptanoin or MCT).

In an aspect, a disclosed formulation can comprise a disclosed small molecule. In an aspect, a disclosed small molecule can assist in restoring the functionality and/or structural integrity of a missing, deficient, and/or mutant protein or enzyme. In an aspect, a disclosed formulation can comprise one or more fibrates. In an aspect, a disclosed formulation can comprise bezafibrate, fenofibrate, ciprofibrate, gemfibrozil, clofibrate, an analog thereof, or a combination thereof.

In an aspect, a disclosed pharmaceutical formulation can comprise one or more excipients and/or pharmaceutically acceptable carriers. Excipients and/or pharmaceutically acceptable carriers are known to the art and are discussed supra.

In an aspect, a disclosed pharmaceutical formulation can be used in a gene editing application. In an aspect, a disclosed pharmaceutical formulation can be used with a disclosed donor vector. In an aspect, a disclosed pharmaceutical formulation can be used to preferentially drive expression of one or more gene products in kidney tissue, heart tissue, skeletal muscle tissue, liver tissue, or any combination thereof. In an aspect, a disclosed pharmaceutical formulation can be used to increase (protein or gene) expression of GAA or G6PC in kidney tissue, heart tissue, skeletal muscle tissue, liver tissue, or any combination thereof. In In an aspect, a disclosed pharmaceutical formulation can be used to decrease glycogen content in kidney tissue, heart tissue, skeletal muscle tissue, liver tissue, or any combination thereof. In an aspect, a disclosed pharmaceutical formulation can be used to decrease the level of triglycerides in kidney tissue, heart tissue, skeletal muscle tissue, liver tissue, or any combination thereof. In an aspect, a disclosed pharmaceutical formulation can be used to improve hepatocyte morphology and/or can decrease hepatocyte vacuolation. In an aspect, a disclosed pharmaceutical formulation can be used to restore normal blood glucose levels in a patient or a subject in need thereof. In an aspect, a disclosed pharmaceutical formulation can be used to reduce the subject's or patient's risk of developing long-term adverse effects. In an aspect, a disclosed pharmaceutical formulation can be used to increase survival of a patient or a subject in need thereof.

In an aspect, a disclosed pharmaceutical formulation can be used to correct and/or redress and/or reverse and/or treat a movement disorder. In an aspect, movement disorder can include neurological diseases or disorders that involve the motor and movement systems, resulting in a range of abnormalities that affect the speed, quality, and ease of movement. Movement disorders are often caused by or related to abnormalities in brain structure and/or function. Movement disorders include, but are not limited to (i) tremors: including, but not limited to, the tremor associated with Parkinson's Disease, physiologic tremor, benign familial tremor, cerebellar tremor, rubral tremor, toxic tremor, metabolic tremor, and senile tremor; (ii) chorea, including, but not limited to, chorea associated with Huntington's Disease, Wilson's Disease, ataxia telangiectasia, infection, drug ingestion, or metabolic, vascular or endocrine etiology (e.g., chorea gravidarum or thyrotoxicosis); (iii) ballism (defined herein as abruptly beginning, repetitive, wide, flinging movements affecting predominantly the proximal limb and girdle muscles); (iv) athetosis (defined herein as relatively slow, twisting, writhing, snake-like movements and postures involving the trunk, neck, face and extremities); (v) dystonia (defined herein as a movement disorder consisting of twisting, turning tonic skeletal muscle contractions, most, but not all of which are initiated distally); (vi) paroxysmal choreoathetosis and tonic spasm; (vii) tics (defined herein as sudden, behaviorally related, irregular, stereotyped, repetitive movements of variable complexity); (viii) tardive dyskinesia; (ix) akathesia, (x) muscle rigidity, defined herein as resistance of a muscle to stretch; (xi) postural instability; (xii) bradykinesia; (xiii) difficulty in initiating movements; (xiv) muscle cramps; (xv) dyskinesias and (xvi) myoclonus. In an aspect, a movement disorder comprises a dystonia.

In an aspect, a disclosed pharmaceutical formulation can be used to restore the functionality and/or structural integrity of a missing, deficient, and/or mutant protein or enzyme (such as, for example, G6PC or GAA). In an aspect, a disclosed gene editing system can restore one or more aspects of cellular homeostasis and/or cellular functionality and/or metabolic dysregulation (such as, for example, glycogen accumulation). In an aspect, restoring one or more aspect of cellular homeostasis and/or cellular functionality and/or metabolic dysregulation comprises restoring the functionality and/or structural integrity of a missing, deficient, and/or mutant protein or enzyme (such as, for example, GAA or G6PC).

In an aspect, restoring one or more aspects of cellular homeostasis and/or cellular functionality can comprise one or more of the following: (i) correcting cell starvation in one or more cell types; (ii) improving, enhancing, restoring, and/or preserving mitochondrial functionality and/or structural integrity; (iii) improving, enhancing, restoring, and/or preserving organelle functionality and/or structural integrity; (iv) correcting enzyme dysregulation; (v) reversing, inhibiting, preventing, stabilizing, and/or slowing the rate of progression of the multi-systemic manifestations of a genetic disease or disorder; (vi) reversing, inhibiting, preventing, stabilizing, and/or slowing the rate of progression of a genetic disease or disorder, (vii) normalizing aspects of the autophagy pathway (such as, for example, correcting, preventing, reducing, and/or ameliorating autophagy), or (viii) any combination thereof. In an aspect, restoring one or more aspects of cellular homeostasis can comprise improving, enhancing, restoring, and/or preserving one or more aspects of cellular structural and/or functional integrity.

In an aspect, restoring the activity and/or functionality of a missing, deficient, and/or mutant protein or enzyme (such as, for example, G6PC or GAA) can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme such as, for example, G6PC or GAA). In an aspect, restoration can be a partial or incomplete restoration. In an aspect, restoration can be complete or near complete restoration such that the level of expression, activity, and/or functionality is similar to that of a wild-type or control level.

In an aspect, a disclosed pharmaceutical formulation can be used to correct and/or redress and/or reverse and/or treat a pathogenetic defect in skeletal muscle of a subject. In an aspect, a disclosed pharmaceutical formulation can be used to correct and/or redress and/or reverse and/or treat a pathogenetic defect in cardiac tissue of a subject. In an aspect, a disclosed pharmaceutical formulation can be used to correct and/or redress and/or reverse and/or treat a pathogenetic defect in kidney tissue and/or liver tissue of a subject.

In an aspect, a disclosed pharmaceutical formulation can drive supraphysiologic GAA expression in a subject's liver and/or a subject's skeletal muscle. In an aspect, a disclosed pharmaceutical formulation can drive supraphysiologic G6PC expression in a subject's liver and/or a subject's skeletal muscle. In an aspect, a disclosed pharmaceutical formulation can restore normal glycogen content in a subject's heart, diaphragm, quadriceps, or any combination thereof. In an aspect, a disclosed pharmaceutical formulation can improve a subject's muscle strength.

In an aspect, a disclosed pharmaceutical formulation does not elicit an immune response.

In an aspect, a disclosed pharmaceutical formulation can ensure persistent transgene expression.

### 6. Plasmids

Disclosed herein is a plasmid comprising one or more disclosed isolated nucleic acid molecules. Disclosed herein is a plasmid comprising the nucleic acid sequence for one or more G6PC promoters. Disclosed herein is a plasmid comprising the nucleic acid sequence for one or more hybrid promoters. Disclosed here are plasmids used in methods of making a disclosed composition such as, for example, a disclosed isolated nucleic acid molecule, a disclosed vector, or a disclosed pharmaceutical formulation. Plasmids and using plasmids are known to the art.

Disclosed herein is a plasmid comprising the sequence set forth in any one of SEQ ID NO:49-SEQ ID NO:55. Disclosed herein is a plasmid comprising a sequence having at least 40%, 50%, 60%, 70%, 80%, 85%, 86%, 87%, 88%, 89%, 90%, 91%, 92%, 93%, 94%, 95%, 96%, 97%, 98%, 99% identity to the sequence set forth in any one of SEQ ID NO:49-SEQ ID NO:55 or a fragment thereof. Disclosed herein is a plasmid comprising a sequence having at least 40%-60%, at least 60%-80%, at least 80%-90%, or at least 90%-100% identity to the sequence set forth in any one of SEQ ID NO:49-SEQ ID NO:55 or a fragment thereof.

In an aspect, a disclosed plasmid can restore one or more aspects of cellular homeostasis and/or cellular functionality and/or metabolic dysregulation. In an aspect, a disclosed plasmid can improve the efficiency of gene editing. In an aspect, a disclosed plasmid can be used to stably integrate a transgene (such as, for example, G6PC or GAA) into one or more disclosed cells. In an aspect, a disclosed plasmid can be in a method of treating a GSD patient or in a method of validating a gene editing system. In an aspect, a disclosed plasmid can restore one or more aspects of cellular homeostasis and/or cellular functionality and/or metabolic dysregulation (such as, for example, glycogen accumulation). In an aspect, a disclosed plasmid can restore the functionality and/or structural integrity of a missing, deficient, and/or mutant protein or enzyme (such as, for example, a mutant or defective G6PC or GAA).

### 7. Cells

Disclosed herein are cells comprising a disclosed isolated nucleic acid molecule, a disclosed vector, and/or a disclosed plasmid. Disclosed herein are cells transduced by one or more disclosed viral vectors. Disclosed herein are cells transfected with one or more disclosed isolated nucleic acid molecules. Cells are known to the art and include liver, muscle, kidney, cardiac, and brain cells. In an aspect, a disclosed cell can be any cell having excessive glycogen or any cell affected by a defective G6PC gene or GAA gene. In an aspect, a disclosed cell has been transfected with one or more disclosed nucleic acid sequences. In an aspect, a disclosed cell has been transduced with a disclosed donor vector. In an aspect, a disclosed cell has been transduced with a disclosed donor vector and a disclosed CRISPR vector. Techniques to achieve transfection and transduction are known to the art and using transfected or transduced cells are known to the art. In an aspect, disclosed herein are human immortalized cells lines transduced by one or more disclosed viral vectors or transfected with one or more disclosed isolated nucleic acids or disclosed plasmids. In an aspect, disclosed herein are human immortalized cells lines contacted with one or more disclosed pharmaceutical formulations. Disclosed herein are cells obtained for a subject treated with one or more disclosed isolated nucleic acid molecule, one or more disclosed vectors, one or more disclosed plasmids, or one or more disclosed pharmaceutical formulations.

### 8. Animals

Disclosed herein are animals treated with one or more disclosed isolated nucleic acid molecules, one or more disclosed donor vectors, one or more disclosed CRISPR vectors, one or more disclosed pharmaceutical formulations, and/or one or more disclosed plasmids. Disclosed herein are animals treated with a disclosed donor vector. Disclosed herein are animals treated with a disclosed donor vector and a disclosed CRISPR vector. Disclosed herein are animals treated with a disclosed gene editing system. Transgenic animals are known to the art as are the techniques to generate transgenic animals.

### D. Methods of Repairing a Defective Gene

Disclosed herein is a method of repairing a defective gene, the method comprising contacting cells with a disclosed donor nucleic acid molecule and a disclosed CRISPR nucleic acid molecule, wherein, following expression of the nucleic acid molecules, the defective gene is repaired in the cells. Donor vectors and CRISPR vectors are disclosed supra.

Disclosed herein is a method of repairing a defective gene, the method comprising contacting cells with a disclosed donor vector and a disclosed CRISPR vector, wherein, following expression of the nucleic acid sequences, the defective gene is repaired in the cells. Donor vectors and CRISPR vectors are disclosed supra.

Disclosed herein is a method of repairing a defective gene, the method comprising contacting cells with a disclosed gene editing system, wherein, following expression of the nucleic acid sequences, the defective gene is repaired in the cells. Gene editing systems are disclosed supra.

In an aspect, the disclosed cells can comprise any cells. In an aspect, the disclosed cells can preferentially comprise cells in kidney tissue, heart tissue, skeletal muscle tissue, liver tissue, or any combination thereof. In an aspect, the disclosed cells can preferentially comprise kidney cells, heart cells, skeletal muscle cells, liver cells, or any combination thereof.

In an aspect, the disclosed cells can comprise any cells affected by a defective GAA gene. Defects in the GAA gene are known to the skilled person in the art. In an aspect, the disclosed cells can comprise any cells affected by a defective G6PC gene. Defects in the G6PC gene are known to the skilled person in the art.

In an aspect, the disclosed cells can be any cells having a high and/or excess level of glycogen. In an aspect, the disclosed cells can be any cells experiencing dysfunctional glycogenolysis pathways and/or dysfunctional gluconeogenesis pathways. In an aspect, a disclosed transgene can be expressed in dividing cells. In an aspect, a disclosed transgene can be expressed in non-dividing cells. In an aspect, a disclosed transgene can be expressed in both dividing and non-dividing cells. In an aspect, expression of a disclosed transgene can be effected in dividing cells and/or non-dividing cells.

In an aspect, the disclosed cells can be in a subject. In an aspect, a disclosed subject can be diagnosed with or is suspected of having a genetic disease or a genetic disorder. In an aspect, a disclosed subject can be diagnosed with or is suspected of having GSDIa. In an aspect, a disclosed subject can be diagnosed with or is suspected of having GSDII. In an aspect, a subject can have late onset GSDII or can have infantile onset of GSDII.

In an aspect, a disclosed subject can be diagnosed with or is suspected of having an inherited protein deficiency or inherited protein disorder. In an aspect, a disclosed subject can be diagnosed with or is suspected of having alpha-1-antitrypsin deficiency, phenylketonuria, mitochondrial trifunctional protein deficiency, hemophilia, or muscle dystrophy. In an aspect, a disclosed subject can be an adult at the time of receiving a disclosed donor vector, a disclosed CRISPR vector, a disclosed gene editing system, a disclosed donor nucleic acid molecule, a disclosed CRISPR nucleic acid molecule, or a disclosed pharmaceutical formulation.

In an aspect, a disclosed subject can be an infant at the time of receiving a disclosed donor vector, a disclosed CRISPR vector, a disclosed gene editing system, a disclosed donor nucleic acid molecule, a disclosed CRISPR nucleic acid molecule, or a disclosed pharmaceutical formulation.

In an aspect, a disclosed subject can be an adult at the time of a disclosed method. In an aspect, a disclosed subject can be an infant at the time of a disclosed method.

In an aspect, a disclosed method can generate a depot of the therapeutic expression of a transgene. In an aspect, a disclosed method can generate a depot of the therapeutic expression of a transgene in a subject's liver and/or in a subject's skeletal muscle. In an aspect, a disclosed method can improve hypoglycemia and/or decrease hyperlipidemia in a subject.

In an aspect, a disclosed method can be used to correct and/or redress and/or reverse and/or treat a pathogenetic defect in skeletal muscle of a subject. In an aspect, a disclosed method can be used to correct and/or redress and/or reverse and/or treat a pathogenetic defect in cardiac tissue of a subject. In an aspect, a disclosed method can be used to correct and/or redress and/or reverse and/or treat a pathogenetic defect in kidney tissue and/or liver tissue of a subject.

In an aspect, a disclosed method of repairing a defective gene (e.g., GAA or G6PC) can restore the functionality and/or structural integrity of a missing, deficient, and/or mutant protein or enzyme (e.g., GAA or G6PC). In an aspect, a disclosed method can comprise restoring one or more aspects of cellular homeostasis and/or cellular functionality and/or metabolic dysregulation (such as, for example, glycogen accumulation). In an aspect, restoring one or more aspect of cellular homeostasis and/or cellular functionality and/or metabolic dysregulation comprises restoring the functionality and/or structural integrity of a missing, deficient, and/or mutant protein or enzyme (such as, for example, G6PC or GAA).

In an aspect, restoring one or more aspects of cellular homeostasis and/or cellular functionality can comprise one or more of the following: (i) correcting cell starvation in one or more cell types; (ii) improving, enhancing, restoring, and/or preserving mitochondrial functionality and/or structural integrity; (iii) improving, enhancing, restoring, and/or preserving organelle functionality and/or structural integrity; (iv) correcting enzyme dysregulation; (v) reversing, inhibiting, preventing, stabilizing, and/or slowing the rate of progression of the multi-systemic manifestations of a genetic disease or disorder; (vi) reversing, inhibiting, preventing, stabilizing, and/or slowing the rate of progression of a genetic disease or disorder, (vii) normalizing aspects of the autophagy pathway (such as, for example, correcting, preventing, reducing, and/or ameliorating autophagy), or (viii) any combination thereof. In an aspect, restoring one or more aspects of cellular homeostasis can comprise improving, enhancing, restoring, and/or preserving one or more aspects of cellular structural and/or functional integrity.

In an aspect, restoring the activity and/or functionality of a missing, deficient, and/or mutant protein or enzyme (such as, for example, G6PC or GAA) can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme such as, for example, GAA). In an aspect, restoration can be a partial or incomplete restoration. In an aspect, restoration can be complete or near complete restoration such that the level of expression, activity, and/or functionality is similar to that of a wild-type or control level.

In an aspect of a disclosed method of repairing a defective gene (such as, for example, G6PC or GAA), techniques to monitor, measure, and/or assess the restoring one or more aspects of cellular homeostasis and/or cellular functionality can comprise qualitative (or subjective) means as well as quantitative (or objective) means. These means are known to the skilled person.

In an aspect, a disclosed method of repairing a defective gene can improve and/or eliminate and/or diminish one or more symptoms associated with the genetic disease or genetic disorder. In an aspect, a disclosed method can improve a subject's ability to ambulate.

In an aspect of a disclosed method of repairing a defective gene, a disclosed vector can be administered systemically or directly to the subject. In an aspect, a disclosed vector can be intravenously, subcutaneously, or intramuscularly administered to the subject.

In an aspect, a therapeutically effective amount of a disclosed donor vector can comprise a range of about 1×10¹⁰ vg/kg to about 2×10¹⁴ vg/kg. In an aspect, for example, a disclosed donor vector can be administered at a dose of about 1×10¹¹ to about 8×10¹³ vg/kg or about 1×10¹² to about 8×10¹³ vg/kg. In an aspect, a disclosed donor vector can be administered at a dose of about 1×10¹³ to about 6×10¹³ vg/kg. In an aspect, a disclosed donor vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed donor vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed donor vector can be administered at a dose of about 1×10¹² vg/kg. In an aspect, a disclosed donor vector can be administered at a dose of about 1×10¹¹ vg/kg. In an aspect, a disclosed donor vector can be administered in a single dose, or in multiple doses (such as 2, 3, 4, 5, 6, 7, 8, 9 or 10 doses) as needed for the desired therapeutic results.

In an aspect, a therapeutically effective amount of a disclosed CRISPR vector can comprise a range of about 1×10¹⁰ vg/kg to about 2×10¹⁴-vg/kg. In an aspect, for example, a disclosed CRISPR vector can be administered at a dose of about 1×10¹¹ to about 8×10¹³ vg/kg or about 1×10¹² to about 8×10¹³ vg/kg. In an aspect, a disclosed CRISPR vector can be administered at a dose of about 1×10¹³ to about 6×10¹³ vg/kg. In an aspect, a disclosed CRISPR vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed CRISPR vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed CRISPR vector can be administered at a dose of about 1×10¹² vg/kg. In an aspect, a disclosed CRISPR vector can be administered at a dose of about 1×10¹¹ vg/kg. In an aspect, a disclosed CRISPR vector can be administered in a single dose, or in multiple doses (such as 2, 3, 4, 5, 6, 7, 8, 9 or 10 doses) as needed for the desired therapeutic results.

In an aspect, a disclosed method of repairing a defective gene can comprise administering to the subject one or more additional therapeutic agents. In an aspect, a disclosed therapeutic agent can comprise enzyme replacement therapy, gene therapy, mRNA therapy, small molecule therapy, substrate reduction therapy, or any combination thereof.

In an aspect, a disclosed method of repairing a defective gene can comprise treating the subject. In an aspect, treating the subject can comprise administering to the subject one or more agents that modulate the level of one or more differentially present cellular metabolites. In an aspect, treating the subject can comprise implementing a change in the subject's dietary intake of carbohydrates. Implementing a change in the subject's dietary intake of carbohydrates can comprise adding carbohydrates to the subject's diet, or removing carbohydrates from the subject's diet, or changing the type of carbohydrates in the subject's diet, or changing the frequency of carbohydrates consumed by the subject. In an aspect, treating the subject can comprise administering cornstarch to the subject, or administering glycoside to the subject, or administering one or more anaplerotic agents to the subject.

In an aspect of a disclosed method of repairing a defective gene (such as, for example, G6PC or GAA), a disclosed Cas9 nuclease can create a double-strand breaks (DSB) within or near a mutation or defect or a sequence variant in the gene locus that results in a permanent integration of the repair template.

In an aspect, a disclosed method of repairing a defective gene can improve the efficiency of gene editing. In an aspect, a disclosed method can be used to stably integrate a transgene (such as, for example, G6PC or GAA) into one or more disclosed cells. In an aspect, a disclosed method can improve the efficiency of gene editing. In an aspect, a disclosed transgene can be a full-length transgene. In an aspect, a disclosed method can be used in a method of treating a patient or in a method of validating a gene editing system. In an aspect, a disclosed method can comprise validating the expression of the transgene. In an aspect, a disclosed method can comprise confirming that the Cas9 created a double-strand break (DSB) on both sides of a mutation or defect in the GAA locus that results in a permanent integration of the repair template, thereby repairing the defect underlying the genetic disease or disorder (such as, GSDIa or GSDII).

In an aspect, a disclosed method can further comprise measuring the level or amount of one or more biomarkers (e.g., glucose, ALT, creatinine, glycogen, hepatocellular vacuolation, etc.), one or more indicators of the subject's metabolomic health, or any combination thereof.

In an aspect, validating the efficacy of a gene editing system can comprise measuring the expression of the transgene in the edited cells; and comparing the resulting transgene expression level in the edited cells to the transgene expression level in control cells, wherein the gene editing system is effective when the transgene expression in the edited cells is greater than the transgene expression level in control cells.

In an aspect, validating the efficacy of a gene editing system can comprise measuring the expression of a reporter gene in the edited cells; and comparing the resulting expression level of the reporter gene in the edited cells to the expression level of the reporter gene in control cells, wherein the gene editing system is effective when the expression level of the reporter gene in the edited cells is greater than the expression level of the reporter gene in control cells.

In an aspect, validating the efficacy of a gene editing system in a subject can comprise administering to the subject a disclosed vector comprising a disclosed isolated nucleic acid molecule; obtaining a biological sample of cells targets for editing; measuring the expression of a reporter gene in the edited cells; and comparing the resulting expression level of the reporter gene in the edited cells to the expression level of the reporter gene in control cells, wherein the gene editing system is effective when the expression level of the reporter gene in the edited cells is greater than the expression level of the reporter gene in control cells.

In an aspect, validating the efficacy of a gene editing system in a subject can comprise administering to the subject a disclosed vector comprising a disclosed isolated nucleic acid molecule; obtaining a biological sample of cells targets for editing; measuring the expression of a reporter gene in the edited cells; and comparing the resulting expression level of the reporter gene in the edited cells to the expression level of the reporter gene in control cells, wherein the gene editing system is effective when the expression level of the reporter gene in the edited cells is greater than the expression level of the reporter gene in control cells.

In a disclosed method, the disclosed control cells can comprise unedited cells. In an aspect, the disclosed control cells can comprise the subject's cells prior to administration of a disclosed vector, a disclosed nucleic acid molecule, or a disclosed pharmaceutical formulation. In an aspect, the disclosed control cells can comprise cells treated with a disclosed nucleic acid molecule or a disclosed vector having a scrambled gRNA.

In an aspect, measuring the expression of a disclosed transgene and/or the reporter gene can comprise measuring the protein concentration of the transgene and/or the reporter gene or measuring the mRNA level of transgene and/or the reporter gene. For example, in an aspect, measuring the protein concentration of transgene and/or the reporter gene comprises a protein chip analysis, an immunoassay, a ligand binding assay, a MALDI-TOF (Matrix Assisted Laser Desorption/Ionization Time of Flight Mass Spectrometry) analysis, a SELDI-TOF (Sulface Enhanced Laser Desorption/Ionization Time of Flight Mass Spectrometry) analysis, a radioimmunoassay, a radioimmunodiffusion assay, an octeroni immunodiffusion method, rocket immunoelectrophoresis, tissue immunostaining, a complement fixation assay, 2D by electrophoretic analysis, liquid chromatography-Mass Spectrometry (LC-MS), liquid chromatography-Mass Spectrometry/Mass Spectrometry (LC-MS/MS), Western blotting, ELISA (enzyme linked immunosorbent assay), or any combination thereof. Similarly, in an aspect, measuring the mRNA level of GAA and/or the reporter gene comprises a reverse transcription polymerase reaction (RT-PCR), a competitive reverse transcription polymerase reaction (Competitive RT-PCR), a real-time reverse transcription polymerization, an enzyme reaction (Real-time RT-PCR), an RNase protection assay (RPA), Northern blotting, a DNA chip, or any combination thereof.

In an aspect, a disclosed method of repairing a defective gene can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria. Such methods are known to the skilled person. In an aspect, a disclosed method can further comprise repeating a monitoring step.

In an aspect, a disclosed method of repairing a defective gene can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method of repairing a defective gene can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed method of repairing a defective gene can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method of repairing a defective gene can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method of repairing a defective gene (e.g., GAA or G6PC) can further comprise repeating a disclosed administering step such as, for example, repeating the administering of a disclosed isolated nucleic acid molecule, a disclosed vector, a disclosed pharmaceutical formulation, a disclosed therapeutic agent, a disclosed immune modulator, a disclosed proteasome inhibitor, a disclosed immunosuppressive agent, a disclosed compound that exerts a therapeutic effect against B cells and/or a disclosed compound that targets or alters antigen presentation or humoral or cell mediated immune response.

In an aspect, a disclosed method of repairing a defective gene can further comprise administering a β2 agonist. For example, in an aspect, a disclosed method can comprises administering a β2 agonist to increase the expression of one or more receptors for a lysosomal enzyme. In an aspect, β2 agonists include but are not limited to albuterol, clenbuterol, formoterol, indacaterol, olodaterol, salmeterol, vilanterol, and any combination thereof, growth hormones (e.g., human growth hormone), autocrine glycoprotein (e.g., Follistatin), or any combination thereof (see, e.g., U.S. Pat. No. 8,679,478 for a discussion of appropriate β2 agonists, which patent is incorporated by reference it its entirety for these teachings).

In an aspect, a disclosed method of repairing a defective gene can further comprise administering to a subject or patient one or more fibrates. In an aspect, a disclosed fibrate can comprise bezafibrate, fenofibrate, ciprofibrate, gemfibrozil, clofibrate, an analog thereof, or a combination thereof. In an aspect, a disclosed method can comprise repeating the administering of one or more fibrates one or more times. In an aspect, a therapeutically effective amount of one or more fibrates can comprise at least about 20 mg/day to at least 500 mg/day. In an aspect, a therapeutically effective amount of one or more fibrates can comprise at least about 20 mg/day, at least about 20 mg/day, about 30 mg/day, about 40 mg/day, at least about 50 mg/day, at least about 60 mg/day, at least about 70 mg/day, at least about 80 mg/day, at least about 90 mg/day, at least about 100 mg/day, at least about 120 mg/day, at least about 140 mg/day, at least about 160 mg/day, at least about 180 mg/day, at least about 200 mg/day, at least about 220 mg/day, at least about 240 mg/day, at least about 260 mg/day, at least about 280 mg/day, at least about 300 mg/day, at least about 320 mg/day, at least about 340 mg/day, at least about 360 mg/day, at least about 380 mg/day, at least about 400 mg/day, at least about 420 mg/day, at least about 440 mg/day, at least about 460 mg/day, at least about 480 mg/day, or at least about 500 mg/day.

In an aspect, a disclosed method of repairing a defective gene can further comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, one or more disclosed nucleic acid molecules, donor vectors, CRISPR vectors, pharmaceutical formulations, or any combination thereof can be administered concurrently or sequentially.

In an aspect, a disclosed method can further comprise diagnosing a subject with a genetic defect using one or more known methods to the skilled person, such as, for example, genotyping.

In an aspect, a disclosed method can drive supraphysiologic GAA expression in a subject's liver and/or a subject's skeletal muscle. In an aspect, a disclosed method can drive supraphysiologic G6PC expression in a subject's liver and/or a subject's skeletal muscle. In an aspect, a disclosed method can restore normal glycogen content in a subject's heart, diaphragm, quadriceps, or any combination thereof. In an aspect, a disclosed method can improve a subject's muscle strength.

In an aspect, a disclosed method does not elicit an immune response. In an aspect, a disclosed method can ensure persistent transgene expression.

In an aspect of a disclosed method, a disclosed donor vector can achieve episomal expression of a disclosed transgene.

### E. Methods of Treating a Subject Having a Genetic Disease or Disorder

Disclosed herein is an in vivo method for treating a subject having a genetic disease or genetic disorder, the method comprising administering to the subject a therapeutically effective amount of a disclosed donor vector; and administering to the subject therapeutically effective amount of a disclosed vector; wherein the Cas9 creates a double-strand break (DSB) on both sides of a mutation or defect in the gene locus that results in a permanent integration of a disclosed transgene, thereby repairing the defect underlying the genetic disease or disorder.

Disclosed herein is an in vivo method for treating a subject having GSDIa, the method comprising administering to the subject a therapeutically effective amount of a disclosed donor vector; and administering to the subject therapeutically effective amount of a disclosed vector; wherein the Cas9 creates a double-strand break (DSB) on both sides of a mutation or defect in the G6PC locus that results in a permanent integration of a disclosed G6PC transgene, thereby repairing the defect underlying GSDIa.

Disclosed herein is an in vivo method for treating a subject having GSDII, the method comprising administering to the subject a therapeutically effective amount of a disclosed donor vector; and administering to the subject therapeutically effective amount of a disclosed vector; wherein the Cas9 creates a double-strand break (DSB) on both sides of a mutation or defect in the GAA gene locus that results in a permanent integration of a disclosed GAA transgene, thereby repairing the defect underlying GSDII.

Disclosed herein is an in vivo method for treating a subject having a genetic disease or a genetic disorder, the method comprising administering to the subject a therapeutically effective amount of a disclosed donor vector; wherein the expression of the transgene provides therapeutic effects to the subject.

Disclosed herein is an in vivo method for treating a subject having GSDIa, the method comprising administering to the subject a therapeutically effective amount of a disclosed donor vector; wherein the expression of the G6PC transgene provides therapeutic effects to the subject.

Disclosed herein is an in vivo method for treating a subject having GSDII, the method comprising administering to the subject a therapeutically effective amount of a disclosed donor vector; wherein the expression of the GAA transgene provides therapeutic effects to the subject.

In an aspect, a disclosed method can comprise administering to the subject only a disclosed donor vector. In an aspect, administering only the disclosed donor vector can comprise gene therapy. In an aspect, a disclosed method can comprise administering to the subject a disclosed donor vector and a disclosed CRISPR vector. In an aspect, administering a disclosed donor vector and a disclosed CRISPR vector can comprise gene editing.

In an aspect, the disclosed cells can comprise any cells. In an aspect, the disclosed cells can preferentially comprise cells in kidney tissue, heart tissue, skeletal muscle tissue, liver tissue, or any combination thereof. In an aspect, the disclosed cells can preferentially comprise kidney cells, heart cells, skeletal muscle cells, liver cells, or any combination thereof.

In an aspect, the disclosed cells can comprise any cells affected by a defective GAA gene. Defects in the GAA gene are known to the skilled person in the art. In an aspect, the disclosed cells can comprise any cells affected by a defective G6PC gene. Defects in the G6PC gene are known to the skilled person in the art.

In an aspect, the disclosed cells can be any cells having a high and/or excess level of glycogen. In an aspect, the disclosed cells can be any cells experiencing dysfunctional glycogenolysis pathways and/or dysfunctional gluconeogenesis pathways.

In an aspect, a disclosed transgene can be expressed in dividing cells. In an aspect, a disclosed transgene can be expressed in non-dividing cells. In an aspect, a disclosed transgene can be expressed in both dividing and non-dividing cells. In an aspect, expression of a disclosed transgene can be effected in dividing cells and/or non-dividing cells.

In an aspect, the disclosed cells can be in a subject. In an aspect, a disclosed subject can be diagnosed with or is suspected of having a genetic disease or a genetic disorder. In an aspect, a disclosed subject can be diagnosed with or is suspected of having GSDIa. In an aspect, a disclosed subject can be diagnosed with or is suspected of having GSDII. In an aspect, a subject can have late onset GSDII or can have infantile onset of GSDII.

In an aspect, a disclosed subject can be diagnosed with or is suspected of having an inherited protein deficiency or inherited protein disorder. In an aspect, a disclosed subject can be diagnosed with or is suspected of having alpha-1-antitrypsin deficiency, phenylketonuria, mitochondrial trifunctional protein deficiency, hemophilia, or muscle dystrophy.

In an aspect, a disclosed subject can be an adult at the time of receiving a disclosed donor vector, a disclosed CRISPR vector, a disclosed gene editing system, a disclosed donor nucleic acid molecule, a disclosed CRISPR nucleic acid molecule, or a disclosed pharmaceutical formulation. In an aspect, a disclosed subject can be an infant at the time of receiving a disclosed donor vector, a disclosed CRISPR vector, a disclosed gene editing system, a disclosed donor nucleic acid molecule, a disclosed CRISPR nucleic acid molecule, or a disclosed pharmaceutical formulation.

In an aspect, a disclosed subject can be an adult at the time of a disclosed method. In an aspect, a disclosed subject can be an infant at the time of a disclosed method. In an aspect, a disclosed method can generate a depot of the therapeutic expression of a transgene. In an aspect, a disclosed method can generate a depot of the therapeutic expression of a transgene in a subject's liver and/or in a subject's skeletal muscle. In an aspect, a disclosed method can be used to correct and/or redress and/or reverse and/or treat a pathogenetic defect in skeletal muscle of a subject. In an aspect, a disclosed method can be used to correct and/or redress and/or reverse and/or treat a pathogenetic defect in cardiac tissue of a subject. In an aspect, a disclosed method can be used to correct and/or redress and/or reverse and/or treat a pathogenetic defect in kidney tissue and/or liver tissue of a subject. In an aspect, a disclosed method can improve hypoglycemia and/or decrease hyperlipidemia in a subject.

In an aspect, a disclosed method of treating a subject (e.g., GAA or G6PC) can restore the functionality and/or structural integrity of a missing, deficient, and/or mutant protein or enzyme (e.g., GAA or G6PC). In an aspect, a disclosed method can comprise restoring one or more aspects of cellular homeostasis and/or cellular functionality and/or metabolic dysregulation (such as, for example, glycogen accumulation). In an aspect, restoring one or more aspect of cellular homeostasis and/or cellular functionality and/or metabolic dysregulation comprises restoring the functionality and/or structural integrity of a missing, deficient, and/or mutant protein or enzyme (such as, for example, G6PC or GAA).

In an aspect, restoring one or more aspects of cellular homeostasis and/or cellular functionality can comprise one or more of the following: (i) correcting cell starvation in one or more cell types; (ii) improving, enhancing, restoring, and/or preserving mitochondrial functionality and/or structural integrity; (iii) improving, enhancing, restoring, and/or preserving organelle functionality and/or structural integrity; (iv) correcting enzyme dysregulation; (v) reversing, inhibiting, preventing, stabilizing, and/or slowing the rate of progression of the multi-systemic manifestations of a genetic disease or disorder; (vi) reversing, inhibiting, preventing, stabilizing, and/or slowing the rate of progression of a genetic disease or disorder, (vii) normalizing aspects of the autophagy pathway (such as, for example, correcting, preventing, reducing, and/or ameliorating autophagy), or (viii) any combination thereof. In an aspect, restoring one or more aspects of cellular homeostasis can comprise improving, enhancing, restoring, and/or preserving one or more aspects of cellular structural and/or functional integrity.

In an aspect, restoring the activity and/or functionality of a missing, deficient, and/or mutant protein or enzyme (such as, for example, G6PC or GAA) can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme such as, for example, GAA). In an aspect, restoration can be a partial or incomplete restoration. In an aspect, restoration can be complete or near complete restoration such that the level of expression, activity, and/or functionality is similar to that of a wild-type or control level.

In an aspect of a disclosed method of repairing a defective gene (such as, for example, G6PC or GAA), techniques to monitor, measure, and/or assess the restoring one or more aspects of cellular homeostasis and/or cellular functionality can comprise qualitative (or subjective) means as well as quantitative (or objective) means. These means are known to the skilled person.

In an aspect, a disclosed method of repairing a defective gene can improve and/or eliminate and/or diminish one or more symptoms associated with the genetic disease or genetic disorder. In an aspect, a disclosed method can improve a subject's ability to ambulate.

In an aspect of a disclosed method of repairing a defective gene, a disclosed vector can be administered systemically or directly to the subject. In an aspect, a disclosed vector can be intravenously, subcutaneously, or intramuscularly administered to the subject.

In an aspect, a therapeutically effective amount of a disclosed donor vector can comprise a range of about 1×10¹⁰ vg/kg to about 2×10¹⁴-vg/kg. In an aspect, for example, a disclosed donor vector can be administered at a dose of about 1×10¹¹ to about 8×10¹³ vg/kg or about 1×10¹² to about 8×10¹³ vg/kg. In an aspect, a disclosed donor vector can be administered at a dose of about 1×10¹³ to about 6×10¹³ vg/kg. In an aspect, a disclosed donor vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed donor vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed donor vector can be administered at a dose of about 1×10¹² vg/kg. In an aspect, a disclosed donor vector can be administered at a dose of about 1×10¹¹ vg/kg. In an aspect, a disclosed donor vector can be administered in a single dose, or in multiple doses (such as 2, 3, 4, 5, 6, 7, 8, 9 or 10 doses) as needed for the desired therapeutic results.

In an aspect, a therapeutically effective amount of a disclosed CRISPR vector can comprise a range of about 1×10¹⁰ vg/kg to about 2×10¹⁴-vg/kg. In an aspect, for example, a disclosed CRISPR vector can be administered at a dose of about 1×10¹¹ to about 8×10¹³ vg/kg or about 1×10¹² to about 8×10¹³ vg/kg. In an aspect, a disclosed CRISPR vector can be administered at a dose of about 1×10¹³ to about 6×10¹³ vg/kg. In an aspect, a disclosed CRISPR vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed CRISPR vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed CRISPR vector can be administered at a dose of about 1×10¹² vg/kg. In an aspect, a disclosed CRISPR vector can be administered at a dose of about 1×10¹¹ vg/kg. In an aspect, a disclosed CRISPR vector can be administered in a single dose, or in multiple doses (such as 2, 3, 4, 5, 6, 7, 8, 9 or 10 doses) as needed for the desired therapeutic results.

In an aspect, a disclosed method of repairing a defective gene can comprise administering to the subject one or more additional therapeutic agents. In an aspect, a disclosed therapeutic agent can comprise enzyme replacement therapy, gene therapy, mRNA therapy, small molecule therapy, substrate reduction therapy, or any combination thereof.

In an aspect, a disclosed method of repairing a defective gene can comprise treating the subject. In an aspect, treating the subject can comprise administering to the subject one or more agents that modulate the level of one or more differentially present cellular metabolites. In an aspect, treating the subject can comprise implementing a change in the subject's dietary intake of carbohydrates. Implementing a change in the subject's dietary intake of carbohydrates can comprise adding carbohydrates to the subject's diet, or removing carbohydrates from the subject's diet, or changing the type of carbohydrates in the subject's diet, or changing the frequency of carbohydrates consumed by the subject. In an aspect, treating the subject can comprise administering cornstarch to the subject, or administering glycoside to the subject, or administering one or more anaplerotic agents to the subject.

In an aspect of a disclosed method of repairing a defective gene (such as, for example, G6PC or GAA), a disclosed Cas9 nuclease can create a double-strand breaks (DSB) within or near a mutation or defect or a sequence variant in the gene locus that results in a permanent integration of the repair template.

In an aspect, a disclosed method of repairing a defective gene can improve the efficiency of gene editing. In an aspect, a disclosed method can be used to stably integrate a transgene (such as, for example, G6PC or GAA) into one or more disclosed cells.

In an aspect, a disclosed method can improve the efficiency of gene editing.

In an aspect, a disclosed method can be used in a method of treating a patient or in a method of validating a gene editing system. In an aspect, a disclosed method can comprise validating the expression of the transgene. In an aspect, a disclosed method can comprise confirming that the Cas9 created a double-strand break (DSB) on both sides of a mutation or defect in the GAA locus that results in a permanent integration of the repair template, thereby repairing the defect underlying the genetic disease or disorder (such as, GSDIa or GSDII).

In an aspect, a disclosed method can further comprise measuring the level or amount of one or more biomarkers (e.g., glucose, ALT, creatinine, glycogen, hepatocellular vacuolation, etc.), one or more indicators of the subject's metabolomic health, or any combination thereof.

In an aspect, validating the efficacy of a gene editing system can comprise measuring the expression of the transgene in the edited cells; and comparing the resulting transgene expression level in the edited cells to the transgene expression level in control cells, wherein the gene editing system is effective when the transgene expression in the edited cells is greater than the transgene expression level in control cells.

In an aspect, validating the efficacy of a gene editing system can comprise measuring the expression of a reporter gene in the edited cells; and comparing the resulting expression level of the reporter gene in the edited cells to the expression level of the reporter gene in control cells, wherein the gene editing system is effective when the expression level of the reporter gene in the edited cells is greater than the expression level of the reporter gene in control cells.

In an aspect, validating the efficacy of a gene editing system in a subject can comprise administering to the subject a disclosed vector comprising a disclosed isolated nucleic acid molecule; obtaining a biological sample of cells targets for editing; measuring the expression of a reporter gene in the edited cells; and comparing the resulting expression level of the reporter gene in the edited cells to the expression level of the reporter gene in control cells, wherein the gene editing system is effective when the expression level of the reporter gene in the edited cells is greater than the expression level of the reporter gene in control cells.

In an aspect, validating the efficacy of a gene editing system in a subject can comprise administering to the subject a disclosed vector comprising a disclosed isolated nucleic acid molecule; obtaining a biological sample of cells targets for editing; measuring the expression of a reporter gene in the edited cells; and comparing the resulting expression level of the reporter gene in the edited cells to the expression level of the reporter gene in control cells, wherein the gene editing system is effective when the expression level of the reporter gene in the edited cells is greater than the expression level of the reporter gene in control cells.

In a disclosed method, the disclosed control cells can comprise unedited cells. In an aspect, the disclosed control cells can comprise the subject's cells prior to administration of a disclosed vector, a disclosed nucleic acid molecule, or a disclosed pharmaceutical formulation. In an aspect, the disclosed control cells can comprise cells treated with a disclosed nucleic acid molecule or a disclosed vector having a scrambled gRNA.

In an aspect, measuring the expression of a disclosed transgene and/or the reporter gene can comprise measuring the protein concentration of the transgene and/or the reporter gene or measuring the mRNA level of transgene and/or the reporter gene. For example, in an aspect, measuring the protein concentration of transgene and/or the reporter gene comprises a protein chip analysis, an immunoassay, a ligand binding assay, a MALDI-TOF (Matrix Assisted Laser Desorption/Ionization Time of Flight Mass Spectrometry) analysis, a SELDI-TOF (Sulface Enhanced Laser Desorption/Ionization Time of Flight Mass Spectrometry) analysis, a radioimmunoassay, a radioimmunodiffusion assay, an octeroni immunodiffusion method, rocket immunoelectrophoresis, tissue immunostaining, a complement fixation assay, 2D by electrophoretic analysis, liquid chromatography-Mass Spectrometry (LC-MS), liquid chromatography-Mass Spectrometry/Mass Spectrometry (LC-MS/MS), Western blotting, ELISA (enzyme linked immunosorbent assay), or any combination thereof. Similarly, in an aspect, measuring the mRNA level of GAA and/or the reporter gene comprises a reverse transcription polymerase reaction (RT-PCR), a competitive reverse transcription polymerase reaction (Competitive RT-PCR), a real-time reverse transcription polymerization, an enzyme reaction (Real-time RT-PCR), an RNase protection assay (RPA), Northern blotting, a DNA chip, or any combination thereof.

In an aspect, a disclosed method of repairing a defective gene can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria. Such methods are known to the skilled person. In an aspect, a disclosed method can further comprise repeating a monitoring step.

In an aspect, a disclosed method of repairing a defective gene can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method of repairing a defective gene can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed method of repairing a defective gene can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method of repairing a defective gene can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method of repairing a defective gene (e.g., GAA or G6PC) can further comprise repeating a disclosed administering step such as, for example, repeating the administering of a disclosed isolated nucleic acid molecule, a disclosed vector, a disclosed pharmaceutical formulation, a disclosed therapeutic agent, a disclosed immune modulator, a disclosed proteasome inhibitor, a disclosed immunosuppressive agent, a disclosed compound that exerts a therapeutic effect against B cells and/or a disclosed compound that targets or alters antigen presentation or humoral or cell mediated immune response.

In an aspect, a disclosed method of repairing a defective gene can further comprise administering a 32 agonist. For example, in an aspect, a disclosed method can comprises administering a 32 agonist to increase the expression of one or more receptors for a lysosomal enzyme. In an aspect, 32 agonists include but are not limited to albuterol, clenbuterol, formoterol, indacaterol, olodaterol, salmeterol, vilanterol, and any combination thereof, growth hormones (e.g., human growth hormone), autocrine glycoprotein (e.g., Follistatin), or any combination thereof (see, e.g., U.S. Pat. No. 8,679,478 for a discussion of appropriate 32 agonists, which patent is incorporated by reference it its entirety for these teachings).

In an aspect, a disclosed method of repairing a defective gene can further comprise administering to a subject or patient one or more fibrates. In an aspect, a disclosed fibrate can comprise bezafibrate, fenofibrate, ciprofibrate, gemfibrozil, clofibrate, an analog thereof, or a combination thereof. In an aspect, a disclosed method can comprise repeating the administering of one or more fibrates one or more times. In an aspect, a therapeutically effective amount of one or more fibrates can comprise at least about 20 mg/day to at least 500 mg/day. In an aspect, a therapeutically effective amount of one or more fibrates can comprise at least about 20 mg/day, at least about 20 mg/day, about 30 mg/day, about 40 mg/day, at least about 50 mg/day, at least about 60 mg/day, at least about 70 mg/day, at least about 80 mg/day, at least about 90 mg/day, at least about 100 mg/day, at least about 120 mg/day, at least about 140 mg/day, at least about 160 mg/day, at least about 180 mg/day, at least about 200 mg/day, at least about 220 mg/day, at least about 240 mg/day, at least about 260 mg/day, at least about 280 mg/day, at least about 300 mg/day, at least about 320 mg/day, at least about 340 mg/day, at least about 360 mg/day, at least about 380 mg/day, at least about 400 mg/day, at least about 420 mg/day, at least about 440 mg/day, at least about 460 mg/day, at least about 480 mg/day, or at least about 500 mg/day.

In an aspect, a disclosed method of repairing a defective gene can further comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, one or more isolated nucleic acid molecules or one or more vectors can be administered concurrently or sequentially.

In an aspect, a disclosed method can further comprise diagnosing a subject with a genetic defect using one or more known methods to the skilled person, such as, for example, genotyping.

In an aspect, a disclosed method can drive supraphysiologic GAA expression in a subject's liver and/or a subject's skeletal muscle. In an aspect, a disclosed method can drive supraphysiologic G6PC expression in a subject's liver and/or a subject's skeletal muscle. In an aspect, a disclosed method can restore normal glycogen content in a subject's heart, diaphragm, quadriceps, or any combination thereof. In an aspect, a disclosed method can improve a subject's muscle strength.

In an aspect, a disclosed donor vector does not elicit an immune response.

In an aspect, a disclosed method can ensure persistent transgene expression.

In an aspect of a disclosed method, a disclosed donor vector can achieve episomal expression of a disclosed transgene.

### F. Kits

Disclosed herein is a kit comprising a disclosed nucleic acid molecule, a disclosed promoter, a disclosed hybrid promoter, a disclosed donor vector, a disclosed CRISPR vector, a disclosed pharmaceutical formulation, or any combination thereof. Disclosed herein is a kit comprising one or more disclosed nucleic acid molecules, disclosed promoters, disclosed hybrid promoters, disclosed donor vectors, disclosed CRISPR vectors, disclosed pharmaceutical formulations for use with a disclosed method. In an aspect, a kit can comprise a disclosed nucleic acid molecule, a disclosed vector, a disclosed pharmaceutical formulation, or a combination thereof, and one or more agents. “Agents” and “Therapeutic Agents” are known to the art and are described supra.

In an aspect, the one or more agents can treat, prevent, inhibit, and/or ameliorate one or more comorbidities in a subject. In an aspect, one or more active agents can treat, inhibit, prevent, and/or ameliorate cellular and/or metabolic complications related to a missing, deficient, and/or mutant protein or enzyme (such as, for example, G6PC or GAA).

In an aspect, a disclosed kit can comprise at least two components constituting the kit. Together, the components constitute a functional unit for a given purpose (such as, for example, treating a subject diagnosed with or suspected of having a disease or disorder). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed isolated nucleic acid molecule, a disclosed vector, a disclosed pharmaceutical formulation, or a combination thereof, and a label or package insert with instructions for use. In an aspect, suitable containers include, for example, bottles, vials, syringes, blister pack, etc. The containers can be formed from a variety of materials such as glass or plastic. The container can hold one or more disclosed nucleic acid molecules, disclosed promoters, disclosed hybrid promoters, disclosed donor vectors, disclosed CRISPR vectors, disclosed pharmaceutical formulations, and can have a sterile access port (for example the container may be an intravenous solution bag or a vial having a stopper pierceable by a hypodermic injection needle). The label or package insert can indicate that one or more disclosed nucleic acid molecules, disclosed promoters, disclosed hybrid promoters, disclosed donor vectors, disclosed CRISPR vectors, disclosed pharmaceutical formulations can be used for treating, preventing, inhibiting, and/or ameliorating a disease or disorder or complications and/or symptoms associated with a genetic disease or genetic disorder. A kit can comprise additional components necessary for administration such as, e.g., other buffers, diluents, filters, needles, and syringes.

In an aspect, a disclosed kit can be used in any disclosed method. In an aspect, a disclosed kit can be used to repair a defective GAA gene or any genetic defect. In an aspect, a disclosed kit can be used to treat a subject having Pompe disease. In an aspect, a disclosed kit can be used to validating the efficacy of a gene editing system. In an aspect, a disclosed kit can be used to treat and/or prevent a genetic disease or disorder.

Table of Sequences

| ID# | Description |
|---|---|
| 1. | G6PC Promoter (Human) |
| 2. | G6PC Min Promoter |
| 3. | G6PC Min Promoter |
| 4. | hSk-CRM4 enhancer |
| 5. | hSk-CRM4 enhancer |
| 6. | hSk-CRM4 enhancer + G6PC mini promoter |
| 7. | hSk-CRM4 enhancer + G6PC mini promoter |
| 8. | hSk-CRM4 enhancer + G6PC mini promoter |
| 9. | hSk-CRM4 enhancer + G6PC Min Promoter |
| 10. | G6PC mini promoter + hSk-CRM4 enhancer |
| 11. | G6PC mini promoter + hSk-CRM4 enhancer |
| 12. | G6PC mini promoter + hSk-CRM4 enhancer |
| 13. | G6PC mini promoter + hSk-CRM4 enhancer |
| 14. | G6PC gene (Mus Musculus) (Accession NC_000077.7) (Gene |
| | ID 14377) |
| 15. | G6PC mRNA (Mus Musculus) (Accession NM_008061.4) |
| 16. | G6PC protein (Mus Musculus) (Accession NP_032087.2) |
| 17. | G6PC gene (Human) (Accession NG_011808.1) (Gene ID 2538) |
| 18. | G6PC mRNA (Human) (Accession NM_000151.4) |
| 19. | G6PC protein (Human) (Accession NP_000142.2) |
| 20. | Glucose 6-phosphatase (G6PC) |
| 21. | Glucose 6-phosphatase (G6PC) |
| 22. | Glucose 6-phosphatase (G6PC) |
| 23. | Glucose 6-phosphatase (G6PC) |
| 24. | Glucose 6-phosphatase (G6PC) |
| 25. | Glucose 6-phosphatase (G6PC) |
| 26. | GAA gene (Mus Musculus) (Accession No. NC_000077.7) |
| 27. | GAA mRNA (Mus Musculus) (Accession No. NM_008064.4) |
| 28. | GAA protein (Mus Musculus) (Accession No. NP_032090.3) |
| 29. | GAA gene (human) (Accession No. NG_009822.1) (Gene ID |
| | 2548) |
| 30. | GAA mRNA (human) (Accession No. NM_000152.5) |
| 31. | GAA protein (human) (Accession No. NP_000143) |
| 32. | Acid alpha glucosidase (GAA) |
| 33. | Acid alpha glucosidase (GAA) |
| 34. | Acid alpha glucosidase (GAA) |
| 35. | Acid alpha glucosidase (GAA) |
| 36. | Acid alpha glucosidase (GAA) |
| 37. | GAA Fragment/Repair Template |
| 38. | GAA Fragment/Repair Template |
| 39. | GAA Fragment/Repair Template |
| 40. | GAA Fragment/Repair Template |
| 41. | Cas9 (Streptococcus Pyogenes) |
| 42. | Cas9 (Staphylococcus Aureus) |
| 43. | Cas9 (Staphylococcus Aureus) |
| 44. | Cas9 (Streptococcus Pyogenes |
| 45. | Cas9 (Staphylococcus Aureus) |
| 46. | Cas9 (Staphylococcus Aureus) |
| 47. | Spacer or Linker between G6PC and hSk-CRM4 |
| 48. | Sequence between MinPromoter and GCC |
| 49. | AAV-G6PC(303)-hGAA |
| 50. | AAV-G6PC(356)-hGAA |
| 51. | AAV-CRM4-G6PC(356)-hGAA |
| 52. | AAV-G6PC(303)-hGAA |
| 53. | AAV-G6PC(303)-Cas9 |
| 54. | Donor Vector |
| 55. | Cas9 Vector |
| 56. | gRNA for mG6PC |
| 57. | gRNA to hG6PC |
| 58. | gRNA to hG6PC |
| 59. | gRNA to hG6PC |
| 60. | gRNA to hG6PC |
| 61. | gRNA to hG6PC |
| 62. | gRNA to hG6PC |
| 63. | gRNA to hG6PC |
| 64. | gRNA to hG6PC |
| 65. | gRNA to hG6PC |
| 66. | gRNA to hG6PC |
| 67. | gRNA to mGAA |
| 68. | gRNA to mGAA |
| 69. | gRNA to hGAA |
| 70. | gRNA to hGAA |
| 71. | gRNA to delT525 |
| 72. | gRNA to IVS1 |
| 73. | gRNA to m1826DupA |
| 74. | gRNA to m1826DupA |
| 75. | gRNA to m1826DupA + PAM |
| 76. | gRNA to m1826DupA + PAM |
| 77. | gRNA to mIVS1 |
| 78. | gRNA to mIVS1 + PAM |
| 79. | SaCas9 PAM |
| 80. | SaCas9 PAM |
| 81. | SaCas9 PAM |
| 82. | SaCas9 PAM |
| 83. | SaCas9 mutant PAM |
| 84. | SaCas9 mutant PAM |
| 85. | SaCas9 mutant PAM |
| 86. | SaCas9 mutant PAM |
| 87. | 5′ Homology Arm − mG6PC |
| 88. | 3′ Homology Arm − mG6PC |
| 89. | 5′ Homology Arm − mGAA |
| 90. | 3′ Homology Arm − mGAA |
| 91. | 5′ Homology Arm − hG6PC |
| 92. | 5′ Homology Arm − hG6PC |
| 93. | 5′ Homology Arm + Promoter − hG6PC |
| 94. | 3′ Homology Arm − hG6PC |
| 95. | 3′ Homology Arm − hG6PC |
| 96. | 3′ Homology Arm − hG6PC |
| 97. | 5′ Homology Arm − hGAA |
| 98. | 5′ Homology Arm − hGAA |
| 99. | 5′ Homology Arm − hGAA |
| 100. | 3′ Homology Arm − hGAA |
| 101. | 3′ Homology Arm − hGAA |
| 102. | 3′ Homology Arm − hGAA |
| 103. | gRNA scaffold |
| 104. | gRNA scaffold |
| 105. | Poly(A) Signal |
| 106. | Poly(A) Signal |
| 107. | Poly(A) Signal |
| 108. | Poly(A) Signal |
| 109. | Poly(A) Signal |
| 110. | Poly(A) Signal |
| 111. | HA |
| 112. | HA |
| 113. | Inverted Terminal Repeat (ITR) |
| 114. | Inverted Terminal Repeat (ITR) |
| 115. | Inverted Terminal Repeat (ITR) |
| 116. | Inverted Terminal Repeat (ITR) |
| 117. | Inverted Terminal Repeat (ITR) Fragment |
| 118. | Inverted Terminal Repeat (ITR) Fragment |
| 119. | Inverted Terminal Repeat (ITR) Fragment |
| 120. | U6 Promoter for gRNA |
| 121. | U6 Promoter for gRNA |
| 122. | U6 Promoter for gRNA |
| 123. | hG6PC Forward Primer |
| 124. | hG6PC Reverse Primer |
| 125. | SpCas9 Forward Primer |
| 126. | SpCas9 Reverse Primer |
| 127. | Mouse b-actin Forward Primer |
| 128. | Mouse b-actin Reverse Primer |
| 129. | β-actin Forward Primer |
| 130. | β-actin Reverse Primer |
| 131. | mousesurveyor Forward Primer |
| 132. | mousesurveyor Reverse Primer |
| 133. | tRNA |
| 134. | chRNA |
| 135. | Primer 1 |
| 136. | M1 Primer |
| 137. | M2 Primer |
| 138. | M3 Primer |
| 139. | M4 Primer |
| 140. | GAA Forward Primer |
| 141. | GAA Reverse Primer |
| 142. | GAA Reverse Primer |
| 143. | Nucleoplasmin NLS |
| 144. | SV40 NLS |
| 145. | Rosa 26 Fragment |
| 146. | G6P5r |
| 147. | MCS (fragment) |
| 148. | Kozak Sequence |
| 149. | Liver Promoter |
| 150. | Liver Promoter |
| 151. | Liver Promoter |
| 152. | CMV Promoter |
| 153. | CMV Promoter |
| 154. | CMV Promoter |
| 155. | CMV Enhancer |
| 156. | CMV Enhancer/Promoter |
| 157. | CMV Enhancer Fragment |
| 158. | CMV Enhancer Fragment |
| 159. | CMV Enhancer Fragment/CMV Promoter |
| 160. | CMV Enhancer Fragment/CMV Promoter |

### EXAMPLES

Glycogen storage disease type Ia (GSD Ia) is an autosomal recessive genetic disorder resulting from pathogenic variants in the glucose-6-phosphatase catalytic subunit (G6PC) gene (Kern A, et al. (2003) J. Virol. 77:11072-11081; Opie S R, et al. (2003) J. Virol. 77:6995-7006). This metabolic disease results in excess glycogen accumulation in the liver and kidneys. GSD Ia patients develop life threatening hypoglycemia, as well as hepatomegaly, lactic acidosis, hyperlipidemia, and hyperuricemia. Long-term effects include impaired growth, renal failure, and hepatocellular adenomas (HCA) with a risk for hepatocellular carcinoma (HCC) formation. Current therapy involves dietary supplementation with uncooked cornstarch to prevent hypoglycemia (Kern A, et al. (2003) J. Virol. 77:11072-11081; Opie S R, et al. (2003) J. Virol. 77:6995-7006).

Gene therapy is being explored as an alternative treatment for GSD Ia. Multiple groups have demonstrated liver directed gene therapy that delivered a G6PC transgene to hepatocytes and expressed G6Pase. However, expression of the therapeutic transgene was unstable, because episomal AAV vector genomes were lost due to cell division in the liver of both mice and dogs with GSD Ia (Govindasamy L, et al. (2006) J. Virol. 80:11556-11570; O'Donnell J, et al. (2009) Virology. 385:434-443; Shen S, et al. (2013) J. Biol. Chem. 288:28814-28823; Afione S, et al. (2015) J. Virol. 89:1660-1672). Strategies to overcome this limitation of AAV vector-mediated gene therapy have included higher doses of AAV or re-administration of AAV (Huang L-Y, et al. (2016) J. Virol. 90:5219-5230; Zhang R, et al. (2019) Nat. Commun. 10:3760; Zhang R, et al. (2019) Nat. Microbiol. 4:675-682). These approaches have not been successful at preventing episomal vector genome loss and sustaining transgene expression. This limitation is particularly obvious when treating infant mice with gene therapy as AAV vector genomes are rapidly lost from the liver early in life (Huang L-Y, et al. (2016) J. Virol. 90:5219-5230; Zhang R, et al. (2019) Nat. Microbiol. 4:675-682; Meyer N L, et al. (2019) Elife. 8:e44707; Gurda B L, et al. (2012) J. Virol. 86:7739-7751).

Genome editing has been proposed as an alternative method to correct pathogenic variants and promote stable, long-term expression of transgenes. As detailed below, this study aimed to investigate the benefit of CRISPR/Cas9 based genome editing in mice with GSD Ia. Additionally, the drug bezafibrate was administered to stimulate fatty acid oxidation and to restore autophagy in the GSD Ia liver (Venkatakrishnan B, et al. (2013) J. Virol. 87:4974-4984), which previously increased AAV vector-mediated genome editing in the GSD Ia liver (Lins-Austin B, et al. (2020) Viruses. 12:66). The current study evaluated the benefits of CRISPR/Cas9 based genome editing accompanied by transgene integration and expression of G6PC in infantile, 12 day-old G6pc −/− mice with GSD Ia. Biochemical correction was evaluated by quantification of G6Pase activity and glycogen content. The combination of genome editing with drug treatment achieved improved survival and normalized blood glucose levels in infant mice with GSD Ia in association with transgene integration and G6PC expression in the liver.

### Materials and Methods

### 1. Sex as a Biological Variable

GSD Ia is an autosomal recessive disorder that affects both sexes, and therefore both male and female mice were included in each group. Mice were treated as infants prior to the ability to assign sex.

### 2. AAV Production and Administration

The AAV9 serotype has been described (Gao G P, et al. (2002) Proc Natl Acad Sci USA. 99(18):11854-11859). The AAV vector plasmid (pAAV-CRISPR—SEQ ID NO:55) contained a vector gene containing a terminal repeat (TR) at each end flanking a minimal G6PC promoter expressing Cas9 from S. pyogenes with a FLAG tag and bovine growth hormone genomic polyadenylation sequence. The second AAV vector plasmid (pAAV-mDonor—SEQ ID NO:54) contained a TR at each end flanking two transgenes: (1) the human G6PC cDNA flanked upstream by the homology arm containing 5′ UTR genomic sequence of mouse G6pc, including a 297 bp minimal G6PC promoter, and downstream by the human growth hormone genomic polyadenylation sequence followed by the homology arm containing intron 1 genomic sequence of mouse G6pc; and (2) the U6 promoter expressing a gRNA specific for the exon 1/intron 1 boundary of G6pc. Vectors were packaged, purified, and quantified by Southern blot as described (Pénzes J J, et al. (2015) J. Gen. Virol. 96:2769-2779). Vectors were administered by retro-orbital injection on day 12 (+/−1) of life.

### 3. Quantification of Vector DNA in Tissues

AAV vector genome copy number was measured by quantitative real-time PCR with liver genomic DNA and normalized to β-actin. Plasmid DNA corresponding to 0.01 to 100 copies of G6PC (in 500 ng genomic DNA) was used in a standard curve. qPCR was performed on a Lightcycler 480 (Roche Diagnostics, Basel, Switzerland) using SYBR Green mix (Thermo Fisher Scientific, Waltham, MA) and the following primers: hG6PC Fwd (5′-GCAGTTCCCTGTAACCTGTGAG-3′) (SEQ ID NO:123), hG6PC Rev (5′-GGTCGGCTTTATCTTTCCCTG-3′) (SEQ ID NO: 124), SpCas9 Fwd (5′-AGTACAGCATCGGCCTGGAC-3′) (SEQ ID NO: 125), SpCas9 Rev (5′-GGGCTCCGATCAGGTTCTTC-3′) (SEQ ID NO:126), mouse β-actin Fwd (5′-GGCTGTATTCCCCTCCATCG-3′) (SEQ ID NO: 127), mouse β-actin Rev (5′-CCAGTTGGTAACAATGCCATGT-3′) (SEQ ID NO:128). Cycling conditions were 95° C. for 5 minutes, followed by 45 cycles of 95° C. for 10 seconds, 60° C. for 10 seconds, and 72° C. for 20 seconds followed by acquisition.

### 4. Biochemical Analysis

Enzyme analysis was performed as described (Govindasamy L, et al. (2006) J. Virol. 80:11556-11570). Briefly, tissues were flash-frozen and stored at −70° C. Glycogen content was measured by complete digestion of polysaccharide using amyloglucosidase (Sigma Chemical Co., St. Louis, MO). The structure of the polysaccharide was inferred by using phosphorylase free of the debranching enzyme to measure the yield of glucose-1-phosphate. Specific G6Pase activity was measured by using glucose-6-phosphate as substrate after subtraction of nonspecific phosphatase activity as estimated by β-glycerophosphate.

### 5. Fasting Study

Glucose curves for monitoring hypoglycemia were performed by fasting the mice for up to 8 hours and monitoring blood glucose every 2 hours. If blood glucose dropped below 50-60 mg/dL or clinical signs of hypoglycemia occurred, then the curve was stopped, and mice were given dextrose therapy as needed and fed. Blood glucose was measured by a point of care glucometer, either the AlphaTRAK or AlphaTRAK2 (Zoetis, Parsippany, NJ).

### 6. Glucose Tolerance Test

All mice were given an intraperitoneal (IP) injection of 1 mg of dextrose per gram of body weight after a 4 hour fast. Tail clip blood samples were drawn during fasting prior to the IP injection, and again at 15 minutes, 30 minutes, 60 minutes, and 120 minutes post injection. This test provided information on the amount of glucose uptake into insulin sensitive tissues throughout the course of the 120 minutes post-injection. Plasma glucose levels were analyzed by Alphatrak glucose monitoring (Zoetis, Parsippany, NJ).

### 7. ELISA Determination of Murine α-SpCas9 Humoral Response

Recombinant SpCas9 (catalog number: CAS9PROT, Millipore Sigma, St. Louis, MO) was diluted to 0.05 μg/mL in 1×PBS and used to coat a 96-well MaxiSorp plate (catalog number: 442404, Thermo Fisher Scientific, Waltham, MA). Wells were coated overnight at 4° C. with 100 μL/well of coating solution. All subsequent incubations were carried out at room temperature on an orbital shaker and all wash steps were repeated 3 times. Plates were then washed with 1×PBS+0.05% Tween-20 (PBST). Plates were blocked for 2 hours with 200 μL/well of PBST+3% w/v non-fat dry milk (catalog number: 1706404; Bio-Rad, Berkeley, CA). Dilutions of heat-inactivated mouse serum were prepared in PBST+1% w/v BSA (catalog number: AP-4510-80; SeraCare, Milford, MA) in a separate plate. Serum diluted 1:100 was then added to the plate at 100 μL/well and incubated for 1 hour. Plates were then washed with PBST. Goat anti-mouse IgG conjugated HRP secondary antibody (catalog number: 1030-05; SouthernBiotech, Birmingham, AB) was diluted 1:1000 in PBST. 100 μL of secondary antibody solution was added per well and incubated for 30 minutes. Plates were washed a final time with PBST. Plates were developed for 30 minutes with 50 μL/well of TMB ELISA substrate (catalog number: 34028; Thermo Fisher Scientific, Waltham, MA). Development was stopped by the addition of 50 μL/well of 0.16 M sulfuric acid (catalog number: N600; Thermo Fisher Scientific, Waltham, MA). Absorbance was recorded at 450 nm for all samples.

The positive control for the SpCas9 ELISA was generated by vaccination with recombinant SpCas9 (catalog number: CAS9PROT, Millipore Sigma, St. Louis, MO) was purchased as lyophilized protein and reconstituted in sterile 0.9% saline (catalog number: NDC 0409-4888-03; Hospira, Lake Forest, IL) to a concentration of 1 μg/μL. 30 μL of SpCas9 solution was mixed 1:1 with room temperature Addavax adjuvant (catalog number: vac-adx-10; Invivogen, San Diego, CA) via pipet. Then, 25 μL of SpCas9/Addavax solution was injected into each gastrocnemius of a 10 week-old, male G6pc (+/−) mouse for a total dose of 50 μL. Blood was collected via cheek bleed immediately before vaccination and 2-weeks post-vaccination. Serum was isolated using a serum separator tube (catalog number: 450472, Thermo Fisher Scientific, Waltham, MA) via standard protocol. Collected serum was heat-inactivated at 56° C. for 30 minutes and then stored frozen until analysis.

### 8. CD3 Lymphocyte Staining

Rabbit monoclonal antibody CD3 (SP7) was from (catalog number: RM-9107s; Thermo Fisher Scientific, Waltham, MA). CD3 was used at 1:50, with Discovery Ab Diluent (cat #760-108). IHC tests were performed using the Ultra Discovery automated staining platform. The tissue sections were pretreated (epitope retrieval) with Roche cell conditioning solution CC1 (catalog number: 950-124; Roche, Basel, Switzerland) for 56 minutes. Rabbit monoclonal CD3 was applied and incubated for 60 minutes at 37° C. Rabbit IgG, substituted for the primary antibody, was used as the negative control. After binding of the primary antibody, anti-rabbit HQ (catalog number: 760-4815; Roche, Basel, Switzerland) were applied and incubated for 12 minutes, followed by 12 minutes incubation with anti-HQ HRP. For visualization, Disc. ChromoMap DAB (catalog number: 760-159; Roche, Basel, Switzerland) was applied and incubated for 5 minutes. Immunoreactive lymphoid cells were counted in ten random fields at 400× by a pathologist using an Olympus BX51 microscope.

### 9. Quantification of Vacuolation in Liver

All counts were made using the Fiji: ImageJ. Images from stained liver sections were first converted to 8-bit greyscale and then thresholded to 180:255. The built-in watershed function was applied. Finally, images were counted using the Analyze Particles within a range of 50-10,000 pixels. Fiji automatically displayed a total count of vacuoles along with the average size.

### 10. Surveyor Nuclease Detection Assay

Using purified DNA, the murine G6pc locus was PCR amplified using the primers mousesurveyorFwd (5′-TGACCTACAGACTGAATCCAGG-3′) (SEQ ID NO:131) and mousesurveyorRev (5′-TAACATCTGTGCTCAGGAGCTG-3′) (SEQ ID NO:132). The PCR product was analyzed using the Surveyor Mutation Detection Kit (Integrated DNA Technologies, Coralville, IA) according to manufacturer's instructions. The PCR products were also sequenced using Sanger sequencing methods (Eton Biosciences, Durham, NC).

### 11. DNA Integration Quantification

A synthetic DNA fragment was generated by PCR with primers M1 (5′-CAGCCGCACAAGAAGTCGTTG-3′) (SEQ ID NO:136) and M4 (5′-TCTGGGAATCAGGGACTGGG-3′) (SEQ ID NO:139) in the first round of PCR, followed by primers M2 (5′-CCACTCCCACTGTCCTTTCC-3′) (SEQ ID NO:137) and M3 (5′-GGCTCAGTAGATCAAGTGCCTGC-3′) (SEQ ID NO:138). The PCR fragment contained the junction fragment from the 3′ end of the human G6PC cDNA in the transgene to the intron 1 G6pc sequence in mouse genomic DNA. Serial dilutions of the synthetic DNA templates were prepared and used as the starting template for each PCR reaction to generate the standard curve. Mouse genomic DNA was amplified simultaneously to measure the level of integrated transgene and the G6pc locus.

### 12. Off-Target Analysis

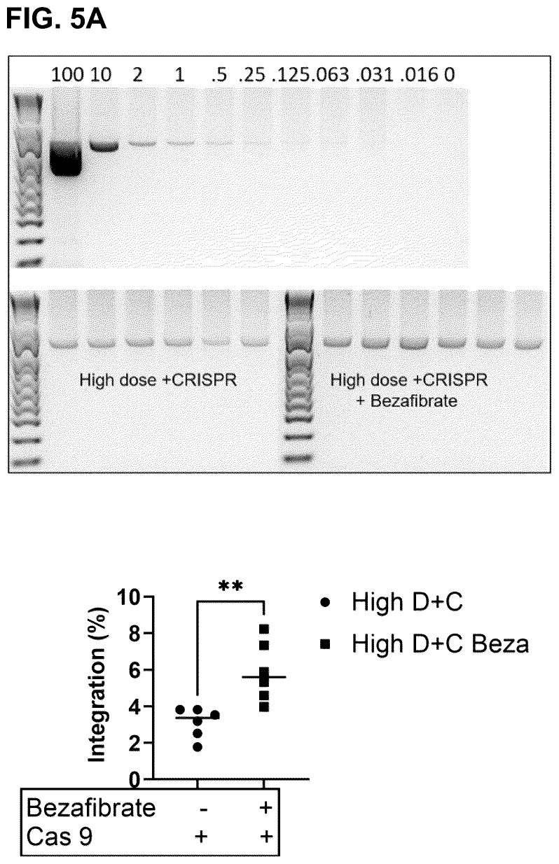

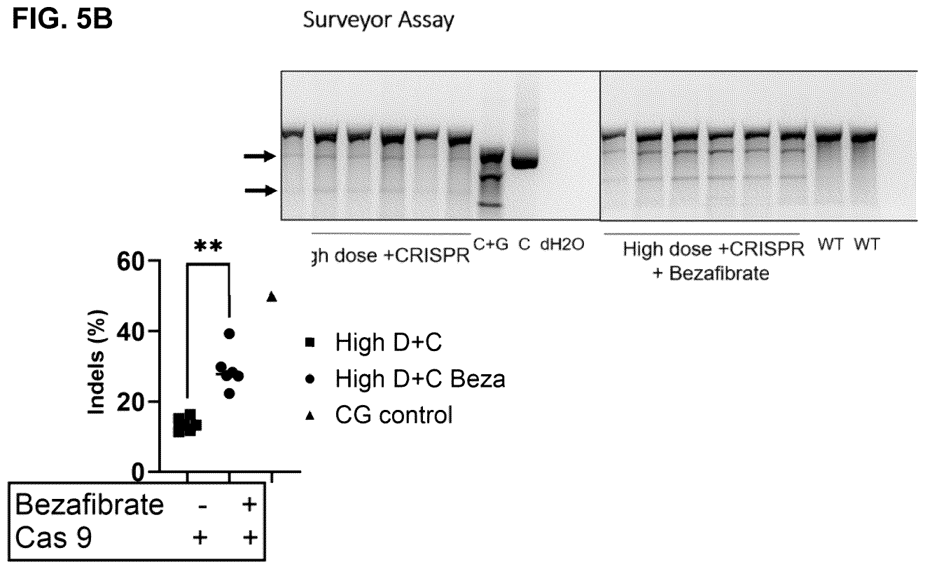

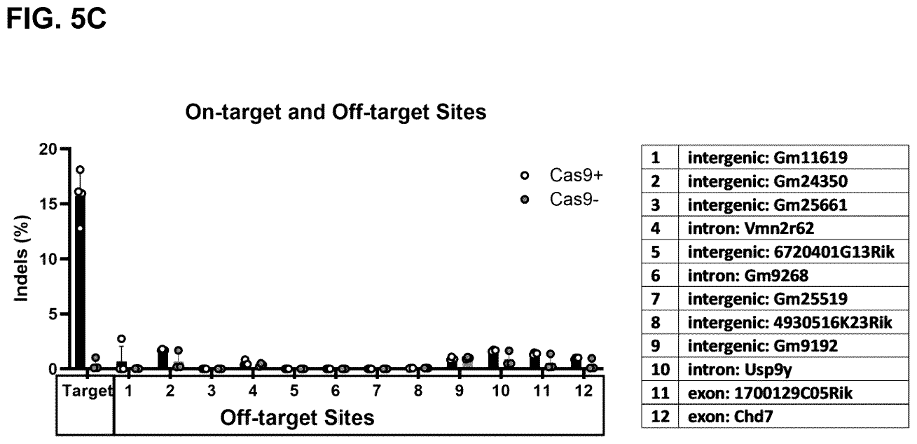

The web-based tool CRISPOR.org was used to predict potential off-target sites for gRNAs. No off-target sites were found with no or one mismatch in the gRNA. With two mismatches 9 target sites were identified, and all were in intronic (3) or intergenic (7) regions. With 3 mismatches two exonic regions outside the target exon were identified. Primers were designed to target sites (FIG. 5C). PCR products were extracted from the gel using the Qiagen Gel Extraction Kit (catalogue number 28704; Germantown, MD). Illumina NGS technology was used, and data was analyzed by the Azenta Lifesciences bioinformatics team (Azenta, Burlington, MA). NGS sequencing of off-target sites 3, 5, 6, and 7 was complicated by the co-amplification paralogous genomic regions during PCR. To remove paralog amplicons, fastq files for sites 3, 5, 6, and 7 were first aligned against the GRCm38 reference and then to the target amplicon using CRISPRessoPooled analysis.

### 13. Statistics

Statistical analysis was performed using GraphPad Prism 10 (GraphPad, Boston, MA). Statistical significance was determined by one-way ANOVA with Dunnett's multiple comparisons test, or by homoscedastic t test for comparisons of two groups. The statistical significance of comparisons is indicated as follows: * means p<0.05, ** means p<0.01, means p<0.001, and **** means p<0.0001.

### Example 1

This study aimed to investigate whether genome editing from the addition of a vector containing a Cas9 transgene (CRISPR vector) would enhance the efficacy of a vector containing the G6PC transgene vector (Donor vector; FIG. 1A). The addition of the Cas9-expressing (CRISPR) vector was expected to increase efficacy, because homology directed repair (HDR) at the double-stranded break induced by Cas9-mediated DNA cleavage is predicted to enhance integration of the transgene, resulting in the retention of more G6PC transgenes and more robust G6Pase expression. Two recombinant AAV9 (rAAV9) vectors were generated for this study. The CRISPR vector contains the S. pyogenes Cas9 gene driven by a minimal G6PC promoter (FIG. 1A). The Donor vector contains a human G6PC transgene with minimal G6PC promoter flanked by homology arms consisting of 5′ mouse G6pc exon 1 sequence and 3′ mouse G6pc intron 1 sequence. The Donor vector also contains a U6 promoter expressing a gRNA targeting the exon 1/intron 1 boundary of the endogenous mouse G6pc gene (FIG. 1A). When CRISPR/Cas9 cleaves the murine G6pc locus, the transgene is predicted to insert via HDR resulting in an integrated full length G6PC transgene in the G6pc locus (FIG. 1B).

Initially, G6pc (−/−) mice with GSD Ia were treated at 12 days of age with two different dosages of vectors: low dose (Donor, 2×10¹² vg/kg; +/−CRISPR 4×10¹¹ vg/kg), or medium dose (Donor 8×10¹² vg/kg; +/−CRISPR 1.6×10¹² vg/kg). Two weeks following vector administration the G6pc (−/−) mice that received the low dose of both Donor+CRISPR vectors had increased blood glucose concentrations during fasting (FIG. 1C), in comparison with mice treated with low dose Donor vector alone. A glucose tolerance test was performed four weeks after treatment to further evaluate glucose metabolism. In the glucose tolerance test, low dose Donor+CRISPR vector administration improved blood glucose at Baseline following 4 hours fasting (FIG. 1D) and at 120 minutes following glucose administration (FIG. 1E), in comparison with Donor vector alone. Biochemical correction of the G6pc (−/−) mouse livers was evaluated 4 weeks following treatment by analyzing G6Pase activity and glycogen content. G6Pase activity was similar between groups (FIG. 1F). However, liver glycogen was significantly decreased for GSD Ia mice receiving both medium dose Donor+CRISPR vectors, in comparison with mice receiving medium dose Donor vector only (FIG. 1G). Additionally, medium dose Donor+CRISPR treated mice had hepatic glycogen content similar to the level observed for wildtype mice (104+/−10% of wildtype). Furthermore, copies of the Donor vector trended higher in the liver for the CRISPR+Donor treated group, in comparison with the Donor treated mice (FIG. 1H). Expression of the Donor transgene in the liver was higher in both the low and medium dose Donor+CRISPR treated groups, in comparison with mice receiving the Donor vector only (FIG. 1I), based on quantification of Donor vector RNA.

### Example 2

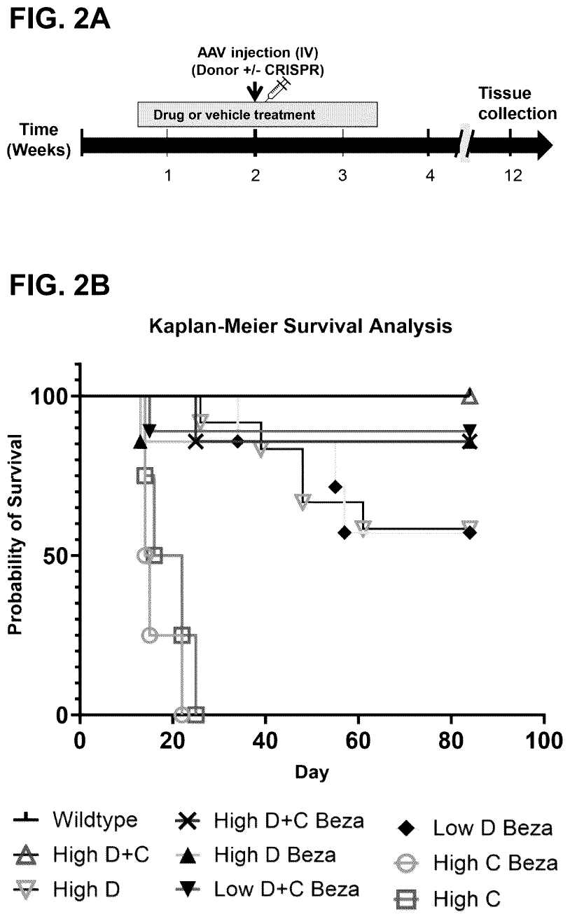

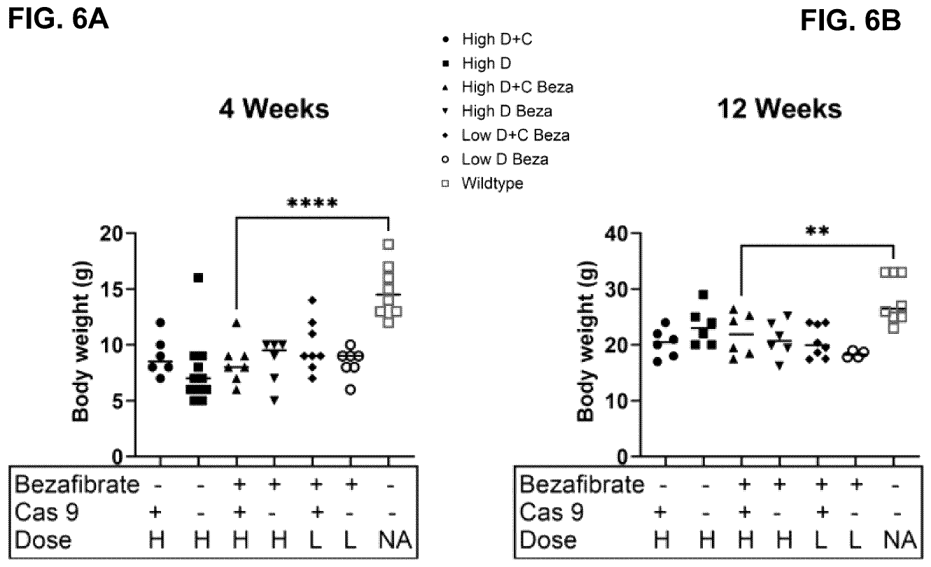

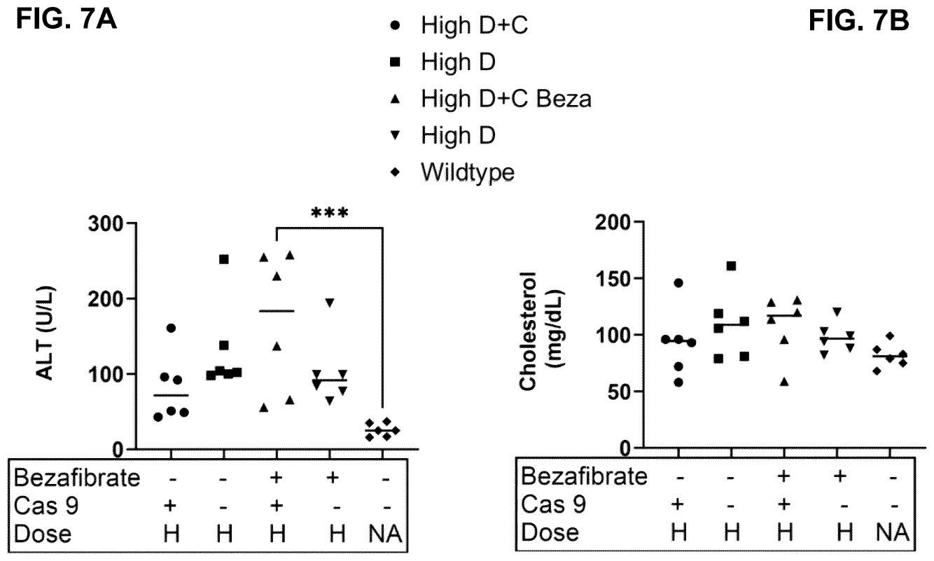

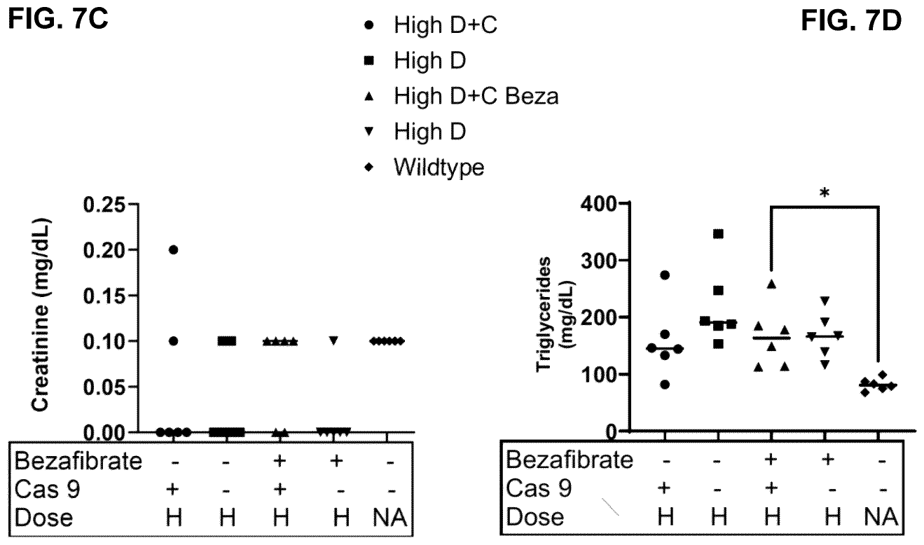

Given the demonstrated benefits from the addition of the Cas9-containing CRISPR vector, the study was extended to 12 weeks to determine the most efficacious treatment. The high and low dose vector treatment with and without administration of the drug bezafibrate was compared. Bezafibrate is a pan-agonist of peroxisome proliferator-activated receptors (PPARs) that enhances the expression of genes involved in lipid homeostasis and energy metabolism (FIG. 2A) Enzyme analysis was performed as described (Govindasamy L, et al. (2006) J. Virol. 80:11556-11570). Bezafibrate can lower liver triglycerides and glycogen in GSD Ia mice while also increasing the transduction of AAV and expression of transgenes (Venkatakrishnan B, et al. (2013) J. Virol. 87:4974-4984; Lins-Austin B, et al. (2020) Viruses. 12:66). Survival analysis revealed increased survival for GSD Ia mice receiving the Donor and CRISPR vectors at low or high dose with bezafibrate, in comparison with mice treated with high dose Donor and no bezafibrate, or with low dose Donor and bezafibrate (FIG. 2B). Monitoring body weight and blood testing (serum alanine aminotransferase (ALT), creatinine, cholesterol, and triglycerides), revealed a lack of toxicity from vector administration. Although GSD Ia-related differences were observed in comparison with wildtype mice, no differences based upon vector dose or CRISPR vector administration were demonstrated (FIG. 6A—FIG. 6B, FIG. 7A-FIG. 7D). Additionally, mice treated with CRISPR vector only did not survive past weaning at 3 weeks of age, confirming the need for a therapeutic G6PC transgene expression to sustain G6pc (−/−) mice early in life.

### Example 3

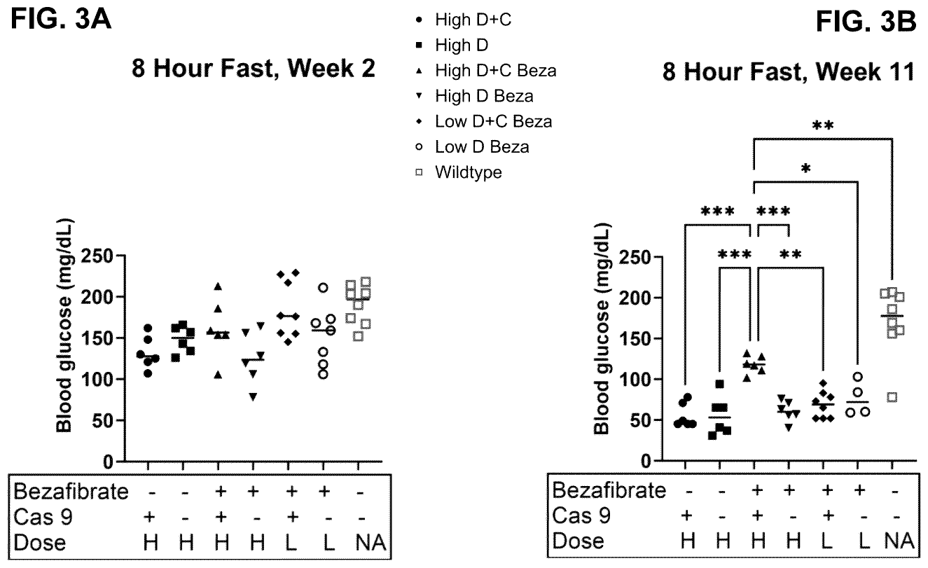

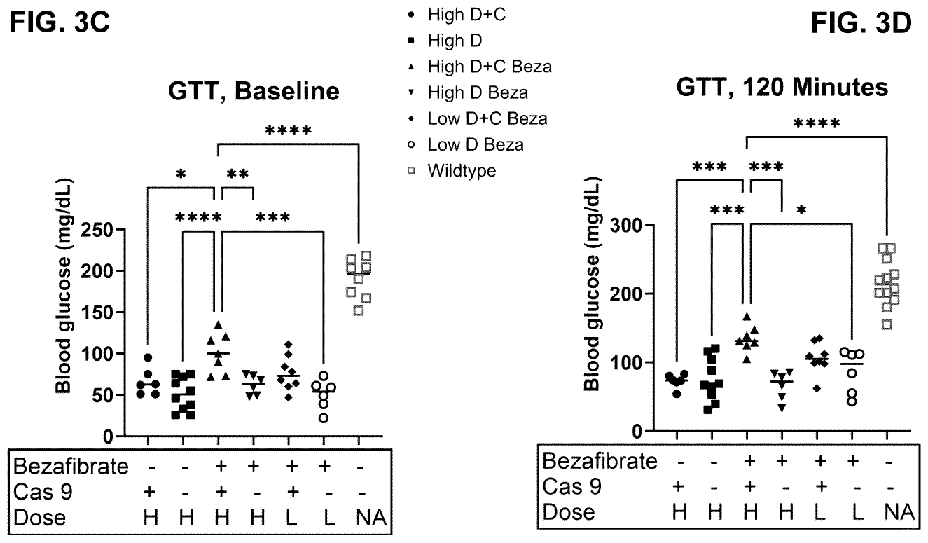

Blood glucose concentrations during fasting were measured two weeks following vector administration. No differences were observed among treatment groups, and blood glucose was similar to the concentrations observed for wild type mice (FIG. 3A). However, by eleven weeks after treatment the fasting blood glucose of mice receiving bezafibrate and high dose Donor+CRISPR vectors was significantly higher compared with all other groups of G6pc (−/−) mice (FIG. 3B). Additionally, the glucose tolerance test performed at 4 weeks following treatment revealed that the mice in the high dose Donor+CRISPR vectors with bezafibrate group had elevated blood glucose at baseline and 120 minutes following glucose administration compared with all other treatment groups except bezafibrate plus low dose Donor+CRISPR vectors (FIG. 3C, FIG. 3D).

### Example 4

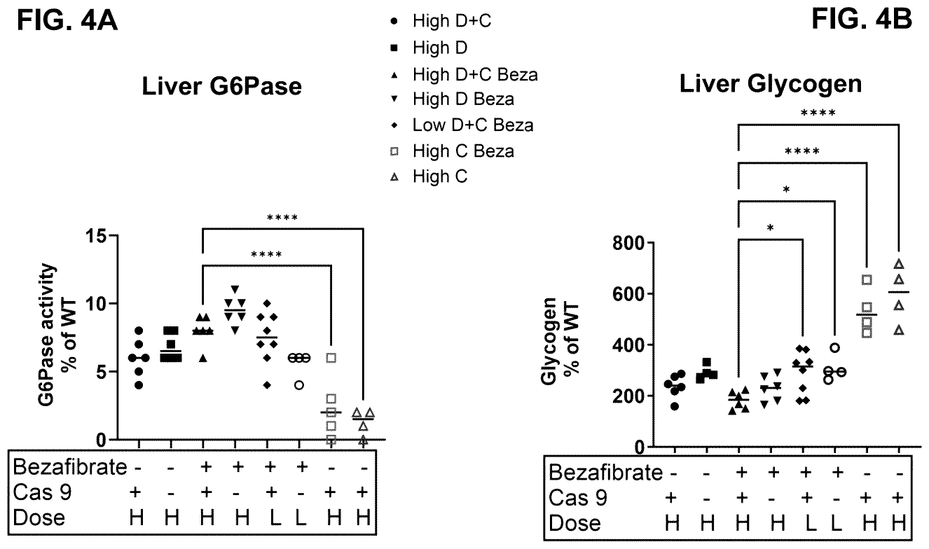

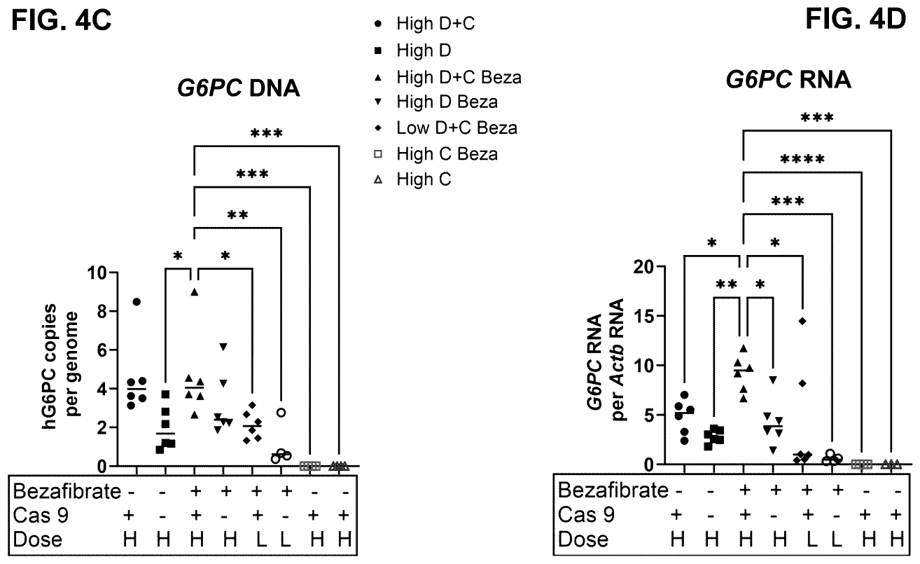

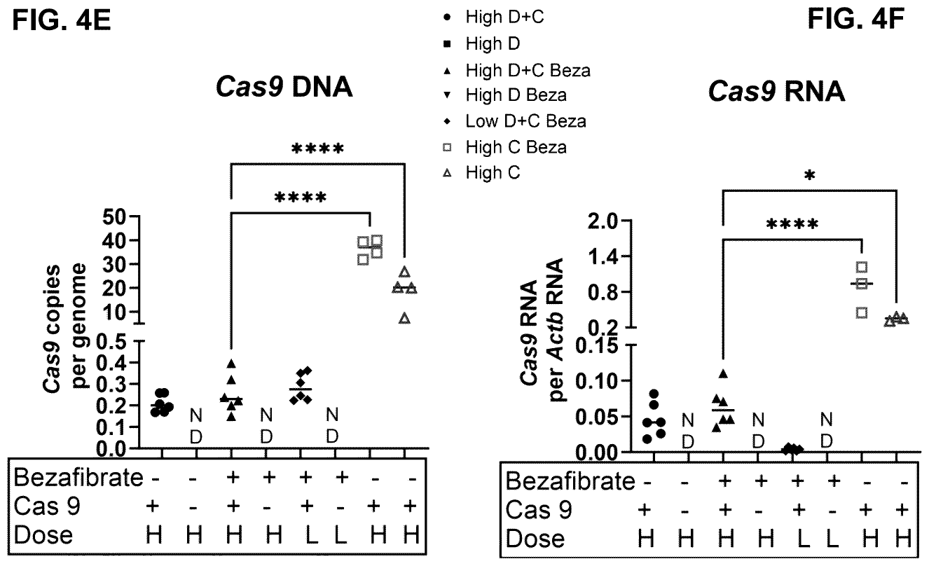

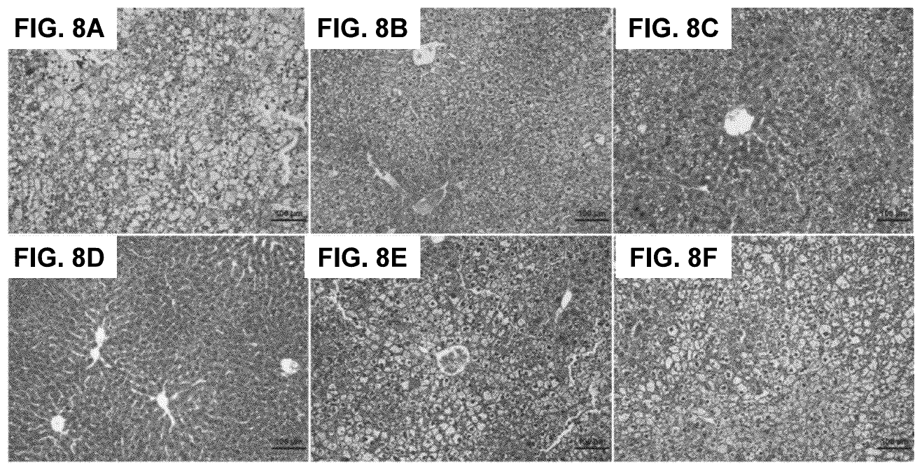

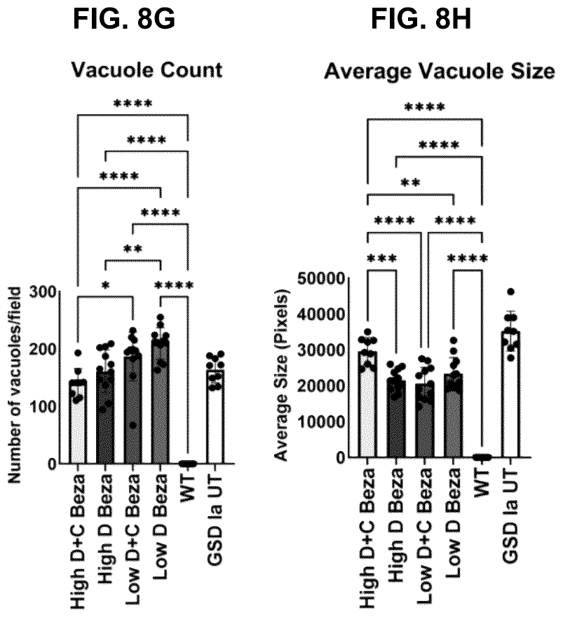

Analyzing the biochemical correction in the liver revealed that the administration of bezafibrate increased G6Pase activity for G6pc (−/−) mice treated with high dose Donor+CRISPR vectors from 6.0%±1.1 to 8.0%±1.1% of WT (FIG. 4A). The high dose Donor+CRISPR vectors with bezafibrate group had the lowest liver glycogen content, which was significantly lower than for the group receiving low dose Donor+CRISPR vectors with or without bezafibrate (FIG. 4B). Liver histology revealed decreased numbers of vacuoles associated with stored glycogen in mice treated with high dose Donor+CRISPR vectors, in comparison with other groups (FIG. 8A-FIG. 8H). Quantification of the Donor vector genomes revealed statistically significantly increased copy number for the high dose Donor+CRISPR vectors plus bezafibrate group, in comparison with groups treated with the low dose of the Donor or with high Donor and no bezafibrate (FIG. 4C). The Donor transgene RNA expression was also highest in the high dose Donor+CRISPR vectors with bezafibrate group, and significantly increased compared with all other treatment groups, confirming that this was the best treatment (FIG. 4D).

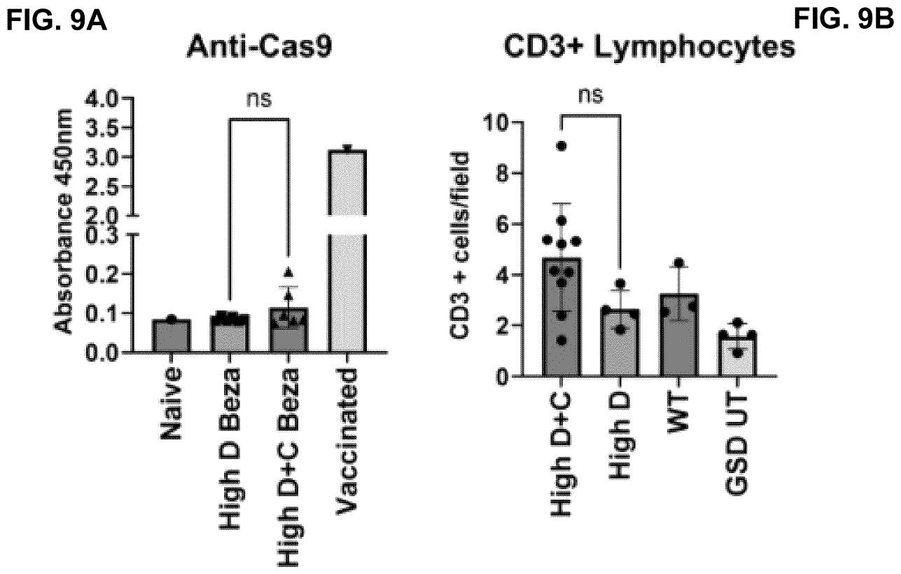

Quantification of the Cas9 transgene demonstrated low copy number at 12 weeks following treatment (FIG. 4E). Importantly, the Cas9 transgene was 120-fold higher at 3 days (High C Beza group; 36 copies/genome), in comparison with 12 weeks (High D+C+Beza group; 0.3 copies/genome) for groups that received high dose CRISPR vector (FIG. 4E). Similarly, Cas9 RNA was 17-fold higher at 3 days, in comparison with 12 weeks (FIG. 4F). At the end of Week 12 the G6PC transgene was sustained at 18-fold higher copy number than the Cas9 transgene for the best-treated, High D+C+Beza group (FIG. 4C, FIG. 4E; 4.7+/−2.2 vs. 0.25+/−0.09), despite the Donor vector being dosed only 5-fold higher than the CRISPR vector. Furthermore, the G6PC RNA was 144-fold higher than the Cas9 RNA (FIG. 4D, FIG. 4F; High D+C+Beza group, 9.2+/−1.8 vs 0.07+/−.0.03). Together these data confirmed stability of the G6PC transgene relative to the Cas9 transgene. Given the risk for immune responses to Cas9, antibody and T cell responses were evaluated without detecting evidence for anti-Cas9 responses (FIG. 9A-FIG. 9B). Thus, the loss of the Cas9 transgene could be attributed to cell division and the loss of the episomal CRISPR vector genomes.

### Example 5

Treatment with bezafibrate increased nuclease activity as reflected by transgene integration and by the generation of indels. Bezafibrate administration increased transgene integration from 3.1±0.8% to 5.9%±1.7% for mice treated with high dose Donor+CRISPR vectors (FIG. 5A). Furthermore, the integrated transgene could be detected only in mice treated with Donor+CRISPR, and no integrated transgene was observed in mice receiving the Donor vector only (FIG. 5A). The Surveyor nuclease assay was used to detect indels introduced at the exon 1/intron 1 boundary of G6pc by CRISPR/Cas9 and repaired by NHEJ. The Surveyor assay detected increased indels in the high dose Donor+CRISPR with bezafibrate treated mice (30%±5.6%) compared with high dose Donor+CRISPR alone (16%±2.4%; FIG. 5B). No indels were detected in mice not treated with the CRISPR vector (FIG. 5B, WT). These data confirmed the activity of CRISPR/Cas9 in the G6pc (−/−) mouse liver, resulting in HDR-mediated transgene integration, which was increased by treatment with bezafibrate. Finally, off-target analysis was performed with next generation sequencing of sites that were predicted to be at risk for cleavage based upon homology to the guide sequence's target site, and the presence of indels at off-target sites were similar between mice treated with CRISPR and control mice (FIG. 5C).

### Summary of GSD1a Examples

This study demonstrates the efficacy of CRISPR/Cas9 based genome editing that can integrate a full-length therapeutic transgene into a defined chromosomal location through HDR in the liver of mice with GSD Ia. The highest total vector dose of 2E+13 vector genomes (vg)/kg was relatively low, in comparison with other studies of genome editing in the liver, even those that treated neonatal mice that are most responsive to genome editing (Zidori Z, et al. (2001) Dev. Cell. 1:291-302; Girod A, et al. (2002) J. Gen. Virol. 83:973-978; Grieger J C, et al. (2006) J. Virol. 80:5199-5210; Dudek A M, et al. (2020) Mol. Ther. 28:367-381; Bossis I, et al. (2003) J. Virol. 77:6799-6810). Administering a vector that expresses Cas9, along with a Donor vector containing a functional G6PC transgene and guide RNA, improved the therapeutic efficacy in young mice. This study demonstrated the benefit of targeted nuclease-dependent genome editing, because administering CRISPR/Cas9 improved the efficacy of the Donor transgene but had no benefits by itself. Cas9 was required to achieve appreciable transgene integration in the G6pc locus. This observation supports the HDR mediated integration depends on nuclease activity in this model, despite reports that Donor templates can integrate spontaneously or independent of nuclease activity (Girod A, et al. (2002) J. Gen. Virol. 83:973-978; Farkas S L, et al (2004) J. Gen. Virol. 85:555-561). Addition of CRISPR/Cas9 increased HDR by 26-fold in mice with Crigler-Najjar (Wang J, et al. (2011) Arch. Virol. 156:71-77), consistent with our data. Furthermore, adding bezafibrate, a drug known to increase transgene expression and editing efficacy, improved integration frequency and biochemical correction in mice long term (Venkatakrishnan B, et al. (2013) J. Virol. 87:4974-4984; Lins-Austin B, et al. (2020) Viruses. 12:66). Stable transgene integration was demonstrated in a study with the GSD Ia canine model; however, the associated biochemical improvement was minimal and attributable to the remaining episomal vector genomes (Afione S, et al. (2015) J. Virol. 89:1660-1672). Similarly to previous studies of gene therapy (Afione S, et al. (2015) J. Virol. 89:1660-1672), puppies with GSD Ia that were treated with genome editing as neonates eventually developed hypoglycemia and required rescue doses of gene replacement therapy (Pénzes J J, et al. (2015) J. Gen. Virol. 96:2769-2779), In the current study with G6pc (−/−) mice the combination of high dose genome editing vectors with bezafibrate resulted in the most efficacious outcome, which correlated with transgene integration. This observation will inform the design of future preclinical studies with the mice or canine models of GSD Ia.

This study surprisingly achieved higher rates of editing in vivo compared with previous studies in GSD Ia. The level of hepatic correction needed to successfully treat GSD I has been estimated at 3%. In the above-mentioned canine GSD Ia study, 0.5-1.0% of alleles were edited to contain the therapeutic transgene. The current study achieved 3.1% transgene integration for mice receiving both Donor and CRISPR vectors, which increased to 5.9% for mice that also received bezafibrate treatment (FIG. 5A). The nearly 6-fold higher frequency of HDR in the current study was associated with significantly improved fasting blood glucose, increased liver G6Pase enzyme activity and decreased liver glycogen content in G6pc (−/−) mice. Furthermore, the efficacious vector dose in the current study was 5-fold lower than that administered by Arnaoutova and colleagues (Nam H-J, et al. (2011) J. Virol. 85:11791-11799). Expression of the G6PC transgene expression was well above the 3% threshold that prevented tumor formation from the effects of GSD Ia in G6pc (−/−) mice (Vibin J, et al. (2020) Sci. Rep. 10:12800), given that 8% of normal G6Pase activity was demonstrated for the best treated group (FIG. 4A).

Benefits of the current genome editing strategy include the ability to treat GSD Ia without regard to the underlying pathogenic variant, as opposed to methods that will only correct a specific mutation. As noted above, the Donor vector expressed the G6PC transgene as an episome. This feature addresses the need for high G6PC expression in young G6pc (−/−) mice (Yates V J, et al. (1981) Am. J. Epidemiol. 113:542-545) to prevent mortality by including a G6PC promoter upstream of the transgene, which has the immediate benefit of gene therapy coupled with the long-term sustained expression of the integrated transgene.

Transgene expression from the episomal Donor vector genomes complicated the detection of benefits from genome editing, especially in the initial 4-week study. Improvement from the addition of Cas9 in the 8-hour fasting and glucose tolerance tests was observed in the low dose group, but not in the medium dose group. No additional benefits from adding Cas9 were observed in the medium dose groups due to higher numbers of Donor vector genomes accompanied by episomal expression, which masked any benefit from transgene integration associated with Cas9 expression. Only the medium dose group had decreased glycogen with Cas9 at 4 weeks, but this could be attributed to low transgene expression from the low dose of Donor vector.

Extending the study to 12 weeks and adding bezafibrate confirmed the advantages of high vector dosages and demonstrated a dose-response. High dose Donor+CRISPR vector-treated mice had improved blood glucose during fasting, increased G6Pase activity, and decreased liver glycogen, in comparison with low dose vector. As expected, following more transgene integration following high dose vector administration, more transgene copies were present in the liver accompanied by higher G6PC expression.

The dramatic loss of the Cas9 transgene provides increased safety by decreasing the potential risks of prolonged nuclease activity (FIG. 4E, FIG. 4F). This transgene loss might be attributed to the high rate of apoptosis in the untreated GSD Ia liver (Naumer M, et al. (2012) J. Virol. 86:13038-13048), which also helps to explain the transience of episomal AAV vectors in GSD Ia (Govindasamy L, et al. (2006) J. Virol. 80:11556-11570; O'Donnell J, et al. (2009) Virology. 385:434-443; Shen S, et al. (2013) J. Biol. Chem. 288:28814-28823; Afione S, et al. (2015) J. Virol. 89:1660-1672). The Donor vector genomes could be stabilized by integration of the G6PC transgene, as well as a previously described potential selective advantage for corrected cells following GSD Ia genome editing (Sonntag F, et al. (2006) J. Virol. 80:11040-11054). At the end of the study G6PC transcripts were 144-fold higher than Cas9 transcripts, confirming a high therapeutic to risk ratio with regard to transgene expression (FIG. 4D, FIG. 4F). The risk of prolonged CRISPR/Cas9 activity has been emphasized by previous studies integration of AAV vector genomes into double-stranded DNA breaks, further increasing the risk of persistent expression of nuclease activity from an integrated vector containing CRISPR/Cas9 (Tse L V., et al. (2018) J. Virol. 92:1-18). The current data indicate that this risk is much lower for genome editing in GSD Ia.

This study was designed to identify conditions for genome editing that could be translated clinically to treat young patients with GSD Ia. The rapid loss of episomal AAV vector genomes from liver, especially in GSD Ia (Govindasamy L, et al. (2006) J. Virol. 80:11556-11570; Maurer A C, et al. (2018) Cell Rep. 23:1817-1830), preempts treatment of affected children early in life. Genome editing provides an alternative approach to genetic therapy with high potential to treat stably, given that genetic modifications of hepatocytes will be impervious to cell division (Zidori Z, et al. (2001) Dev. Cell. 1:291-302).

Three factors were optimized to increase suitability for clinical translation of the current genome editing approach: age of treatment, dose, and vector design. Infant mice were treated at 12 days of age, when the stage of development approximates that for human infants (hair present, eyes open) and clinical features of GSD Ia are present (Govindasamy L, et al. (2006) J. Virol. 80:11556-11570; Sonntag F, et al. (2011) J. Virol. 85:12686-12697). The highest total vector dose was 1.9E+13 vg/kg, and even a 10-fold higher dose has been administered to human infants to treat spinal muscular atrophy through transduction of motor neuron s (Sonntag F, et al. (2010) Proc. Natl. Acad. Sci. USA. 107:10220-10225). A higher clinical dose might be needed due to the lower tropism of AAV9 for human hepatocytes, in comparison with mice (Earley L F, et al. (2015) J. Virol. 89:3038-3048). In contrast, similar genome editing studies in neonatal mice at 2 to 3 days of age used vector doses of 1-2E+14 vg/kg that could not be readily implemented in clinical trial s (Zidori Z, et al. (2001) Dev. Cell. 1:291-302; Girod A, et al. (2002) J. Gen. Virol. 83:973-978; Grieger J C, et al. (2006) J. Virol. 80:5199-5210; Dudek A M, et al. (2020) Mol. Ther. 28:367-381; Bossis I, et al. (2003) J. Virol. 77:6799-6810). The vector design featured a functional G6PC transgene that is effective in GSD Ia both due to early expression from episomal Donor genomes and treats without regard to the pathogenic variants the cause GSD Ia.

The degree of correction in this study was sufficient to treat GSD Ia based upon improved survival and blood glucose concentrations. Liver G6Pase reached 8% of normal, and HDR-mediated transgene integration was approximately 6% following administration of high dose vectors with bezafibrate. The threshold for prevention of hypoglycemia and hepatocellular carcinoma formation in GSD Ia has been established at 3% of normal G6Pase (Shen S, et al. (2013) J. Biol. Chem. 288:28814-28823; Su X N, et al. (2017) Poult. Sci. 96:3867-3871).

The observed HDR-mediated integration frequency of approximately 6% for infant, 12-day old mice with GSD Ia was higher than might have been expected given the age at treatment. The great majority of similar studies treated neonatal affected mice with various inherited metabolic disorders to accomplish genome editing of the liver (Zidori Z, et al. (2001) Dev. Cell. 1:291-302; Girod A, et al. (2002) J. Gen. Virol. 83:973-978; Grieger J C, et al. (2006) J. Virol. 80:5199-5210; Dudek A M, et al. (2020) Mol. Ther. 28:367-381; Bossis I, et al. (2003) J. Virol. 77:6799-6810), which have a higher rate of hepatocyte division that promotes HDR. A recent study of genome editing in neonatal mice with citrullinemia type I utilized a promoter-less transgene and Staph aureus CRISPR/Cas9 to achieve up to 15% HDR-mediated transgene integration (Wistuba A, et al. (1995) J. Virol. 69:5311-5319). Several studies documented lower frequency genome editing when older mice were treated. For OTC deficiency, 10% gene correction was observed for neonatal mice, while adult-treated mice had 0.3% correction (Zidori Z, et al. (2001) Dev. Cell. 1:291-302). A study in mice with Wilson disease demonstrated 7% HDR-mediated integration when 3 week-old mice were treated in the presence of a selective advantage; however, wildtype mice treated in parallel had undetectable HDR (Wobus C E, et al. (2000) J. Virol. 74:9281-9293). Mice with PKU had a baseline HDR-mediated integration frequency of 1%, which was increased to 13% by adding vanillin treatment to suppress the competing process of NHEJ. It is possible that GSD Ia mice feature a selective advantage for corrected cells, given the presence of a degree of hepatoxicity in absence of effective gene therapy as shown by increased liver transaminases (Wistuba A, et al. (1997) J. Virol. 71:1341-1352). A selective advantage based upon increased allele modification in GSD Ia mice was observed when compared with their normal littermates, in a previous study of ZFN-mediated genome editing (Sonntag F, et al. (2006) J. Virol. 80:11040-11054).

### Example 6

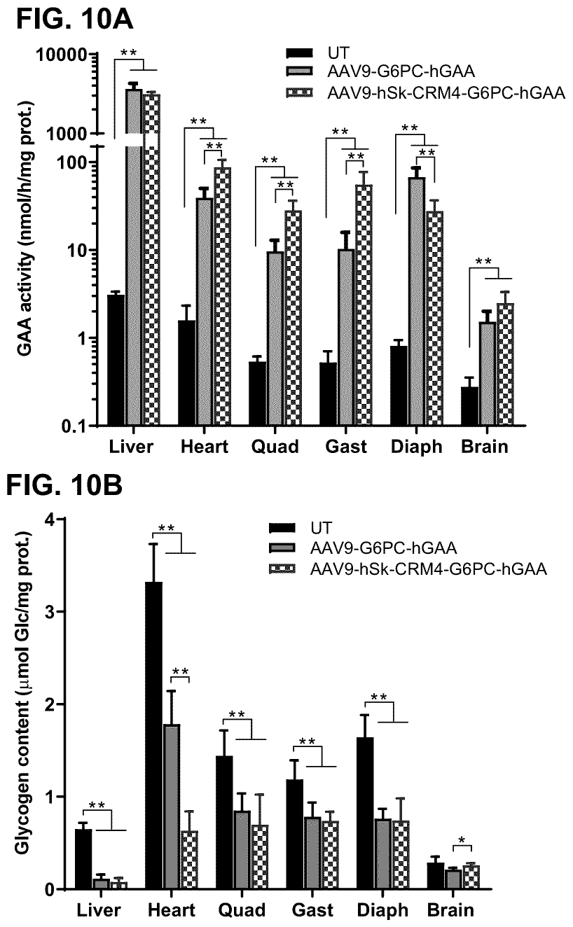

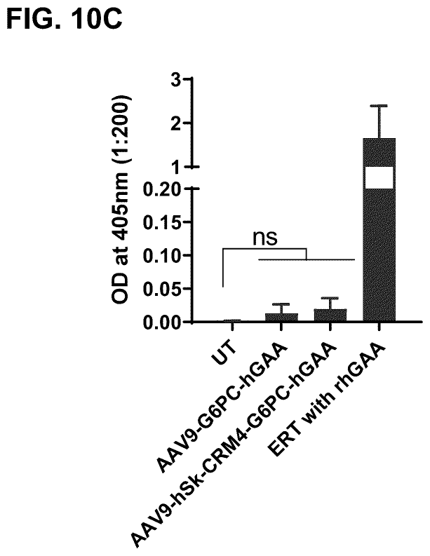

Some of the experiments described in Examples 1-5 were repeated with GAA rather than G6PC. FIG. 10A-FIG. 10C shows three-month-old GAA-KO mice that were injected intravenously (1E13 vector genomes (vg)/kg body weight) with the AAV9-G6PC-hGAA or AAV9-hSk-CRM4-G6PC-hGAA vector and analyzed after 3 weeks. Control group was untreated (UT) GAA-KO mice. FIG. 10A shows GAA activity in tissues. FIG. 10B shows glycogen content in tissues. FIG. 10C shows anti-hGAA antibody detection by ELISA. Mice treated with enzyme replacement therapy (ERT) were used as positive controls.

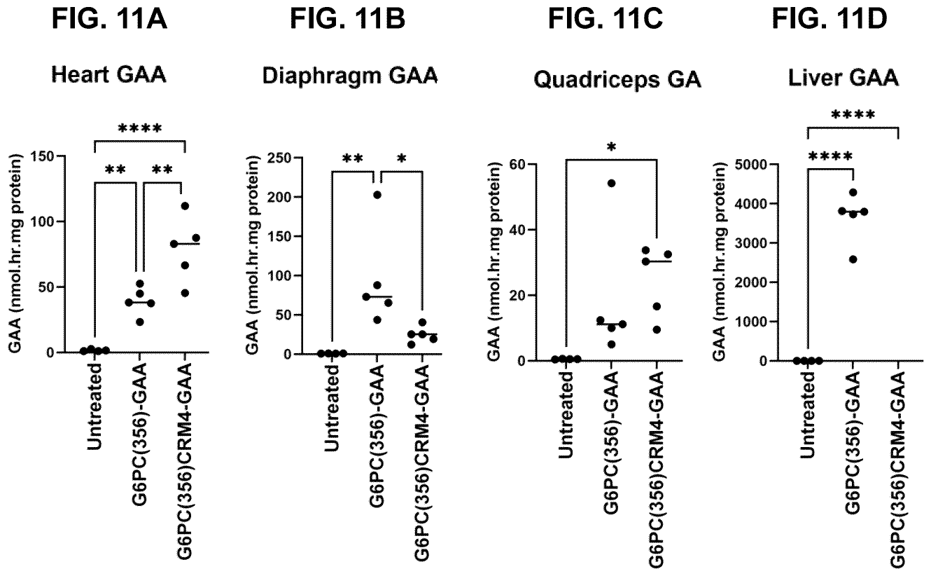

FIG. 11A-FIG. 11D show GAA activities in 4 different tissues 3 weeks after I.V. AAV injection (1×10¹³ vg/kg). Mice were 3 months old at injection. N=4 for untreated controls, n=5 for AAV-G6Pc(356)-GAA treated and AAV-CRM4-G6Pc(356)-GAA treated mice; data for heart GAA (FIG. 11A), diaphragm GAA (FIG. 11B), quadriceps GAA (FIG. 11C), and liver GAA (FIG. 11D) are shown as mean±SD; one-way ANOVA was performed for each tissue. *p<0.05, **p<0.01, ***p<0.001, ****p<0.0001. Wild-type (WT, n=4) values are for reference and are not included in statistic analysis. Addition of CRM4 enhancer elevated GAA activity in heart but not skeletal muscles.

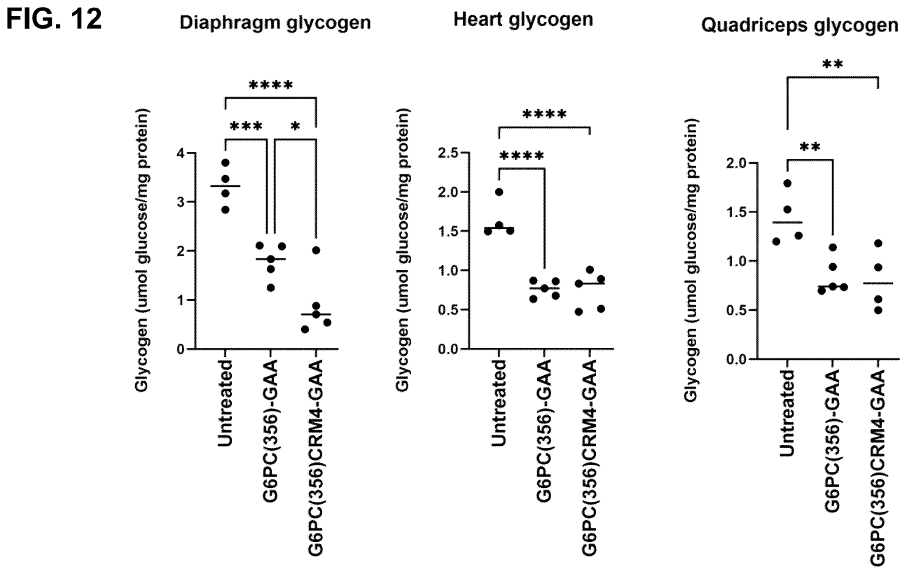

FIG. 12 shows the effect of AAV treatment in glycogen reduction in tissues (diaphragm (left), heart (middle), and quadriceps (right) examined 3 weeks after I.V. AAV injection (1×10¹³ vg/kg). Mice were 3 months old at injection. n=4 for untreated controls and wild-type controls, n=5 for AAV-G6Pc(356)-GAA treated and AAV-CRM4-G6Pc(356)-GAA treated mice; data are shown as mean±SD; one-way ANOVA was performed for each tissue. *p<0.05, **p<0.01, ***p<0.001, ****p<0.0001. Addition of CRM4 enhancer enhanced glycogen reduction in heart but not in skeletal muscles.

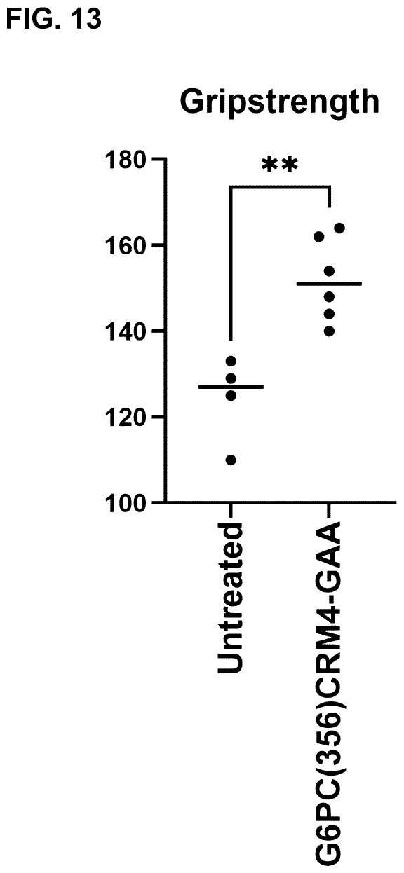

FIG. 13 shows that AAV-CRM4-G6Pc(356)-GAA treatment improved muscle strength as indicated by grip strength test in a 12-week study.

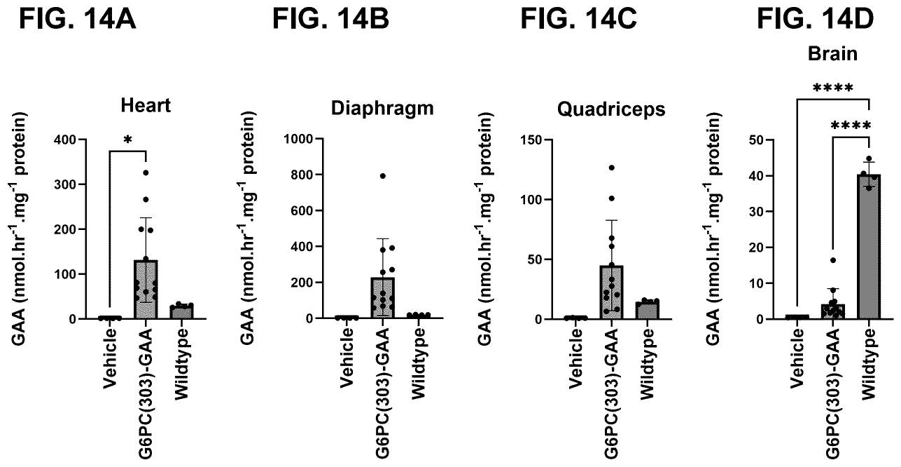

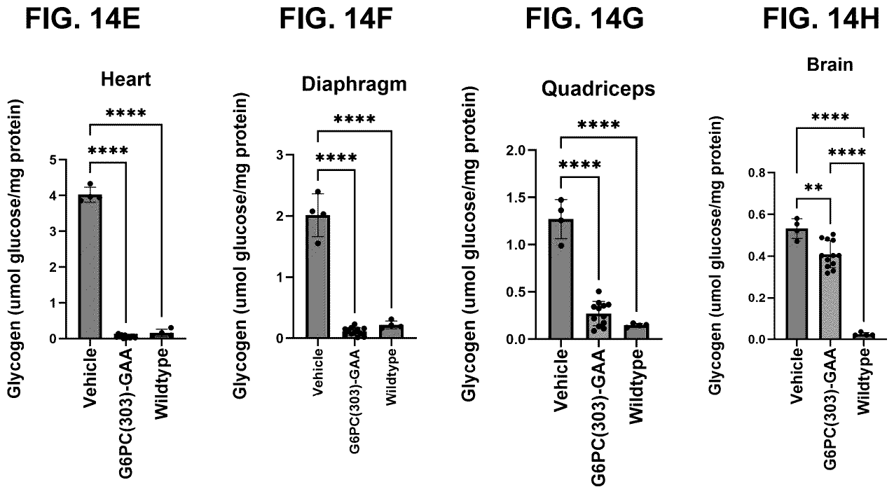

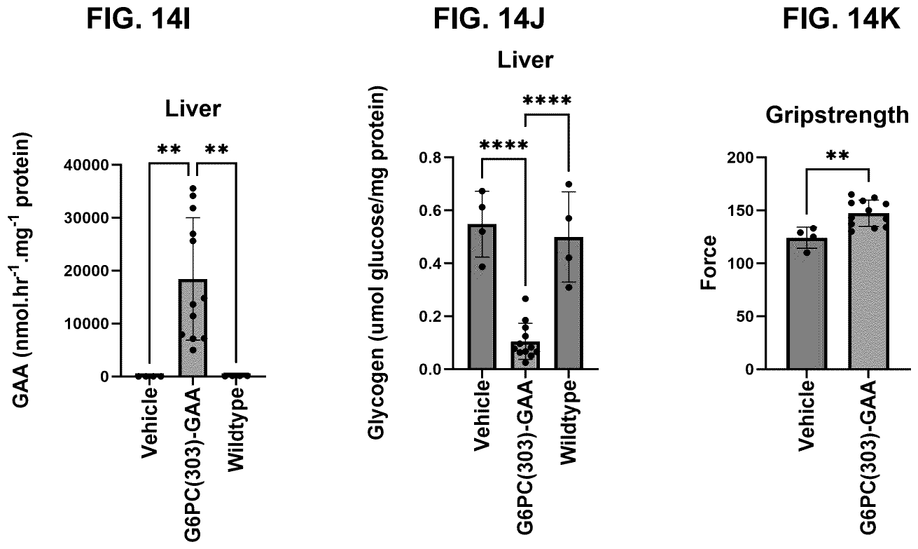

FIG. 14A-FIG. 14D show GAA activity in tissues following AAV9-G6PC(303)-GAA vector administration injection (1×10¹³ vg/kg). Mice were 3 months old at injection. Heart (FIG. 14A), diaphragm (FIG. 14B), quadriceps (FIG. 14C), and brain (FIG. 14D) are shown. N=4 for untreated controls and wild-type controls, n=12 for AAV-G6Pc(303)-GAA treated GAA-KO mice. FIG. 14E-FIG. 14H show the glycogen content in tissues following AAV9-G6PC(303)-GAA vector administration injection (1×10¹³ vg/kg). Mice were 3 months old at injection. Heart (FIG. 14E), diaphragm (FIG. 14F), quadriceps (FIG. 14G), and brain (FIG. 14H) are shown. N=4 for untreated controls and wild-type controls, n=12 for AAV-G6Pc(303)-GAA treated GAA-KO mice. FIG. 14I-FIG. 14K show liver analysis and muscle function testing following AAV9-G6PC(303)-GAA vector administration injection (1×10¹³ vg/kg). Mice were 3 months old at injection. Liver GAA activity (FIG. 14I), and glycogen content (FIG. 14J) are shown. Grip strength (FIG. 14K) was performed at 12 weeks. N=4 for untreated controls and wild-type controls, n=12 for AAV-G6Pc(303)-GAA treated GAA-KO mice. Data are shown as mean±SD; one-way ANOVA was performed for each tissue. *p<0.05, **p<0.01, ***p<0.001, ****p<0.0001. Wild-type (WT and n=4) values are for reference and are not included in statistical analysis.

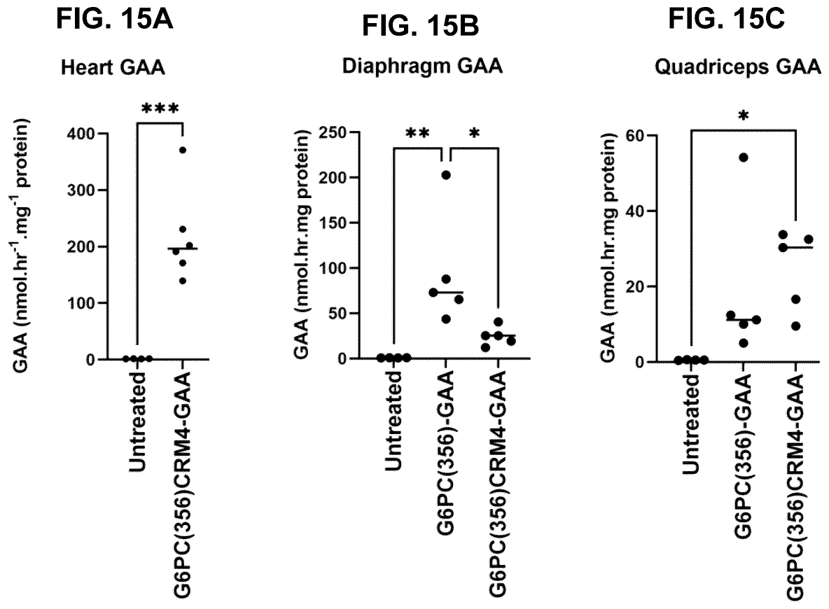

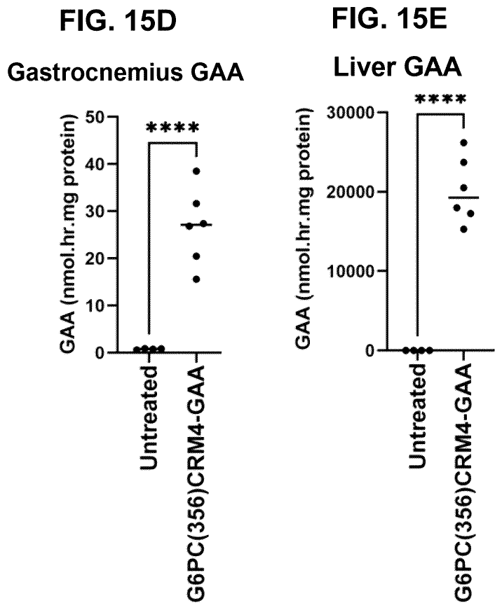

FIG. 15A-FIG. 15E show GAA activity in various tissues (heart (FIG. 15A), diaphragm (FIG. 15B), quadriceps (FIG. 15C), gastrocnemius (FIG. 15D), and liver (FIG. 15E)) following AAV9-G6PC(356)CRM4-GAA vector administration injection (1×10¹³ vg/kg) and/or AAV-G6PC(356)-GAA vector administration injection (1×10¹³ vg/kg).

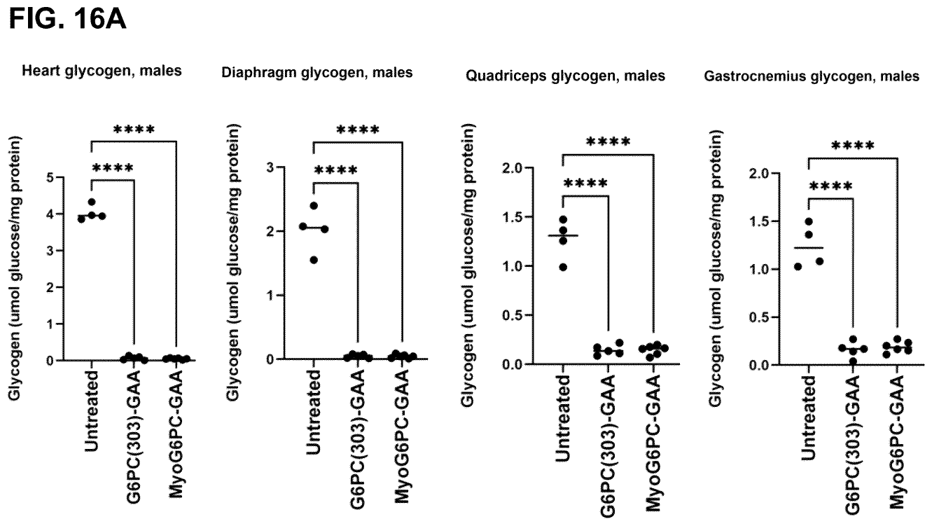

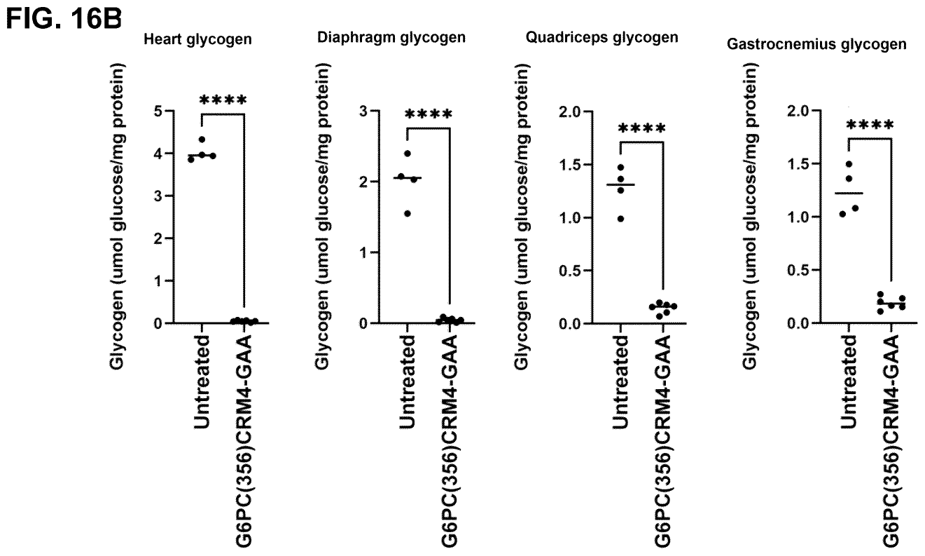

FIG. 16A shows GAA activities in tissues 12 weeks after I.V. AAV injection (1×10¹³ vg/kg). Mice were 3 months old at injection. N=4 for untreated controls, n=5 for AAV-G6Pc(303)-GAA treated mice and n=6 for AAV-MyoG6Pc-GAA (AAV-CRM4-G6Pc(356)-GAA) treated mice; data are shown as mean±SD; one-way ANOVA was performed for each tissue. *p<0.05, **p<0.01, ***p<0.001, ****p<0.0001. AAV-MyoG6Pc-GAA did not enhance GAA expression in heart or skeletal muscles. There was no effect in the brain. FIG. 16B shows AAV treatment reduce glycogen in muscles to wild-type level 12 weeks after I.V. AAV injection (1×10¹³ vg/kg). Mice were 3 months old at injection. N=4 for untreated controls, n=6 for AAV-MyoG6Pc-GAA (AAV-CRM4-G6Pc(356)-GAA) treated mice; data are shown as mean±SD; one-way ANOVA was performed for each tissue. *p<0.05, **p<0.01, ***p<0.001, ****p<0.0001. There was no detectable effect in the brain.

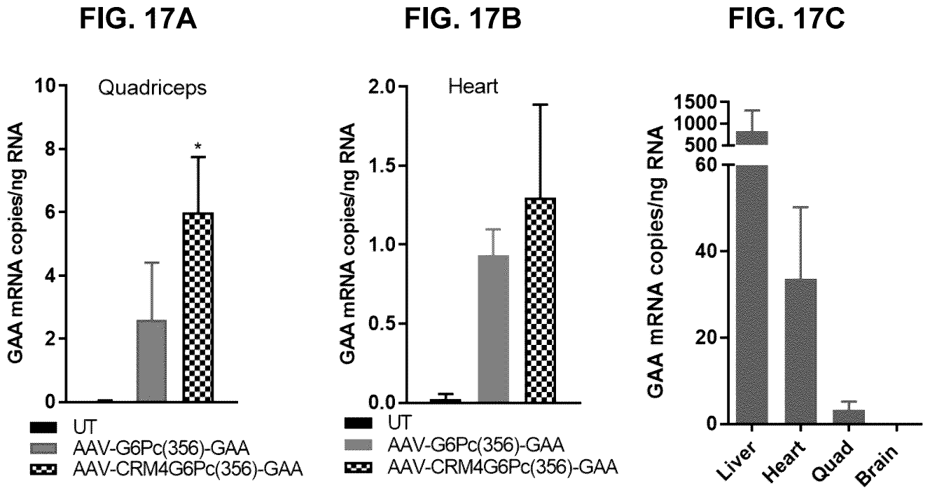

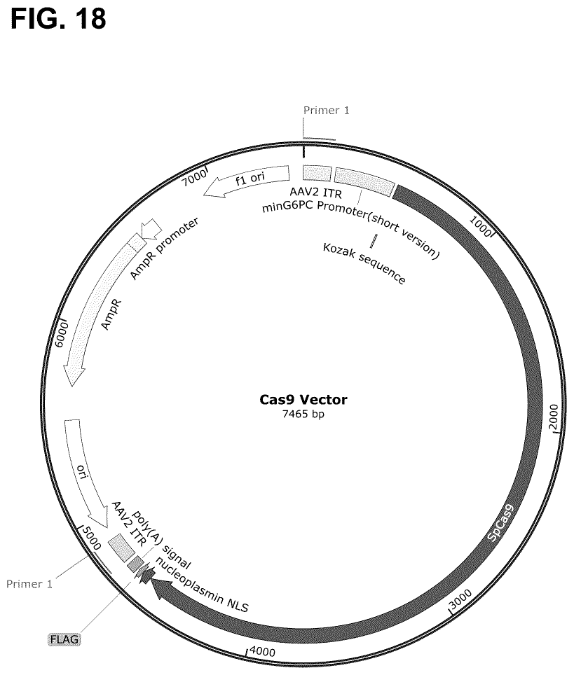

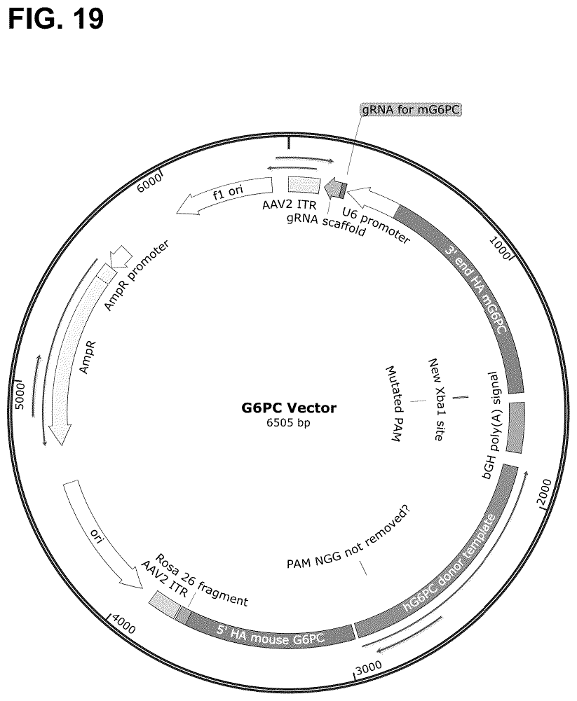

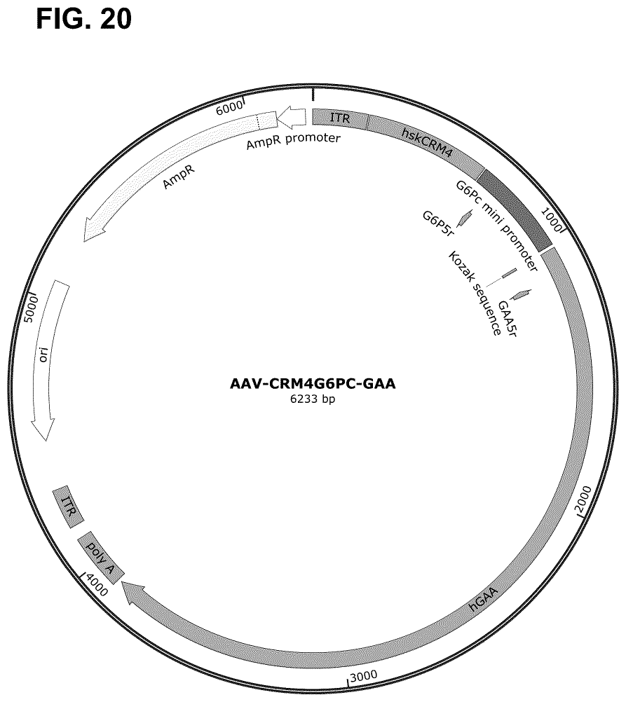

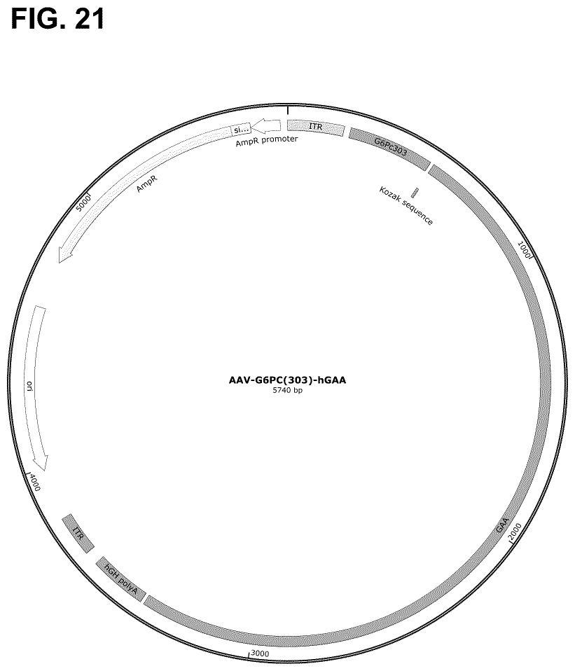

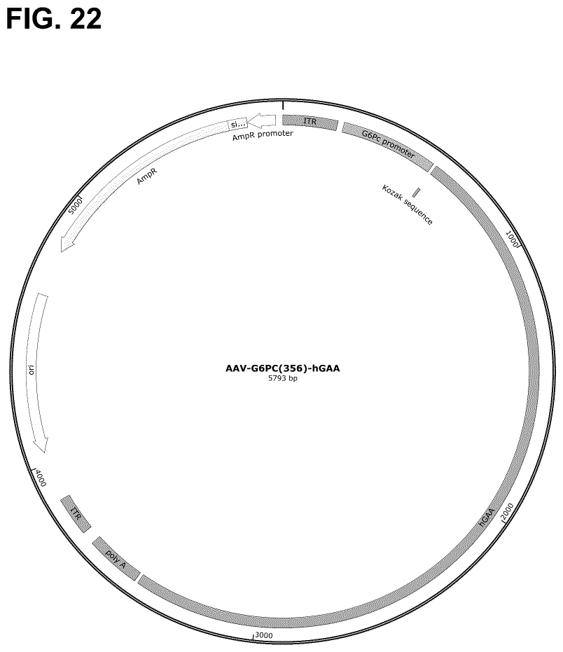

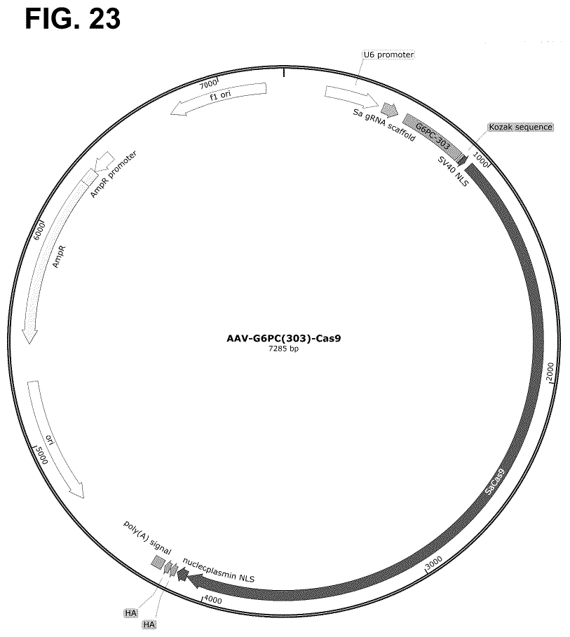

FIG. 17A-FIG. 17C shows GAA mRNA in tissues of (male) mice treated with AAV. FIG. 17A shows GAA mRNA copies in quadriceps 3 weeks after AAV injection (n=4 each group). FIG. 17B shows GAA mRNA copies in heart 3 weeks after AAV injection (n=4 each group). FIG. 17C shows GAA mRNA copies in tissues 12 weeks after injection of AAV-G6Pc303-GAA. N=3 each. RNA was extracted using Qiagen RNeasy Mini Kit; first strand cDNA was synthesized using Invitrogen M-MLY reverse transcriptase and random primer; real-time PCR was performed with iTaq Universal SRBR Green Supermix (BioRad) and primers to GAA (forward: cagagcagtgcccacacag (SEQ ID NO:140), reverse: gtagcccatttcagaggagctc (SEQ ID NO:141) and primers to β-actin (AGAGGGAAATCGTGCGTGAC (SEQ ID NO:129); CAATAGTGATGACCTGGCCGT (SEQ ID NO:130). A linearized plasmid was used for generating a standard curve for calculating GAA mRNA copy number.

These experiments show that Donor vectors having a disclosed G6PC min promoter and a GAA transgene (or fragment/repair template) (i) generated supraphysiologic level of GAA in liver and striated muscle, (ii) restored normal glycogen content in the heart, diaphragm, and quadriceps, and (iii) improved grip strength, thereby demonstrating that the disclosed G6PC min promoters and disclosed G6PC min hybrid promoters (when used in a Donor vector) can be used in a disclosed method of gene therapy.

This study aims to investigate whether genome editing from the addition of a vector containing a Cas9 transgene (CRISPR vector) would enhance the efficacy of a vector containing the GAA transgene vector (see, e.g., Donor vector; FIG. 1A). Two recombinant AAV9 (rAAV9) vectors are generated for this study. The CRISPR vector contains the S. pyogenes Cas9 gene driven by a minimal G6PC promoter (FIG. 1A). The Donor vector contains a human GAA transgene with minimal G6PC promoter flanked by homology arms corresponding to GAA. The Donor vector also contains a U6 promoter expressing a gRNA targeting the GAA. When CRISPR/Cas9 cleaves the GAA locus, the transgene is predicted to insert via HDR resulting in an integrated full length GAA transgene in the GAA locus (FIG. 1B).

Initially, GAA (−/−) mice with GSD II are treated at 12 days of age with two different dosages of vectors: low dose (Donor, 2×10¹² vg/kg; +/−CRISPR 4×10¹¹ vg/kg), or medium dose (Donor 8×10¹² vg/kg; +/−CRISPR 1.6×10¹² vg/kg). Two weeks following vector administration the GAA (−/−) mice that received the low dose of both Donor+CRISPR vectors had increased blood glucose concentrations during fasting, in comparison with mice treated with low dose Donor vector alone. A glucose tolerance test is performed four weeks after treatment to further evaluate glucose metabolism. In the glucose tolerance test, low dose Donor+CRISPR vector administration improves blood glucose at Baseline following 4 hours fasting and at 120 minutes following glucose administration, in comparison with Donor vector alone. Biochemical correction of the GAA (−/−) mouse livers is evaluated 4 weeks following treatment by analyzing GAA activity and glycogen content.

The study is extended to 12 weeks to determine the most efficacious treatment. The high and low dose vector treatment with and without administration of the drug bezafibrate was compared. Bezafibrate is a pan-agonist of peroxisome proliferator-activated receptors (PPARs) that enhances the expression of genes involved in lipid homeostasis and energy metabolism. Enzyme analysis is performed as described (Govindasamy L, et al. (2006) J. Virol. 80:11556-11570). Monitoring body weight and blood testing (serum alanine aminotransferase (ALT), creatinine, cholesterol, and triglycerides) is performed.

Blood glucose concentrations during fasting is measured two weeks following vector administration. Additionally, the glucose tolerance test is performed at 4 weeks following treatment.

Liver histology is performed to identify the numbers of vacuoles associated with stored glycogen in mice treated with high dose Donor+CRISPR vectors, in comparison with other groups.

Quantification of the Cas9 transgene is performed. Finally, off-target analysis is performed with next generation sequencing of sites that are predicted to be at risk for cleavage based upon homology to the guide sequence's target site.

What is claimed is:

1. A glucose-6-phosphatase (G6PC) minimal promoter comprising the sequence set forth in SEQ ID NO:02 or SEQ ID NO:03.

2. The G6PC minimal promoter of claim 1, further linked to a skeletal muscle-specific transcriptional cis-regulatory module comprising Sk-CRM4, and wherein the Sk-CRM4 regulatory module comprises the sequence set forth in SEQ ID NO:04 or SEQ ID NO:05.

3. A viral vector, comprising: the G6PC minimal promoter of claim 1 operably linked to a nucleic acid sequence encoding a transgene, a nucleic acid sequence encoding a guide RNA (gRNA) targeting a gene locus having one or more defects, a second promoter operably linked to the gRNA, and a pair of sequences flanking the sequence encoding the transgene, wherein the pair of sequences are homologous to a portion of the gene locus having the one or more defects.

4. A viral vector, comprising: the G6PC minimal promoter of claim 1 operably linked to a nucleic acid sequence encoding a Cas9 endonuclease.

5. A gene editing system, comprising:
a viral vector comprising a glucose-6-phosphatase (G6PC)minimal promoter operably linked to a nucleic acid sequence encoding a transgene, a nucleic acid sequence encoding a guide RNA (gRNA) targeting a gene locus having one or more defects, a second promoter operably linked to the gRNA, and a pair of sequences flanking the nucleic acid sequence encoding the transgene,
wherein the pair of sequences are homologous to a portion of the gene locus having one or more defects;
a viral vector comprising a G6PC minimal promoter operably linked to a nucleic acid sequence encoding a Cas9 endonuclease.

6. The gene editing system of claim 5, wherein the viral vectors comprise recombinant adeno-associated virus (AAV) vectors (rAAVs).

7. The gene editing system of claim 5, wherein the Cas9 endonuclease is SaCas9.

8. The gene editing system of claim 5, wherein the G6PC minimal promoter comprises the sequence set forth in SEQ ID NO:02 or SEQ ID NO:03.

9. The gene editing system of claim 5, wherein the G6PC minimal promoter is linked to a skeletal muscle-specific transcriptional cis-regulatory module comprising Sk-CRM4, and wherein the Sk-CRM4 regulatory module comprises the sequence set forth in SEQ ID NO:04 or SEQ ID NO:05.

10. A method of delivering gene therapy, the method comprising:
administering to a subject in need thereof a therapeutically effective amount of the viral vector of claim 3, wherein following transduction, the encoded transgene is expressed in one or more cells of the subject.

11. The method of claim 10, wherein (i) the subject has glycogen storage disease II (GSDII) and the transgene is acid alpha-glucosidase (GAA), or (ii) the subject has glycogen storage disease Ia (GSDIa) and the transgene is glucose-6-phosphatase (G6PC).

12. The method of claim 10, further comprising administering to the subject a therapeutically effective dose of bezafibrate.

13. The method of claim 10, wherein the expression of the encoded transgene increases in the subject's skeletal muscle, liver, kidneys, heart, or any combination thereof.

14. A method of repairing one or more defects in a gene, the method comprising:
administering to a subject in need thereof a therapeutically effective amount of the gene editing system of claim 5, wherein following transduction with the viral vectors, the one or more defects in the gene are repaired in one or more cells in the subject.

15. The method of claim 14, wherein the Cas9 endonuclease creates a double-strand break (DSB) at or near the one or more defects in the gene locus that results in a permanent integration of the encoded transgene.

16. The method of claim 14, wherein the (i) subject has GSDII, the transgene is GAA, and the gRNA targets the one or more defects in the GAA gene, or (ii) subject has GSDIa, the transgene is G6PC, and the gRNA targets the one or more defects in the G6PC gene.

17. The method of claim 14, further comprising administering to the subject a therapeutically effective dose of bezafibrate.

18. The method of claim 14, wherein the expression of the encoded transgene increases in the subject's skeletal muscle, liver, kidneys, heart, or any combination thereof.

19. The method of claim 14, wherein the expression of the encoded transgene minimizes one or more symptoms associated with the one or more defects in the gene.

20. The method of claim 16, further comprising implementing a change in the subject's dietary intake of carbohydrates, and wherein implementing a change in the subject's dietary intake of carbohydrates comprises adding carbohydrates to the subject's diet, or removing carbohydrates from the subject's diet, or changing the type of carbohydrates in the subject's diet, or changing the frequency of carbohydrates consumed by the subject.


SEQUENCE LISTING (160 sequences)

SEQ ID NO: 1 - DNA, 1284 bp, Homo sapiens
  source 1..1284: mol_type = genomic DNA
agctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttc

SEQ ID NO: 2 - DNA, 303 bp, synthetic construct
  source 1..303: mol_type = other DNA
ctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagc

SEQ ID NO: 3 - DNA, 356 bp, synthetic construct
  source 1..356: mol_type = other DNA
ctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttc

SEQ ID NO: 4 - DNA, 434 bp, synthetic construct
  source 1..434: mol_type = other DNA
ttctgagtcctctaaggtccctcactcccaactcagccccatgtcctgtcaattcccactcagtgtctgatctccttctcctcacctttcccatctcccgtttgacccagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagagg

SEQ ID NO: 5 - DNA, 452 bp, synthetic construct
  source 1..452: mol_type = other DNA
cttaagctcgagttctgagtcctctaaggtccctcactcccaactcagccccatgtcctgtcaattcccactcagtgtctgatctccttctcctcacctttcccatctcccgtttgacccagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggctcgag

SEQ ID NO: 6 - DNA, 814 bp, synthetic construct
  source 1..814: mol_type = other DNA
cttaagctcgagttctgagtcctctaaggtccctcactcccaactcagccccatgtcctgtcaattcccactcagtgtctgatctccttctcctcacctttcccatctcccgtttgacccagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggctcgagtctagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttc

SEQ ID NO: 7 - DNA, 808 bp, synthetic construct
  source 1..808: mol_type = other DNA
cttaagctcgagttctgagtcctctaaggtccctcactcccaactcagccccatgtcctgtcaattcccactcagtgtctgatctccttctcctcacctttcccatctcccgtttgacccagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggctcgagctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttc

SEQ ID NO: 8 - DNA, 761 bp, synthetic construct
  source 1..761: mol_type = other DNA
cttaagctcgagttctgagtcctctaaggtccctcactcccaactcagccccatgtcctgtcaattcccactcagtgtctgatctccttctcctcacctttcccatctcccgtttgacccagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggctcgagtctagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagc

SEQ ID NO: 9 - DNA, 755 bp, synthetic construct
  source 1..755: mol_type = other DNA
cttaagctcgagttctgagtcctctaaggtccctcactcccaactcagccccatgtcctgtcaattcccactcagtgtctgatctccttctcctcacctttcccatctcccgtttgacccagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggctcgagctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagc

SEQ ID NO: 10 - DNA, 814 bp, synthetic construct
  source 1..814: mol_type = other DNA
ctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttctctagacttaagctcgagttctgagtcctctaaggtccctcactcccaactcagccccatgtcctgtcaattcccactcagtgtctgatctccttctcctcacctttcccatctcccgtttgacccagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggctcgag

SEQ ID NO: 11 - DNA, 724 bp, synthetic construct
  source 1..724: mol_type = other DNA
ctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttccttaagctcgagttctgagtcctctaaggtccctcactcccaactcagccccatgtcctgtcaattcccactcagtgtctgatctccttctcctcacctttcccatctcccgtttgacccagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtctt

SEQ ID NO: 12 - DNA, 761 bp, synthetic construct
  source 1..761: mol_type = other DNA
ctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagctctagacttaagctcgagttctgagtcctctaaggtccctcactcccaactcagccccatgtcctgtcaattcccactcagtgtctgatctccttctcctcacctttcccatctcccgtttgacccagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggctcgag

SEQ ID NO: 13 - DNA, 755 bp, synthetic construct
  source 1..755: mol_type = other DNA
ctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagccttaagctcgagttctgagtcctctaaggtccctcactcccaactcagccccatgtcctgtcaattcccactcagtgtctgatctccttctcctcacctttcccatctcccgtttgacccagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggctcgag

SEQ ID NO: 14 - DNA, 10188 bp, Mus musculus
  source 1..10188: mol_type = other DNA
agcagagggatcaaggccaaccgggctggactcactgcacgggctctgctggcagcttcctgaggtaccaagggaggaaggatggaggaaggaatgaacattctccatgactttgggatccagtcgactcgctatctccaagtgaattaccaagactcccaggactggttcatccttgtgtctgtgattgctgacctgaggaacgccttctatgtcctctttcccatctggttccatcttaaagagactgtgggcatcaatctcctctgggtggcagtggtcggagactggttcaacctcgtcttcaagtggtaagaaccatcccgaggggtgatgggtagagaaatggctgtcctccagcaggggtaccccatactgttcaccaggctacaggtaagtccccattggcttgagagtctctacagaatcccacctgtggctggcaatgaggaatggctattttgtcagacctgagggatggaaagacagctgagtttaatgggaagtctgagtgtagcctttactctggtttatttgactttattatttagagttagggtcttgtgtagcccaggctggcctcaaacttacaaattaagagacaacgaccttgaattgctcaacttctgagtgctgagattacaggtgtgtgttcccacgtgtagtttagcatgctagggattgaacctggggcttggagcatgagagcaaggactcccccatgaggctatatcccagctcctgagcacagatgttaaaaaaacccttttactactatttctatgggtgctatagaaacagatgtgggaaagaaggggggggggagtgatttctaaccttttggttaagcggtgaaaattgatacggatagccaggaccttccaactggtgaattacataaactcactattctagttttagaatctataaactttggttctctccaggatcttaataatatgcacaatcaacgtttattgatcagctactatgtgccaggctcgcactgtctctccctgtgtctgtctcagtctgtgtgtgtgtttgtgtgtgtgcgcacgtgttcatctgcgtacatgcatgtggctgtgtttgtggaagccagtgcagtcattcctcaaaacttattaagataaagtctctgattgaactggagctccggagtaaatttgaccaatgagcactggaggtctacctgcctgtattcccatagctatatgccaccatgcccaggatttctttgataggatggggatggaattcaggttttctgtttcatagcaggcacttgatctactgagccttcacccagtccctgattcccagatttgcttgtggattgtttgtctgtttgtttgtttgttttgagacagaatctcccctggctgtcctgaaattttactgtgtagaccaggctagcctcaaactcacagagatctgcctgcttctgattccagagtgttgggattaaagatgtgcaccaacaagcctggcttttgcgtgtgctttttaagatggaggatggggtctcttctcattctgtaactcagcctggcctggaactgattaaatagtcctgtctggtctcaacacacgcctgttctcaagtgctgggattacaggtgtgagccaccatgcctagcgtgtttgctttaagttctagatcctgagtatagaccctgtacatgctctttgttactgaactataccctcagccccatgagatttttttttcttataagacaaggtctcttgctgtgtaacctagactagcgtccagtttataatcctcaagcctcagactgcagagggattagaggtgtgtataactctccccagccccagggcatttttgttttgttttgtttttgagtcaaacatgtggatgacatgggttggcctgaaagtagctgataatcactgtggatttcccattctctttcatcagactcaggctagaattacaggtgtgttcctttctaccaggtgtgtgtgtgtgtgtgtgtgtgtgtgtgtgtgtgtgtgtgtgtgtttgtgtgtgtgtttacacatatgcatacgtgcacgaattcttatacatatggacctcagaagacagcttacaggaatctattatctcctttcaccatgtgggtctcaaaaacagaactcaggtcatcaggtttgtgggcaagcacctttacatgttagccatctttctgctggcctgacatcttcagatctcacattctttgcccataaatagtaaaagtccttaaaagaagaacaaatggggttcaagactgaatccagggcctcatatatactaatccagtgatataccactgagatacacccccagccctcttttaatttatctttattatgaagtaggtccttggggttgaggtcatggctcagccactgtttgtcttataaacccaagttcttgaaccgctgggcacaatagtatgcatttgtaaccccaatagtggtaaggtgaagaaggccagtggagccgatccctggagctaatctaacctgattggtgagcttcattcagatcaatgtgagacccgatgtcaaaagagacaggtggcctttctaagtttgtcctctggcctccacaccacttctcctcgtgcacatacacacatgagatttggaaagggcatgggcaggtgaagagggatagagaaagggaagggcaggacctaggctagctccaaagtcttagtctcaagtgatctttctgtgtcagcctccccaatagctaagaccaccactgcatcgaacttctgaagctctatgtttaggtttgagtagataggacattcattcactgttcaaatttcagaaatcatctacttatcatacatagcacccccagacctctccccaggcacctccaaaggcaatacacagtcaagtttccttctctccttgaaactgtgggcttccgataaaatcatgtactgactcactccagacattggctctgctctccacaggattctgtttggacaacgcccgtattggtgggtcctggacaccgactactacagcaacagctccgtgcctataataaagcagttccctgtcacctgtgagaccggaccaggtgagagtcccaaccctcactgaggtggggcagacgttgttcttcatagactggcactaacgtcatgtgtgtgggtctgcactgaccttgggggtggggggtagggtggggtttcaggacagccattggtttgttctattgtcacagtcaatttgtctttcatgtagcacaaattcaccttaaatcatggcaccagggctttgcattgctagtttggaaatctaaatgatgctcccaggattaaagttactactccaaaagaggcaatggtacacccacgggcaatggctagttagagtaactgagccagagcactgtatccagagaggagacagtggtcgacagaagcctgaggccttgagtgtccttcctagcagcctgatccagatcgttccatacctaatgctacctgcagtttttgtcttatttaggggaggtggtttctttgttttgagggatgtcctcatatagtccagactggtcctaaattcactatgtaattgaagatgaccttgaacttctgatcctccttcctccataacaaatcagtaaagcaatatattagaagatcgccaggatctacttggcatctcacagctgcctgtaactctagtttcaggggatccaattcttctgtgaatactgcatgtttgtggtgaacagacatacatgtaggaaaggcacgcatacacatgattttttgtttgtttgtttgtttgtttgtttgtttttgtttctgagacagggtttctctgtatagccctgagacagggtttctctgtatagccctggctgcctggaactcactctgtagaccaggctggcctcgaactcagaaatccacctgtctctgcctcccaagtgctgggactaaaggcgtgtgctaccactgcccggcccacatgattattttttaaataggagagagagaaagagagagaaaaagagagaatatatttgtctacaagaccttctgaatgttgtatggagtgtagatgagagagcggttgatacccaccagaaccacaattgtttggggaagggatgtgctaatgggctgatgaaggccagttgaggctaaacctcacatgacccccacaaagaacgcccaactagagatttctcttcaaaaattgttctgaacaggttgagggcaccagcctttatggcatgatacagactagtcatgggaaacaggctatacttaggtatagggtataccattgaccaagatagctgtcttcagcccaaagacatgcccataaaaggctaagagttaaggagcacccacaactacactcttaccagctggaaaactatcactccatccaggatggggtgtggatagcaggtcaggagtgagaagcaacttaaattggttggagtgggtcttattgaaaaagtgagtctgagacaagatttgaagaagccagggagttgaccagaggaactttgggagaaaagcatccgtgattcatttaggggcaggcaatgagacagtgtggctagagaagagagagaaggaaaatataggggagctgagggtggacggatcggtgtgtaacttacaccctcactgtccggattcctgtgcaatctgcaaactgtctgcaacctcaagagcaatggaggacctctctggtctgagcacagtcctcccacacccctgcacaatgcttggcacacggtgtgaattccatttgttaaataagtgaattacagtaaaggagtggatagtgagatgggtggatagctagttagacggaaggcatgatgactggatgggcttgatggatggatggggagacgtgtggctgatgccttgacagtctctcagattaacgtctttccatcgtaagtccattcatctcttttcttccatgtaggaagtccctctggccatgccatgggcgcagcaggtgtatactatgttatggtcacttctactcttgctatctttcgaggaaagaaaaagccaacgtatggattccggtaagaactcaccactggggctgggggtgaacaggaagctgcacactctgtagctgacccagaggggttcttccaccttgctagctcattctcacacctacagttggcactagtaagaggtgtgacatttctgctgtgagttagcatcacaggatgcttgggccagaagggatccactagggtagcccttgttttagactcttatgggtttggttctgggtatcaaacttggggctttgtgcatgttgtataactagtctatcacagagctatatgtctagcattaaaaaaaaacccttttctgtatcatcctggagtcacctcatataggttaaaataaacagacataagctcctaatgtggggatcctgggtatgcagacatagtgtaaatccttagttattaaatggttaaaactgggcagttaagatgcatacaaaggggagcaatggctcagtagttaggaacactggaagctcttccggaggacttggactcagttcccagcaccccctcggtggttcacagcagcctagaaccccagttccaggagatctgattccacccctgtggactccaggcacacacgtggtatacacacatccatgcaggcaaacacacatacacatagaaatggaagtaaatgatgcaggtgtgatggctggcacctgagaactggaggaggtctccttggttacctagtgagtttgaagccaacctaggctatataaagctctccagaagaaaaattaactattaaaaaacaaaaacaaaaacaaaaacaaaaacaaccctcagggctggagagatggctcagtcagtaagacccttactctgcagtcctcatgaaggtgaaggagaaaaccattgccactatggtcctctaactttttttttaagatttattatttattatatgtaagtacactgaagctgtcttcagacacaccagaagagggagtcagatcttgttacagatggttgtgagccaccatgtggttgctgggatttgaactcaggaccttcggaagagcagtcgggtgctcttacctgctgagccatctcaccagccccctctaactttcatacatgtgctgtcacacatgcatcccacgcccaccgcggcagagaagcatgggaacaggaacatctgagagctcacgtgttaaactgcaagcaccaagcagagagaacaggctcagagtctctctctctcacacacacacacacacagagaaaatctctctctctctctctctctctctctcacacacacacacacacacacacacacacgcacacttacacacagagagacacacagaatttctcatacacacacgtatacacacacagaatcactctcacacacacatacacagagccctccatctctcacacacactcatacacagatacacaaagaatccctcttacacacacagagaatctctctctctgtctctctctctgtctctctctctgtctctctctctctctctctcacacacacacagtctttaagatctcaaaatctgcaagaccacacctcctaatcctacccaaaaagagaccctaactggagaccaagtattcaaatacctgagaatgggggctgggggatttcattcaaaacatgagaagcaccatgcattttttagcttttgtgtaagacttccagtttctaaaagttcctctgacggagcctggcaaacccacgggacttttctttgctgaaggatctccattttggttctggagtggttgcctattgtcttgcaggtgtttgaacgtcatcttgtggttgggattctgggctgtgcagctgaacgtctgtctgtcccggatctaccttgctgctcactttccccaccaggtcgtggctggagtcttgtcaggtatcagcagctccgattccttccccacccacttcctatttcttctggatctttcttgtttagcctttcagtagtttcagcatcaaaggccagaggttgggaggctaagggcatggttttgtctgtaatgagaacctggtttgtagatttacctgcctgtattccagggtaggcagaggtgggaggatccccagagttcaggaacaagccagcctaggctaattggtgagccccaggtctcagtgacagactatacacacacacacacacacacacacacacacacacacacacacacacacacacacacggtggatgctccaaagaacaatacctgaggctgacctctggctctgcatgcacatacatgcacacacacaatttgaaattattagtttccacagcatgcccagaactatacagctaaagtgtcccaggaaagttgcattgcatcgagctaaactcactcaactgatgcatacaaaggggagctagtgctaaagtctccaagcttctgacaatctttgtctccgatcttcaatttgaactggctctcaagcttcttttcagagaatctcccaaatgtacagtgtcttggggagagctcgttattgaaatacattcatttgcagacctaacatgtctgcagccgatgttttctcagagaggtttgagcgtttgcaatttctgagatccagttaggaagagaaagaaaagatttaggttaagcattaaaaccaaggcaaccaaacaaacaaaaacattgtctagatttggagctgggggagggagcagaacagatcagttgcaagcctgaggatcagagttcggtccctggaatccacaaggcagaaaaaaaccccaccccaaccaactatcctctgactcccacatgtgtgctatggcacacatgcccacacacatacacacaaagacagaataaacaaaatgtgtaaataaataaataaaaatgcctggtgagtgagcatacccttggagaggctatgtcacacaacaaagagtaacgtcgtcagctgtgtgacaacttactagcttctgtggaaggaactgagagcaataggtagcattcaaggacaacagtgatgtgatctgatggctcagtgggctacctcatttgttaccaagcctgacaacctgagtttgagcctgggccccacatggtagaaagaaccaactcttgtcacttgccttttgacctccacacagataccgtgtcacacgtgtgcctgcccacaaagagacatacctacatacacatacccaataaatatatagatgtgaggacattttaagtaaataaataggataagtccattcaattaggttgtctgggggactactctctgcacccccatcttgagaatatgcaagatgaaacaggatccaacacacacaacaggaaacttagacttggtgggaagatgtatgtgatacaattcttccatccgaaagacactatgctgggaccttccactttggtgtgtgatttagattcatctgtggtatgatggcatgtcacccatagccgaccctactcctcgtgcctctttctcccacacaggcattgctgtggctgaaactttcagccacatccggggcatctacaatgccagcctccggaagtattgtctcatcaccatcttcttgtttggtttcgcgcttggattctacctgctactaaaagggctaggggtggacctcctgtggactttggagaaagctaagagatggtgtgagcggccagaatgggtccaccttgacactacaccctttgccagcctcttcaaaaacctgggaaccctcttggggttggggctggccctcaactccagcatgtaccggaagagctgcaagggagaactcagcaagttgctcccattccgcttcgcctgcattgtggcttccttggtcctcctgcatctctttgactctctgaagcccccatcccaggttgagttgatcttctacatcttgtctttctgcaagagcgcaacagttccctttgcatctgtcagtcttatcccatactgcctagcccggatcctgggacagacacacaagaagtctttgtaaggcatgcagagtctttggtatttaaagtcaaccgccatgcaaaggactaggaacaactaaagcctctgaaacccattgtgaggccagaggtgttgacatcggccctggtagccctgtctttctttgctatcttaaccaaaaggtgaatttttacaaagcttacagggctgtttgaggaaagtgtgaatgctggaaactgagtcattctggatggttccctgaagattcgcttaccagcctcctgtcggatacagaagagcaagcccaggctagagatcccaactgagaatgctcttgcggtgcagaatcttccggctgggaaaaggaaaagagcaccatgcatttgccaggaagagaaagaaggatcaggaggagggagagtgttttatgtatcgagcaaaccagatgcaatctatgtctaaccggcttcagttgtgtctgtgtttttagatacgacacactcaataataataatagaccaactagtgtaatgagtagccagttaaaggcgattaattctgcttccagatagtctccactgtacataaaaatcacactgtgtgcttgcattcctgtatggtagtggtgactgtctctcacaccaccttctctatcacgtcacagttttctcctcctcagcctatgtctgcattccccagaattctccacttgttccctggccctgctgctggaccctgctgtgtctggtaggcaactgtttgttggtgcttttgtagggttaagttaaactctgagatcttgggcaaaatggcaaggagacccaggattcttctctccaaaggtcactccgatgttatttttgattcctggggcagaaatatgactcctttccctagcccaagccatccaagagctctcattcttagaagaaaaggcagccccttggtgactgtcctcctgcctcggctgatttgcagagtacttcttcaaaaagaaaaaaatggtaaagctatttattaaaaattctttgttttttgctacaaatgatgcatatattttcacccacaccaagcactttgtttctaatatctttgataagaaaactacatgtgcagtattttattaaagcaacattttatttaa

SEQ ID NO: 15 - RNA, 2274 nt, Mus musculus
  source 1..2274: mol_type = mRNA
agcagagggatcaaggccaaccgggctggactcactgcacgggctctgctggcagcttcctgaggtaccaagggaggaaggatggaggaaggaatgaacattctccatgactttgggatccagtcgactcgctatctccaagtgaattaccaagactcccaggactggttcatccttgtgtctgtgattgctgacctgaggaacgccttctatgtcctctttcccatctggttccatcttaaagagactgtgggcatcaatctcctctgggtggcagtggtcggagactggttcaacctcgtcttcaagtggattctgtttggacaacgcccgtattggtgggtcctggacaccgactactacagcaacagctccgtgcctataataaagcagttccctgtcacctgtgagaccggaccaggaagtccctctggccatgccatgggcgcagcaggtgtatactatgttatggtcacttctactcttgctatctttcgaggaaagaaaaagccaacgtatggattccggtgtttgaacgtcatcttgtggttgggattctgggctgtgcagctgaacgtctgtctgtcccggatctaccttgctgctcactttccccaccaggtcgtggctggagtcttgtcaggcattgctgtggctgaaactttcagccacatccggggcatctacaatgccagcctccggaagtattgtctcatcaccatcttcttgtttggtttcgcgcttggattctacctgctactaaaagggctaggggtggacctcctgtggactttggagaaagctaagagatggtgtgagcggccagaatgggtccaccttgacactacaccctttgccagcctcttcaaaaacctgggaaccctcttggggttggggctggccctcaactccagcatgtaccggaagagctgcaagggagaactcagcaagttgctcccattccgcttcgcctgcattgtggcttccttggtcctcctgcatctctttgactctctgaagcccccatcccaggttgagttgatcttctacatcttgtctttctgcaagagcgcaacagttccctttgcatctgtcagtcttatcccatactgcctagcccggatcctgggacagacacacaagaagtctttgtaaggcatgcagagtctttggtatttaaagtcaaccgccatgcaaaggactaggaacaactaaagcctctgaaacccattgtgaggccagaggtgttgacatcggccctggtagccctgtctttctttgctatcttaaccaaaaggtgaatttttacaaagcttacagggctgtttgaggaaagtgtgaatgctggaaactgagtcattctggatggttccctgaagattcgcttaccagcctcctgtcggatacagaagagcaagcccaggctagagatcccaactgagaatgctcttgcggtgcagaatcttccggctgggaaaaggaaaagagcaccatgcatttgccaggaagagaaagaaggatcaggaggagggagagtgttttatgtatcgagcaaaccagatgcaatctatgtctaaccggcttcagttgtgtctgtgtttttagatacgacacactcaataataataatagaccaactagtgtaatgagtagccagttaaaggcgattaattctgcttccagatagtctccactgtacataaaaatcacactgtgtgcttgcattcctgtatggtagtggtgactgtctctcacaccaccttctctatcacgtcacagttttctcctcctcagcctatgtctgcattccccagaattctccacttgttccctggccctgctgctggaccctgctgtgtctggtaggcaactgtttgttggtgcttttgtagggttaagttaaactctgagatcttgggcaaaatggcaaggagacccaggattcttctctccaaaggtcactccgatgttatttttgattcctggggcagaaatatgactcctttccctagcccaagccatccaagagctctcattcttagaagaaaaggcagccccttggtgactgtcctcctgcctcggctgatttgcagagtacttcttcaaaaagaaaaaaatggtaaagctatttattaaaaattctttgttttttgctacaaatgatgcatatattttcacccacaccaagcactttgtttctaatatctttgataagaaaactacatgtgcagtattttattaaagcaacattttatttaaaaaaaaaaaaaaaaa

SEQ ID NO: 16 - protein, 357 aa, Mus musculus
MEEGMNILHDFGIQSTRYLQVNYQDSQDWFILVSVIADLRNAFYVLFPIWFHLKETVGINLLWVAVVGDWFNLVFKWILFGQRPYWWVLDTDYYSNSSVPIIKQFPVTCETGPGSPSGHAMGAAGVYYVMVTSTLAIFRGKKKPTYGFRCLNVILWLGFWAVQLNVCLSRIYLAAHFPHQVVAGVLSGIAVAETFSHIRGIYNASLRKYCLITIFLFGFALGFYLLLKGLGVDLLWTLEKAKRWCERPEWVHLDTTPFASLFKNLGTLLGLGLALNSSMYRKSCKGELSKLLPFRFACIVASLVLLHLFDSLKPPSQVELIFYILSFCKSATVPFASVSLIPYCLARILGQTHKKSL

SEQ ID NO: 17 - DNA, 13637 bp, Homo sapiens
  source 1..13637: mol_type = genomic DNA
atagcagagcaatcaccaccaagcctggaataactgcaagggctctgctgacatcttcctgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggtaagaaccatatagagaggagatcagcaagaaaagaggctggcattcgctctcgcaatgtctgtccatcagaagttgctttccccaggctattcaggaagccacgggctactcatgcttccaacccctctctctgactttggatcatctacataaagggggaagacagaaaaaatcctaccagtgagttgaaaatacaggaaagcctatttcatatgggttaaagggtaggacagttgaatttcgtgaaaagtctgagttatataggctttgagcaaagagttttattagtatgaagcagaagaggtaacataaagaaagatgtatggggccaggcatggtggctcacacctgtaatcccagcactttgggaggccgaggtgggcgaatcactcctgggtgaactcaggagttcaagaccagcctgggcaacatggcgaaactccatctctacaaaaacattacgaaaattagctgggcgtgttggtgctgtagtcccagctactcaggaggctgaggtgagaggcggaggaggttgcagtgagtcaagatcatgccactgcactccagcctgggcaacagagtaagaccctgtctcaaaaaaaaaaaaaagatagatgatgtatgctgtatgaaaaaaggaaacacacagatgattcaacagcctgttttgtggggtaatgaaaagtcaccctgggaactgggctccagccctcgttctgccacccaccaactacatgtccttggcaagtcatatcaattatctgagtttctgttttataatctacaaataggttatctctggcagcttaataataatcagggttaacatttattaaacagtgtgtgccagtccatgtgctatgtgcttttctgtgaggtagttactgctatttacagaaacagtagatgcagagaccaaggtgctgagttaaatgattaggccaacaaggttagtacatgccgagccaggatggaagcccaggtaggcaggctggcttccgcggcaatgctcttatgaactatgttacgtccagtgctgataaactgactctctggggagcaggggaaagccctgagtttagcatttgccaatttctatcacgtaaacattcccattctggccactttctttctttctttcttttgtttgtttgtttgagatggagtctcgcactgttgcctggctggagtgcaatggtgcaatctcagctcactgcaacctctgcctctccggttcaagtgattctcctgcctcagcctcccaagtagctgggattacaggtgcccgccaccatgcccagctaattttttttgtatttttagtagagacatggtttcactatgttgactaggctggtctcgaactcctgacctcatgatctgcctgccttggcctccctaagtgctaggattacaggcgtgagccactacacccagccgcatgattctaaaaaataaaaagatgaagtgttattccaaacatctgatctccattgaagaaccatgcaatctctctgggttgatagaggccagagttagtggctctccctgatttcggtgagaaatcactattccaccatcacgggataaaaggcatcctgactggcggttgacacctatttccacagtgaaagatatatctagtacttttaaaggggaagtggtttgtctgagatactctgtttcaaagtagagaggatacagaacaagcatctgaagctatatacatccttacagagagcaattctgatggaaatgcaggccatgtttccctggggggggctcgtcctaggggctggagtgcattctctgatgtcagaggaaatgcaagattccctgaggcctgagggaacccatggtatatgcaagtccaagtttcaaactgtagttccatatgcattcttccaggacaaatacttcttgaggttaaaaaaaaaaagtcacatagctgccattttatggatttcaggatttttttttttttttttttgagatggagtcttgctctgtcacccagcctgtagtgcagtggcataatctcggctcacggcaacctccgcctcccaggttcaagcgattctcttgccttagcctcccgagtagctgggattacagtcacgcaccaccacatctggctaattctttatattttttggtagaaacggtgtttcaccatgttggccaggctggtctcaaactcctgacctcatgtgatctgcctgccttggcctcccaaagtgctgagattacaggtgtgagccaccgcgcctgcctggagttcagaatcttgggcttcattatttgtgtttaaatagatcatacagtcaggcacggtggctcatgcctgtaatcccagcactttgggaggctgaggtgggaggattgcctgagttcaggagatggagaccagcctgggcaacatggtgaaaccccgtctctactaaaaatacaaaaactagctggatgtggtggcacacacctgtagtcccagctattcaggaggctgaggtgggaggatcccaggaggtagaggtcacaatgagccgagattgcgccactgcactccaggctgggttactgagccagatcctgtctcaaaaaaaaaaaagataatacattcaaacagttcaaaatgcaaaagttacatacataaggaagtgtcatgaaatatctccctctcacacttctccccagccacccagttctcccttctagaggcaacatgtgaaatccttctcaggctacactcttcttgaaggtgtaggctttgggcaaaagcattcattcagtaaccccagaaacttgttctgtttttccataggattctctttggacagcgtccatactggtgggttttggatactgactactacagcaacacttccgtgcccctgataaagcagttccctgtaacctgtgagactggaccaggtaagcgtcccagcccctgcagacagaagctgagtggacctcgtttacctgttatggatgaaactgaccttgaggggacatgaggagagccattcctttgtacttttgtcatgctcttcaattggcacaaattaattcacttctgcaatactttcctgaatagcacagtagtattggaaatctgcctattacagaacctggatggagtccagagaggcacgggcatccatgggcaaagggctcgtgagagtcaccgccctgcagcgctgtgtcctgagaaaggagggggcagaagcctgagcttctgggggtccttcccaatggcctggcccactggatgtgccctcctgagctgaccgtccaatcccttgccctctctgtgcctacgttttattagttacagccagatggttactgtcaaatcaaatgatagatttcattttcagtatgtaataggaagcccctccctcaccctaaagtctcagctgccctctaagactagtactctctaaggtactagtatcccttcctcagagaccctttccctgaccccaaaactagggaaggtcccttagttatttgctctcacagaccacgcatttacctcagagcatattcactcattcagctgttacttaccaagcacctactgggagctatacactgttctatgtgctagggatacctctgtcagtgaacaacacagacacaaagatccctgcccttgtggagctgaaatctgaatagaggaggtgaaatatacaaaaattataataaataagtaaactaggccagttgtggttgctcatgcctgtaatcccagcactttgggaagccaaggtaggtagatcacctgaggtcaggagttcaaaaccagcctggccaacattgcaaaatcctgtctttactaaaaatggaaaaattggtcaggcgtgatggcacacgcctgtagtctcagctacctgggaggctgaggcaggagaatcgcttgaacctgggaggcagaggttgcagtgaaccgagatcggaccactgcactccagcctgaatgacagaacgagactctgtctcaaaaaaaaagtaaactattaatatgtaggataggccaggcacggtggctcaccctgtaatcccagcactttgggaggctgaggcgggtggatcacctgaggtgaggagttcaagaccagcctggccaacatggcaaaaccctgtctctactaaaaatacaaaaattagctgggtgtcctggtgcatgcctgtaatctgagctactcaggaggctaaggcaggagaatcgcttgaacctgggaggtggtgagccaagattgcgccattgcactccagcctgggcgacaaaatgagacaccatctgaaaaaaaaaaaaaaatatatatatatatacacacacacacacacacacacacacacacacatataatactagaaaatgattgtttataggcaaaaaaaaaaaaaaagaagaagaagaagaaaaggaaaggagaaggaaagaaggaccaaacatcttttgtagaaatatgtttgctttcatcataacagcttgttatcaaggatgaatttctccctgaaattaatggaggcacagactggaaagtttaaagtggctttaagaggttattttatttagtcctctgtcttaatagaagcaaattattatctctgctccttaggtagagtagctaaggctcagaaagtaggccgggcgcggtggctcacgcctgtaatcctagcactttgggaggccaacgcaggtggatcacctgaggtcaggagtttgagaccagcctggccaacatggtgaaacctcgtcactaataaaaaaatacaaaaacttagccaggcatggtggcgggcgcctgtaatcccagctacccaggaggctgcggcaggagaatcacttcaacccgggaggcagaggttgcagtgagctgaaatcacaccactgcactccagccttggtgacagagaaagattctgtcaggaaaaaaaaaaaaaagtttaaatgaattacccaaggtatataattgttagtgttagaaggaagaagaagggagggaggaaggaagggagaaagaaagggaaggaggaagggagggagggaagaaagcctttatttatctatggggttccctggaaagcaggctgaaatggagattcacgtgcaggagtttagatactctggggaactatacttgtagaagggaaggaacaggaacagggcagaaggagaggtccggttgtgattctgcctcatccaaccccacagcgagctctgaagctggggatggctcctcagagttggtccaagttgggacaagggaatcagaccctggggagagcgtaaccttgatcaaggcgactctctttagcccagggcaatgccaggagaaggctgagagcagaaagccatctaccatcacactctcaacagctacgaaataagtcctgcagttcaggagggaggtctgggcggcacatctcaggaccctctatctctcagggtagaggaattaagaatgggatgggaaccagacgggccatggtggctcacacctataatcccaacactttgggaggccaagggtaggaggattgcttgagcccaagagttcaaaaccagcctgggcaaaaacaatcaaacaaacaaacaaaacacatttaaaaaatttgctgtgtgtggtggtgtgcacctgtggtcccagctactcagggggctgaggtgggaggattgcttgagtccaggaggtcgaggctgcagtgagctatgatcatggcactgcattgcagcctaggagacaaagcaagacactgtctctaaaaaaacaaaaaacaaacaaataaaaaaacggaaccggttgcaagcagggttaaatagcgtggtcagagtaggactcactgagaatatgagatctgagtcaagtcttcaaggatgtgaggaagtaagtttctggcagaagagctgtgaagggctgtctggccagagaagattgcaatgcaaaagccctgaggtgggaacgtgtttggtgtgtttaaaggaaagcaatgaggccagtgtagccagaacagagtgtgcaaggagagaaggaacagaagatgtggagggcagatcagtttgtaattgtacgcccagtatgctgattctttgtgtaatctccagactgtattaaactgcaagagcagggcccctctctggctttgctcatcattgtattcccagagccttgcacaatgcttggtgcataggagatggaaatttgttaaataaatgaattatggataacgaatggatggtaagatgggtggatggatggggggtgaacggatggatggggggtgaatggatggatgaatgggtagatgggtggatagggggatggctgggtggctgggtagatgatgcactgtctcccagatgaggaccttttcacctttactccattctctttcctgccctttagggagcccctctggccatgccatgggcacagcaggtgtatactacgtgatggtcacatctactctttccatctttcagggaaagataaagccgacctacagatttcggtaagaactcaccactggggtgtaggtggtggagggcaggaggcagctctctctgtagctgacacaccacgtattcttcctcacatccccctagcccgctcccacacctgggcagccgctgattaagagttgtggcactttggatagggataaacctcagagtcagggaatgtttgggctgaaagggatccagtagtgcaatccgttgttttacagataaggaaacaaagcccaacaccatgaagggacttataaaaataaggtagtgaagtagcagcagggcttaaataaaaacccatgtctgtaccaaccacagagtcacccatccaggttaaaataaccagagaaacagaagatattcctactacagagaattccgggtgtgcagccacagtgcaaatcctttttatttttatttttgagatgcagtctcgctctgtcatccaggctgaagtgcagtggcacgatcatgtctcgctgcaacctctgcctcccaggctcaagcgatcctcccacctcagccatctgagtagctgggaccacaggccacacaccacacccagctaatttctcgtatctttttgtagagacagagttctgctatgttgcccaggctcaggctggtcttgatctcaagcaattggcttgcctcagcctcctaaaatattgggattacaggcatgagccaccgcgccagccatgcaaatccttaattatcaaacagataaaatagggaagttaaaattcatatacacaagggttaaccacttgccacaggcattttttttttttttttgagacggaatctcgctctgttgcccaggctggagtgcagtggcgccatctcgcctcactgcaacctccgcttcctgggttcaagctattcttctgcctcagcctaccgagtagctgggactacaggcacgtgccaccacacctggctaatttttttatttttagtagagatggggtttcaccatattggccaggctggtcttgaactcctgacctagtgatccatccgcctcagcctcccaaagtgctgggattgcaggcatgagccaccgcgcctggccttttttttttttttttgagacggagttttgctcttgttgcccaggctagagtgcagtggcgcagtctcggctcactgtaacctccacctcctgagttcaagcaattctcctgcctcagcctctcaaatagctgggattacaggcgtgagccaccccacctggctaattttgtaattttttttttagtagagatggggtttcacctgttgatcaggctggtctcaaactcctgacctcaagtgatccacccacctcggcctcccaaagtgctgggattacaagcataagccaccgtgcctggtcaattttgatcttttttaaagagacaggggtcttgctatgttgcccagactagtcttgaactcctggcctcaagtgatcctctcacctcggcctcccaaagtattgggattacaggtctgagccgctgcacccagcccccaacaggcatctttggacttttgagtactggctttaatttacaaaaattccactgagagcacctaagtttgccaggctccaacatttctgcaggggctgttttctttgctgaaggatctgcacctgtgttctgttatggttgcctcttctgttgcaggtgcttgaatgtcattttgtggttgggattctgggctgtgcagctgaatgtctgtctgtcacgaatctaccttgctgctcattttcctcatcaagttgttgctggagtcctgtcaggtatgggctgatctgactcccttccttctcccccaaaccccattccgtttctctccctaatcaggacaaaatcccagcattccagccacatcctgtgtgtaatcagtactgttagcatttctgtgggttgaaagtcaagaatgagcaacttgaaatgattaatttctataagagtgcccagatctatagaatgaattgtgtagaagttaccatacatcaaattaacgcaccaaattgaattagcttgaaatctcagagctttttacaatctttatttcttactggtcttcaacaggccctaatttacttttcagggaatctgccaaatttaacaaattaacacgatgtcctaggaaagctgttcatttaaatacattcatttgcaaacctaatagataactgcagttgatctcttttataggttcagagttttgaatatgtttttttttgttttttttttttgagatggagtctcgctctgtgacccaggctagagtgcagtggtgcgatctcggctcactgcaagctccacctcctgggttcacgccattctcctgcctcagcctctccgagtagctgggactacaggcgcccgccaccatgcccggctaattttttgtatttttagcagagacggggtttcaccgtggtcttgatctcctgacctcgtgatccgcccgcctcggcctcccaaagcgctgggattacaagggtgagccaccgcaccctgcctgaatatgtgttttcttagatccaattaacaagggtaagacaagatttaagttaagcataagaaagattttgtgggaggcactggaatataagaccttaacaaaactgtggaatttctcccctggagatttgtaagaacggaacatagcagcattcaaagaagaatgttgagaacaagggagataatggtttcatggtaatcacaaaagtaacacagcatttagtactgggttccatgtttgaggaagaacctggaagccatatcacatgaaaaacctgggaatgtttaggttagagagaataactgtgttcaaatgtgtgacagagggactagattcatcacttactaactcctgcagaaagaactgagaaaaatagacagtattagagggggaccagtttcacacagacaaggaagaactattcagcaatcaattccgttcaaagataaaatggactgttatagtgggggtgagctccctacctctgagggtatttcaagtagagataggaggacctcctggtaggaaatttgcatacggtgggagattgtacgtgatatggcacctccatctgaaagagtctatattgagggcaggctggagtcacacatgggaataagccaggcgaccctcccatctgccatctgtgatttaattccacagtcgcagaacggatggcatgtcacccactcctccaaacccacctctagcaaaggtcccaaatccttcctatctctcacagtcatgctttcttccactcaggcattgctgttacagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaagagatgtggagtcttcggtgtttaaagtcaacaaccatgccagggattgaggaggactactatttgaagcaatgggcactggtatttggagcaagtgacatgccatccattctgccgtcgtggaattaaatcacggatggcagattggagggtcgcctggcttattcccatgtgtgactccagcctgccctcagcacagactctttcagatggaggtgccatatcacgtacaccatatgcaagtttcccgccaggaggtcctcctctctctacttgaatactctcacaagtagggagctcactcccactggaacagcccattttatctttgaatggtcttctgccagcccattttgaggccagaggtgctgtcagctcaggtggtcctcttttacaatcctaatcatattgggtaatgtttttgaaaagctaatgaagctattgagaaagacctgttgctagaagttgggttgttctggattttcccctgaagacttacttattcttccgtcacatatacaaaagcaagacttccaggtagggccagctcacaagcccaggctggagatcctaactgagaattttctacctgtgttcattcttaccgagaaaaggagaaaggagctctgaatctgataggaaaagaaggctgcctaaggaggagtttttagtatgtggcgtatcatgcaagtgctatgccaagccatgtctaaatggctttaattatatagtaatgcactctcagtaatgggggaccagcttaagtataattaatagatggttagtggggtaattctgcttctagtattttttttactgtgcatacatgttcatcgtatttccttggatttctgaatggctgcagtgacccagatattgcactaggtcaaaacattcaggtatagctgacatctcctctatcacattacatcatcctccttataagcccagctctgctttttccagattcttccactggctccacatccaccccactggatcttcagaaggctagagggcgactctggtggtgcttttgtatgtttcaattaggctctgaaatcttgggcaaaatgacaaggggagggccaggattcctctctcaggtcactccagtgttacttttaattcctagagggtaaatatgactcctttctctatcccaagccaaccaagagcacattcttaaaggaaaagtcaacatcttctctctttttttttttttttgagacagggtctcactatgttgcccaggctgctcttgaattcctgggctcaagcagtcctcccaccctaccacagcgtcccgcgtagctgggactacaggtgcaagccactatgtccagctagccaactcctccttgcctgcttttctttttttttctttttttgagacggcgcacctatcacccaggctggagtggagtggcacgatcttggctcactgcaacctcttcctcctggttcaagcgattctcatgtctcagcctcctcagtagctaggactaccggcgtgcaccaccatgccaggctaatttttatatttttagaattttagaagagatgggatttcatcatgttggccaggctggtctcgaactcctgacctcaagtgatccacctgccttggcctcccaaggtgctaggattacaggcatgagccaccgcaccgggccctccttgcctgtttttcaatctcatctgatatgcagagtatttctgccccacccacctaccccccaaaaaaagctgaagcctatttatttgaaagtccttgtttttgctactaattatatagtataccatacattatcattcaaaacaaccatcctgctcataacatctttgaaaagaaaaatatatatgtgcagtattttattaaagcaacattttatttaagaataaagtcttgttaattactatattttagatgcaatgtgatctgaagtttctaattctggcccaactaaatttctagctctgtttccctaaacaaataatttggtttctctgtgcctgcattttccctttggagaagaaaagtgctctctcttgagttgaccgagagtcccattagggatagggagacttaaatgcatccacaggggcacaggcagagttgagcacataaacggaggcccaaaatcagcatagaaccagaaagattcagagttggccaagaatgaacattggctaccagaccacaagtcagcatgagttgctctatggcatcaaattgcaacttgagagtagatgggcagggtcactatcaaattaagcaatcagggcacacaagttgcagtaacacaacaagactaggccagctctggaatccagtaactcagtgtcagcaaggttttgggttatagttcaagaaagtctaaacagagccagtcacagcaccaaggaatgctcaagggagctattgcaggtttctctgctaagagatttatttcatcctgggtgcagggttcgacctccaaaggcctcaaatcatcaccgtatcaatggatttcctgagggtaagctccgctatttcacacctgaactccggagtctgtatattcagggaagattgcattctcctactggatttgggctctcagagggcgttgtgggaaccaggcccctcacagaatcaaatggtcccaaccagggagaaagaaaatagtcttttttttttttttaatagagatgggggtctcactatgctgcccaggctggtcttgaactcctgggttcaagtgatcctcctgcctcagcctcccaaagtgctgggattacagtgtgagccactgcgcttggccagaaatggttttgatctgtctgaactgaaccctactgcttaggcatagccccatccttgataatctatttgctcccaaggaccaagtccaagatccttacaagaaaggtctgccagaaagtaaatactgcccccactccctgaagtttatgaggttgataagaaaacataacagataaagtttattgagtgctaacttta

SEQ ID NO: 18 - RNA, 4164 nt, Homo sapiens
  source 1..4164: mol_type = mRNA
agcagagcaatcaccaccaagcctggaataactgcaagggctctgctgacatcttcctgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttgcagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaagagatgtggagtcttcggtgtttaaagtcaacaaccatgccagggattgaggaggactactatttgaagcaatgggcactggtatttggagcaagtgacatgccatccattctgccgtcgtggaattaaatcacggatggcagattggagggtcgcctggcttattcccatgtgtgactccagcctgccctcagcacagactctttcagatggaggtgccatatcacgtacaccatatgcaagtttcccgccaggaggtcctcctctctctacttgaatactctcacaagtagggagctcactcccactggaacagcccattttatctttgaatggtcttctgccagcccattttgaggccagaggtgctgtcagctcaggtggtcctcttttacaatcctaatcatattgggtaatgtttttgaaaagctaatgaagctattgagaaagacctgttgctagaagttgggttgttctggattttcccctgaagacttacttattcttccgtcacatatacaaaagcaagacttccaggtagggccagctcacaagcccaggctggagatcctaactgagaattttctacctgtgttcattcttaccgagaaaaggagaaaggagctctgaatctgataggaaaagaaggctgcctaaggaggagtttttagtatgtggcgtatcatgcaagtgctatgccaagccatgtctaaatggctttaattatatagtaatgcactctcagtaatgggggaccagcttaagtataattaatagatggttagtggggtaattctgcttctagtattttttttactgtgcatacatgttcatcgtatttccttggatttctgaatggctgcagtgacccagatattgcactaggtcaaaacattcaggtatagctgacatctcctctatcacattacatcatcctccttataagcccagctctgctttttccagattcttccactggctccacatccaccccactggatcttcagaaggctagagggcgactctggtggtgcttttgtatgtttcaattaggctctgaaatcttgggcaaaatgacaaggggagggccaggattcctctctcaggtcactccagtgttacttttaattcctagagggtaaatatgactcctttctctatcccaagccaaccaagagcacattcttaaaggaaaagtcaacatcttctctctttttttttttttttgagacagggtctcactatgttgcccaggctgctcttgaattcctgggctcaagcagtcctcccaccctaccacagcgtcccgcgtagctgggactacaggtgcaagccactatgtccagctagccaactcctccttgcctgcttttctttttttttctttttttgagacggcgcacctatcacccaggctggagtggagtggcacgatcttggctcactgcaacctcttcctcctggttcaagcgattctcatgtctcagcctcctcagtagctaggactaccggcgtgcaccaccatgccaggctaatttttatatttttagaattttagaagagatgggatttcatcatgttggccaggctggtctcgaactcctgacctcaagtgatccacctgccttggcctcccaaggtgctaggattacaggcatgagccaccgcaccgggccctccttgcctgtttttcaatctcatctgatatgcagagtatttctgccccacccacctaccccccaaaaaaagctgaagcctatttatttgaaagtccttgtttttgctactaattatatagtataccatacattatcattcaaaacaaccatcctgctcataacatctttgaaaagaaaaatatatatgtgcagtattttattaaagcaacattttatttaagaataaagtcttgttaattactatattttagatgcaatgtgatctgaagtttctaattctggcccaactaaatttctagctctgtttccctaaacaaataatttggtttctctgtgcctgcattttccctttggagaagaaaagtgctctctcttgagttgaccgagagtcccattagggatagggagacttaaatgcatccacaggggcacaggcagagttgagcacataaacggaggcccaaaatcagcatagaaccagaaagattcagagttggccaagaatgaacattggctaccagaccacaagtcagcatgagttgctctatggcatcaaattgcaacttgagagtagatgggcagggtcactatcaaattaagcaatcagggcacacaagttgcagtaacacaacaagactaggccagctctggaatccagtaactcagtgtcagcaaggttttgggttatagttcaagaaagtctaaacagagccagtcacagcaccaaggaatgctcaagggagctattgcaggtttctctgctaagagatttatttcatcctgggtgcagggttcgacctccaaaggcctcaaatcatcaccgtatcaatggatttcctgagggtaagctccgctatttcacacctgaactccggagtctgtatattcagggaagattgcattctcctactggatttgggctctcagagggcgttgtgggaaccaggcccctcacagaatcaaatggtcccaaccagggagaaagaaaatagtcttttttttttttttaatagagatgggggtctcactatgctgcccaggctggtcttgaactcctgggttcaagtgatcctcctgcctcagcctcccaaagtgctgggattacagtgtgagccactgcgcttggccagaaatggttttgatctgtctgaactgaaccctactgcttaggcatagccccatccttgataatctatttgctcccaaggaccaagtccaagatccttacaagaaaggtctgccagaaagtaaatactgcccccactccctgaagtttatgaggttgataagaaaacataacagataaagtttattgagtgctaactttatgcca

SEQ ID NO: 19 - protein, 357 aa, Homo sapiens
MEEGMNVLHDFGIQSTHYLQVNYQDSQDWFILVSVIADLRNAFYVLFPIWFHLQEAVGIKLLWVAVIGDWLNLVFKWILFGQRPYWWVLDTDYYSNTSVPLIKQFPVTCETGPGSPSGHAMGTAGVYYVMVTSTLSIFQGKIKPTYRFRCLNVILWLGFWAVQLNVCLSRIYLAAHFPHQVVAGVLSGIAVAETFSHIHSIYNASLKKYFLITFFLFSFAIGFYLLLKGLGVDLLWTLEKAQRWCEQPEWVHIDTTPFASLLKNLGTLFGLGLALNSSMYRESCKGKLSKWLPFRLSSIVASLVLLHVFDSLKPPSQVELVFYVLSFCKSAVVPLASVSVIPYCLAQVLGQPHKKSL

SEQ ID NO: 20 - DNA, 1071 bp, synthetic construct
  source 1..1071: mol_type = other DNA
caacgacttcttgtgcggctggcccaggacctgggcgaggcagtaggggatgacactgacggatgccaggggcactaccgcactcttgcagaaggacaagacgtagaagaccagctcgacttgggatgggggtttcaaggagtcaaagacgtgcaggaggacgagggaggctacaatagagctgaggcggaatgggagccacttgctgagtttccccttgcagctctccctgtacatgctggagttgagagccagccccaggccaaagagcgtgcccaggttcttgaggaggctggcaaagggtgtggtgtcaatgtggacccattctggctgctcgcaccacctctgggctttctccagagtccacaggaggtctacacccagtcccttgagcagcagataaaatccgatggcgaagctgaacaggaagaaggtaatgagaaaatatttcttgaggctggcattatagatgctgtggatgtggctgaaagtttctgtaacagcaatgcctgacaggactccagcaacaacttgatgaggaaaatgagcagcaaggtagattcgtgacagacagacattcagctgcacagcccagaatcccaaccacaaaatgacattcaagcaccgaaatctgtaggtcggctttatctttccctgaaagatggaaagagtagatgtgaccatcacgtagtatacacctgctgtgcccatggcatggccagaggggctccctggtccagtctcacaggttacagggaactgctttatcaggggcacggaagtgttgctgtagtagtcagtatccaaaacccaccagtatggacgctgtccaaagagaatccacttaaagacgaggttgagccagtctccaatcacagctacccaaaggagtttaatgcccacagcttcctgaagatggaaccagatggggaagaggacgtagaaggcattcctgaggtctgcgatcacggacaccaagatgaaccagtcctgggagtcttggtaattcacctggaggtaatgtgttgactggatcccaaagtcatggagaacattcattccttcctccat

SEQ ID NO: 21 - protein, 354 aa, synthetic construct
QRLLVRLAQDLGEAVGDDTDGCQGHYRTLAEGQDVEDQLDLGWGFQGVKDVQEDEGGYNRAEAEWEPLAEFPLAALPVHAGVESQPQAKERAQVLEEAGKGCGVNVDPFWLLAPPLGFLQSPQEVYTQSLEQQIKSDGEAEQEEGNEKIFLEAGIIDAVDVAESFCNSNAQDSSNNLMRKMSSKVDSQTDIQLHSPESQPQNDIQAPKSVGRLYLSLKDGKSRCDHHVVYTCCAHGMARGAPWSSLTGYRELLYQGHGSVAVVVSIQNPPVWTLSKENPLKDEVEPVSNHSYPKEFNAHSFLKMEPDGEEDVEGIPEVCDHGHQDEPVLGVLVIHLEVMCLDPKVMENIHSFLH

SEQ ID NO: 22 - DNA, 1182 bp, synthetic construct
  source 1..1182: mol_type = other DNA
tagcagagcaatcaccaccaagcctggaataactgcaagggctctgctgacatcttcctgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttacagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaagagatgtggagtcttcggtgtttaaagtc

SEQ ID NO: 23 - DNA, 1074 bp, synthetic construct
  source 1..1074: mol_type = other DNA
atggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttgcagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaa

SEQ ID NO: 24 - protein, 357 aa, synthetic construct; identical to SEQ ID NO: 19

SEQ ID NO: 25 - DNA, 1125 bp, synthetic construct
  source 1..1125: mol_type = other DNA
tcgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttacagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaagagatgtggagtcttcggtgtttaaagtc

SEQ ID NO: 26 - DNA, 17496 bp, Mus musculus
  source 1..17496: mol_type = genomic DNA
acacggactccgccgcctacgcaggaggtcgtgtgacgaggtccgcgcggacgagccccgcccacagaccacgtgacccacgctgcccgctgagcctgggggtcttcggcctggagggtgattgcgcaggtgagctgggcgagctggtgaaggctgtctccggccccagagcctccggaggagcggcctcctccaggcaggcagggccgactgtcagcgagttcctgctttggagaatcgagcagagacacgcggcataggtgagacccccgacccaggcagccacggacggtgaccttagtaagacagcaggctccggtaaagtccgggattctagttcgtgcccgcagtaccaggcgggcaaagctctgtgtgtatgtgacgcagggggaaactgaggcacagagaaaccgagaaccttcactttgacctcccgtgtctgacaggttggatttggacacgcctgtgcctggtgacgcagcgatccagctgctttccagaaagcaagcttgaggtcaccaggtcagccgtggcctctgagttcagttccttcccatgtttttttctctaaattgtggctgagagaacgggcctcagctgcctgctcccagtttctgggactttgttctaatgcgtgtgtttctgagttgcgccctgcggcctccttggagggtcctggaaaatgcagattcccaccctgccccgcagaatttgagcctgcgagtgtccttagatgctgaatcatggtcagtgtcacttacgtctggaagggccaggtggagctgggccttactcggcagtaaagacaaaatgggtctctggtcttcgagccagatccacacactggatctttctgtggctcattatggtggctcattatggccgctgggcacctgaaagaccaaggcgtgattggagggtaggaaactgcagcctacgcaagcactccaactccctcaagatgctggagaatcactcaccatggtaacggatttcagtcatgcctacgtaaccgaaggcccacagaactgaacagagctggctgtggtctccaatgccagaaagcccagcgtgtaggagatggtggcagggacccaagccatccctggcttcatagcaagttaaggcaagcctgggctactgcagaccctgtctcaacatggcagccacgtgggttcagagaccatcccagttgatcaacacatttgagtgttgggagcctgggagccactcccaaccgttctccacccatttcttcattctggctgttcttgagttacagctttcaaaaccaaccaaccagtaaatataagtgaagtgcttttctgagttctgtgagatagtctagtgaattgccaaacttggtgcatggtgtaggggtcctcggaaccaccatggtctataggcagtccatcagagaggtatggcggcaacctggggcttactggcatctaggcgtggcagagccccagccgggtcatagttaacaaccccctcctggactggaaaactggggagccactagccggagaaagtcacaggaattgtgggtaaacactgccaaccagaagcaccctgactcaccaatggccacagttggtccccaagtgctaatcagtattcacagctgcctcttctgcagccgggccatctcctgagtgtgtctggtgaccctgagaaccgtcctctgtgctgagccccaccacatggagaagtcaccccagccttctacccaagccccatttccagaaggcccacactcctgcagctagtgaaccctgtggcccagtcctgggctcttcactcccaacttcctccccttggggtaggacttgggtgtggtggctcttccaccttcccactcacccttttctcttacacagaaggccctgcgataaagtcctggctccctcacatctgtctgtacctgtgttgtgggcccatgtctgcagagacaccctctgcctctgaggagtctcccattgtctcctgtacctggcttctgtcttcccttccgttgctctggggaagatgctctggctgcctccttccacctcctggcagtgtgtgtgggtgttgtgtgagaaaccccacccacactgatactggctcctcctctcctcaggccttcagaagtattcatgctgccccgaaccaacaggctttcaccatgaatatacggaagcccctctgttcgaactccgtggttggggcctgcacccttatctctctgactacagcggtcatcctgggtcatctcatgcttcgggagttaatgctgcttccccaagaccttcatgagtcctcttcaggactgtggaagacgtaccgacctcaccaccaggaaggttacaagccagggcctctgcacatccaggagcagactgaacagcccaaagaagcacccacacagtgtgatgtgccccccagcagccgctttgactgtgcccccgacaaaggcatctcacaggagcaatgcgaggcccgcggctgctgctatgtcccagcagggcaggtgctgaaggagccgcagatagggcagccctggtgtttcttccctcccagctacccaagctaccgtctagagaacctgagctctacagagtcggggtacacagccaccctgacccgtaccagcccgaccttcttcccaaaggatgtgctgaccttacagctggaggtgctgatggagacagacagccgcctccacttcaaggtgggtgtgggcatggggggcccctggatcgagctgggtcaggcagatgactgtcacgagggaggcctgtgcagagacaggctaggcagagcagagggacaggtgtgtgtggataggtacacatttcctaaggatatgtgcggtatgactgtgtgtggacacttcctgggacaccacgtgtgggcagagtggtgttagaggctctagaaggcaggaacaggcacaggtgggagacaggccagaagtatgtgtatacctcatgcacaggtaagttccacaagtataaaaaacttaacagctatttgtgaggccaggcgggggtagcacacgcctttaatcccagcacttaggaggcaaaggcaggattccaagaggtgagttccaggctgctccggtctacaaggtgagttctagggcggccaggactacacagagaaagcatgtctcgaaaaaccagagttcagtgttggagcatgtgcctgacatgtgcagggtgtgatccccagcagtgagaacacggaagcagaggaccatgcctgtaatcacagcatctggggcgctgttcgggaggatttgtggacctcccctgtaatcagagcattcaggaggctgtacgggaggactgtgagggaaatgtcagcctgggctccagagtgagaaaacctgcctcaaaaattaattaatcgattgattaaaaagataaataaggcaagagaggaaagaacagacagagatctacagttcaggtttagtggatgttctgagtttaagaaccccagggaggtccccttccctccagttatatagttgcagaaacagagactcacagttacacacagtgtgcaatcacccccagacctgagctgccgactatcctggtgccgcagagggactgcagtgagggaaggagccatcgggggagggggcctggctctggaagggctttgagcaccactgtcgcatacctgtctttttagctactttagtggattcttttacctacccttctctaccctcccaacttcatccccacccccacccaagccttagagaggagagaaagaaggggagagggggagtattgatctcgttagactattcctgctgattaaggggcatcaagttccctgcagcaagcccagtcttcgaagtcaggatatccccagtttctttttctcttctctttgctcacaaccacttgacaaccacagcaacaggaaccagcggcagcagagagaagggcggcccctccaggctctggagttttttataccatctcaagagtccccagaattccaaatgtaaactgtcttcagctggcagaatcacgcccctcccagaccacaagacaatcatggttagctgctgtggacagtctgaggcagccccatacccaacaccagggattaaaagaaaaaacgttcccgggctggtgagatggctcagtgggtaagagcgctgacttgtcctcccgaaggtcctaagttcagatcccagcagccacacggtggctcacaaccacccataatgagatctgatgccctctttgggtgcatctgaagacagctacagtgtacttatttattgataataataaatctttgagctggaacaaggagggactgagctagggggccctacctgagcaagcagggttgacgggagcaagtcttggtcctaaatttgataccttacaactacatgaaggctcataaccatctgtacagatacagtgtactcatatacataaaataaataattttttaaaaaagaagccgaagctgagcgtggtggcacatgcctttaaccccagcacttgggaggcagaggcaggtagatttctgagttccaggccagcctggtctatagagtgagttccaggacagccagggctgtacacagaaaccctgtctcggaaaaacaaaaaaagaaaaagaaaaacattcccatcacttaacttttctaaaagatttattatgtatacaacattctgcctgcacttacgtctgcaggccagaagagggcaccagatctcattatagatagtcatgagccaccaggtggttgctgggatttgaactcaggacctctggaagagcagccagtgctctcaacctctgagccatctctccagcccacgtaattgttttaagaagccaaaattctcaccatactgtctcccctctcctagatcaaagatcctgctagtaagcgctacgaagtgcccctggagaccccacgtgtgctgagccaggcaccatccccactttacagcgtggaattctcagaggaaccctttggagtgatcgttcgtaggaagcttggtggccgagtgttgtgagttggttacctgaggctttgactccaccctgagtggccccttagggtcagagcttctgcaatcagcaaacctgtctacctcattccaaaccctgcctttttctgtcttgttttctgttgctgtggtaaacacgaacagaagccacgtggggaagagaaagtggtttacctcacaggttctaggccatgctccatcactgccggggcaggaactgaagcagaggccatggaggagtgctgtttactgccttagtcctcatggctcacttagcttaagttcttatccagcccagaactaccagccctagggtgttaccaccctcagtgagctaggccccacccacaacgaactaggccccacccacagtgagctaggccccacccacaacaagctaggccccacccacagtgtgctaggccctcccacatcagtcaaaataaaaaatactccctcaggttacttacagaccaatctaatggaagtatgttctcagtggagagtcattccctcttctagatggccctcgcttgtatcaagtgatcacccagttcttggcttccagcttcctcattgggtcccagaggttccctctgtggcttcccagtgaggagtggtgcctcccaggagagacaggcagtgtgtggagatgacccaccacagaggatgatccatccaagtgccagggggactaggggaagacagttgtccgtccacagtggcgccccctgcaggcggcacactctgccagccgcctctcttaagccttgtgtttctccaaggtccatgtcccccagacaggacctcatgctggtacccctgtgccctctgacctggggtgacatgggacccccttgggcatgcctgctccggcttctcccactgtcttcagatgctgagagcctctgggctctatgtggccgttgtcccagggttgtaatctggcgtccccccacaggctgaacacaaccgtggcccccctgttcttcgctgaccagttcctgcagctgtccacttccctgccctcccagcacatcacaggcctgggggaacacctcagcccactcatgctcagcaccgactgggctcgtatcaccctctggaaccgggacacaccaccctcggtaagagacgggcagtgggtgggagggtggaggctgggatcctgagtgcaaggcgttgtctcttgtcaccccagcaaggtaccaacctctacgggtcacatcctttctacctggcactggaggacggtggcttggctcacggtgtcttcttgctaaacagcaatgccatgggtgagtcaccagactggggctcctcagcggtcacttccgtgtgagagcttgcccgctccagagtctgcctgtctccaagcgacctccgtggagagagtcccgtgagcagagtccccctcatcctcggaacctaaggaggtcctgggtgattctccaggctcacatgttcacgtccttcaggccttgctatgttgagctcagtacagctgcctcaggcccaccctgaggacatctccccacaattcatattggaagagccacactccacccacaggttgagtcccccatctgtggggcacttgttcctgagcccaaacacttctttccagatgtcatcctgcaacccagcccagccctaacctggaggtcaacgggcgggatcctggatgtgtatgtgttcctaggcccagagcccaagagcgttgtgcaacaatacctggatgttgtgggtaggcctggcccctgcccactgagctcagacccctgccccttgctccttgccccatgtccccagtccctgctcactcgcagtactggtcctcaggataccccttcatgcctccatactggggcctcggcttccacctctgccgctggggctactcctcgaccgccattgtccgccaggtagtggagaacatgaccaggacacacttcccgctggtgagtggcagtcagggtgagggcggccccagcttccgtgctcttggctcccgacctcagcatcagctcgcgtcctctgcttcaggacgtgcaatggaatgacctggactacatggacgcccgaagagacttcaccttcaaccaggacagctttgccgacttcccagacatggtgcgggagctgcaccaggatggccggcgctacatgatgatcgtggtaagcaggtttgggcacagccattgctgctgcttccgtgtggcctcttcctcctacctgagcagtttcccaggacactctctgatgtgtctgctgcccaggctcctggacacacccatgagctcggggctctttggttaacaggcgggatccgcagaactgctctgccttaaagcctgcggcaggggccgtaacgagctggtactgagctgaggacctagatgtggtttgtgcgtcctcctggccctgctcctctctcaggccagctatgtccatgtgggaaggaaacatacttgccctgtgcctgtccccgcagcaggaaggtccaagaagaccaaacagtcacagccatgggatgtccttgaggggagggagctaagccctggaagatacggctttgtaaggaagggctacgactgcctcgccgccaccaccaccaccaccaccaccacggggcagtgaatagtggtacaggcagtgagggccacccagacattaggcaagccctccaccactgagctgcactgccaactctctttccggtttttatttgaggcagggccacaccagattgcccagccaggccttgaatccataatcctcccacttccgcggtccaggcagctgggatgatgaacctgtatcaccagcccccgtgcagatttaagacagggtctcctcatgtagctctgactgacccatctgtcactgtatagagtagaccaggctggcctcaaactcacccgcctgcctcttagtctctcagcagctggaactaaaggcgtgcgccaccatgcctgggtgctttttattcatctccatttttaaaaagtaagtggggctggaaagatggctgagcagttaaagcacactgctctcgaagaggactggagttcggtgcctgggacccacaccaagcagctgtacttgtcatttcagcagctgctgctggcctctgtgggtcacgtgcattcgcgtgcgtgcgcgcgcgcgcacacacacacacagacacacacacacagacacacaagtaataataagctggacagtgtggtaaacacctgttttttttttattatttatttatttatttatttgagcacactggagctgtcttcagacacaccagaagagggcatcagatcccattacagatgtttgtgagctaccatgtggttttggggatttgaactcaggacctctggaagagcagcagtcagtgctcttaacctctgagccatctccccagcgcatacagtgaacacctttaatcacagctctcgggaggcagaggcaggtgcatctcttgagttctgtgccaacccggtctacagagtgagtttcaaggcagccaagaatatacaaagaaaccccatctttaaaattaaaaataaaataaaaagagcaaatataagcaactgctgtaactctggaaagcctgagaacaggaggatcacacgtctagacttgctcacccctgctgcccccaacaggcttcgcacagcctgacctatgccgctttcctgtataggccatctctgcctcggccagctctcttctctgggacctgagcttctacctctgccgctccggctgctcctccttctgggctttggggctaggctcttcactgttggcttctccccccaggaccctgccatcagcagcgcaggccctgctgggagttacaggccctacgacgagggtctgcggaggggcgtgttcatcaccaacgagactgggcagccgctgattgggaaggtagcgagggggatgggagctgtggtggtggtggtggtggtggggtcctcactgggggtaactcctgacagtcgcctgaggttttctaagtggagggtgttctcaggcatcctgagtagctgggcccggcttaagcctagcagacctttcgtgaggccttggagattggggggaggggagtcttccttgtccctgggggccttctgggagttctgagtcccagcctccccatcctaagcagccctggccatgggagaagtgagggcttgtgtctggtggtcagggatcaatggcactatctcaacctctttcccatgccctggttcatggctcatctgccccacggccttatgtgtagggctggcctttgctctcccaaggctgaaggcccctatgccctgaggcattgtttgatattgggcttgaggctctctgatatcatcattggcttagagcgaggctgctgccttgcttgaggcatgaggcccaacagggctctggcgcaggatctgtgtggtattggttcttccctctgggctcttgttcaggtttggcctggaaccaccgccttccctgatttcaccaaccctgagacccttgactggtggcaggacatggtgtctgagttccacgcccaggtgcccttcgatggcatgtggctcgtaagtgcagtcccatccctccatgggtacacccccagcctctggggaccaacctagctcacatctctgcctcttccaggacatgaacgaaccgtccaacttcgttagaggctctcagcagggctgccccaacaatgaactggagaaccccccctatgtgcccggtgagccctgcttgcctgcccggaatggggatagtgctagagttagtggcttaaaccagggagtgtgcgcctctctgtggtttgggggatgtaaagctctccatttttttggccgtcaatagacacagcagcttcgggacacagaacccaggagtgaagtcctacagtgtgtgacacagacatggtcacagctagccagtgcatcaggggaagggggttaggcccggatgacagacacttaggatgagtcaagagaaggtggatgtggagactgtaggtttaggaggaccgtctttcgtacacctgtctttggactagcagaagtgtatagtgatgtggggggatggggatgggaccagtttcaggaggctccgccctgggcagaacccaaggaggtgtcaatcaaagcagagggtgtgggccaaggagagggcacagggaaccctggagagagttacacctaaaactagtcctcctctcgcctaggggtggttggcgggatcttgcaggcagccaccatctgtgcctccagccaccaattcctctccacacactacaacctccacaacctgtacggcctcactgaagctatcgcctccagcaggtgaggctctgccggctctgcaccagccagcctgccctgcaatcacagtgggggtcgagaggtgggctggctcatgtcagggtcatgaaaggaagggaaagaggttaccataccacccatgggccagcgatgcctgcacgtctgctgaaatggttgctgttttccccctccccccccccagcatggagccggcttctcccccctatgcccagaccagcccagatgttaagtggggatgtccaaagggactccatgtctctggcaaggcttagagtggtgactcacctgtaaatgacttaagagtcattggggcaggcctgactgtacctagcatactttaaagatggcctctctttcccacccattgtgatctactaaactatcagttcctcttggccctgatccccttgctgctggcccccatgctctgttccagcacacgggtgtgtatgacccaggtgagcccaggtctctgtgttctcatcccactcagggccctggtcaagactcggggaacacgaccctttgtgatctcccgctcaaccttctcgggccacggccggtacgctggtcactggacaggggatgtgcggagctcttgggagcatcttgcatactctgtgccaggtgagtacaagctgcctgcagggctctggggtcttttctgggggtctttgggctgtgggctcaccactgcagatggtattgtgatagcttggggacatgcgccatctcatgtggcacatgaagcctgctgctggtccttactcacctgtgtcagcagcgcacctgtgcagtatggctcgcctgggcacctttcttctcaggatccctgtgtctgtttcaccctgaccactgcttgaaaagccctcagactctcgccagagaccggttgttctctggtcacctcatccggagacacaggctagctgttccacttgcttttggggttttgtttgtttttcaagacagggtctctctctctctctctctgtatagccatgaatgtcctggaactggctttgtagaccagtctggcctcaaattcatagaagtctgcctgcctctgcctcccaagtgctgggattaaagacgtttataaccacagcccagctgcctgatctacttgcaaaaaggagtttggtttttattccaagaacttgaatgccattggcccatgttaggtcagaaagtcaggagccctagagaacagactagggaaggagagggagccaggcctcctcatgggcacctaaggggcgtgactaagagtttggaaccttctaacttctggaagactggcttctgtataggatatgctcacagacctatgaagggccccagctggctgactcctcagaggtggagctggctacctctgcctcatgggctgcagctcactgcaggcaggcctggtaggccaggagccagactcagagcagccttgcagagctgagtctggtctttctccttctcttttggcctcaggtctcttaatgtcctgaggctgccagagctgagggcagctttctggggacttaatgtcataggaatccatctgtggatagtgactggaccataggtctgcagccaaggagtgacagagccacacccccttgccccaaggtgtggctcggtgcccctcacagccttgggaacccttcattctgctctaggccctctgggcgttgctgctgggatgtatctccatcctcacagggctatcgtcccatgtgacttttaagtgtcccacgacattttcttcaggaccacatcaaacccaccttagtgccattacatctggagagctcctgtttccaaatgaggttacagtcacacatcctgcaccccagtatcttgggctctgggctactctgctgcccacaggcgttaggacaaatggaaagcagagaactatgggctcttgcccgtcaggccgggatggcaaggctgggttcaacctgcccagcaggcctgtctgtccagctgctgtcttggtaacctggtaccactctgcctctgtacccagacatcctgcagttcaacctgctgggcgtgcccctggtcggggcggacatctgcggcttcataggagacacgtcagaagagctgtgtgtgcgctggacccagttgggggccttctaccccttcatgcggaaccacaatgacctgaatagcgtggtaggacggagtggcagggagtccccacccacctagaccctctcctggttaatcccatacctgctggaagagctggtccgtgccctcctctccacacaggatggccaaggagctgtaggctctgggggcagaaattctgttgaacccaaggcctaggagcccagacacttcctgttctagtgtgctctccggctaccaaggccaaaagcaacttgggcagaaagggtttattccatccttcacctcacagttgtgaaggaaagtcagggcaggaactaaagcagaaaccacggaggaacactccatgttccttgcttcccaccccccccccccacacacacacacagctctcagcttgctttttaattcaactcaggaccccctgcccagggggtaccacccatggtggcctaatgctcccacacccctgccttaaaggccagtctggtggggctgtttttctcaagtgaggttccctttttccagatgattgttagtctgtggacaaaatgctaaacagctcctcctttgtcctgtctgcccacacagcctcaggagccgtacaggttcagcgagacggcgcagcaggccatgaggaaggccttcgccttacgctatgcccttctgccctacctgtacactctcttccaccgcgcccacgtcagaggagacacggtggcccggcccctcttcctggagtgagtcaccagggtcagggattgacagcctacatgcgtctcagggaggactgaccagaagtcttccagtgcccgagaagtccctcctgctaggggtgttctcacaacccaagcaggctgcagctgagtacagcaggagtgcctcgtgctcgcaggcccctggacctcagctcaactcaggacatcagagtgtagacaactggctggggtcatcagagtatagacacaggcttgtagctgacaaagctttgatgaaacagacatgactgatccgtttgggggtggcttaagagaggaggacaagcaagaaatgtcccatctgtgtcgccggacaggaccgcttacatgggaggcatctctgatgggcagacaggcagaagacagttcttgggtaggaggggaagcagctcactctacagagagaagcactctctcgtaccacacacagatgagttccagacagactggatggttaaatggccattgggctagaaatggctcagttttgataacagtgcttgcttgccttagaagcgtgaggggtcaagttggattcccagtgctttaagaagctgggcacgtggcatgtggccatagtggcactcaggaggcagaagcaggtggatctcttatcagtttgaggccagcctggtcacaaaaacactggctcaaacaactgaccaaatcaacctgggcatggtggctcttgtttagatccagcacggggagacagctatactcttgggtcttcactggctactggccagtcagcctggccttgtcaccctgtctccaaagaccaagttgatgacactaaggaacagtgctagaggctgtccttggcctccacatagacgcacaggcgtgtgcagcacacaagctcagggttttaagcaggtataatttatttacctggaagcattataaggcctgcccagggatgaaagtgaccaaggctgtcaaagactcaaggggggaggtactcagcattcgtattccacgcagatgcatgaggacacagctgtgcacactgagggtatttttggctccctggtgcatgccttagtttcacaaatggagttctctgaaaagcataatgatcaatcgtgtccctagcattgcgatcttgcctcaacattggatcagagcagagcccaggagtgatgaggtatatgaggacagctgggggtctgccaggtattcagagtggctgcagtgagaggacagctggggtctgccaggtgttcagagtggctgcagtgagaggacagctggggtctaccaggtgttcagagtggctgcagtgagaggacagctggggtctaccaggtgttcagagtggctgcagtgagaggacagctggggtctgccaggtgttcagagtggctgcagtgagaggacagctggggtctaccaggtgttcagagtggctgcaatgagaggacagctgggggtctgccaggtgttcagagtggctgcagtgagaggacagctggggtctgccaggtgttcagagtggctgcagtgagaggacagctggggtctgccaggtgttcagagtggctgcagtgagaggacagctggggtctgccaggtgttacatggtggctgttggtcagtgaaattttagggaatgtttcctggtcattgatgcttgcttttctgcattttacaaacttttgattggatggatggcatcaccattgtgagattcaggaaactgagcaaatccagaggctcctcaatgcttcatgcctgagcctggagagtcagcaaagcaataattgctctgtgctctggcacaagtgtcttagatcctccggttcctgtggtttctcagacacctggatttcctgactctgtgcaattgaacagctgacacctggccctcactcctatctctcccatgaaggttccctgaggatcccagcacctggtctgtggaccgccagctcttgtgggggccggccctgctcatcacacctgtgcttgagcctgggaaaactgaagtgacgggctacttccccaagggcacgtggtacaacatgcagatggtgagtcccgaggtccctcgtacacaggcgaagacgagaattcatacatcagtctgtgatcctcttacctgtcagctttttgccgtagtccccacttctagaagaagaaattaagtctggagtaggtagcttccagggcctgacagccattgaggctgagctgagccagacattccctgctcaggccaacaccaagtccttctctggggtcacagccagcttgtcccaagagcacgcatagaccaccacacatagccttgggggacaggaaaacatggctctgagttccagcatggtagcttgggtgctacctgggacccttctgatgtgaccccttcctccaggtgtcagtggattccctcggtactctcccttctccatcatcggcttcatccttcagatctgctgtccagagcaaggggcagtggctgacactggaagccccactggataccatcaacgtgcacctgagggaggggtacatcataccgctgcaggtacacagacccagcagctccagggcctcaggaaggcctgggacgggcttggagagggtgggagagggagctggcccagggcgcagtctgctttccctccctggaactgtcaggatacacccctggcagtcagggtgacaccaaaaagaacaaacttgggtctacgcggcactaggtcaaggtagtcctcgtgtcatgcaagtgtccagggtctgaggtatgacatgtgccccatcctcttcccagggtcccagcctcacaaccacggagtcccgaaagcagcccatggctctggctgtggcattaacagcaagcggcgaggccgatggggagctgttctgggacgacggggagagcctcgcggttctggagcgtggggcctacacactggtcaccttctcagccaagaacgtgagtcccaaggcctgtccaggctggtgggtggaaacatgggggatgggattgtcttgacctgggggagggaaggactgggccagagtacccagtggccagcatgtctcttgtgtctgcacagaggaggctgcctgtgacgcttgctttatagtctctatcctggtgggctcacggtcatggtccagtgacactcctttttcttttctcttgacattaccagaataccattgtgaacaagttagtgcgtgtgaccaaggagggagctgagctacaactgagggaggtgaccgtcttgggagtggccacagctcctacccaggtcctttccaacggcatccctgtctccaatttcacctacagccctgacaacaaggtatgagggcaggctggggattggggatcaggcaggagcgaagagggtgctaaggactcccttggtgcagccaactcagacacgctcctgggagggagacagggcatttggacctcacagagtagaatgggccgagtggatcctcacccctcaccatatctaccctgagatacagctccaatggtggaggctataccctgagatacagctccaatggtggaggctatgagctggtaaacagagccttgctcccgatggaatccaatgctgattccaaggcactggactctctaccttggggagggatggccaggtgtgtgctcatcacctgcctgtctccagaccatctggagcctggctgggggagggaaccacttgccattgaagcttctttctgacctccagagcctggccatccctgtctcactgctgatgggagagctgtttcaaatcagctggtcctaggagagtccgtcgtttacagaggcctccagggaggcagagggagcttgagctggctctggctggtggctcctgtaaggacctgcgtcctgctctcctgacacatctttgagcttttcccaccgtgttactgcatgcgcccctgaagctctgtgttcttaggagagtgaggctcgcctcacctgccccaccccagctgtctgtccctcacctggcactagagaatgtggagctcggcgtggggacatcgtgtctgcaccaacatcaggctgtgcagccactgcagccgcaaccctgcagagacagagctggtgccttcaccaggttcccaagactcgagaaacttactgtgaagtgtacttacttttaataaaaaggatattgtttggaagcagtt

SEQ ID NO: 27 - RNA, 3579 nt, Mus musculus
  source 1..3579: mol_type = mRNA
acacggactccgccgcctacgcaggaggtcgtgtgacgaggtccgcgcggacgagccccgcccacagaccacgtgacccacgctgcccgctgagcctgggggtcttcggcctggagggtgattgcgcaggtgagctgggcgagctggtgaaggctgtctccggccccagagcctccggaggagcggcctcctccaggcaggcagggccgactgtcagcgagttcctgctttggagaatcgagcagagacacgcggcataggccttcagaagtattcatgctgccccgaaccaacaggctttcaccatgaatatacggaagcccctctgttcgaactccgtggttggggcctgcacccttatctctctgactacagcggtcatcctgggtcatctcatgcttcgggagttaatgctgcttccccaagaccttcatgagtcctcttcaggactgtggaagacgtaccgacctcaccaccaggaaggttacaagccagggcctctgcacatccaggagcagactgaacagcccaaagaagcacccacacagtgtgatgtgccccccagcagccgctttgactgtgcccccgacaaaggcatctcacaggagcaatgcgaggcccgcggctgctgctatgtcccagcagggcaggtgctgaaggagccgcagatagggcagccctggtgtttcttccctcccagctacccaagctaccgtctagagaacctgagctctacagagtcggggtacacagccaccctgacccgtaccagcccgaccttcttcccaaaggatgtgctgaccttacagctggaggtgctgatggagacagacagccgcctccacttcaagatcaaagatcctgctagtaagcgctacgaagtgcccctggagaccccacgtgtgctgagccaggcaccatccccactttacagcgtggaattctcagaggaaccctttggagtgatcgttcgtaggaagcttggtggccgagtgttgctgaacacaaccgtggcccccctgttcttcgctgaccagttcctgcagctgtccacttccctgccctcccagcacatcacaggcctgggggaacacctcagcccactcatgctcagcaccgactgggctcgtatcaccctctggaaccgggacacaccaccctcgcaaggtaccaacctctacgggtcacatcctttctacctggcactggaggacggtggcttggctcacggtgtcttcttgctaaacagcaatgccatggatgtcatcctgcaacccagcccagccctaacctggaggtcaacgggcgggatcctggatgtgtatgtgttcctaggcccagagcccaagagcgttgtgcaacaatacctggatgttgtgggataccccttcatgcctccatactggggcctcggcttccacctctgccgctggggctactcctcgaccgccattgtccgccaggtagtggagaacatgaccaggacacacttcccgctggacgtgcaatggaatgacctggactacatggacgcccgaagagacttcaccttcaaccaggacagctttgccgacttcccagacatggtgcgggagctgcaccaggatggccggcgctacatgatgatcgtggaccctgccatcagcagcgcaggccctgctgggagttacaggccctacgacgagggtctgcggaggggcgtgttcatcaccaacgagactgggcagccgctgattgggaaggtttggcctggaaccaccgccttccctgatttcaccaaccctgagacccttgactggtggcaggacatggtgtctgagttccacgcccaggtgcccttcgatggcatgtggctcgacatgaacgaaccgtccaacttcgttagaggctctcagcagggctgccccaacaatgaactggagaaccccccctatgtgcccggggtggttggcgggatcttgcaggcagccaccatctgtgcctccagccaccaattcctctccacacactacaacctccacaacctgtacggcctcactgaagctatcgcctccagcagggccctggtcaagactcggggaacacgaccctttgtgatctcccgctcaaccttctcgggccacggccggtacgctggtcactggacaggggatgtgcggagctcttgggagcatcttgcatactctgtgccagacatcctgcagttcaacctgctgggcgtgcccctggtcggggcggacatctgcggcttcataggagacacgtcagaagagctgtgtgtgcgctggacccagttgggggccttctaccccttcatgcggaaccacaatgacctgaatagcgtgcctcaggagccgtacaggttcagcgagacggcgcagcaggccatgaggaaggccttcgccttacgctatgcccttctgccctacctgtacactctcttccaccgcgcccacgtcagaggagacacggtggcccggcccctcttcctggagttccctgaggatcccagcacctggtctgtggaccgccagctcttgtgggggccggccctgctcatcacacctgtgcttgagcctgggaaaactgaagtgacgggctacttccccaagggcacgtggtacaacatgcagatggtgtcagtggattccctcggtactctcccttctccatcatcggcttcatccttcagatctgctgtccagagcaaggggcagtggctgacactggaagccccactggataccatcaacgtgcacctgagggaggggtacatcataccgctgcagggtcccagcctcacaaccacggagtcccgaaagcagcccatggctctggctgtggcattaacagcaagcggcgaggccgatggggagctgttctgggacgacggggagagcctcgcggttctggagcgtggggcctacacactggtcaccttctcagccaagaacaataccattgtgaacaagttagtgcgtgtgaccaaggagggagctgagctacaactgagggaggtgaccgtcttgggagtggccacagctcctacccaggtcctttccaacggcatccctgtctccaatttcacctacagccctgacaacaagagcctggccatccctgtctcactgctgatgggagagctgtttcaaatcagctggtcctaggagagtccgtcgtttacagaggcctccagggaggcagagggagcttgagctggctctggctggtggctcctgtaaggacctgcgtcctgctctcctgacacatctttgagcttttcccaccgtgttactgcatgcgcccctgaagctctgtgttcttaggagagtgaggctcgcctcacctgccccaccccagctgtctgtccctcacctggcactagagaatgtggagctcggcgtggggacatcgtgtctgcaccaacatcaggctgtgcagccactgcagccgcaaccctgcagagacagagctggtgccttcaccaggttcccaagactcgagaaacttactgtgaagtgtacttacttttaataaaaaggatattgtttggaagcagttaaaaaaaaaaaaaaaaaa

SEQ ID NO: 28 - protein, 953 aa, Mus musculus
MNIRKPLCSNSVVGACTLISLTTAVILGHLMLRELMLLPQDLHESSSGLWKTYRPHHQEGYKPGPLHIQEQTEQPKEAPTQCDVPPSSRFDCAPDKGISQEQCEARGCCYVPAGQVLKEPQIGQPWCFFPPSYPSYRLENLSSTESGYTATLTRTSPTFFPKDVLTLQLEVLMETDSRLHFKIKDPASKRYEVPLETPRVLSQAPSPLYSVEFSEEPFGVIVRRKLGGRVLLNTTVAPLFFADQFLQLSTSLPSQHITGLGEHLSPLMLSTDWARITLWNRDTPPSQGTNLYGSHPFYLALEDGGLAHGVFLLNSNAMDVILQPSPALTWRSTGGILDVYVFLGPEPKSVVQQYLDVVGYPFMPPYWGLGFHLCRWGYSSTAIVRQVVENMTRTHFPLDVQWNDLDYMDARRDFTFNQDSFADFPDMVRELHQDGRRYMMIVDPAISSAGPAGSYRPYDEGLRRGVFITNETGQPLIGKVWPGTTAFPDFTNPETLDWWQDMVSEFHAQVPFDGMWLDMNEPSNFVRGSQQGCPNNELENPPYVPGVVGGILQAATICASSHQFLSTHYNLHNLYGLTEAIASSRALVKTRGTRPFVISRSTFSGHGRYAGHWTGDVRSSWEHLAYSVPDILQFNLLGVPLVGADICGFIGDTSEELCVRWTQLGAFYPFMRNHNDLNSVPQEPYRFSETAQQAMRKAFALRYALLPYLYTLFHRAHVRGDTVARPLFLEFPEDPSTWSVDRQLLWGPALLITPVLEPGKTEVTGYFPKGTWYNMQMVSVDSLGTLPSPSSASSFRSAVQSKGQWLTLEAPLDTINVHLREGYIIPLQGPSLTTTESRKQPMALAVALTASGEADGELFWDDGESLAVLERGAYTLVTFSAKNNTIVNKLVRVTKEGAELQLREVTVLGVATAPTQVLSNGIPVSNFTYSPDNKSLAIPVSLLMGELFQISWS

SEQ ID NO: 29 - DNA, 18325 bp, Homo sapiens
  source 1..18325: mol_type = genomic DNA
acccgcctctgcgcgcccccgggcacgaccccggagtctccgcgggcggccagggcgcgcgtgcgcggaggtgagccgggccggggctgcggggcttccctgagcgcgggccgggtcggtggggcggtcggctgcccgcgcggcctctcagttgggaaagctgaggttgtcgccggggccgcgggtggaggtcggggatgaggcagcaggtaggacagtgacctcggtgacgcgaaggaccccggccacctctaggttctcctcgtccgcccgttgttcagcgagggaggctctgcgcgtgccgcagctgacggggaaactgaggcacggagcgggtgagacacctgacgtctgccccgcgctgccggcggtaacatcccagaagcgggtttgaacgtgcctagccgtgcccccagcctcttcccctgagcggagcttgagccccagacctctagtcctcccggtctttatctgagttcagcttagagatgaacggggagccgccctcctgtgctgggcttggggctggaggctgcatcttcccgtttctagggtttcctttccccttttgatcgacgcagtgctcagtcctggccgggacccgagccacctctcctgctcctgcaggacgcacatggctgggtctgaatccctggggtgaggagcaccgtggcctgagagggggcccctgggccagctctgaaatctgaatgtctcaatcacaaagacccccttaggccaggccaggggtgactgtctctggtctttgtccctggttgctggcacatagcacccgaaacccttggaaaccgagtgatgagagagccttttgctcatgaggtgactgatgaccggggacaccaggtggcttcaggatggaagcagatggccagaaagaccaaggcctgatgacgggttgggatggaaaaggggtgaggggctggagattgagtgaatcaccagtggcttagtcaaccatgcctgcacaatggaaccccgtaagaaaccacagggatcagagggcttcccgccgggttgtggaacacaccaaggcactggagggtggtgcgagcagagagcacagcatcactgcccccacctcacaccaggccctacgcatctcttccatacggctgtctgagttttatcctttgtaataaaccagcaactgtaagaaacgcactttcctgagttctgtgaccctgaagagggagtcctgggaacctctgaatttataactagttgatcgaaagtacaagtgacaacctgggatttgccattggcctctgaagtgaaggcagtgttgtgggactgagcccttaacctgtggagtctgtgctgactccaggtagtgtcaagattgaattgaattgtaggacacccagccgtgtccagaaagttgcagaattgatgggtgtgagaaaaaccctacacatttaatgtcagaagtgtgggtaaaatgtttcaccctccagcccagagagccctaatttaccagtggcccacggtggaacaccacgtccggccgggggcagagcgttcccagccaagccttctgtaacatgacatgacaggtcagactccctcgggccctgagttcacttcttcctggtatgtgaccagctcccagtaccagagaaggttgcacagtcctctgctccaaggagcttcactggccaggggctgctttctgaaatccttgcctgcctctgctccaaggcccgttcctcagagacgcagacccctctgatggctgactttggtttgaggacctctctgcatccctcccccatggccttgctcctaggacaccttcttcctcctttccctggggtcagacttgcctaggtgcggtggctctcccagccttccccacgccctccccatggtgtattacacacaccaaagggactcccctattgaaatccatgcatattgaatcgcatgtgggttccggctgctcctgggaggagccaggctaatagaatgtttgccataaaatattaatgtacagagaagcgaaacaaaggtcgttggtacttgttaaccttaccagcagaataatgaaagcgaacccccatatctcatctgcacgcgacatccttgttgtgtctgtacccgaggctccaggtgcagccactgttacagagactgtgtttcttccccatgtacctcgggggccgggaggggttctgatctgcaaagtcgccagaggttaagtcctttctctcttgtggctttgccacccctggagtgtcaccctcagctgcggtgcccaggattccccactgtggtatgtccgtgcaccagtcaataggaaagggagcaaggaaaggtactgggtccccctaaggacatacgagttgccagaatcacttccgctgacacccagtggaccaagccgcacctttatgcagaagtggggctcccagccaggcgtggtcactcctgaaatcccagcacttcggaaggccaaggggggtggatcacttgagctcaggagttcgagaccagcctgggtaacatggcaaaatcccgtctctacaaaaatacagaaaattagctgggtgcggtggtgtgtgcctacagtcccagctactcaggaggctgaagtgggaggattgcttgagtctgggaggtggaggttgcagtgagccaggatctcaccacagcactctggcccaggcgacagctgtttggcctgtttcaagtgtctacctgccttgctggtcttcctggggacattctaagcgtgtttgatttgtaacattttagcagactgtgcaagtgctctgcactcccctgctggagcttttctcgcccttccttctggccctctccccagtctagacagcagggcaacacccaccctggccaccttaccccacctgcctgggtgctgcagtgccagccgcggttgatgtctcagagctgctttgagagccccgtgagtgccgcccctcccgcctccctgctgagcccgctttcttctcccgcaggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacggtgggcagggcaggggcgggggcggcggccagggcagagggtgcgcgtggacatcgacacccacgcacctcacaagggtggggtgcatgttgcaccactgtgtgctgggcccttgctgggagcggaggtgtgagcagacaatggcagcgcccctcggggagcagtggggacaccacggtgacaggtactccagaaggcagggctcggggctcattcatctttatgaaaaggtgggtcaggtagagtagggctgccagaggttgcgaatgaaaacaggatgcccagtaaacccgaattgcagataccccaggcatgactttgtttttttgtgtaaggatgcaaaatttgggatgtatttatactagaaaagctgcttgttgtttatctgaaattcagagttatcaggtgttctgtattttacctccatcctgggggaggcgtcctcctcctggctctgcagatgagggagccgaggctcagagaggctgaatgtgctgcccatggtcccacatccatgtgtggctgcaccaggacctgacctgtccttggcgtgcgggttgttctctggagagtaaggtggctgtggggaacatcaataaacccccatctcttctagatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgtgagttctgggctctgtgccagcatgatggggagggcgacgcgcatttctcacacggcagggagggccacacgcgtttgtttctcacacgatgggcagggcgacacatgtttgtttctcacacggcggggagggcgacgggcatttctcacagggcgctccctgggtcttttactcacataggtctaaatcccatgtaaacacgtgttcaggactcaccaagcccctgcttgtcatttaactcaggaaaactctcaggaacgacagcacttggatttgccttaatcttaagagaagttgccttcggaaatgcgtttttctttttttgctcattcatttactcagtgtccacgcactgaccctccgtgccgggtggtttggatcctgctcccggggacagacacacagtgaggggaagccataagcaagtccatgcagacacagcgtcagggagtggtcatgcagagagcacgctagaagccagctgtgcagacacggggcagggaggtcccctctagaagccagctgtgcagacgcaggggacagggatggcctctctggaagccagctgtgcagatgttgggggcaggggtggcctctctggaagccagctgtgcaggagtggggggtggggaggccactctggaaaccagctgtgcagatgcaggggacaggggtggcctctctgagctgacctctgagtagagagacccaagagaagtttctcaaagcatcttatcaagctaggtatggtggttcatgtctgcaattccagcactttgggaggccaaggcgagagggtcacttgagcccaggagttcaagaccatcctgggcaacatagcaagaccccatctcttaaaaaataaaaataaaaaattagctgggaattgtggcacatgcctgtggtcccagctactcaggaggctgaggcaagaggatcccttgagcccaggggttcgaggttgcagtgaaccatgattttgccactgcacttcagccttgctgaagaccccgtctcaaaaaacaaacaacaaacaggcatcttatcagatctcggtcttgaaagcactcagcgtagtcttgcccaggggagggtgggtgcggtgtgagcccgtcctgcgaaattagctgtgctgtgttaacagaggacgcgtcttcctgtggaccgggtttatctgcggctttcatttctcggaggtgctgtttgccttgcacttgacccccagcaaacctcaggggtccttctcaggcatggctgggctgggatctgggaggactttggccacaagctcctaggcctggaaaggttctgttcagcccctgcccagccttgcttggggtcatgggacaggcatgtgtgccagttccggtaccagccagttcctggaggtcagccccttgggggcccctcaggggtggtgtgggcccagccaggcggtgcgcctcttctgatatgccctgagagttgatcacgctggtgccagggtgccaagggctgcagggctcggcacggccgcctgtcccagggtcagtgtgctgcagggctggccaggccactccgccctcccagggcaccagggcccgggggtgctctctgggtgctctcaggctcgtgtggccccttgggtgtgagcaagcctggctggcctctgtcccgcaggctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacggtacagcggcgggcggcgggcgggggcactgagctggggagcgcaggtgctgaagcgccgtctcctgcatgtcccagcccggtgcgaacctctacgggtctcaccctttctacctggcgctggaggacggcgggtcggcacacggggtgttcctgctaaacagcaatgccatgggtaagctgcccgccgcccagcgcccgggccggggtctcctccgtgctgcctgccctggagactggaggtccgcatgaggggccctgggcacggtgctgggccttgtgttttctgggaaatgagtcctatgggctgatgcctctcccaactctggccttctgtgctcctaaggagggttctggggccctgcctggaggtgggctggcaccacatatctttccgtcccatgccaggttcctcctgagtcaggcttagcacggcttccccaggccactctgagctcctcgtggggagagagcctcaactctccgcctgtgattggcccatctgtggggtgcagagccctccaagtgaagaatctgtcccccaaccccagagctgcttcccttccagatgtggtcctgcagccgagccctgcccttagctggaggtcgacaggtgggatcctggatgtctacatcttcctgggcccagagcccaagagcgtggtgcagcagtacctggacgttgtgggtagggcctgctccctggccgcggcccccgccccaaggctccctcctccctccctcatgaagtcggcgttggcctgcaggatacccgttcatgccgccatactggggcctgggcttccacctgtgccgctggggctactcctccaccgctatcacccgccaggtggtggagaacatgaccagggcccacttccccctggtgagttggggtggtggcaggggaggcaaggggctggccgggacgcgtctcctcaggccccagcagacggtcccgtgttgtggctgcaggacgtccagtggaacgacctggactacatggactcccggagggacttcacgttcaacaaggatggcttccgggacttcccggccatggtgcaggagctgcaccagggcggccggcgctacatgatgatcgtggtgtgtgcccccacactgtgggtctttgggaagggggccgcccggtgcccagtggctccttctctgtgcagcgtcatcctcgtgcctgtgtggtcgccgaggatgttttctgagggtctttgtgatatcgagggaatatcaagaagtttgcaggcttggccccagctgtccagggaggtcgggtttgagggtccccagaaatggccgggtgctactcagggttctgtcagatgtaggttacttgaactgccttaaagcaaaaggccaggggcatgataaactgatgtcacctggtcctggaaagtggagggcccggtgggcctgggcatgggtatcgctggaactgtggaggctccgtgtgccttctggccgtgcctctccttctggccggctctgaatccctggaaaggacggcgtgagtgagggcagcttccagccctcatgctggcaccacagagcggagacttcttcccatcagctcccatagaaaagtcccaaagcaggactcttgagtcacccagcacaaagaggcccttccctgagccagtcccacagccagaaggatgcagtttgggggctggtccagcccgagtctggtgtccggcacgatggccagaggaggaggtgggaggcagggcgagctgaaaagatccaacagttcctgcccggaagatccacttcagcagaggaagcacagatgagatgtggggctgtgctgatgctgcctgtttccatccctgccttctgcaggcagcaaacagtagtagcccttaagagcaggagtggaaacacagacttttttctttctcacatttttttaattataaaagaaaagtgattactgtagaacacttgggaaactctagaggtttaaagaaaaggtaaaggtaaagcttccattccggcgcgcccctcatcagccagctggtcctgactcgcccggccctggctcctctccaggcaggcgtgtgcaggcatgtgcaggtacacaggcaggcatgctgtacacacgcatgatgtcatccccagcctcatcctctcactgtctcagttttccccgtggctggcgccagggctctgggccaccctcaccttgacaggtttccctcttcccaggatcctgccatcagcagctcgggccctgccgggagctacaggccctacgacgagggtctgcggaggggggttttcatcaccaacgagaccggccagccgctgattgggaaggtagggcgagggtccaggggacgggggttagaaagcagaggcctccagccagggggagccggcagctgctcaggaagacggtgggatttgaggagccatcacgcccagtgggacagctgagaggaatgggccacagtggcccgtgacgatggtggctcctacaaggaatggccccgtgagttcttccatcagcaggcctttgacttcatgggcagctgggcctggcccaggcacaagccctgcagaccctcagtgaggccttagggtcctccttgtcctcccagccccccaggggcctccaggcagggcccccgctgagggagcagctagggagggtctggtgcggatgtgaggctgcctggcagggcttgcacggggccgtctccgctgcccttctccctgacgctctctggttctgcagcccagcccctgggtggacgtgttgggggtgacccctcgttttcccagggttgaggccccttggccccgcatcagtgccttgtggagaaagagctgctcattgacctccagggtgcaggtctctcagatttgcaaatgtgggcgtccactaagagtgaggctgcccctctgctcaggctgaggctcagtggggcttccatgcaggccctgggtggggccgggtctccccactgcagcctctcgttgtccaggtatggcccgggtccactgccttccccgacttcaccaaccccacagccctggcctggtgggaggacatggtggctgagttccatgaccaggtgcccttcgacggcatgtggattgtaagtgtggccccctcctgagcatccccaaggcctctggggactaccccaccctcctcactctgggcagagtcacctaccagcagcgcttctcttgcaggacatgaacgagccttccaacttcatcaggggctctgaggacggctgccccaacaatgagctggagaacccaccctacgtgcctggtcagctcgccccccacctaccctggggacttaatcaaatcagagactcccttgtctggcctgggagacttagcaccctcatctctgagaagcagatgggccagcggggaaaggggcgggggggggatccccaggagaaaggctcaggctgggagactcagccaagcagtgcagacagggtgggtgcagaggcacaggccctgccggaggagacgccgctcacaggtgcttgccagagcacagtgaggccgactcgactcagagccgtctcgataggcgcagggaccatgcagcggagacctacccacccgtggggagaggtcaggcccaactcgaatgcagcacgggcaagtggatttctagccagggagcagggtgggctcagaggcaggaattaccaagaagaagcatgggggtcagggggattctggctgaactgacccagcaggattcttgctgaaggcaggccagggtgaccagacatcgcctgaggggtggtggaggttggggcttctcgccaaactgtcttagcaggaatggcagaaactgggttttacaaggaagtacaaggatgggcctgggagaaggtttgggggcctgaggctatagtttggcccagcaaagaatcagtgagaggatggggttttgggcttaggtaaacaggcaggggagtgcttgaaatgggccaagagacggtggatgtgaagtctgggggtctgcagagcccaggctccagcacccgcccagccctgtcttagaagcagtggagatgattacccaggttcccgggtaacgccagccccacagaggcgtggggagcggctgcaggtgcacctccagggccagcctgaagaggcagcgacctgcacaggggctcctgggaggtggggggcagggagggcaccttggagcctgccgggaggaagctccctggaaaccagcccccgcctcttccaggggtggttggggggaccctccaggcggccaccatctgtgcctccagccaccagtttctctccacacactacaacctgcacaacctctacggcctgaccgaagccatcgcctcccacaggtgagggccacgtcccgccccactgggctctgccctcacagcctgtcctacaaggttggggcctctgcagggcctcagggaggaggaaaagcggaggcccagaccacccggggcccgctggcggcccgagtgctctccccacccgctgcctgcaccccagcctgaagctggagcgctccttcccacttcatgcctggggcttggagaggaaggaccctggatgctgacaggagtctgcatcagcggggacctcatgactcctgtgaggctggggggggtcctggctcacctacaggcatcaggtggcccagacagaggcaactgtgcccgcagacatgggcagtagcctcgccgtcctcctccccagcctctgcctcatcccagaaagctccttgctcccagctctgccctgctggtgacagggttcccgagtgaccccgctccacacagccctcacggtgtcccccaccaccccagggcgctggtgaaggctcgggggacacgcccatttgtgatctcccgctcgacctttgctggccacggccgatacgccggccactggacgggggacgtgtggagctcctgggagcagctcgcctcctccgtgccaggtgagctcctaccaggaggggctgctcagcagagtagagccgggggcctctatgggaggcttgccggggccccccacccacttagcaggtggggctctgggtcacttggcctgagctggctctgctgcagcagcctgaggaccagcctgactctgccctcccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctggtagggtgggggtggcggcatggcaggtgggcgatcccacccacccaagactctcccctgggaatcccacccctgctggagaagcaccccatgctgggtggctgagaagtgcagctctcccgaggcggggactccaggggaccgcggccccagcacccaagtgcttcctttgcccccgcctgccctgcagccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagtgagtgacctaggcaggggcggtggcccatgtgtgccctgggggaggggcacgtaactcccaggcagccctgtcctgctgtgggctgtgttccccaggacccagcaggttgccgctgagtgagacaacatttgggcctggcttaagggggaagggcagcaagaaaacccagtaatatcccccagacaggccgtagtacacacgaggagttcctaacaacagccctgcacatcagtgtgttgagggaggattcccagagagtgaggtgattagttaactatttgcagaagtcaatttatttttcttgcataccatagaaacataagttccagataaattaaatagttaatgtcaaaaatcaagctgtggaggccaggcacggtggctaacgcctgtaatcccagaactttgggaggctgaggcgagtggatcacctgaggtcaagagttcgaggccagcctggccaacatggtgaaacccatctctactaaaaatacaaaaattagccgtgcatggtggtgggcgcctgtagtccctgctactcaggaggctgaggccagagaatcccttgaacctgggaggaggagattgcagtgagccgagatcgcgccactgtactccagcctgtgtgactccatctcaaaaaaaaaaaaccaagctgtgaaagactccacaggaaaagataagtgaatgtgtatctctggggaaaggtttcatttagagaaaaaaaaaaaaaaaaggtgaactttatttgaccatagcagaaaaaaaaatgtaaatctctgaatataaacacaaaaagattaaaactgctctgaacatggactgtaacagcagagaaggcattgtcaataaaacagcaaatagctactcttcctaataggtagagaactcatacgttggtaagaccaagactaagaaccaaattggaggaacactgtttgcaaaacaggagatacgaatggtatccacacattcttcacccggaaatccaagaaacgcaatttgtgttttaattactattttaatgaccttgttgtttctgtcactacacttttttttttttttttttagtggttgccctagggattacaattaacatcttaattttagcctcgttggaactgatgccaacttgctttcaatagcataaaaaagctttgcttctatgtggtttccattgcctctcgttcctctgtgctgttacacgtctgtatccatgatgagcccatccacgcagatttataatgaccgccttattcaattatcttttaagtcaaataggagggaaaaatgggttacaaacaaaaagtacatacacactgtctgccttttatatttactgatgtagttacctttacccatggttttatttcttcatgtggctttgacttcttgtctaacatctttcatttacagcagaaggactccctttagtatttattgtagggcagttctgctagtgatgaattctctcagtttttgttgacttcagagtctcttaatttctccttcattttttgaaagttctgctcaacgtagggttcttgactggcggtcgtttctctgaacactttgaacttgtcagcccatggcctccgtggtttctgctgagtagcttctgtcttgcctcttcccagattctctgtctttggcctttgaagtcccgatggtgtgtctaggtgtagatctctgagtttatcttactgggagtttgttgagcttcttggacgtatgcattgatgtttttcatcaaacttgggcggtttttcagccattacttcataaaatattttttctgcccctttctgtcttctactctgggacttccgttccacacattggtatgcttgacggtgcccaggggtctttgcagctctgttgactccttcctcgttgtgttttctttctgttcttcagactgcttagtctgactgccctgtcttcaaggtcactgattcttttttccaccagttctcatctgctgttgagcccctctagtgaacttctcattttagttgctctcttttcaactccagaatttctgtttcattcctttacgtttctctcgttaaggatattccctatctggtgagtcatgttctcacactttcctttagttcttcagacacggtttcgctgagctctttgaacacatttaaagtagctgacgtaaactctttgtctagtaagtccaatgtctgggcttctctagggacaatttttattaactactgtccccccatctgtgggccatactttcttgtttctttgcgtgtctcatacatttttgtttcagactggacattttaaacgcagctgctgtggtcatcagattccccatccccctcaggtctcgttgctattgctgattgttttgtgactttcttgagctaattccataaggtctgtgttcttcattgtgtgtggccaccaaagtctctgcttgactagcttagtggacagccaataattggtcagatatccttcacagatggcatccgtgagtctcccagcctttgctggggcgaggtggggagcaccgtcaacacttagctaggccaaggattcttgctgttcttacaaagattcagccattttgttaaatacatgctccccagatggctgcaaacccttggttagtttcaagagtcctaaaaatgtcaactctgaccatttttgccgatgttcctgttttcacagaggagaggatcttgagaggggcttactccaccatcttcactgacatcactccaagaaatgtattttgcaagaaatattttgaagcagaaagacaccatttattttgcccatgaattagaatacattagagaaaataagactatcccttgctggcaagaacacagtgacacagtagggtggaatataaattggcacatttgtggaaagcaacagtacatatcagtcaatgtttttgagattcacattgatgcgttaattttacaaatagaaatagggcctaaagaagtcacctcaaaaggcatgaacgctccatgaatgtatccatgcagggatcatagctgagcactggatgccccctgcacgtccgggggcaggaaacaggacagggcagagctgcgtcacagggcaggacagtctccgttagacggagaatcctccgtagagctgcttgcacatgtacattcatctttttgtcagatgttaattcaagttgcctttggttgtgggactgggaggatcttttctctttgttgatactttttcgtactttccaaatacttgactgatgagcacatgctgccttggttaccggaggataagtgagcgagcaaagtgaggccagtgctgtgtccatcctggtgcctcaagcacaagcccctattcctgccctgagcccagctgccggcatgtccggggagaaggcttctcccagctccggcattgacttctatctgctggaatcatccctgcccgtctgacctgagtcctccaagtcctccggcaccttgagctccagagagcagaattcagcctcttcctgtgcctccccagggtgggcatatgagccagccccatcccattcatcacccgtatgcctgtgtgcccatcccccttgcaggttccccaaggactctagcacctggactgtggaccaccagctcctgtggggggaggccctgctcatcaccccagtgctccaggccgggaaggccgaagtgactggctacttccccttgggcacatggtacgacctgcagacggtgagtctggggaccctaagccctggggagacgggagaccagagcagccctcccacctgccccctccacccagttggtgtgaccaggtggcggaaagaggaacgtatgtgttgagtcccggccatgtgccaggcccccacccggctgctccgcacccatcagcctctccgctcctcacaccatccccatttcccagatgagcagactgaggcctgcttgcagaacctggccaagtcccacggccatcacaggctgtgcctgtgctgagctggcatacccaggcctctcaggcactgtccccactcagtagccaggagggtccctacctacagtgagccctgagtctgcgcctgaagtcacagttcagcccgtctgtgccaggcctcctaggcctccacgtggagccccgggagatggagagcgtggttcctgaggacagcatgggggcctcggcacggcccagaatcctcaaagcaacatctccctccaggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcaggtacctgggccaggcggctatggtgggggtgtggacagcacactgcagagctgggggaggcacagggagatggtgggggagaggcccaggtggggcttctgaggggccgccccccgcagtgtaggttatcaaggagccagccaggccagtgaggtggggagggcacagccccacaaaggcgtggagcatggccggcaggagctcagtggtctgcatggtggaggttctgccgggcccggcctcgggcagccgtgggatagcacttgaggtggggaaggtcttgggtcatcaccacggggttccagcccctgcggccgcaggtgttcctgcagatcctagttactggcagcctggtgctgtaccagcctagcattcccgggccctggaggcctccacctccaccagggtggggatgatgacatcacgtgtccttccctttccagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaatgtgagtcctggggctgctcaggctggtgggcaggggccggctcggggttgagaaggggtgaggggacctgggcttgggggtcccacgatggctacctgccactaggacactctagcaggtggcctggggtcctagagtgagcagtggggccgtgcactctgccctttcgtgtacacagagggaggtcacctccctgatgccatcatgagtccctgttctcatgggtgttcctgccccagctgtctgctgacacctccacattctctgccttttcatctctctctgctcggcccagaacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggcaagagggcccagagtggcacagggatcgcgtcccccagccgtggtgcagggggcagaaggtgctgggcgtcctggtgaccgatgccaggaacagaggatgctgggacctcccaagggggtctttggggaggagtgggaagggtcaggccacacaggctgtgcctttcctcctcctgtgtctacacgtgggtgatggggccacaatgacgacctctgagccgtgttgaagcagcaccgcgtttctggcgtgcgttaaggtgacccgcactgagagccggggtccccctgcgcctgccggggaggaaccgggtgcgaagcatcccagggccagacggagctgccccctgagcgccgggcctcgctgctgctgggatctcggggccagatggagccgccttctgagcgctggggtctcactgctgctgggatctcgggctgctccatttgtgctctctcttttccaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggccctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgctgcccggctgcccagagggctggatgcctgccggtccccgagcaagcctgggaactcaggaaaattcacaggacttgggagattctaaatcttaagtgcaattatttttaataaaaggggcatttggaatca

SEQ ID NO: 30 - RNA, 3751 nt, Homo sapiens
  source 1..3751: mol_type = genomic RNA
cgaccccggagtctccgcgggcggccagggcgcgcgtgcgcggaggtgagccgggccggggctgcggggcttccctgagcgcgggccgggtcggtggggcggtcggctgcccgcgcggcctctcagttgggaaagctgaggttgtcgccggggccgcgggtggaggtcggggatgaggcagcaggtaggacagtgacctcggtgacgcgaaggaccccggccacctctaggttctcctcgtccgcccgttgttcagcgagggaggctctgcgcgtgccgcagctgacggggaaactgaggcacggagcgggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggccctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgctgcccggctgcccagagggctggatgcctgccggtccccgagcaagcctgggaactcaggaaaattcacaggacttgggagattctaaatcttaagtgcaattatttttaataaaaggggcatttggaatcag

SEQ ID NO: 31 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 32 - DNA, 3204 bp, synthetic construct
  source 1..3204: mol_type = other DNA
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgttaagtgcaattatttttaataaaaggggcatttggaatcaaagaattctgcagatctgtggcttctagctgcccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtttggagcaaggggcccaagttgg

SEQ ID NO: 33 - DNA, 2869 bp, synthetic construct
  source 1..2869: mol_type = other DNA
atctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 34 - DNA, 2863 bp, synthetic construct
  source 1..2863: mol_type = other DNA
atctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgt

SEQ ID NO: 35 - DNA, 2859 bp, synthetic construct
  source 1..2859: mol_type = other DNA
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 36 - DNA, 2856 bp, synthetic construct
  source 1..2856: mol_type = other DNA
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgt

SEQ ID NO: 37 - DNA, 339 bp, synthetic construct
  source 1..339: mol_type = other DNA
atgggagtgaggcacccgccctgctcccaccggctcctggccgtctgcgccctcgtgtccttggcaaccgctgcactcctggggcacatcctactccatgatttcctgctggttccccgagagctgagtggatcctccccagtcctggaggagacgcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgct

SEQ ID NO: 38 - DNA, 623 bp, synthetic construct
  source 1..623: mol_type = other DNA
ctagacagcagggcaacacccaccctggccaccttaccccacctgcctgggtgctgcagtgccagccgcggttgatgtctcagagctgctttgagagccccgtgagtgccgcccctcccgcctccctgctgagcccgctttcttctcccgcaggcctgtaggagctgtccaggccatctccaaccatgggagtgaggcacccgccctgctcccaccggctcctggccgtctgcgccctcgtgtccttggcaaccgctgcactcctggggcacatcctactacatgatttcctgctggttccccgagagctgagtggatcctccccagtcctggaggagacgcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatg

SEQ ID NO: 39 - DNA, 400 bp, synthetic construct
  source 1..400: mol_type = other DNA
ccgcaggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggatcctccccagtcctggaggagacgcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagccca

SEQ ID NO: 40 - DNA, 404 bp, synthetic construct
  source 1..404: mol_type = other DNA
gcctgactgtacctagcatactttaaagatggcctctctttcccacccattgtgatctactaaactatcagttcctcttggccctgatccccttgctgctggcccccatgctctgttccagcacacgggtgtgtatgacccaggtgagcccaggtctctgtgttctcatcccactcagggccctggtcaagacgcggggaacacgaccgtttgtgatctcccgctcaaccttctcgggccacggaagatacgctggtcactggactggggatgtgcggagctcttgggagcatcttgcatactctgtgccaggtgagtacaagctgcctgcagggctctggggtcttttctgggggtctttgggctgtgggctcaccactgcagatggtattgtgatagcttgg

SEQ ID NO: 41 - DNA, 4104 bp, Streptococcus pyogenes
  source 1..4104: mol_type = genomic DNA
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaggatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 42 - DNA, 3255 bp, Staphylococcus aureus
  source 1..3255: mol_type = genomic DNA
atggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaa

SEQ ID NO: 43 - DNA, 3156 bp, Staphylococcus aureus
  source 1..3156: mol_type = genomic DNA
aagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgaactacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaaggga

SEQ ID NO: 44 - protein, 1368 aa, Streptococcus pyogenes [sequence omitted]

SEQ ID NO: 45 - protein, 1085 aa, Staphylococcus aureus
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 46 - protein, 952 aa, Staphylococcus aureus
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 47-48 - skipped (2 entries)

SEQ ID NO: 49 - DNA, 5950 bp, synthetic construct
  source 1..5950: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctggaggggtggagtcgtgacgtgaattacgtcatagggttagggaggtcggccgctctagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcggtaccgagctcggatccactagtaacggccgccagtgtgctggaattcggctttccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgagttgttgggattccaggcatcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggacagatctgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgcagatctgggccactccctct

SEQ ID NO: 50 - DNA, 5793 bp, synthetic construct
  source 1..5793: mol_type = other DNA
cagatctttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcggccgcttaagctcgagtctagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcggtacccgccaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttaggaattctgcagatctgtggcttctagctgcccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtttggagcaaggggcccaagttgggaagacacaccatattggtcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatctgcgacgcgaggctggatggccttccccattatgattcttctcgcttccggcggcatcgggatgcccgcgttgcaggccatgctgtccaggcaggtagatgacgaccatcagggacagcttcagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 51 - DNA, 6233 bp, synthetic construct
  source 1..6233: mol_type = other DNA
cagatctttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcggccgcttaagctcgagttctgagtcctctaaggtccctcactcccaactcagccccatgtcctgtcaattcccactcagtgtctgatctccttctcctcacctttcccatctcccgtttgacccagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggctcgagtctagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcggtacccgccaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttaggaattctgcagatctgtggcttctagctgcccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtttggagcaaggggcccaagttgggaagacacaccatattggtcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatctgcgacgcgaggctggatggccttccccattatgattcttctcgcttccggcggcatcgggatgcccgcgttgcaggccatgctgtccaggcaggtagatgacgaccatcagggacagcttcagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 52 - DNA, 7246 bp, synthetic construct
  source 1..7246: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacctccggacgtacgtctagattcaagtgtctacctgccttgctggtcttcctggggacattctaagcgtgtttgatttgtaacattttagcagactgtgcaagtgctctgcactcccctgctggagcttttctcgcccttccttctggccctctccccagtctagacagcagggcaacacccaccctggccaccttaccgcacctgcctggctgctgcagtgccagccgcggacgcgtctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcaagcttggtaccgaattcaggtatcgccaagtctgtaatagctgcaaaggctcagctgtagcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgttaagtgcaattatttttaataaaaggggcatttggaatcaaagaattctgcagatctgtggcttctagctgcccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtttggagcaaggggcccaagttggactagtttgatgtctcagagctgctttgagagccccgtcagtgccgcccctcccgcctccctgctgagcccgctttcttctcccgcaggcctgtaggagctgtccaggccatctccaaccttgggagtgaggcacccgccctgctcccaccggctcctggccgtctgcgccctcgtgtccttggcaaccgctgcactcctggggcacatcctactccagcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 53 - DNA, 7285 bp, synthetic construct
  source 1..7285: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacctccggacgtacgtctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacccagagctgctttgagagccccgttttagtactctggaaacagaatctactaaaacaaggcaaaatgccgtgtttatctcgtcaacttgttggcgagattttttctcgaggtcggaggatccacaaaggctagcctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 54 - DNA, 6505 bp, synthetic construct
  source 1..6505: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccccaccgcggcaaaaaagcaccgactcggtgccactttttcaagttgataacggactagccttattttaacttgctatttctagctctaaaacgatgggaaagaggacatagacggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcggtaccttaattaaccaaactgccgcggcatggtggcatatagctatgggaatacaggcaggtagacctccagtgctcattggtcaaatttactccggagctccagttcaatcagagactttatcttaataagttttgaggaatgactgcactggcttccacaaacacagccacatgcatgtacgcagatgaacacgtgcgcacacacacaaacacacacacagactgagacagacacagggagagacagtgcgagcctggcacatagtagctgatcaataaacgttgattgtgcatattattaagatcctggagagaaccaaagtttatagattctaaaactagaatagtgagtttatgtaattcaccagttggaaggtcctggctatccgtatcaattttcaccgcttaaccaaaaggttagaaatcactcccccccccttctttcccacatctgtttctatagcacccatagaaatagtagtaaaagggtttttttaacatctgtgctcaggagctgggatatagcctcatgggggagtccttgctctcatgctccaagccccaggttcaatccctagcatgctaaactacacgtgggaacacacacctgtaatctcagcactcagaagttgagcaattcaaggtcgttgtctcttaatttgtaagtttgaggccagcctgggctacacaagaccctaactctaaataataaagtcaaataaaccagagtaaaggctacactcagacttcccattaaactcagctgtctttccatccctcaggtctgacaaaatagccattcctcattgccagccacaggtgggattctgtagagactctcaagccaatggggacttacctgtagcctggtgaacagtatggggtacccctgctggaggacagccatttctctacccatcacccctcgggatggttcttaccacttgaagacgaggttgaaccagtctccgaccactgccacccagaggagattgatgcccacagtctctttacgcgtgatctagactcagatctgcagaattccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgaattcgactttaaacaccgaagactccacatctcttacaacgacttcttgtgcggctggcccaggacctgggcgaggcagtaggggatgacactgacggatgccaggggcactaccgcactcttgcagaaggacaagacgtagaagaccagctcgacttgggatgggggtttcaaggagtcaaagacgtgcaggaggacgagggaggctacaatagagctgaggcggaatgggagccacttgctgagtttccccttgcagctctccctgtacatgctggagttgagagccagccccaggccaaagagcgtgcccaggttcttgaggaggctggcaaagggtgtggtgtcaatgtggacccattctggctgctcgcaccacctctgggctttctccagagtccacaggaggtctacacccagtcccttgagcagcagataaaatccgatggcgaagctgaacaggaagaaggtaatgagaaaatatttcttgaggctggcattatagatgctgtggatgtggctgaaagtttctgtaacagcaatgcctgacaggactccagcaacaacttgatgaggaaaatgagcagcaaggtagattcgtgacagacagacattcagctgcacagcccagaatcccaaccacaaaatgacattcaagcaccgaaatctgtaggtcggctttatctttccctgaaagatggaaagagtagatgtgaccatcacgtagtatacacctgctgtgcccatggcatggccagaggggctccctggtccagtctcacaggttacagggaactgctttatcaggggcacggaagtgttgctgtagtagtcagtatccaaaacccaccagtatggacgctgtccaaagagaatccacttaaagacgaggttgagccagtctccaatcacagctacccaaaggagtttaatgcccacagcttcctgaagatggaaccagatggggaagaggacgtagaaggcattcctgaggtctgcgatcacggacaccaagatgaaccagtcctgggagtcttggtaattcacctggaggtaatgtgttgactggatcccaaagtcatggagaacattcattccttcctccatcctcatttccttggcacctcgagcttcctcccttggtacctcaggaagctgccagcagagcccgtgcagtgagtccaagcccggttggcttgatccctctgctatcagtctgtgccttgcccctgttttatatgccctggattcagtctgtaggtcaacccagccctgatctttggactcaaaaaaccacttttgtctaaaatctattgatcaaaggtgcatcatcagtaggttgatgcaaacatgttcagggtgatttacgtaaaatagcaaaacaggcacacaaaaacagcctgatcgccattggcagagccaattattaacttgggcacactgcttgacagtgctagaggctgccctccttcctggtccttgaagccaggccagaggcccagggcagagtctgtttcaaccatgactcttgcaagggcatctgttttttgaagtctgcaattatatctatttgaagacaaagggaagtttgctactgccggaagtgaccttttaagttattaatttaaaatatacaaaatacacagcagtagggctttgttgtcctggaaatgctcactctcttggactttcaccaagttcacgtggatcacagttggagataggtactttaagccaaaagaagttaaaagtataacttaaaggagaccataaagacagagccctagtgtaatagaaccatttgctctgacgaagaggaagggatacctctgacctcctgtttctctccacacacagaataaaagtgatttgggaacaaactgagtggttaactgacaggcgccttacgccaaccaaaacacgccatttgtgttttcagggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 55 - DNA, 7465 bp, synthetic construct
  source 1..7465: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccaattcgagctcggtaccctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagctctagagcgctgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaggatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagtgagtcgacaataaaatatctttattttcattacatctgtgtgttggttttttgtgtgtgcatgctggggacggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 56 - RNA, 20 nt, synthetic construct
  source 1..20: mol_type = other RNA
gatgggaaagaggacataga

SEQ ID NO: 57 - RNA, 21 nt, synthetic construct
  source 1..21: mol_type = other RNA
tgatggacagacattgcgaga

SEQ ID NO: 58 - RNA, 21 nt, synthetic construct
  source 1..21: mol_type = other RNA
gcatgagtagcccgtggcttc

SEQ ID NO: 59 - RNA, 21 nt, synthetic construct
  source 1..21: mol_type = other RNA
tcagagagaggggttggaagc

SEQ ID NO: 60 - RNA, 21 nt, synthetic construct
  source 1..21: mol_type = other RNA
agatgatccaaagtcagagag

SEQ ID NO: 61 - RNA, 21 nt, synthetic construct
  source 1..21: mol_type = other RNA
agatgatccaaagtcagagag

SEQ ID NO: 62 - RNA, 20 nt, synthetic construct
  source 1..20: mol_type = other RNA
ttgctgatctcctctctata

SEQ ID NO: 63 - RNA, 20 nt, synthetic construct
  source 1..20: mol_type = other RNA
gtggtaagaaccatatagag

SEQ ID NO: 64 - RNA, 20 nt, synthetic construct
  source 1..20: mol_type = other RNA
gaggagatcagcaagaaaag

SEQ ID NO: 65 - RNA, 20 nt, synthetic construct
  source 1..20: mol_type = other RNA
agatcagcaagaaaagaggc

SEQ ID NO: 66 - RNA, 20 nt, synthetic construct
  source 1..20: mol_type = other RNA
ctggggaaagcaacttctga

SEQ ID NO: 67 - RNA, 21 nt, synthetic construct
  source 1..21: mol_type = other RNA
aggagccagtatcagtgtggg

SEQ ID NO: 68 - RNA, 22 nt, synthetic construct
  source 1..22: mol_type = other RNA
taggagccagtatcagtgtggg

SEQ ID NO: 69 - RNA, 21 nt, synthetic construct
  source 1..21: mol_type = other RNA
cagagctgctttgagagcccc

SEQ ID NO: 70 - RNA, 22 nt, synthetic construct
  source 1..22: mol_type = other RNA
tcagagctgctttgagagcccc

SEQ ID NO: 71 - RNA, 21 nt, synthetic construct
  source 1..21: mol_type = other RNA
ggctccctgctggtgagctgg

SEQ ID NO: 72 - RNA, 21 nt, synthetic construct
  source 1..21: mol_type = other RNA
cagagctgctttgagagcccc

SEQ ID NO: 73 - RNA, 21 nt, synthetic construct
  source 1..21: mol_type = other RNA
gatcacaaagggtcgtgttcc

SEQ ID NO: 74 - RNA, 21 nt, synthetic construct
  source 1..21: mol_type = other RNA
aaggttgagcgggagatcaca

SEQ ID NO: 75 - RNA, 27 nt, synthetic construct
  source 1..27: mol_type = other RNA
gatcacaaagggtcgtgttccccgagt

SEQ ID NO: 76 - RNA, 27 nt, synthetic construct
  source 1..27: mol_type = other RNA
aaggttgagcgggagatcacaaagggt

SEQ ID NO: 77 - RNA, 21 nt, synthetic construct
  source 1..21: mol_type = other RNA
ttccacctcctggcagtgtgt

SEQ ID NO: 78 - RNA, 27 nt, synthetic construct
  source 1..27: mol_type = other RNA
ttccacctcctggcagtgtgtgtgggt

SEQ ID NO: 79-86 - skipped (8 entries)

SEQ ID NO: 87 - DNA, 764 bp, synthetic construct
  source 1..764: mol_type = other DNA
cctcgagcttcctcccttggtacctcaggaagctgccagcagagcccgtgcagtgagtccaagcccggttggcttgatccctctgctatcagtctgtgccttgcccctgttttatatgccctggattcagtctgtaggtcaacccagccctgatctttggactcaaaaaaccacttttgtctaaaatctattgatcaaaggtgcatcatcagtaggttgatgcaaacatgttcagggtgatttacgtaaaatagcaaaacaggcacacaaaaacagcctgatcgccattggcagagccaattattaacttgggcacactgcttgacagtgctagaggctgccctccttcctggtccttgaagccaggccagaggcccagggcagagtctgtttcaaccatgactcttgcaagggcatctgttttttgaagtctgcaattatatctatttgaagacaaagggaagtttgctactgccggaagtgaccttttaagttattaatttaaaatatacaaaatacacagcagtagggctttgttgtcctggaaatgctcactctcttggactttcaccaagttcacgtggatcacagttggagataggtactttaagccaaaagaagttaaaagtataacttaaaggagaccataaagacagagccctagtgtaatagaaccatttgctctgacgaagaggaagggatacctctgacctcctgtttctctccacacacagaataaaagtgatttgggaacaaactgagtg

SEQ ID NO: 88 - DNA, 1029 bp, synthetic construct
  source 1..1029: mol_type = other DNA
ggtaccttaattaaccaaactgccgcggcatggtggcatatagctatgggaatacaggcaggtagacctccagtgctcattggtcaaatttactccggagctccagttcaatcagagactttatcttaataagttttgaggaatgactgcactggcttccacaaacacagccacatgcatgtacgcagatgaacacgtgcgcacacacacaaacacacacacagactgagacagacacagggagagacagtgcgagcctggcacatagtagctgatcaataaacgttgattgtgcatattattaagatcctggagagaaccaaagtttatagattctaaaactagaatagtgagtttatgtaattcaccagttggaaggtcctggctatccgtatcaattttcaccgcttaaccaaaaggttagaaatcactcccccccccttctttcccacatctgtttctatagcacccatagaaatagtagtaaaagggtttttttaacatctgtgctcaggagctgggatatagcctcatgggggagtccttgctctcatgctccaagccccaggttcaatccctagcatgctaaactacacgtgggaacacacacctgtaatctcagcactcagaagttgagcaattcaaggtcgttgtctcttaatttgtaagtttgaggccagcctgggctacacaagaccctaactctaaataataaagtcaaataaaccagagtaaaggctacactcagacttcccattaaactcagctgtctttccatccctcaggtctgacaaaatagccattcctcattgccagccacaggtgggattctgtagagactctcaagccaatggggacttacctgtagcctggtgaacagtatggggtacccctgctggaggacagccatttctctacccatcacccctcgggatggttcttaccacttgaagacgaggttgaaccagtctccgaccactgccacccagaggagattgatgcccacagtctctttacgcgtgatctaga

SEQ ID NO: 89 - DNA, 212 bp, synthetic construct
  source 1..212: mol_type = other DNA
cttttctcttacacagaaggccctgcgataaagtcctggctccctcacatctgtctgtacctgtgttgtgggcccatgtctgcagagacaccctctgcctctgaggagtctcccattgtctcctgtacctggcttctgtcttcccttccgttgctctggggaagatgctctggctgcctccttccacctcctggcagtgtgtgtcggtgttg

SEQ ID NO: 90 - DNA, 212 bp, synthetic construct
  source 1..212: mol_type = other DNA
tgtgagaaaccgcaccgacactgatactggctcctcctctcctcaggccttcagaagtattcatgctgccccgaaccaataggctttcaccatgaatatacggaagcccctctgttcgaactccgtggttggggcctgcacccttatctctctgactacagcggtcatcctgggtcatctcatgcttcgggagttaatgctgcttccccaag

SEQ ID NO: 91 - DNA, 1537 bp, synthetic construct
  source 1..1537: mol_type = other DNA
gagctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcctgaggtgccaaggaaatgaggatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtg

SEQ ID NO: 92 - DNA, 2000 bp, synthetic construct
  source 1..2000: mol_type = other DNA
gaatgctgtcaacttttgccacagattcattagctctgagtttcttttttctgtccccagctaccccttacatgtcaatatggacttaatgatgggaaattcaggcaagtttttaaacattttattccccctggctcttatcctcaaaaaatgcatgaatttggaggcagtggctcatgcctgtaatcccaatgctttgctaggttgaggcgggaggatcacttgaagccaggaatttgagaccagcctgggccgcatagtgagaccccgtttctacaaaaataaataaataaataataaataatagtgatatgaagcatgattaaatagccctattttttaaaatgcatgagttcgttacctgattcattccctggttcctttcacagtcctccgtgacccaagtgttagggttttggtctctctactatttgtaggctgatatatagtatacacacacacacacacacacatatacacacacacagtgtatcttgagctttcttttgtatatctacacacatatgtataagaaagctcaagatatagaagccctttttcaaaaataactgaaagtttcaaactctttaagtctccagttaccattttgctggtattcttatttggaaccatacattcatcatattgttgcacagtaagactatacattcattattttgcttaaacgtatgagttaaaacacttggccaggcatggtggttcacacctgtaatcccagagctttgggaagccaagactggcagatctcttgagctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcat

SEQ ID NO: 93 - DNA, 1306 bp, synthetic construct
  source 1..1306: mol_type = other DNA
agctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcctgaggtgccaaggaaatgagg

SEQ ID NO: 94 - DNA, 1042 bp, synthetic construct
  source 1..1042: mol_type = other DNA
gtaagaaccatatagagaggagatcagcaagaaaagaggctggcataggctctcgcaatgtctgtccatcagaagttgctttccccaggctattcaggaagccacgggctactcatgcttccaacccctctctctgactttggatcatctacataaagggggaagacagaaaaaatcctaccagtgagttgaaaatacaggaaagcctatttcatatgggttaaagggtaggacagttgaatttcgtgaaaagtctgagttatataggctttgagcaaagagttttattagtatgaagcagaagaggtaacataaagaaagatgtatggggccaggcatggtggctcacacctgtaatcccagcactttgggaggccgaggtgggcgaatcactcctgggtgaactcaggagttcaagaccagcctgggcaacatggcgaaactccatctctacaaaaacattacgaaaattagctgggcgtgttggtgctgtagtcccagctactcaggaggctgaggtgagaggcggaggaggttgcagtgagtcaagatcatgccactgcactccagcctgggcaacagagtaagaccctgtctcaaaaaaaaaaaaaagatagatgatgtatgctgtatgaaaaaaggaaacacacagatgattcaacagcctgttttgtggggtaatgaaaagtcaccctgggaactgggctccagccctcgttctgccacccaccaactacatgtccttggcaagtcatatcaattatctgagtttctgttttataatctacaaataggttatctctggcagcttaataataatcagggttaacatttattaaacagtgtgtgccagtccatgtgctatgtgcttttctgtgaggtagttactgctatttacagaaacagtagatgcagagaccaaggtgctgagttaaatgattaggccaacaaggttagtacatgccgagccaggatggaagcccaggtaggcaggctggcttccgcggcaatgctcttatgaactatgttacgtccagtgctg

SEQ ID NO: 95 - DNA, 1007 bp, synthetic construct
  source 1..1007: mol_type = other DNA
gtaagaaccatatagagaggagatcagcaagaaaagaggctggcattcgctctcgcaatgtctgtccatcagaagttgctttccccaggctattcaggaagccacgggctactcatgcttccaacccctctctctgactttggatcatctacataaagggggaagacagaaaaaatcctaccagtgagttgaaaatacaggaaagcctatttcatatgggttaaagggtaggacagttgaatttcgtgaaaagtctgagttatataggctttgagcaaagagttttattagtatgaagcagaagaggtaacataaagaaagatgtatggggccaggcatggtggctcacacctgtaatcccagcactttgggaggccgaggtgggcgaatcactcctgggtgaactcaggagttcaagaccagcctgggcaacatggcgaaactccatctctacaaaaacattacgaaaattagctgggcgtgttggtgctgtagtcccagctactcaggaggctgaggtgagaggcggaggaggttgcagtgagtcaagatcatgccactgcactccagcctgggcaacagagtaagaccctgtctcaaaaaaaaaaaaaagatagatgatgtatgctgtatgaaaaaaggaaacacacagatgattcaacagcctgttttgtggggtaatgaaaagtcaccctgggaactgggctccagccctcgttctgccacccaccaactacatgtccttggcaagtcatatcaattatctgagtttctgttttataatctacaaataggttatctctggcagcttaataataatcagggttaacatttattaaacagtgtgtgccagtccatgtgctatgtgcttttctgtgaggtagttactgctatttacagaaacagtagatgcagagaccaaggtgctgagttaaatgattaggccaacaaggttagtacatgccgagccaggatggaagcccaggtaggcaggctggcttccgcgg

SEQ ID NO: 96 - DNA, 1001 bp, synthetic construct
  source 1..1001: mol_type = other DNA
ggtaagaaccatatagagaggagatcagcaagaaaagaggctggcattcgctctcgcaatgtctgtccatcagaagttgctttccccaggctattcaggaagccacgggctactcatgcttccaacccctctctctgactttggatcatctacataaagggggaagacagaaaaaatcctaccagtgagttgaaaatacaggaaagcctatttcatatgggttaaagggtaggacagttgaatttcgtgaaaagtctgagttatataggctttgagcaaagagttttattagtatgaagcagaagaggtaacataaagaaagatgtatggggccaggcatggtggctcacacctgtaatcccagcactttgggaggccgaggtgggcgaatcactcctgggtgaactcaggagttcaagaccagcctgggcaacatggcgaaactccatctctacaaaaacattacgaaaattagctgggcgtgttggtgctgtagtcccagctactcaggaggctgaggtgagaggcggaggaggttgcagtgagtcaagatcatgccactgcactccagcctgggcaacagagtaagaccctgtctcaaaaaaaaaaaaaagatagatgatgtatgctgtatgaaaaaaggaaacacacagatgattcaacagcctgttttgtggggtaatgaaaagtcaccctgggaactgggctccagccctcgttctgccacccaccaactacatgtccttggcaagtcatatcaattatctgagtttctgttttataatctacaaataggttatctctggcagcttaataataatcagggttaacatttattaaacagtgtgtgccagtccatgtgctatgtgcttttctgtgaggtagttactgctatttacagaaacagtagatgcagagaccaaggtgctgagttaaatgattaggccaacaaggttagtacatgccgagccaggatggaagcccaggtaggcaggctggct

SEQ ID NO: 97 - DNA, 212 bp, synthetic construct
  source 1..212: mol_type = other DNA
ttcaagtgtctacctgccttgctggtcttcctggggacattctaagcgtgtttgatttgtaacattttagcagactgtgcaagtgctctgcactcccctgctggagcttttctcgcccttccttctggccctctccccagtctagacagcagggcaacacccaccctggccaccttaccgcacctgcctggctgctgcagtgccagccgcgg

SEQ ID NO: 98 - DNA, 210 bp, synthetic construct
  source 1..210: mol_type = other DNA
cctggggacattctaagcgtgtttgatttgtaacattttagcagactgtgcaagtgctctgcactcccctgctggagcttttctcgcccttccttctggccctctccccagtctagacagcagggcaacacccaccctggccaccttaccccacctgcctgggtgctgcagtgccagccgcggttgatgtctcagagctgctttgagagc

SEQ ID NO: 99 - DNA, 212 bp, synthetic construct
  source 1..212: mol_type = other DNA
ttgatgtctcagagctgctttgagagccccgtcagtgccgcccctcccgcctccctgctgagcccgctttcttctcccgcaggcctgtaggagctgtccaggccatctccaaccttgggagtgaggcacccgccctgctcccaccggctcctggccgtctgcgccctcgtgtccttggcaaccgctgcactcctggggcacatcctactcca

SEQ ID NO: 100 - DNA, 212 bp, synthetic construct; identical to SEQ ID NO: 99
  source 1..212: mol_type = other DNA

SEQ ID NO: 101 - DNA, 210 bp, synthetic construct
  source 1..210: mol_type = other DNA
cccgtcagtgccgcccctcccgcctccctgctgagcccgctttcttctcccgcaggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagag

SEQ ID NO: 102 - DNA, 212 bp, synthetic construct; identical to SEQ ID NO: 97
  source 1..212: mol_type = other DNA

SEQ ID NO: 103 - DNA, 76 bp, synthetic construct
  source 1..76: mol_type = other DNA
gcaccgactcggtgccactttttcaagttgataacggactagccttattttaacttgctatttctagctctaaaac

SEQ ID NO: 104 - DNA, 76 bp, synthetic construct
  source 1..76: mol_type = other DNA
gttttagtactctggaaacagaatctactaaaacaaggcaaaatgccgtgtttatctcgtcaacttgttggcgaga

SEQ ID NO: 105 - DNA, 49 bp, synthetic construct
  source 1..49: mol_type = other DNA
aataaaatatctttattttcattacatctgtgtgttggttttttgtgtg

SEQ ID NO: 106 - DNA, 204 bp, synthetic construct
  source 1..204: mol_type = other DNA
ctgtggcttctagctgcccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtttggagcaaggggcccaagttgggaagacacacc

SEQ ID NO: 107 - DNA, 166 bp, synthetic construct
  source 1..166: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggc

SEQ ID NO: 108 - DNA, 185 bp, synthetic construct
  source 1..185: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctg

SEQ ID NO: 109 - DNA, 207 bp, synthetic construct
  source 1..207: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctggagttgttgggattccaggcat

SEQ ID NO: 110 - DNA, 225 bp, synthetic construct
  source 1..225: mol_type = other DNA
ccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacag

SEQ ID NO: 111 - DNA, 27 bp, synthetic construct
  source 1..27: mol_type = other DNA
tatccctatgacgtcccggactatgca

SEQ ID NO: 112 - DNA, 27 bp, synthetic construct
  source 1..27: mol_type = other DNA
tacccatacgatgttcctgactatgcg

SEQ ID NO: 113 - DNA, 146 bp, synthetic construct
  source 1..146: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaa

SEQ ID NO: 114 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 115 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 116 - DNA, 200 bp, synthetic construct
  source 1..200: mol_type = other DNA
cagatctttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtc

SEQ ID NO: 117 - DNA, 22 bp, synthetic construct
  source 1..22: mol_type = other DNA
aggaacccctagtgatggagtt

SEQ ID NO: 118 - DNA, 28 bp, synthetic construct
  source 1..28: mol_type = other DNA
ctgcgcgctcgctcgctcactgaggccg

SEQ ID NO: 119 - DNA, 28 bp, synthetic construct
  source 1..28: mol_type = other DNA
cggcctcagtgagcgagcgagcgcgcag

SEQ ID NO: 120 - DNA, 241 bp, synthetic construct
  source 1..241: mol_type = other DNA
gtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctc

SEQ ID NO: 121 - DNA, 240 bp, synthetic construct
  source 1..240: mol_type = other DNA
tttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 122 - DNA, 249 bp, synthetic construct
  source 1..249: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 123 - DNA, 22 bp, synthetic construct
  source 1..22: mol_type = other DNA
gcagttccctgtaacctgtgag

SEQ ID NO: 124 - DNA, 21 bp, synthetic construct
  source 1..21: mol_type = other DNA
ggtcggctttatctttccctg

SEQ ID NO: 125 - DNA, 20 bp, synthetic construct
  source 1..20: mol_type = other DNA
agtacagcatcggcctggac

SEQ ID NO: 126 - DNA, 20 bp, synthetic construct
  source 1..20: mol_type = other DNA
gggctccgatcaggttcttc

SEQ ID NO: 127 - DNA, 20 bp, synthetic construct
  source 1..20: mol_type = other DNA
ggctgtattcccctccatcg

SEQ ID NO: 128 - DNA, 22 bp, synthetic construct
  source 1..22: mol_type = other DNA
ccagttggtaacaatgccatgt

SEQ ID NO: 129 - DNA, 20 bp, synthetic construct
  source 1..20: mol_type = other DNA
agagggaaatcgtgcgtgac

SEQ ID NO: 130 - DNA, 21 bp, synthetic construct
  source 1..21: mol_type = other DNA
caatagtgatgacctggccgt

SEQ ID NO: 131 - DNA, 22 bp, synthetic construct
  source 1..22: mol_type = other DNA
tgacctacagactgaatccagg

SEQ ID NO: 132 - DNA, 22 bp, synthetic construct
  source 1..22: mol_type = other DNA
taacatctgtgctcaggagctg

SEQ ID NO: 133 - RNA, 72 nt, synthetic construct
  source 1..72: mol_type = other RNA
gggttccatggtgtaatggttagcactctggactctgaatccagcgatccgagttcaaatctcggtggaacc

SEQ ID NO: 134 - RNA, 82 nt, synthetic construct
  source 1..82: mol_type = other RNA
gttttagtactctggaaacagaatctactaaaacaaggcaaaatgccgtgtttatctcgtcaacttgttggcgagatttttt

SEQ ID NO: 135 - DNA, 141 bp, synthetic construct; identical to SEQ ID NO: 114
  source 1..141: mol_type = other DNA

SEQ ID NO: 136 - DNA, 21 bp, synthetic construct
  source 1..21: mol_type = other DNA
cagccgcacaagaagtcgttg

SEQ ID NO: 137 - DNA, 20 bp, synthetic construct
  source 1..20: mol_type = other DNA
ccactcccactgtcctttcc

SEQ ID NO: 138 - DNA, 23 bp, synthetic construct
  source 1..23: mol_type = other DNA
ggctcagtagatcaagtgcctgc

SEQ ID NO: 139 - DNA, 20 bp, synthetic construct
  source 1..20: mol_type = other DNA
tctgggaatcagggactggg

SEQ ID NO: 140 - DNA, 19 bp, synthetic construct
  source 1..19: mol_type = other DNA
cagagcagtgcccacacag

SEQ ID NO: 141 - DNA, 22 bp, synthetic construct
  source 1..22: mol_type = other DNA
gtagcccatttcagaggagctc

SEQ ID NO: 142 - DNA, 21 bp, synthetic construct
  source 1..21: mol_type = other DNA
caatagtgatgacctggccgt

SEQ ID NO: 143 - DNA, 48 bp, synthetic construct
  source 1..48: mol_type = other DNA
aagcgacctgccgccacaaagaaggctggacaggctaagaagaagaaa

SEQ ID NO: 144 - DNA, 21 bp, synthetic construct
  source 1..21: mol_type = other DNA
cccaagaagaagcggaaagtt

SEQ ID NO: 145 - DNA, 51 bp, synthetic construct
  source 1..51: mol_type = other DNA
gttaactgacaggcgccttacgccaaccaaaacacgccatttgtgttttca

SEQ ID NO: 146 - DNA, 22 bp, synthetic construct
  source 1..22: mol_type = other DNA
ccttgcactgccaagaagcatg

SEQ ID NO: 147 - DNA, 17 bp, synthetic construct
  source 1..17: mol_type = other DNA
aattcgagctcggtacc

SEQ ID NO: 148 - DNA, 10 bp, synthetic construct
  source 1..10: mol_type = other DNA
gccaccatgg

SEQ ID NO: 149 - DNA, 573 bp, synthetic construct
  source 1..573: mol_type = other DNA
gtgcactgatcacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggcaattgaaaccggtctctaaggtaaatataaaatttttaagtgtataatgtgttaaactactgattctaattgtttctctcttttagattccaacctttggaactgaattctcgaggtgcac

SEQ ID NO: 150 - DNA, 250 bp, synthetic construct
  source 1..250: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccg

SEQ ID NO: 151 - DNA, 556 bp, synthetic construct
  source 1..556: mol_type = other DNA
gtgcactgatcacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggcaattgaaaccggtctctaaggtaaatataaaatttttaagtgtataatgtgttaaactactgattctaattgtttctctcttttagattccaacctttggaact

SEQ ID NO: 152 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgtttcggcaccaaaatcaacgggactttccaaaatgtcgtaacaactccgccccattgacgcaaatgggcggtaggcgtgtacgggtgggaggtctatataagcagagc

SEQ ID NO: 153 - DNA, 226 bp, synthetic construct
  source 1..226: mol_type = other DNA
tcgaggtgagccccacgttctgcttcactctccccatctcccccccctccccacccccaattttgtatttatttattttttaattattttgtgcagcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 154 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgtttcggcaccaaaatcaacgggactttccaaaatgtcgtaacaactccgccccattgacgcaaatgggcggtaggcgtgtacggtgggaggtctatataagcagagct

SEQ ID NO: 155 - DNA, 304 bp, synthetic construct
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 156 - DNA, 535 bp, synthetic construct
  source 1..535: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 157 - DNA, 110 bp, synthetic construct
  source 1..110: mol_type = other DNA
ttgacgtcaatgacggtaaatggcccgcctggcattatgcccagtacatgaccttatgggactttcctacttggcagtacatctacgtattagtcatcgctattaccatg

SEQ ID NO: 158 - DNA, 109 bp, synthetic construct
  source 1..109: mol_type = other DNA
tgacgtcaatgacggtaaatggcccgcctggcattatgcccagtacatgaccttatgggactttcctacttggcagtacatctacgtattagtcatcgctattaccatg

SEQ ID NO: 159 - DNA, 315 bp, synthetic construct
  source 1..315: mol_type = other DNA
ttgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgtttcggcaccaaaatcaacgggactttccaaaatgtcgtaacaactccgccccattgacgcaaatgggcggtaggcgtgtacgggtgggaggtctatataagcagagct

SEQ ID NO: 160 - DNA, 313 bp, synthetic construct
  source 1..313: mol_type = other DNA
tgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgtttcggcaccaaaatcaacgggactttccaaaatgtcgtaacaactccgccccattgacgcaaatgggcggtaggcgtgtacggtgggaggtctatataagcagagct